(19) United States
(12) Patent Application Publication
(10) Pub. No.: US 2023/0183378 A1
(43) Pub. Date: Jun. 15, 2023

(54) MULTIMERS, TETRAMERS & OCTAMERS

(71) Applicant: Quadrucept Bio Limited, London (GB)

(72) Inventors: Hanif ALI, London (GB); Terence Rabbitts, London (GB); Christian Grøndahl, London (GB); Jasper Clube, London (GB)

(21) Appl. No.: 17/817,912

(22) Filed: Aug. 5, 2022

Related U.S. Application Data
(62) Division of application No. 16/333,583, filed on Mar. 14, 2019, now Pat. No. 11,453,726.

(30) Foreign Application Priority Data
Aug. 20, 2017 (GB) 1713342.2
Aug. 21, 2017 (GB) 1713351.3

(51) Int. Cl. C07K 16/46; C07K 14/55; C07K 14/725; C07K 14/47; C07K 16/28; C07K 16/24; C07K 16/22

(52) U.S. Cl. CPC C07K 16/46; C07K 14/55; C07K 14/7051; C07K 14/4702; C07K 16/2863; C07K 16/2896; C07K 16/241; C07K 16/22; C07K 2319/02; C07K 2319/21; C07K 2319/735; C07K 2317/55; C07K 2319/30; C07K 2319/00; C07K 2319/43; C07K 2319/35; C07K 2317/622; C07K 2319/31; C07K 2317/569

(57) ABSTRACT

The invention relates to multimers such as tetramers of polypeptides and tetramers and octamers of effector domains, such as antigen binding sites (eg, antibody or TCR binding sites that specifically bind to antigen or pMHC, or variable domains thereof) or peptides such as incretin, insulin or hormone peptides.

### CROSS REFERENCE TO RELATED APPLICATIONS

This application is a divisional of co-pending U.S. patent application Ser. No. 16/333,583, internationally filed on Sep. 18, 2017, which is a national stage application under 35 U.S.C. § 371 of International Application No. PCT/EP2017/073527, filed on Sep. 18, 2017, which claims priority benefit to United Kingdom Application No. 1615768.7, filed Sep. 15, 2016, United Kingdom Application No. 1713342.2, filed Aug. 20, 2017, and United Kingdom Application No. 1713351.3, filed Aug. 21, 2017, the contents of each of which are incorporated herein by reference in their entireties.

### REFERENCE TO AN ELECTRONIC SEQUENCE LISTING

The contents of the electronic sequence listing (165132000110SEQLIST.xml; Size: 235,522 bytes; and Date of Creation: Aug. 4, 2022) is herein incorporated by reference in its entirety.

### TECHNICAL FIELD

The invention relates to multimers such as tetramers of polypeptides and tetramers and octamers of effector domains, such as antigen binding sites (eg, antibody or TCR binding sites that specifically bind to antigen or pMHC, or variable domains thereof) or peptides such as incretin, insulin or hormone peptides.

### BACKGROUND

Multimers of effector domains have recognized utility in medical and non-medical applications for combining and multiplying the activity and presence of effector domains, eg, to provide for higher avidity of antigen binding (for effector domains that are antibody or TCR binding domains, for example) or for enhancing biological or binding activity, such as for providing bi- or multi-specific targeting or interaction with target ligands in vivo or in vitro.

Multimerisation domains which cause self-assembly of protein monomers into multimers are known in the art. Examples include domains found in transcription factors such as p53, p63 and p73, as well as domains found in ion channels such as TRP cation channels. The transcription factor p53 can be divided into different functional domains: an N-terminal transactivation domain, a proline-rich domain, a DNA-binding domain, a tetramerization domain and a C-terminal regulatory region. The tetramerization domain of human p53 extends from residues 325 to 356, and has a 4-helical bundle fold (Jeffrey et al., Science (New York, N.Y.) 1995, 267(5203):1498-1502). The TRPM tetramerization domain is a short anti-parallel coiled-coil tetramerization domain of the transient receptor potential cation channel subfamily M member proteins 1-8. It is held together by extensive core packing and interstrand polar interactions (Fujiwara et al., Journal of Molecular Biology 2008, 383(4):854-870). Transient receptor potential (TRP) channels comprise a large family of tetrameric cation-selective ion channels that respond to diverse forms of sensory input. Another example is the potassium channel BTB domain. This domain can be found at the N terminus of voltage-gated potassium channel proteins, where represents a cytoplasmic tetramerization domain (T1) involved in assembly of alpha-subunits into functional tetrameric channels (Bixby et al., Nature Structural Biology 1999, 6(1):38-43). This domain can also be found in proteins that are not potassium channels, like KCTD1 (potassium channel tetramerization domain-containing protein 1; Ding et al., DNA and Cell Biology 2008, 27(5):257-265).

Multimeric antibody fragments have been produced using a variety of multimerisation techniques, including biotin, dHLX, ZIP and BAD domains, as well as p53 (Thie et al., Nature Boitech., 2009:26, 314-321). Biotin, which is efficient in production, is a bacterial protein which induces immune reactions in humans.

Human p53 (UniProtKB-P04637 (P53_HUMAN)) acts as a tumor suppressor in many tumor types, inducing growth arrest or apoptosis depending on the physiological circumstances and cell type. It is involved in cell cycle regulation as a trans-activator that acts to negatively regulate cell division by controlling a set of genes required for this process. Human p53 is found in increased amounts in a wide variety of transformed cells. It is frequently mutated or inactivated in about 60% of cancers. Human p53 defects are found in Barrett metaplasia a condition in which the normally stratified squamous epithelium of the lower esophagus is replaced by a metaplastic columnar epithelium. The condition develops as a complication in approximately 10% of patients with chronic gastroesophageal reflux disease and predisposes to the development of esophageal adenocarcinoma.

Nine isoforms of p53 naturally occur and are expressed in a wide range of normal tissues but in a tissue-dependent manner. Isoform 2 is expressed in most normal tissues but is not detected in brain, lung, prostate, muscle, fetal brain, spinal cord and fetal liver. Isoform 3 is expressed in most normal tissues but is not detected in lung, spleen, testis, fetal brain, spinal cord and fetal liver. Isoform 7 is expressed in most normal tissues but is not detected in prostate, uterus, skeletal muscle and breast. Isoform 8 is detected only in colon, bone marrow, testis, fetal brain and intestine. Isoform 9 is expressed in most normal tissues but is not detected in brain, heart, lung, fetal liver, salivary gland, breast or intestine.

### SUMMARY OF THE INVENTION

The invention provides:—

In a First Configuration

A protein multimer of at least first, second, third and fourth copies of an effector domain (eg, a protein domain or a peptide), wherein the multimer is multimerised by first, second, third and fourth self-associating tetramerization domains (TDs) which are associated together, wherein each tetramerization domain is comprised by a respective engineered polypeptide comprising one or more copies of said protein domain or peptide.

In a Second Configuration

An isolated tetramer or octamer of a TCR binding site, insulin peptide, incretin peptide or peptide hormone; or a plurality of said tetramers or octamers.
An isolated tetramer or octamer of an antibody binding site or an antibody variable domain (eg, a single variable domain); or a plurality of said tetramers or octamers.
In an example the tetramer or octamer is soluble in aqueous solution (eg, aqueous eukaryotic cell culture medium). In an example the tetramer or octamer is expressible in a eukaryotic cell. Exemplification is provided below.

In a Third Configuration

A tetramer or octamer of
(a) TCR V domains or TCR binding sites, wherein the tetramer or octamer is soluble in aqueous solution (eg, an aqueous eukaryotic cell growth medium or buffer);
(b) antibody single variable domains, wherein the tetramer or octamer is soluble in aqueous solution (eg, an aqueous eukaryotic cell growth medium or buffer);
(c) TCR V domains or TCR binding sites, wherein the tetramer or octamer is capable of being intracellularly and/or extracellularly expressed by HEK293 cells; or
(d) antibody variable domains (eg, antibody single variable domains), wherein the tetramer or octamer is capable of being intracellularly and/or extracellularly expressed by HEK293 cells.

In a Fourth Configuration

An engineered polypeptide or monomer of a multimer, tetramer or octamer of the invention.

In a Fifth Configuration

An engineered (and optionally isolated) engineered polypeptide (P1) which comprises (in N- to C-terminal direction):—
(a) TCR V1-TCR C1-antibody CH1 (eg, IgG CH1)-optional linker-TD, wherein
   (i) V1 is a Vα and C1 is a Cα;
   (ii) V1 is a Vβ and C1 is a Cβ;
   (iii) V1 is a Vγ and C1 is a Cγ; or
   (iv) V1 is a Vδ and C1 is a Cδ;
      or
(b) TCR V1-antibody CH1 (eg, IgG CH1)-optional linker-TD, wherein
   (i) V1 is a Vα;
   (ii) V1 is a Vβ;
   (iii) V1 is a Vγ; or
   (iv) V1 is a Vδ;
      or
(c) antibody V1-antibody CH1 (eg, IgG CH1)-optional linker-TD, wherein
   (i) V1 is a VH; or
   (ii) V1 is a VL (eg, a Vλ or a Vκ);
      or
(d) antibody V1-optional antibody CH1 (eg, IgG CH1)-antibody Fc (eg, an IgG Fc)-optional linker-TD, wherein
   (i) V1 is a VH; or
   (ii) V1 is a VL (eg, a Vλ or a Vκ);
      or
(e) antibody V1-antibody CL (eg, a Cλ or a Cκ)-optional linker-TD, wherein
   (i) V1 is a VH; or
   (ii) V1 is a VL (eg, a Vλ or a Vκ);
      or
(f) TCR V1-TCR C1-optional linker-TD, wherein
   (i) V1 is a Vα and C1 is a Cα;
   (ii) V1 is a Vβ and C1 is a Cβ;
   (iii) V1 is a Vγ and C1 is a Cγ; or
   (iv) V1 is a Vδ and C1 is a Cδ.

In a Sixth Configuration

A nucleic acid encoding an engineered polypeptide or monomer of the invention, optionally wherein the nucleic acid is comprised by an expression vector for expressing the polypeptide.

In a Seventh Configuration

Use of a nucleic acid or vector of the invention in a method of manufacture of protein multimers for producing intracellularly expressed and/or secreted multimers, wherein the method comprises expressing the multimers in and/or secreting the multimers from eukaryotic cells comprising the nucleic acid or vector.

In an Eighth Configuration

A method producing
(a) TCR V domain multimers, the method comprising the soluble and/or intracellular expression of TCR V-TD (eg, NHR2 TD or TCR V-p53 TD) fusion proteins expressed in eukaryotic cells, the method optionally comprising isolating a plurality of said multimers;
(b) antibody V domain multimers, the method comprising the soluble and/or intracellular expression of antibody V (eg, a single variable domain)-TD (eg, V-NHR2 TD or V-p53 TD) fusion proteins expressed in eukaryotic cells, the method optionally comprising isolating a plurality of said multimers;
(c) incretin peptide (eg, GLP-1, GIP or insulin) multimers, the method comprising the soluble and/or intracellular expression of incretin peptide-TD (eg, incretin peptide-NHR2 TD or incretin peptide-p53 TD) fusion proteins expressed in eukaryotic cells, such as HEK293T cells; the method optionally comprising isolating a plurality of said multimers; or
(d) peptide hormone multimers, the method comprising the soluble and/or intracellular expression of peptide hormone-TD (eg, peptide hormone-NHR2 TD or peptide hormone-p53 TD) fusion proteins expressed in eukaryotic cells, such as HEK293T cells; the method optionally comprising isolating a plurality of said multimers.

In a Ninth Configuration

Use of a nucleic acid or vector of the invention in a method of manufacture of protein multimers for producing glycosylated multimers in eukaryotic cells comprising the nucleic acid or vector.

In a Tenth Configuration

Use of self-associating tetramerization domains (TD) (eg, NHR2 TD, p53 TD, p63 TD or p73 TD or a homologue or orthologue thereof) in a method of the manufacture of a tetramer of polypeptides, for producing a higher yield of tetramers versus monomer and/or dimer polypeptides.

In a Eleventh Configuration

Use of an engineered polypeptide in a method of the manufacture of a tetramer of a polypeptide comprising multiple copies of a protein domain or peptide, for producing a higher yield of tetramers versus monomer and/or dimer polypeptides, wherein the engineered polypeptide comprises one or more copies of said protein domain or peptide and further comprises a self-associating tetramerization domains (TD) (eg, NHR2 TD, p53 TD, p63 TD or p73 TD or a homologue or orthologue).

In a Twelfth Configuration

Use of self-associating tetramerization domains (TD) (eg, NHR2 TD, p53 TD, p63 TD or p73 TD or a homologue or orthologue thereof) in a method of the manufacture of a tetramer of a polypeptide, for producing a plurality of tetramers that are not in mixture with monomers, dimers or trimers.

In a Thirteenth Configuration

A eukaryotic host cell comprising the nucleic acid or vector for intracellular and/or secreted expression of the multimer, tetramer, octamer, engineered polypeptide or monomer of the invention.

In a Fourteenth Configuration

Use of an engineered polypeptide in a method of the manufacture of a tetramer of a polypeptide comprising multiple copies of a protein domain or peptide, for producing a plurality of tetramers that are not in mixture with monomers, dimers or trimers, wherein the engineered polypeptide comprises one or more copies of said protein domain or peptide and further comprises a self-associating tetramerization domains (TD) (eg, NHR2 TD, p53 TD, p63 TD or p73 TD or a homologue or orthologue).

In a Fifteenth Configuration

A multivalent heterodimeric soluble T cell receptor capable of binding pMHC complex comprising:
(i) TCR extracellular domains;
(ii) immunoglobulin constant domains; and
(iii) an NHR2 multimerisation domain of ETO.

In a Sixteenth Configuration

A multimeric immunoglobulin, comprising
(i) immunoglobulin variable domains; and
(ii) an NHR2 multimerisation domain of ETO.

In a Seventeenth Configuration

A method for assembling a soluble, multimeric polypeptide, comprising:
(a) providing a monomer of the said multimeric polypeptide, fused to an NHR2 domain of ETO;
(b) causing multiple copies of said monomer to associate, thereby obtaining a multimeric, soluble polypeptide.

In an Eighteenth Configuration

A mixture comprising (i) a cell line (eg, a eukaryotic, mammalian cell line, eg, a HEK293, CHO or Cos cell line) encoding a polypeptide of the invention; and (ii) tetramers of the invention.

In a Ninteenth Configuration

A method for enhancing the yield of tetramers of an protein effector domain (eg, an antibody variable domain or binding site), the method comprising expressing from a cell line (eg, a mammalian cell, CHO, HEK293 or Cos cell line) tetramers of a polypeptide, wherein the polypeptide is a polypeptide of the invention and comprises one or more effector domains; and optionally isolating said expressed tetramers.

The invention also provides a pharmaceutical composition, cosmetic, foodstuff, beverage, cleaning product, detergent comprising the multimer(s), tetramer(s) or octamer(s) of the invention.

### BRIEF DESCRIPTION OF THE DRAWINGS

FIG. 1. A schematic drawing representing the stepwise self-assembly of an tetravalent heterodimeric soluble TCR protein complex via a monomer and homodimer, which is aided by NHR2 tetramerization domain.

FIG. 2. A schematic drawing representing the stepwise self-assembly of a octavalent heterodimeric soluble TCR protein complex via a monomer² and homodimer², which is aided by NHR2 tetramerization domain and immunoglobulin hinge domain.

FIG. 3: A schematic drawing of the domain arrangements in the α and β chain used for expressing and assembling is-NY-ESO-1 TCR.

FIG. 4: A schematic drawing of the domain arrangements in the α and β chain used for expressing and assembling os-NY-ESO-1 TCR.

FIG. 5: Amino acid sequence of the α and β chain of the is-NY-ESO-1 TCR protein complex. Amino acid sequences of alternate domains are underlined.

FIG. 6: Amino acid sequence of the α and β chain of the os-NY-ESO-1 TCR protein complex. Amino acid sequences of alternate domains are underlined.

FIG. 7: A schematic drawing of the domain arrangements in the α and β chain used for expressing and assembling is-NY-ESO-1 TCR-IL2 fusion protein complex.

FIG. 8: A schematic drawing of the domain arrangements in the α and β chain used for expressing and assembling os-NY-ESO-1 TCR-IL2 fusion protein complex.

FIG. 9: Amino acid sequence of the α and β chain of the is-NY-ESO-1 TCR-IL2 fusion protein complex. Amino acid sequences of alternate domains are underlined.

FIG. 10: Amino acid sequence of the α and β chain of the os-NY-ESO-1 TCR-IL2 fusion protein complex. Amino acid sequences of alternate domains are underlined.

FIG. 11A: A schematic drawing representing the stepwise self-assembly of a tetravalent single domain antibody (dAb) complex via a monomer and homodimer, which is aided by NHR2 tetramerization domain.

FIG. 11B: A schematic drawing of the domain arrangements for assembly of tetravalent dAbs, including linker and NHR2 domains.

FIG. 12A: A schematic drawing representing the stepwise self-assembly of a tetravalent Fab complex via a monomer and homodimer, which is aided by NHR2 tetramerization domain.

FIG. 12B: A schematic drawing of the domain arrangements for assembly of tetravalent Fabs, including linker and NHR2 domains in the heavy chain, and light chain variable and constant domains.

FIG. 13A: A schematic drawing representing the stepwise self-assembly of an octavalent Fab complex via a monomer and homodimer, which is aided by NHR2 tetramerization domain and an antibody hinge region linked to CH1 domain.

FIG. 13B: A schematic drawing of the domain arrangements for assembly of octavalent Fabs, including hinge, linker and NHR2 domains in the heavy chain, and light chain variable and constant domains.

FIG. 14 is a schematic of Quad 16 and Quad 17.

FIG. 15A shows Quad16 monomer sequence and configuration, and FIG. 15B shows Quad 17 monomer sequence and configuration.

FIGS. 16A-16B show analysis of secreted proteins using anti-Ig Western Blot: (FIG. 16A) a PAGE gel under SDS denatured conditions—16=Quad16; 17=Quad17; and (FIG. 16B) a PAGE gel under native (ie, non-denatured) conditions—16=Quad16; 17=Quad17.

FIGS. 17A-17B: Western blots prepared from denaturing SDS-PAGE gel probed with anti-human IgG HRP detection antibody (FIG. 17A) Protein samples from Quads 3 and 4 were prepared from whole cell extracts and loaded in lanes 1 and 2 respectively. The expected Mw for Quads 3 and 4 are 46.1 and 46.4 kDa respectively. (FIG. 17B) Protein samples from Quads 12 and 13 were prepared from whole cell extracts and loaded in lanes 1 and 2 respectively. The expected Mw for Quads 12 and 13 are 47.8 and 48.1 kDa respectively.

FIGS. 18A-18B: Western blots prepared from denaturing SDS-PAGE gel probed with anti-human IgG HRP detection antibody (FIG. 18A) Protein samples from Quads 3 and 4 were prepared by concentrating cell supernatant and loaded in lanes 1 and 2 respectively. The expected Mw for Quads 3 and 4 are 46.1 and 46.4 kDa respectively. (FIG. 18B) Protein samples from Quads 12 and 13 were prepared by concentrating cell supernatant and loaded in lanes 1 and 2 respectively. The expected Mw for Quads 12 and 13 are 47.8 and 48.1 kDa respectively.

FIGS. 19A-19D: Western blots prepared from denaturing SDS-PAGE gel probed with anti-HIS HRP detection antibody (FIG. 19A) Protein samples from Quads 14, 15, 18 and 19 were prepared from whole cell extracts and loaded in lanes 1-4, respectively. The expected Mw for Quads 14, 15, 18 and 19 are 22.0, 22.3, 37.4 and 37.7 kDa respectively. (FIG. 19B) Protein samples from Quads 23, 24, 26 and 27 were prepared from whole cell extracts and loaded in lanes 1-4, respectively. The expected Mw for Quads 23, 24, 26 and 27 are 32.1, 32.4, 33.7 and 34.0 kDa respectively. (FIG. 19C) Protein samples from Quads 34, and 38 were prepared from whole cell extracts and loaded in lanes 1-2, respectively. The expected Mw for Quads 34, and 38 are 25.5 and 25.4 kDa respectively. (FIG. 19D) Protein samples from Quads 40,42, 44 and 46 were prepared from whole cell extracts and loaded in lanes 1-4, respectively. The expected Mw for Quads 40, 42, 44 and 46 are 25.4, 37.6, 25.5 and 38.0 kDa respectively. Lane U contains concentrated serum prepared from untransfected HEK293T cells (negative control) and C is a His-tagged protein used as a positive control for the anti-His HRP detection antibody. Serum anti-His background band is highlighted by a black arrow, which can be consistently detected in all for blots.

FIGS. 20A-20B: Western blot prepared from denaturing SDS-PAGE gel (FIG. 20A) and probed with anti-human IgG HRP detection antibody. Protein samples from Quads 14 and 15 were prepared from whole cell extracts and loaded in lanes 1 and 2, respectively. The expected Mw for Quads 14 and 15 are 22.0 and 22.3 kDa respectively. (FIG. 20B) Western blot prepared from Native PAGE gels and probed with anti-human IgG HRP detection antibody. Lanes 1 and 2 contains protein samples from Quads 14 and 15 prepared from whole cell extract.

FIG. 21: Quad polyeptides fused to leader and tag sequences. Where linker is present, the linker is G4S (only 1 G4S). * denotes TCR constant domains with introduced cysteine residue allowing S-S bond formation between TCR alpha and beta chain. Human IgG1 hinge was used. All C regions are human. The TCR V domains are specific for NY-ESO-1. GFP=green fluorescent protein.

All polypeptide schematics and amino acid sequences herein are written N- to C-terminal. All nucleotide sequences herein are written 5′ to 3′.

### DETAILED DESCRIPTION

The invention relates to multimers such as tetramers of polypeptides and tetramers and octamers of effector domains (such as antigen binding sites (eg, antibody or TCR binding sites that specifically bind to antigen or pMHC, or variable domains thereof)) or peptides such as incretin, insulin or hormone peptides. In embodiments, multimers of the invention are usefully producible in eurkaryotic systems and can be secreted from eukaryotic cells in soluble form, which is useful for various industrial applications, such as producing pharmaceuticals, diagnostics, as imaging agents, detergents etc. Higher order multimers, such as tetramers or octamers of effector domains or peptides are useful for enhancing antigen or pMHC binding avidity. This may be useful for producing an efficacious medicine or for enhancing the sensitivity of a diagnostic reagent comprising the multimer, tetramer or octamer. An additional or alternative benefit is enhanced half-life in vivo when the multimers of the invention are administered to a human or animal subject, eg, for treating or preventing a disease or condition in the subject. Usefully, the invention can also provide for multi-specific (eg, bi- or tri-specific) multivalent binding proteins. Specificity may related to specificity of antigen or pMHC binding. By using a single engineered polypeptide comprising binding domains or peptides, the invention in certain examples usefully provides a means for producing multivalent (eg, bi-specific) proteins at high purity. Use of a single species of engineered polypeptide monomer avoids the problem of mixed products seen when 2 or more different polypeptide species are used to produce multi- (eg, bi-) specific or multivalent proteins.

The invention provides the following Clauses, Aspects and Concepts. Any Clause herein can be combined with any Aspect or Concept herein. Any Aspect herein can be combined with any Concept herein.

Aspects:
1. A protein multimer of at least first, second, third and fourth copies of an effector domain (eg, a protein domain) or a peptide, wherein the multimer is multimerised by first, second, third and fourth self-associating tetramerization domains (TDs) which are associated together, wherein each tetramerization domain is comprised by a respective engineered polypeptide comprising one or more copies of said protein domain or peptide.

In an example, each TD is a TD of any one of proteins 1 to 119 listed in Table 2. In an example, each TD is a p53 TD or a homologue or orthologue thereof. In an example, each TD is a NHR2 TD or a homologue or orthologue thereof. In an example, each TD is a p63 TD or a homologue or orthologue thereof. In an example, each TD is a p73 TD or a homologue or orthologue thereof. In an example, each TD is not a NHR2 TD. In an example, each TD is not a p53 TD. In an example, each TD is not a p63 TD. In an example, each TD is not a p73 TD. In an example, each TD is not a p53, 63 or 73 TD. In an example, each TD is not a NHR2, p53, 63 or 73 TD.

By being “associated together”, the TDs in Aspect 1 multimerise first, second, third and fourth copies of the engineered polypeptide to provide a multimer protein, for example, a multimer that can be expressed intracellulary in a eukaryotic or mammalian cell (eg, a HEK293 cell) and/or which can be extracellularly secreted from a eukaryotic or mammalian cell (eg, a HEK293 cell) and/or which is soluble in an aqueous medium (eg, a eukaryotic or mammalian cell (eg, a HEK293 cell) culture medium). Examples are NHR TD, p53 TD, p63 TD and p73 TD (eg, human NHR TD, p53 TD, p63 TD and p73 TD) or an orthologue or homologue thereof.

In an example, the TD is not a p53 TD (or homologue or orthologue thereof), eg, it is not a human p53 TD (or homologue or orthologue thereof). In an example, the TD is a NHR2 TD or a homologue or orthologue thereof, but excluding a p53 TD or a homologue or orthologue thereof. In an example, the TD is a human NHR2 TD or a homologue or orthologue thereof, but excluding a human p53 TD or a homologue or orthologue thereof. In an example, the TD is human NHR2. In an example, the amino acid sequence of the TD is at least 80, 85, 90, 95, 96, 97, 98 or 99% identical to the sequence of human NHR2. In an example, the domain or peptide is not naturally comprised by a polypeptide that also comprise a NHR2 TD.

In an example, all of the domains of the polypeptide are human.

The engineered polypeptide may comprise one or more copies of said domain or peptide N-terminal to a copy of said TD. Additionally or alternatively, the engineered polypeptide may comprise one or more copies of said domain or peptide C-terminal to a copy of said TD. In an example, the engineered polypeptide comprises a first said domain or peptide and a TD, wherein the first domain or peptide is spaced by at least 10, 20, 30, 40, 50, 60, 70, 80, 90, 100, 200, 300, 400,500, 600, 700, 800, 900 or 1000 contiguous amino acids from the TD, wherein there is no further said domain or peptide between the first domain or peptide and the TD.

In an example, the multimer (eg, tetramer of said engineered polypeptide) comprises 4 (but no more than 4) TDs (eg, identical TDs) and 4, 8, 12 or 16 (but no more than said 4, 8, 12 or 16 respectively) copies of said domain or peptide. In an example, each TD and each said domain or peptide is human.

In an example, the multimer, tetramer or octamer comprises first, second, third and fourth identical copies of an engineered polypeptide, the polypeptide comprising a TD and one (but no more than one), two (but no more than two), or more copies of the said protein domain or peptide.

In some embodiments, by requiring just one type of engineered polypeptide to form the multimer, tetramer or octamer of the invention, the invention advantageously provides a format that can be readily isolated in pure (or highly pure, ie >90, 95, 96, 97, 98 or 99% purity) format, as well as a method for producing such a format in pure (or highly pure) form. Purity is indicated by the multimer of the invention not being in mixture in a composition with any other multimer or polypeptide monomer, or wherein the multimer of the invention comprises >90, 95, 96, 97, 98 or 99% of species in a composition comprising the multimer of the invention and other multimers and/or polypeptide monomers which comprise the engineered polyeptide. Thus, mixtures of different types of polypeptide in these embodiments are avoided or minimised. This advantageously also provides, therefore, plurality of multimers (eg, a plurality of tetramers or octamers) that comprise only one (and no more than one) type of engineered polypeptide, wherein the multimers are monospecific (but multivalent) for antigen binding, or alternatively bi- or multi-specific for antigen binding. Thus, the invention provides a plurality of multimers (eg, a plurality of tetramers or octamers, each polypeptide being at least tetra-valent for antigen binding and (i) bi-specific (ie, capable of specifically binding to 2 different antigens) or (ii) mono-specific and at least tetravalent for antigen binding. Herein, where antigen binding is mentioned this can instead be pMHC binding when the domain is a TCR V domain. Advantageously, the plurality is in pure form (ie, not mixed with multimers (eg, tetramers or octamers) that comprise more than one type of polypeptide monomer. In an example, the multimer comprises at least 2 different types of antigen binding site. In an example, the multimer is bi-specific, tri-specific or tetra-specific. In an example, the multimer has an antigen binding site or pMHC binding site valency of 4, 6, 8, 10 or 12, preferably 4 or 8.

In an example, a peptide MHC (pMHC) is a class I or class II pMHC.

By the term “specifically binds,” as used herein, eg, with respect to a domain, antibody or binding site, is meant a domain, antibody or binding site which recognises a specific antigen (or pMHC) with a binding affinity of 1 mM or less as determined by SPR

Target binding ability, specificity and affinity (KD (also termed Kd), K_{off} and/or Kₒₙ) can be determined by any routine method in the art, eg, by surface plasmon resonance (SPR). The term “KD”, as used herein, is intended to refer to the equilibrium dissociation constant of a particular binding site/ligand, receptor/ligand or antibody/antigen interaction. In one embodiment, the surface plasmon resonance (SPR) is carried out at 25° C. In another embodiment, the SPR is carried out at 37° C. In one embodiment, the SPR is carried out at physiological pH, such as about pH7 or at pH7.6 (eg, using Hepes buffered saline at pH7.6 (also referred to as HBS-EP)). In one embodiment, the SPR is carried out at a physiological salt level, eg, 150 mM NaCl. In one embodiment, the SPR is carried out at a detergent level of no greater than 0.05% by volume, eg, in the presence of P20 (polysorbate 20; eg, Tween-20™) at 0.05% and EDTA at 3 mM. In one example, the SPR is carried out at 25° C. or 37° C. in a buffer at pH7.6, 150 mM NaCl, 0.05% detergent (eg, P20) and 3 mM EDTA. The buffer can contain 10 mM Hepes. In one example, the SPR is carried out at 25° C. or 37° C. in HBS-EP. HBS-EP is available from Teknova Inc (California; catalogue number H8022). In an example, the affinity (eg, of a VH/VL binding site) is determined using SPR by using any standard SPR apparatus, such as by Biacore™ or using the ProteOn XPR36™ (Bio-Rad®). The binding data can be fitted to 1:1 model inherent using standard techniques, eg, using a model inherent to the ProteOn XPR36™ analysis software.

In an example, a multimer, tetramer or octamer of the invention is an isolated multimer, tetramer or octamer. In an example, a multimer, tetramer or octamer of the invention consists of copies of said engineered polypeptide. Optionally the multimer, tetramer or octamer of the invention comprises 4 or 8 but not more than 4 or 8 copies respectively of the engineered polypeptide.

By “engineered” is meant that the polypeptide is not naturally-occurring, for example the protein domain or peptide is not naturally comprised by a polypeptide that also comprises said TD.

Each said protein domain or peptide may be a biologically active domain or peptide (eg, biologically active in humans or animals), such as a domain that specifically binds to an antigen or peptide-MHC (pMHC), or wherein the domain is comprised by an antigen or pMHC binding site. In an alternative, the domain or peptide is a carbohydrate, glucose or sugar-regulating agent, such as an incretin or an insulin peptide. In an alternative, the domain or peptide is an inhibitor or an enzyme or an inhibitor of a biological function or pathway in humans or animals. In an alternative, the domain or peptide is an iron-regulating agent. Thus, in an example, each protein domain or peptide is selected from an antigen or pMHC binding domain or peptide; a hormone; a carbohydrate, glucose or sugar-regulating agent; an iron-regulating agent; and an enzyme inhibitor.
2. The multimer of any preceding Aspect, wherein the multimer is a tetramer or an octamer of said domain or peptide.
3. The multimer of any Aspect 1 or 2, comprising a tetramer or octamer of an immunoglobulin superfamily binding site (eg, an antibody or TCR binding site, such as a scFv or scTCR).

The immunoglobulin superfamily (IgSF) is a large protein superfamily of cell surface and soluble proteins that are involved in the recognition, binding, or adhesion processes of cells. Molecules are categorized as members of this superfamily based on shared structural features with immunoglobulins (also known as antibodies); they all possess a domain known as an immunoglobulin domain or fold. Members of the IgSF include cell surface antigen receptors, co-receptors and co-stimulatory molecules of the immune system, molecules involved in antigen presentation to lymphocytes, cell adhesion molecules, certain cytokine receptors and intracellular muscle proteins. They are commonly associated with roles in the immune system.

T-cell receptor (TCR) domains can be Vα (eg. paired with a Vβ), Vβ (eg. paired with a Vα), Vγ (eg, paired with a Vδ) or Vδ (eg, paired with a Vγ).
4. The multimer of Aspect 3, wherein the binding site comprises a first variable domain paired with a second variable domain.

In a first example, the first and second variable domains are comprised by the engineered polypeptide. In another example, the first domain is comprised by the engineered polypeptide and the second domain is comprised a by a further polypeptide that is different from the engineered polypeptide (and optionally comprises a TD or is devoid of a TD).

In the alternative, the domains are constant region domains. In an alternative, the domains are FcAbs. In an alternative, the domains are non-Ig antigen binding sites or comprises by a non-Ig antigen binding site, eg, an affibody.

### Antigen Binding Sites & Effector Domains

In an example, the or each antigen binding site (or effector domain) is selected from the group consisting of an antibody variable domain (eg, a VL or a VH, an antibody single variable domain (domain antibody or dAb), a camelid VHH antibody single variable domain, a shark immunoglobulin single variable domain (NA V), a Nanobody™ or a camelised VH single variable domain); a T-cell receptor binding domain; an immunoglobulin superfamily domain; an agnathan variable lymphocyte receptor (J Immunol; 2010 Aug. 1; 185(3):1367-74; “Alternative adaptive immunity in jawless vertebrates; Herrin BR & Cooper M D.); a fibronectin domain (eg, an Adnectin™); an scFv; an (scFv)₂; an sc-diabody; an scFab; a centyrin and an antigen binding site derived from a scaffold selected from CTLA-4 (Evibody™); a lipocalin domain; Protein A such as Z-domain of Protein A (eg, an Affibody™ or SpA); an A-domain (eg, an Avimer™ or Maxibody™); a heat shock protein (such as and epitope binding domain derived from GroEI and GroES); atransferrin domain (eg, a trans-body); ankyrin repeat protein (eg, a DARPin™); peptide aptamer; C-type lectin domain (eg, Tetranectin™); human γ-crystallin or human ubiquitin (an affilin); a PDZ domain; scorpion toxin; and a kunitz type domain of a human protease inhibitor.

Further sources of antigen binding sites are variable domains and VH/VL pairs of antibodies disclosed in WO2007024715 at page 40, line 23 to page 43, line 23. This specific disclosure is incorporated herein by reference as though explicitly written herein to provide basis for epitope binding moieties for use in the present invention and for possible inclusion in claims herein.

A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain

The phrase “immunoglobulin single variable domain” or “antibody single variable domain” refers to an antibody variable domain (VH, VHH, VL) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004), nurse shark and Camelid VHH immunoglobulin single variable domains. Camelid VHH sre immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are still considered to be “domain antibodies” according to the invention. As used herein “VH includes camelid VHH domains. NA V are another type of immunoglobulin single variable domain which were identified in cartilaginous fish including the nurse shark. These domains are also known as Novel Antigen Receptor variable region (commonly abbreviated to V(NAR) or NARV). For further details see Mol. Immunol. 44, 656-665 (2006) and US20050043519A. CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001). Lipocalins are a family of extracellular proteins which transport small hydrophobic molecules such as steroids, bilins, retinoids and lipids. They have a rigid β-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. Sel. 17, 455-462 (2004) and EP1641818A1. Avimers™ are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins™) are derived from ankyrin which is a family of proteins that mediate attachment of integral membrane proteins to the cytoskeleton. A single ankyrin repeat is a 33 residue motif consisting of two a-helices and β-turn. They can be engineered to bind different target antigens by randomising residues in the first a-helix and a β-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

Fibronectin is a scaffold which can be engineered to bind to antigen. Adnectins™ consist of a backbone of the natural amino acid sequence of the 10th domain of the 15 repeating units of human fibronectin type III (FN3). Three loops at one end of the I3-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can be engineered to include up to 25 amino acids without affecting the overall fold of the microprotein. For further details of engineered knottin domains, see WO2008098796. Other epitope binding moieties and domains include proteins which have been used as a scaffold to engineer different target antigen binding properties include human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006).
5. The multimer of any preceding Aspect, wherein each polypeptide comprises first and second copies of said protein domain or peptide, wherein the polypeptide comprises in (N- to C-terminal direction) (i) a first of said copies-TD-the second of said copies; (ii) TD-and the first and second copies; or (iii) said first and second copies-TD.
6. The multimer of any preceding Aspect, wherein the TDs are NHR2 TDs and the domain or peptide is not a NHR2 domain or peptide; or wherein the TDs are p53 TDs and the domain or peptide is not a p53 domain or peptide.
7. The multimer of any preceding Aspect, wherein the engineered polypeptide comprises one or more copies of a second type of protein domain or peptide, wherein the second type of protein domain or peptide is different from the first protein domain or peptide.

For example, the polypeptide comprises in N-terminal direction (i) P1-TD-P2; or (ii) TD-P1-P2, wherein P1=a copy of a domain or peptide of the first type (ie, the type of domain or peptide of the multimer of Aspect 1); and P2=a copy of a domain or peptide of said second type.
8. The multimer of any preceding Aspect, wherein the domains are immunoglobulin superfamily domains.
9. The multimer of any preceding Aspect, wherein the domain or peptide is an antibody variable or constant domain (eg, an antibody single variable domain), a TCR variable or constant domain, an incretin, an insulin peptide, or a hormone peptide.
10. The multimer of any preceding Aspect, wherein the multimer comprises first, second, third and fourth identical copies of a said engineered polypeptide, the polypeptide comprising a TD and one (but no more than one), two (but no more than two) or more copies of the said protein domain or peptide.
11. The multimer of any preceding Aspect, wherein the engineered polypeptide comprises an antibody or TCR variable domain (V1) and a NHR2 TD.
12. The multimer of Aspect 11, wherein the polypeptide comprises (in N- to C-terminal direction) (i) V1-an optional linker-NHR2 TD; (ii) V1-an optional linker-NHR2 TD-optional linker-V2; or (iii) V1-an optional linker-V2-optional linker-NHR2 TD, wherein V1 and V2 are TCR variable domains and are the same or different, or wherein V1 and V2 are antibody variable domains and are the same or different.
13. The multimer of Aspect 12, wherein V1 and V2 are antibody single variable domains.
14. The multimer of aspect 11, wherein each engineered polypeptide comprises (in N- to C-terminal direction) V1-an optional linker-NHR2 TD, wherein V1 is an antibody or TCR variable domain and each engineered polypeptide is paired with a respective second engineered polypeptide that comprises V2, wherein V2 is a an antibody or TCR variable domain respectively that pairs with V1 to form an antigen or pMHC binding site, and optionally one polypeptide comprises an antibody Fc, or comprises antibody CH1 and the other polypeptide comprises an antibody CL that pairs with the CH1.
15. The multimer of any preceding Aspect, wherein the TD comprises (i) an amino acid sequence identical to SEQ ID NO: 10 or 126 or at least 80% identical thereto; or (ii) an amino acid sequence identical to SEQ ID NO: 120 or 123 or at least 80% identical thereto.
16. The multimer of any preceding Aspect, wherein the multimer comprises a tetramer or octamer of an antigen binding site of an antibody selected from the group consisting of REOPRO®; Abciximab; RITUXAN®; Rituximab; ZENAPAX®; Daclizumab; SIMULECT®; Basiliximab; SYNAGIS®; Palivizumab; REMICADE®; Infliximab; HERCEPTIN®; MYLOTARG®; Gemtuzumab; CAMPATH®; Alemtuzumab; ZEVALIN®; Ibritumomab; HUMIRA®; Adalimumab; XOLAIR®; Omalizumab; BEXXAR®; Tositumomab; RAPTIVA™; Efalizumab; ERBITUX®; Cetuximab; AVASTIN®; Bevacizumab; TYSABRI®; Natalizumab; ACTEMRA®; Tocilizumab;-VECTIBIX®; Panitumumab; LUCENTIS®; Ranibizumab; SOLIRIS®; Eculizumab; CIMZIA®; Certolizumab; SIMPONI®; Golimumab, ILARIS®; Canakinumab; STELARA®; Ustekinumab;-ARZERRA®; Ofatumumab; PROLIA®; Denosumab; NUMAX™; Motavizumab; ABTHRAX™; Raxibacumab; BENLYSTA®; Belimumab; YERVOY®; Ipilimumab; ADCETRIS®; Brentuximab Vedotin; PERJETA®; Pertuzumab; KADCYLA®; Ado-trastuzumab;-KEYTRUDA®, OPDIVO®, GAZYVA® and Obinutuzumab.

For example, a said protein domain of the engineered polypeptide is a V domain (a VH or VL) of an antibody binding site of an antibody selected from said group, wherein the multimer comprises a further V domain (a VL or VH respectively) that pairs with the V domain of the engineered polypeptide to form the antigen binding site of the selected antibody. Advantageously, therefore, the invention provides tetramers or octamers of a binding site of said selected antibody, which beneficially may have improved affinity, avidity and/or efficacy for binding its cognate antigen or for treating or preventing a disease or condition in a human or animal wherein the multimer is administered thereto to bind the cognate antigen in vivo.

For example, the multimer, tetramer or octamer comprises 4 copies of an antigen binding site of an antibody, wherein the antibody is adalimumab, sarilumab, dupilumab, bevacizumab (eg, AVASTIN™), cetuximab (eg, ERBITUX™), tocilizumab (eg, ACTEMRA™) or trastuzumab (HERCEPTIN™). In an alternative the antibody is an anti-CD38 antibody, an anti-TNFa antibody, an anti-TNFR antibody, an anti-IL-4Ra antibody, an anti-IL-6R antibody, an anti-IL-6 antibody, an anti-VEGF antibody, an anti-EGFR antibody, an anti-PD-1 antibody, an anti-PD-L1 antibody, an anti-CTLA4 antibody, an anti-PCSK9 antibody, an anti-CD3 antibody, an anti-CD20 antibody, an anti-CD138 antibody, an anti-IL-1 antibody. In an alternative the antibody is selected from the antibodies disclosed in WO2007024715 at page 40, line 23 to page 43, line 23, the disclosure of which is incorporated herein by reference.

A binding site herein may, for example, be a ligand (eg, cytokine or growth factor, eg, VEGF or EGFR) binding site of a receptor (eg, KDR or Flt). A binding site herein may, for example, be a binding site of Eyelea™, Avastin™ or Lucentis™, eg, for ocular or oncological medical use in a human or animal. When the ligand or antigen is VEGF, the mutlimer, tetramer or octamer may be for treatment or prevention of a caner or ocular condition (eg, wet or dry AMD or diabetic retinopathy) or as an inhibitor of neovascularisation in a human or animal subject.
17. An isolated tetramer or octamer of a TCR binding site, insulin peptide, incretin peptide or peptide hormone; or a plurality of said tetramers or octamers.

Several important peptide hormones are secreted from the pituitary gland. The anterior pituitary secretes three hormones: prolactin, which acts on the mammary gland; adrenocorticotropic hormone (ACTH), which acts on the adrenal cortex to regulate the secretion of glucocorticoids; and growth hormone, which acts on bone, muscle, and the liver. The posterior pituitary gland secretes antidiuretic hormone, also called vasopressin, and oxytocin. Peptide hormones are produced by many different organs and tissues, however, including the heart (atrial-natriuretic peptide (ANP) or atrial natriuretic factor (ANF)) and pancreas (glucagon, insulin and somatostatin), the gastrointestinal tract (cholecystokinin, gastrin), and adipose tissue stores (leptin). In an example, the peptide hormone of the invention is selected from prolactin, ACTH, growth hormone (somatotropin), vasopressin, oxytocin, glucagon, insulin, somatostatin, cholecystokinin, gastrin and leptin (eg, selected from human prolactin, ACTH, growth hormone, vasopressin, oxytocin, glucagon, insulin, somatostatin, cholecystokinin, gastrin and leptin).

In an example, the incretin is a GLP-1, GIP or exendin-4 peptide.

The invention provides, in embodiments, the following engineered tetramers and octamers:—

An isolated tetramer or octamer of an incretin.
An isolated tetramer or octamer of an insulin peptide.
An isolated tetramer or octamer of a GLP-1 (glucagon-like peptide-1 (GLP-1) peptide.
An isolated tetramer or octamer of a GIP (glucose-dependent insulinotropic polypeptide) peptide.
An isolated tetramer or octamer of an exendin (eg, exendin-4) peptide.
An isolated tetramer or octamer of a peptide hormone.
An isolated tetramer or octamer of a prolactin or prolactin peptide.
An isolated tetramer or octamer of a ACTH or ACTH peptide.
An isolated tetramer or octamer of a growth hormone or growth hormone peptide.
An isolated tetramer or octamer of a vasopressin or vasopressin peptide.
An isolated tetramer or octamer of an oxytocin or oxytocin peptide.
An isolated tetramer or octamer of a glucagon or glucagon peptide.
An isolated tetramer or octamer of a insulin or insulin peptide.
An isolated tetramer or octamer of a somatostatin or somatostatin peptide.
An isolated tetramer or octamer of a cholecystokinin or cholecystokinin peptide.
An isolated tetramer or octamer of a gastrin or gastrin peptide.
An isolated tetramer or octamer of a leptin or leptin peptide.
An isolated tetramer or octamer of an antibody binding site (eg, a scFv or Fab).
An isolated tetramer or octamer of a TCR binding site (eg, a scTCR).
An isolated tetramer or octamer of a TCR Vα/Vβ binding site.
An isolated tetramer or octamer of a TCR Vγ/Vδ binding site.
An isolated tetramer or octamer of an antibody single variable domain binding site.
An isolated tetramer or octamer of an FcAb binding site.

In an example of any of these tetramers or octamers, the domain or peptide is human. In an example of any of these tetramers or octamers, the tetramer or octamer comprises a NHR2 TD(eg, a human NHR2). In an example of any of these tetramers or octamers, the tetramer or octamer comprises a p53 TD (eg, a human p53 TD). In an example of any of these tetramers or octamers, the tetramer or octamer comprises a p63 TD (eg, a human p63 TD). In an example of any of these tetramers or octamers, the tetramer or octamer comprises a p73 TD (eg, a human p73 TD). In an example of any of these tetramers or octamers, the tetramer or octamer comprises a tetramer of TDs (eg, human NHR2 TDs), whereby the domains or peptides form a multimer of 4 or 8 domains or peptides.

In an example, the plurality is pure, eg, is not in mixture with multimers of said binding site or peptide wherein the multimers comprise more than one type of polypeptide monomer.
18. The multimer, tetramer or octamer of any preceding Aspect, wherein the mulitmer, tetramer or octamer is
   (a) soluble in aqueous solution (eg, an aqueous eukaryotic cell growth medium or buffer);
   (b) secretable from a eukaryotic cell; and/or
   (c) an expression product of a eukaryotic cell.

In an example the multimer, tetramer or octamer is secretable from a HEK293T (or other eukaryotic, mammalian, CHO or Cos) cell in stable form as indicated by a single band at the molecular weight expected for said multimer, tetramer or octamer on a PAGE gel using a sample of supernatant from such cells and detected using Western Blot.
19. A tetramer or octamer of
   (a) TCR V domains or TCR binding sites, wherein the tetramer or octamer is soluble in aqueous solution (eg, an aqueous eukaryotic cell growth medium or buffer);
   (b) antibody single variable domains, wherein the tetramer or octamer is soluble in aqueous solution (eg, an aqueous eukaryotic cell growth medium or buffer);
   (c) TCR V domains or TCR binding sites, wherein the tetramer or octamer is capable of being intracellularly and/or extracellularly expressed by HEK293 cells; or
   (d) antibody variable domains (eg, antibody single variable domains), wherein the tetramer or octamer is capable of being intracellularly and/or extracellularly expressed by HEK293 cells.

An example of the medium is SFMII growth medium supplemented with L-glutamine (eg, complete SFMII growth medium supplemented with 4 mM L-glutamine). In an example, the medium is serum-free HEK293 cell culture medium. In an example, the medium is serum-free CHO cell culture medium.

For example, a cell herein is a human cell, eg, a HEK293 cell (such as a HEK293T cell).
20. The multimer, tetramer or octamer of any preceding Aspect, wherein the tetramer or octameris bi-specific for antigen or pMHC binding.
21. The multimer, tetramer or octamer of any preceding Aspect, wherein the domains are identical.
22. The multimer, tetramer or octamer of any preceding Aspect, wherein the multimer, tetramer or octamer comprises eukaryotic cell glycosylation.

For example the glycosylation is CHO cell glycosylation. For example the glycosylation is HEK (eg, HEK293, such as HEK293T) cell glycosylation. For example the glycosylation is Cos cell glycosylation. For example the glycosylation is Picchia cell glycosylation. For example the glycosylation is Sacchaaromyces cell glycosylation.
23. The multimer, tetramer or octamer of Aspect 22, wherein the cell is a HEK293 cell.
24. A plurality of multimers, tetramers or octamers of any preceding Aspect.
25. A pharmaceutical composition comprising the multimer(s), tetramer(s) or octamer(s) of any preceding Aspect and a pharmaceutically acceptable carrier, diluent or excipient.
26. A cosmetic, foodstuff, beverage, cleaning product, detergent comprising the multimer(s), tetramer(s) or octamer(s) of any one of Aspects 1 to 24.
27. A said engineered (and optionally isolated) polypeptide or a monomer (optionally isolated) of a multimer, tetramer or octamer of any preceding Aspect.

The monomer is an engineered polypeptide as disclosed herein, comprising a said protein domain or peptide and further comprising a TD.

Optionally, the engineered polypeptide comprises (in N- to C-terminal direction) a variable domain (V1)-a constant domain (C) (eg, a CH1 or Fc)-optional linker-TD.
28. An engineered (and optionally isolated) engineered polypeptide (P1) which comprises (in N- to C-terminal direction):—
   (a) TCR V1-TCR C1-antibody C (eg, CH, CH1 (such as IgG CH1) or CL (such as Cλ or a Cκ))-optional linker-TD, wherein
      (i) V1 is a Vα and C1 is a Cα;
      (ii) V1 is a Vβ and C1 is a Cβ;
      (iii) V1 is a Vγ and C1 is a Cγ; or
      (iv) V1 is a Vδ and C1 is a Cδ;
         or
   (b) TCR V1-antibody C (eg, CH, CH1 (such as IgG CH1) or CL (such as Cλ or a Cκ))-optional linker-TD, wherein
      (i) V1 is a Vα;
      (ii) V1 is a Vβ;
      (iii) V1 is a Vγ; or
      (iv) V1 is a Vδ;
         or
   (c) antibody V1-antibody C (eg, CH, CH1 (such as IgG CH1) or CL (such as Cλ or a Cκ))-optional linker-TD, wherein
      (i) V1 is a VH; or
      (ii) V1 is a VL (eg, a Vλ or a Vκ);
         or
   (d) antibody V1-optional antibody C (eg, CH, CH1 (such as IgG CH1) or CL (such as Cλ or a Cκ))-antibody Fc (eg, an IgG Fc)-optional linker-TD, wherein
      (i) V1 is a VH; or
      (ii) V1 is a VL (eg, a Vλ or a Vκ);
         or
   (e) antibody V1-antibody CL (eg, a Cλ or a Cκ)-optional linker-TD, wherein
      (i) V1 is a VH; or
      (ii) V1 is a VL (eg, a Vλ or a Vκ);
      or
   (f) TCR V1-TCR C1-optional linker-TD, wherein
      (i) V1 is a Vα and C1 is a Cα;
      (ii) V1 is a Vβ and C1 is a Cβ;
      (iii) V1 is a Vγ and C1 is a Cγ; or
      (iv) V1 is a Vδ and C1 is a Cα.

In (a) or (b), in an example, the TCR V is comprised by an single chain TCR binding site (scTCR) that specifically binds to a pMHC, wherein the binding site comprises TCR V-linker-TCRV. In an example, the engineered polypeptide comprises (in N- to C-terminal direction) (i) V1-linker-V-optional C-optional linker-TD, or (ii) Va-linker-V1-optional C-optional linker-TD, wherein Vα is a TCR V domain and C is an antibody C domain (eg, a CH1 or CL) or a TCR C.

Preferably, the antibody C is CH1 (eg, IgG CH1).

In an example the multimer, tetramer or octamer has a size of no more than 155 kDa, eg, wherein said protein domain is an antibody variable domain comprising a CDR3 of at least 16, 17, 18, 19, 20, 21 or 22 amino acids, such as a Camelid CDR3 or bovine CDR3.

In an example, the multimer, tetramer or octamer comprises TCR binding sites and antibody binding sites. For example, each polypeptide comprises a TCR V (eg, comprised by a scTCR that specifically binds a pMHC) and an antibody V (eg, comprised by a scFv or paired with a second V domain comprised by a said second polypeptide to form a V/V paired binding site that specifically binds to an antigen). In an example, the pMHC comprises a RAS peptide. In an example the antigen is selected from the group consisting of PD-1, PD-L1 or any other antigen disclosed herein. For example, the antigen is PD-1 and the pMHC comprises a RAS peptide.
29. The polypeptide of Aspect 28, wherein the engineered polypeptide P1 is paired with a further polypeptide (P2), wherein P2 comprises (in N- to C-terminal direction):—
   (g) TCR V2-TCR C2-antibody CL (eg, a Cλ or a Cκ), wherein P1 is according to (a) recited in Aspect 28 and
      (i) V2 is a Vα and C2 is a Cα when P1 is according to (a)(ii);
      (ii) V2 is a Vβ and C2 is a Cβ when P1 is according to (a)(i);
      (iii) V2 is a Vγ and C2 is a Cγ when P1 is according to (a)(iv); or
      (iv) V2 is a Vδ and C2 is a Cδ when P1 is according to (a)(iii);
         or
   (h) TCR V2-antibody CL (eg, a Cλ or a Cκ), wherein P1 is according to (b) recited in Aspect 28 and
      (i) V2 is a Vα when P1 is according to (b)(ii);
      (ii) V2 is a Vβ when P1 is according to (b)(i);
      (iii) V2 is a Vγ when P1 is according to (b)(iv); or
      (iv) V2 is a Vδ when P1 is according to (b)(iiii);
         or
   (i) Antibody V2-CL (eg, a Cλ or a Cκ), wherein P1 is according to (c) recited in Aspect 28 and
      (i) V2 is a VH when P1 is according to (c)(ii); or
      (ii) V2 is a VL (eg, a Vλ or a Vκ) when P1 is according to (c)(i);
         or
   (j) Antibody V2-optional CL (eg, a Cλ or a Cκ), wherein P1 is according to (d) recited in Aspect 28 and
      (i) V2 is a VH when P1 is according to (d)(ii); or
      (ii) V2 is a VL (eg, a Vλ or a Vκ) when P1 is according to (d)(i);
         or
   (k) Antibody V2-CH1 (eg, IgG CH1), wherein P1 is according to (e) recited in Aspect 28 and
      (i) V2 is a VH when P1 is according to (e)(ii); or
      (ii) V2 is a VL (eg, a Vλ or a Vκ) when P1 is according to (e)(i);
         or
   (l) TCR V2-TCR C2, wherein P1 is according to (f) recited in Aspect 28 and
      (i) V2 is a Vα and C2 is a Cα when P1 is according to (f)(ii);
      (ii) V2 is a Vβ and C2 is a Cβ when P1 is according to (f)(i);
      (iii) V2 is a Vγ and C2 is a Cγ when P1 is according to (f)(iii); or
      (iv) V2 is a Vδ and C2 is a Cδ when P1 is according to (f)(iv).

Optionally, V1 and V2 form a paired variable domain binding site that is capable of specifically binding to an antigen or pMHC. In an example, V1 and V2 are variable domains of an antibody, eg, selected from the group consisting of REOPRO©; Abciximab; RITUXAN©; Rituximab; ZENAPAX®; Daclizumab; SIMULECT®; Basiliximab; SYNAGIS®; Palivizumab; REMICADE®; Infliximab; HERCEPTIN®; MYLOTARG®; Gemtuzumab; CAMPATH®; Alemtuzumab; ZEVALIN®; Ibritumomab; HUMIRA®; Adalimumab; XOLAIR®; Omalizumab; BEXXAR®; Tositumomab; RAPTIVA™; Efalizumab; ERBITUX®; Cetuximab; AVASTIN®; Bevacizumab; TYSABRI®; Natalizumab; ACTEMRA®; Tocilizumab;-VECTIBIX®; Panitumumab; LUCENTIS®; Ranibizumab; SOLIRIS®; Eculizumab; CIMZIA®; Certolizumab; SIMPONI®; Golimumab, ILARIS®; Canakinumab; STELARA®; Ustekinumab;-ARZERRA®; Ofatumumab; PROLIA®; Denosumab; NUMAX™; Motavizumab; ABTHRAX™; Raxibacumab; BENLYSTA®; Belimumab; YERVOY®; Ipilimumab; ADCETRIS®; Brentuximab Vedotin; PERJETA®; Pertuzumab; KADCYLA®; Ado-trastuzumab;-KEYTRUDA®, OPDIVO®, GAZYVA® and Obinutuzumab

In one embodiment, the antibody is AVASTIN®.

In one embodiment, the antibody is ACTEMRA®.

In one embodiment, the antibody is ERBITUX®.

In one embodiment, the antibody is LUCENTIS®.

In one embodiment, the antibody is sarilumab

In one embodiment, the antibody is dupilumab

In one embodiment, the antibody is alirocumab

In one embodiment, the antibody is evolocumab

In one embodiment, the antibody is pembrolizumab

In one embodiment, the antibody is nivolumab

In one embodiment, the antibody is ipilimumab

In one embodiment, the antibody is remicade

In one embodiment, the antibody is golimumab

In one embodiment, the antibody is ofatumumab

In one embodiment, the antibody is BENLYSTA®.

In one embodiment, the antibody is CAMPATH®.

In one embodiment, the antibody is rituximab

In one embodiment, the antibody is HERCEPTIN®.

In one embodiment, the antibody is durvalumab

In one embodiment, the antibody is daratumumab

In an example, V1 is capable (itself when a single variable domain, or when paired with V2) of specifically binding to an antigen selected from the group consisting of ABCF1; ACVR1; ACVR1B; ACVR2; ACVR2B; ACVRL1; ADORA2A; Aggrecan; AGR2; AICDA; AWI; AIG1; AKAP1; AKAP2; AIYIH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLRI (MDR15); BlyS; BM P1; BMP2; BMP3B (GDFIO); BMP4; BMP6; BM P8; BMPRIA; BMPRIB; BM PR2; BPAG1 (plectin); BRCA1; CI9orflO (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-id); CCL16 (HCC-4); CCL17 (TARC); CCL18 (PARC); CCL19 (M IP-3b); CCL2 (MCP-1); MCAF; CCL20 (MIP-3a); CCL21 (MIP-2); SLC; exodus-2; CCL22 (MDC/STC-1); CCL23 (M PIF-1); CCL24 (MPIF-2 I eotaxin-2); CCL25 (TECK); CCL26 (eotaxin-3); CCL27 (CTACK/ILC); CCL28; CCL3 (MIP-la); CCL4 (M IP-lb); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CM KBR5/ChemR13); CCR6 (CMKBR6/CKR-L3/STRL22/DRY6); CCR7 (CKR7/EBI1); CCR8 (CM KBR8/TERI/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p2IWapl/Cipl); CDKN1B (p27Kipl); CDKNIC; CDKN2A (pl6INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); CMKLR1; CMKOR1 (RDC1); CNR1; COL18A1; COL1A1; COL4A3; COL6A1; CR2; CRP; CSFI (M-CSF); CSF2 (GM-CSF); CSF3 (GCSF); CTLA4; CTNNB1 (b-catenin); CTSB (cathepsin B); CX3CL1 (SCYDi); CX3CR1 (V28); CXCL1 (GROl); CXCLIO (IP-10); CXCL11 (1-TAC/IP-9); CXCL12 (SDF1); CXCL13; CXCL14; CXCL16; CXCL2 (GR02); CXCL3 (GR03); CXCL5 (ENA-78 I LIX); CXCL6 (GCP-2); CXCL9 (MIG); CXCR3 (GPR9/CKR-L2); CXCR4; CXCR6 (TYMSTR ISTRL33 I Bonzo); CYB5; CYC1; CYSLTR1; DAB2IP; DES; DKFZp451J0118; DNCL1; DPP4; E2F1; ECGF1; EDG1; EFNAI; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FILl (EPSILON); FILl (ZETA); FU12584; FU25530; FLRTI (fibronectin); FLTI; FOS; FOSLI (FRA-I); FY (DARC); GABRP (GABAa); GAGEBI; GAGECI; GALNAC4S-65T; GATA3; GDF5; GFI1; GGT1; GM-CSF; GNAS1; GNRH1; GPR2 (CCRIO); GPR31; GPR44; GPR81 (FKSG80); GRCCIO (CIO); GRP; GSN (Gelsolin); GSTPI; HAVCR2; HDAC4; EDAC5; HDAC7A; HDAC9; HGF; HIF1A; HIP1;

histamine and histamine receptors; HLA-A; HLA-DRA; HM74; HMOX1; HUMCYT2A; ICEBERG; ICOSL; 1D2; IFN-a; IFNA1; IFNA2; IFNA4; IFNA5; IFNA6; IFNA7; IFNB1; IFNgamma; TFNW1; IGBP1; IGF1; IGF1R; IGF2; IGFBP2; IGFBP3; IGFBP6; IL-1; IL10; IL10RA; IL10RB; IL11; IL1IRA; IL-12; IL12A; IL12B; IL12RB1; IL12RB2; 1L13; IL13RA1; IL13RA2; 1L14; 1L15; IL15RA; IL16; 1L17; IL17B; IL17C; IL17R; 1L18; IL18BP; IL18R1; IL18RAP; 1L19; ILIA; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2 IL1RN; 1L2; 1L20; IL20RA; IL21R; 1L22; 1L22R; 1L22RA2; 1L23; 1L24; 1L25; 1L26; 1L27; 1L28A; 1L28B; 1L29; IL2RA; IL2RB; IL2RG; 1L3; 1L30; IL3RA; 1L4; IL4R; 1L5; IL5RA; 1L6; IL6R; IL6ST (glycoprotein 130); 1L7; TL7R; 1L8; IL8RA; IL8RB; IL8RB; 1L9; IL9R; ILK; INHA; INHBA; INSL3; INSL4; IRAKI; IRAK2; ITGA1; ITGA2; 1TGA3; ITGA6 (a6 integrin); ITGAV; ITGB3; ITGB4 (b 4 integrin); JAG1; JAK1; JAK3; JUN; K6HF; KAIl; KDR; MTLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMA5; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; M IB1; midkine; M IF; M IP-2; MK167 (Ki-67); MMP2; M MP9; MS4A1; MSMB; MT3 (metallothionectin-ifi); MTSS 1; M UC 1 (mucin); MYC; MYD88; NCK2; neurocan; NFKB 1; NFKB2; NGFB (NGF); NGFR; NgR-Lingo; NgR-Nogo66 (Nogo); NgR-p75; NgR-Troy; NM E1 (NM23A); NOX5; NPPB; NROB1; NROB2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PARTI; PATE; PAWR; PCA3; PCNA; PDGFA; PDGFB; PECAMI; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p2IRac2); RARB; RGS1; RGS13; RGS3; RNF110 (ZNF144); ROB02; S100A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINIA3; SERPINB5 (maspin); SERPINE1 (PAT-i); SERPINFI; SHBG; SLA2; SLC2A2; SLC33A1; SLC43A1; SLIT2; SPPl; SPRRIB (Spri); ST6GAL1; STABI; STAT6; STEAP; STEAP2; TB4R2; TBX21; TCPIO; TDGF1; TEK; TGFA; TGFB1; TGFB1I1; TGFB2; TGFB3; TGFBI; TGFBR1; TGFBR2; TGFBR3; TH1L; THBS1(thrombospondin-1); THBS2; THBS4; THPO; TIE (Tie-i); T]MP3; tissue factor; TLRIO; TLR2; TLR3; TLR4; TLR5; TLR6; TLR7; TLR8; TLR9; TNF; TNF-a; TNFAIP2 (B94); TNFAIP3; TNFRSF1 1A; TNFRSF1A; TNFRSF1B; TNFRSF21; TNFRSF5; TNFRSF6 (Fas); TNFRSF7; TNFRSF8; TNFRSF9; TNFSFIO (TRAIL); TNFSF1 1 (TRANCE); TNFSF12 (AP03L); TNFSF13 (April); TNFSF13B; TNFSF14 (HVEM-L); TNFSF1 5 (VEGI); TNFSF1 8; TNFSF4 (0X40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase lia); TP53; TPM 1; TPM2; TRADD; TRAF1; TRAF2; TRAF3; TRAF4; TRAF5; TRAF6; TREM 1; TREM2; TRPC6; TSLP; TWEAK; VEGF; VEGFB; VEGFC; versican; VHL C5; VLA-4; XCL1 (lymphotactin); XCL2 (SCM-lb); XCR1 (GPR5/CCXCR1); YY1; and ZFPM2.

For example in any configuration of the invention, the multimer, tetramer or octamer specifically binds to first and second epitopes or antigens, each of which is selected from the group consisting of EpCAM and CD3; CD19 and CD3; VEGF and VEGFR2; VEGF and EGFR; CD138 and CD20; CD138 and CD40; CD20 and CD3; CD38 and CD138; CD38 and CD20; CD38 and CD40; CD40 and CD20; CD19 and CD20; CD-8 and IL-6; PDL-1 and CTLA-4; CTLA-4 and BTN02; CSPGs and RGM A; IGF1 and IGF2; IGF1 and/or 2 and Erb2B; IL-12 and IL-18; IL-12 and TWEAK; IL-13 and ADAM8; IL-13 and CL25; IL-13 and IL-1beta; IL-13 and IL-25; IL-13 and IL-4; IL-13 and IL-5; IL-13 and IL-9; IL-13 and LHR agonist; IL-13 and MDC; IL-13 and MIF; IL-13 and PED2; IL-13 and SPRR2a; IL-13 and SPRR2b; IL-13 and TARC; IL-13 and TGF-beta; IL-1 alpha and IL-1 beta; MAG and RGM A; NgR and RGM A; NogoA and RGM A; OMGp and RGM A; RGM A and RGM B; Te38 and TNF alpha; TNF alpha and IL-12; TNF alpha and IL-12p40; TNF alpha and IL-13; TNF alpha and IL-15; TNF alpha and IL-17; TNF alpha and IL-18; TNF alpha and IL-1 beta; TNF alpha and IL-23; TNF alpha and M IF; TNF alpha and PEG2; TNF alpha and PGE4; TNF alpha and VEGF; and VEGFR and EGFR; TNF alpha and RANK ligand; TNF alpha and Blys; TNF alpha and GP130; TNF alpha and CD-22; and TNF alpha and CTLA-4

For example, the first epitope or antigen is selected from the group consisting of CD3; CD16; CD32; CD64; and CD89; and the second epitope or antigen is selected from the group consisting of EGFR; VEGF; IGF-1R; Her2; c-Met (aka HGF); HER3; CEA; CD33; CD79a; CD19; PSA; EpCAM; CD66; CD30; HAS; PSMA; GD2; ANG2; IL-4; IL-13; VEGFR2; andVEGFR3.

In an example, V1 is capable (itself when a single variable domain, or when paired with V2) of specifically binding to an antigen selected from the group consisting of human IL-IA, IL-Iβ, IL-1RN, IL-6, BLys, APRIL, activin A, TNF alpha, a BMP, BMP2, BMP7, BMP9, BMP10, GDF8, GDF11, RANKL, TRAIL, VEGFA, VEGFB or PGF; optionally the multimer comprises a cytokine amino acid sequence (eg, C-terminal to a TD), such as IL-2 or an IL2-peptide; and the multimer, tetramer or octamer is for treating or preventing a cancer in a human subject. In an example the said effector or protein domain is capable of binding to such an antigen; optionally the multimer comprises a cytokine amino acid sequence (eg, C-terminal to a TD), such as IL-2 or an IL2-peptide; and the multimer, tetramer or octamer is for treating or preventing a cancer in a human subject.
30. A multimer (eg, a dimer, trimer, tetramer or octamer) of P1 as defined in Aspect 28; or of P1 paired with P2 as defined in Aspect 29; or a plurality of said multimers, optionally wherein the multimer is according to any one of aspects 1 to 24.

Preferably the multimer is a tetramer of the engineered polypeptide and/or effector domain. In one example, the plurality of tetramers are not in mixture with monomers, dimers or trimers of the polypeptide,

In one example the multimer, eg, tetramer, is a capable of specifically binding to two different pMHC.
31. A nucleic acid encoding an engineered polypeptide or monomer of any one of Aspects 27 to 29, optionally wherein the nucleic acid is comprised by an expression vector for expressing the polypeptide.

In an example, the nucleic acid is a DNA, optionally operably connected to or comprising a promoter for expression of the polypeptide or monomer. In another example the nucleic acid is a RNA (eg, mRNA).
32. A eukaryotic host cell comprising the nucleic acid or vector of Aspect 31 for intracellular and/or secreted expression of the multimer, tetramer, octamer, engineered polypeptide or monomer of any one of Aspects 1 to 24.
33. Use of a nucleic acid or vector according to aspect 31 in a method of manufacture of protein multimers for producing intracellularly expressed and/or secreted multimers, wherein the method comprises expressing the multimers in and/or secreting the multimers from eukaryotic cells comprising the nucleic acid or vector.
34. Use of a nucleic acid or vector according to aspect 31 in a method of manufacture of protein multimers for producing glycosylated multimers in eukaryotic cells comprising the nucleic acid or vector.

Mammalian glycosylation of the invention is useful for producing medicines comprising or consisting of the multimers, tetramers or octamers of the invention for medical treatment or prevention of a disease or condition in a mammal, eg, a human. The invention thus provides such a method of use as well as the multimer, tetramer or octamer of the invention for this purpose. Similarly, intracellular and/or secreted expression in one or more host cells (or cell lines thereof) that are mammalian according to the invention is useful for producing such medicines. Particularly useful is such expression in HEK293, CHO or Cos cells as these are commonly used for production of medicaments.

In an embodiment, the invention comprises a detergent or personal healthcare product comprising a multimer, tetramer or octamer of the invention. In an embodiment, the invention comprises a foodstuff or beverage comprising a multimer, tetramer or octamer of the invention.

In an example, the multimer, monomer, dimer, trimer, tetramer, octamer, polypeptide, composition, mixture, use or method of the present invention is for an industrial or domestic use, or is used in a method for such use. For example, it is for or used in agriculture, oil or petroleum industry, food or drink industry, clothing industry, packaging industry, electronics industry, computer industry, environmental industry, chemical industry, aeorspace industry, automotive industry, biotechnology industry, medical industry, healthcare industry, dentistry industry, energy industry, consumer products industry, pharmaceutical industry, mining industry, cleaning industry, forestry industry, fishing industry, leisure industry, recycling industry, cosmetics industry, plastics industry, pulp or paper industry, textile industry, clothing industry, leather or suede or animal hide industry, tobacco industry or steel industry.
35. A mixture comprising (i) a eukaryotic cell line encoding an engineered polypeptide according to any one of Aspects 27 to 29; and (ii) multimers, tetramers or octamers as defined in any one of Aspects 1 to 24.
36. The mixture of Aspect 35, wherein the cell line is in a medium comprising secretion products of the cells, wherein the secretion products comprise said multimers, tetramers or octamers.
37. The multimer, tetramer or octamer of any one of aspects 1 to 24 for medical use.
38. A method producing
   (a) TCR V domain multimers, the method comprising the soluble and/or intracellular expression of TCR V-NHR2 TD or TCR V-p53 TD fusion proteins expressed in eukaryotic cells, the method optionally comprising isolating a plurality of said multimers;
   (b) antibody V domain multimers, the method comprising the soluble and/or intracellular expression of antibody V (eg, a single variable domain)-NHR2 TD or V-p53 TD fusion proteins expressed in eukaryotic cells, the method optionally comprising isolating a plurality of said multimers;
   (c) incretin peptide (eg, GLP-1, GIP or insulin) multimers, the method comprising the soluble and/or intracellular expression of incretin peptide-NHR2 TD or incretin peptide-p53 TD fusion proteins expressed in eukaryotic cells, such as HEK293T cells; the method optionally comprising isolating a plurality of said multimers; or
   (d) peptide hormone multimers, the method comprising the soluble and/or intracellular expression of peptide hormone-NHR2 TD or peptide hormone-p53 TD fusion proteins expressed in eukaryotic cells, such as HEK293T cells; the method optionally comprising isolating a plurality of said multimers. 39. Use of self-associating tetramerization domains (TD) (eg, NHR2 TD, p53 TD, p63 TD or p73 TD or a homologue or orthologue thereof) in a method of the manufacture of a tetramer of polypeptides, for producing a higher yield of tetramers versus monomer and/or dimer polypeptides.
40. Use of an engineered polypeptide in a method of the manufacture of a tetramer of a polypeptide comprising multiple copies of a protein domain or peptide, for producing a higher yield of tetramers versus monomer and/or dimer polypeptides, wherein the engineered polypeptide comprises one or more copies of said protein domain or peptide and further comprises a self-associating tetramerization domains (TD) (eg, NHR2 TD, p53 TD, p63 TD or p73 TD or a homologue or orthologue).
41. Use of self-associating tetramerization domains (TD) (eg, NHR2 TD, p53 TD, p63 TD or p73 TD or a homologue or orthologue thereof) in a method of the manufacture of a tetramer of a polypeptide, for producing a plurality of tetramers that are not in mixture with monomers, dimers or trimers.
42. Use of an engineered polypeptide in a method of the manufacture of a tetramer of a polypeptide comprising multiple copies of a protein domain or peptide, for producing a plurality of tetramers that are not in mixture with monomers, dimers or trimers, wherein the engineered polypeptide comprises one or more copies of said protein domain or peptide and further comprises a self-associating tetramerization domains (TD) (eg, NHR2 TD, p53 TD, p63 TD or p73 TD or a homologue or orthologue).
43. The use of any one of Aspects 39 to 42, wherein the yield of tetramers is at least 10, 20, 30, 40 or 50× the yield of monomers and/or dimers.
44. The use of any one of Aspects 39 to 43, wherein the ratio of tetramers produced:monomers and/or dimers produced in the method is at least 90:10 (eg, at least 95:5 or 98:2, or 99:1).
45. The use of any one of Aspects 39 to 44, wherein each monomer has a size of no more than 40, 35, 30, 25 or 20 kDa.
46. The use of any one of Aspects 39 to 45, wherein each tetramer has a size of no more than 200, 160, 155 or 150 kDa.
47. The use of any one of Aspects 39 to 46, wherein the method comprises expressing the tetramers from a eukaryotic cell line.
48. A multivalent heterodimeric soluble T cell receptor capable of binding pMHC complex comprising:
   (i) TCR extracellular domains;
   (ii) immunoglobulin constant domains; and
   (iii) an NHR2 multimerisation domain of ETO.
49. A multimeric immunoglobulin, comprising
   (i) immunoglobulin variable domains; and
   (ii) an NHR2 multimerisation domain of ETO.
50. A method for assembling a soluble, multimeric polypeptide, comprising:
   (a) providing a monomer of the said multimeric polypeptide, fused to an NHR2 domain of ETO;
   (b) causing multiple copies of said monomer to associate, thereby obtaining a multimeric, soluble polypeptide.

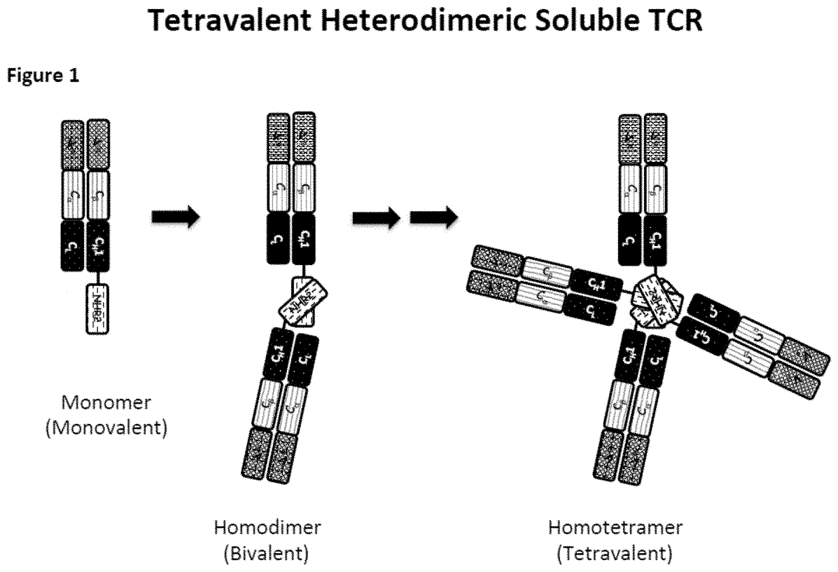

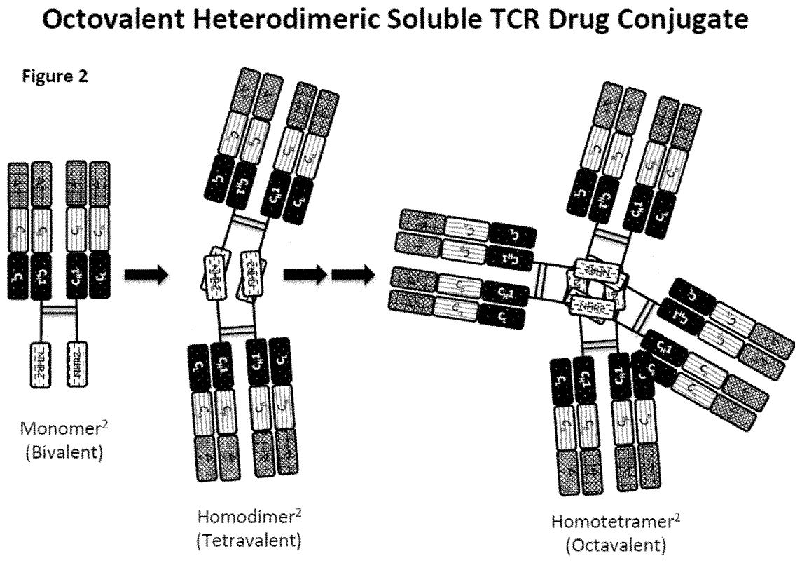

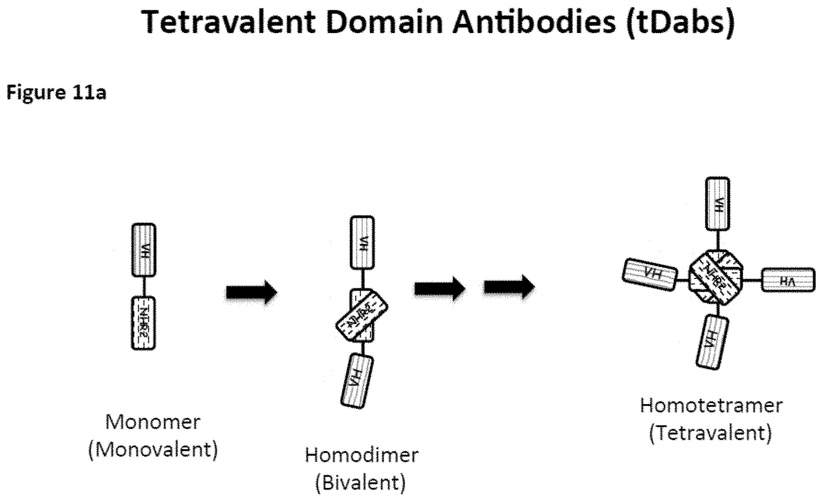

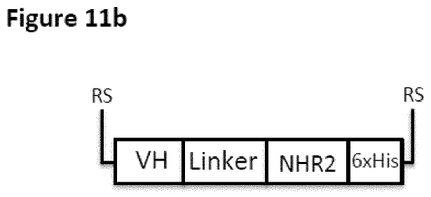

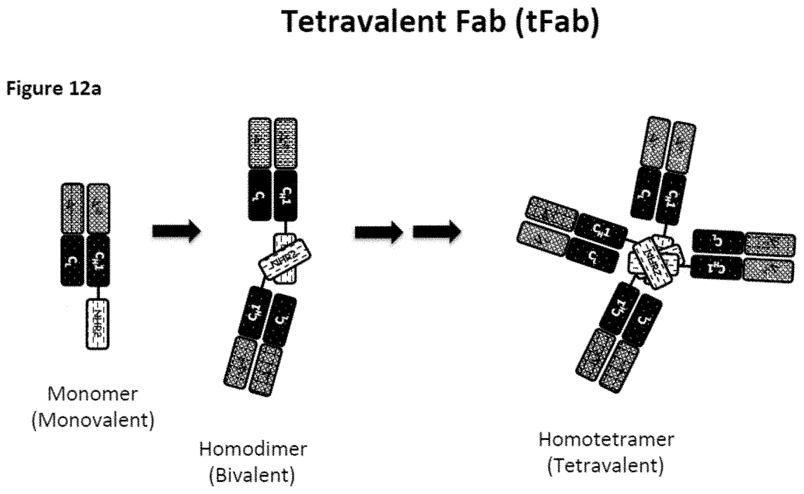

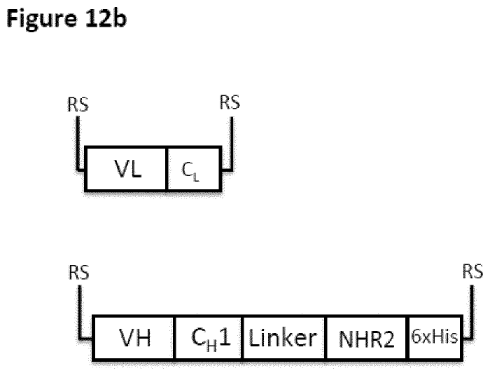

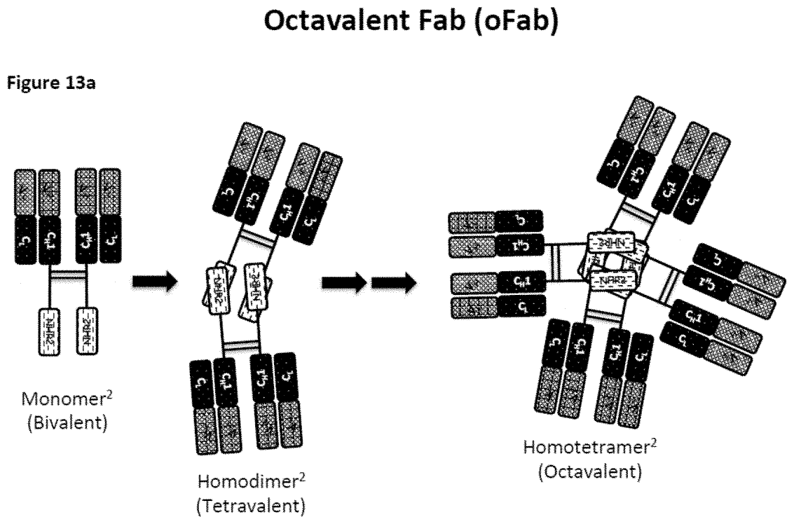

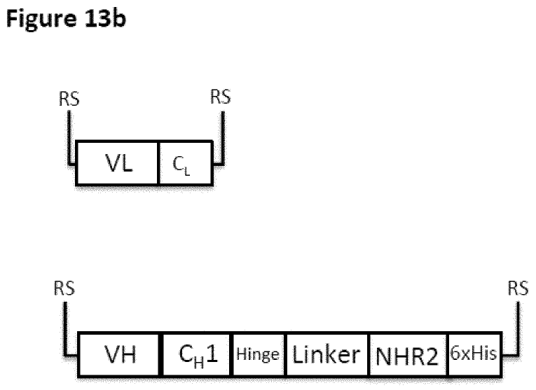

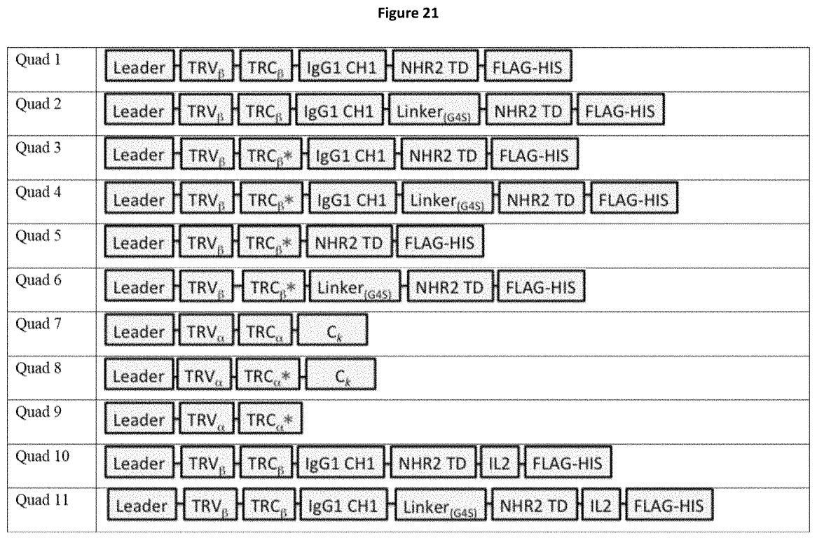

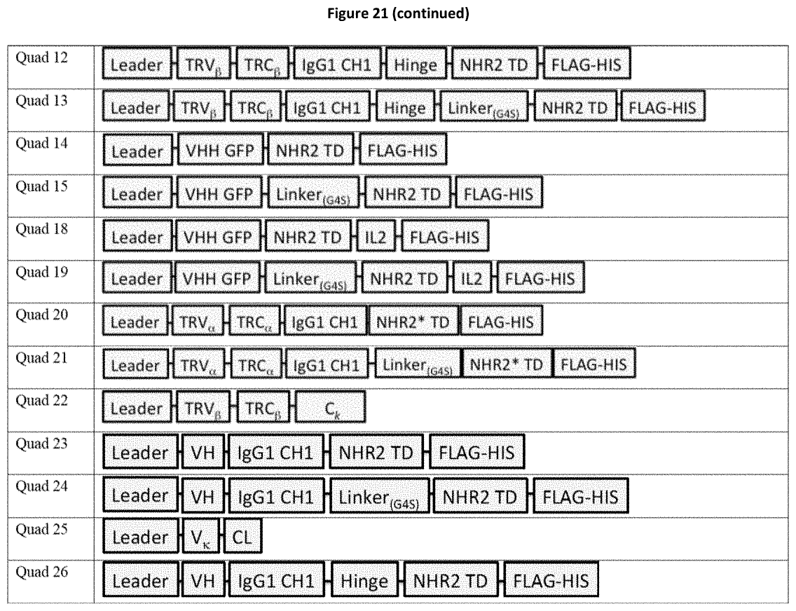

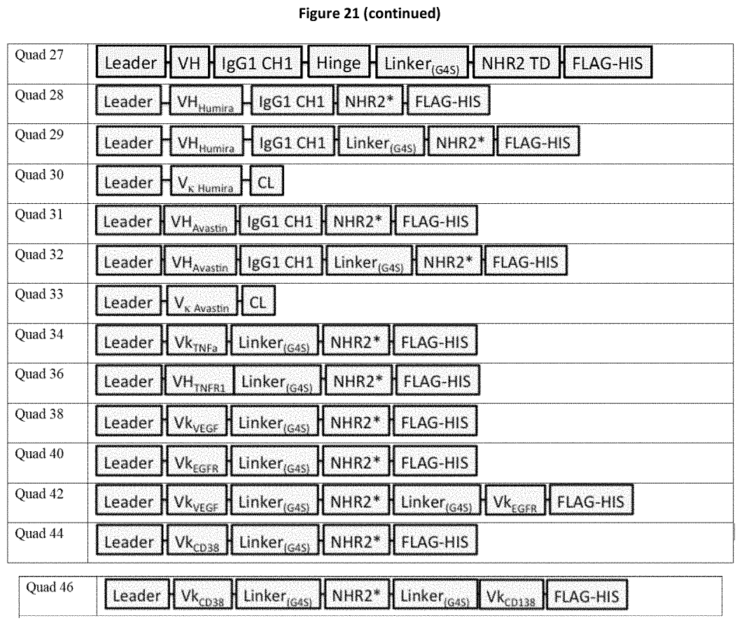

The invention further provides
(i) A monomer as shown in FIG. 1;
(ii) A homodimer as shown in FIG. 1;
(iii) A homotetramer as shown in FIG. 1;
(iv) A monomer² as shown in FIG. 2;
(v) A homodimer² as shown in FIG. 2;
(vi) A homotetramer² as shown in FIG. 2;
(vii) A monomer as shown in FIG. 11A;
(viii) A homodimer as shown in FIG. 11A;
(ix) A homotetramer as shown in FIG. 11A;
(x) A monomer as shown in FIG. 12A;
(xi) A homodimer as shown in FIG. 12A;
(xii) A homotetramer as shown in FIG. 12A;
(xiii) A monomer² as shown in FIG. 13A;
(xiv) A homodimer² as shown in FIG. 13A;
(xv) A homotetramer² as shown in FIG. 13A; or
(xvi) A multimeric protein comprising any one of (i) to (xv) (eg, any one of Quads 3, 4, 12, 13, 14, 15, 16 and 17) or a multimer of any protein shown in FIG. 21 (excluding any leader or tag).
(xvii) A plurality of multimers of (xvi); or
(xviii) A pharmaceutical composition comprising any one of (i) to (xvii) and a pharmaceutically acceptable carrier, diluent or excipient.

The invention also provides (i) A tetravalent or octavalent antibody V molecule;
(ii) A tetravalent or octavalent antibody Fab molecule;
(iii) A tetravalent or octavalent antibody dAb molecule;
(iv) A tetravalent or octavalent antibody scFv molecule;
(v) A tetravalent or octavalent antibody TCR V molecule; or
(vi) A tetravalent or octavalent antibody scFv molecule; Wherein the molecule is
(a) soluble in aqueous solution (eg, a solution or cell culture medium disclosed herein) and/or;
(b) capable of being intracellularly and/or extracellularly expressed by HEK293 cells.

The invention provides a claim multimer (eg, tetramer) of NHR2 or p53 (or another TD disclosed herein) fused at its N- and/or C-terminus to an amino acid sequence (eg, a peptide, protein domain or protein) that is not an NHR2 sequence. For example, sequence is selected from a TCR (eg, TCRα, TCRβ, Cα or Cβ), cytokine (eg, interleukin, eg, IL-2, IL-12, IL-12 and IFN), antibody fragments (eg, scFv, dAb or Fab) and a antibody domain (eg, V or C domain, eg, VH, VL, Vκ, Vλ, CH, CH1, CH2, CH3, hige, Cκ or Cλ domain). Optionally, the multimer is the molecule is
(i) soluble in aqueous solution (eg, a solution or cell culture medium disclosed herein) and/or;
(ii) capable of being intracellularly and/or extracellularly expressed by HEK293 cells.

The invention provides:—
(i) Use of NHR2 or p53 (or another TD disclosed herein) for the manufacture of a polypeptide for soluble expression of a multimer of the polypeptide from a cell, eg, a eukaryotic cell, eg, a mammalian, HEK293, CHO or Cos cell.
(ii) Use of NHR2 or p53 (or another TD disclosed herein) for the manufacture of a polypeptide for intracellular expression of a multimer of the polypeptide in a cell, eg, a eukaryotic cell, eg, a mammalian, HEK293, CHO or Cos cell.
(iii) A cell comprising an intracellular expression product, wherein the product comprises a multimer of a polypeptide comprising NHR2 or p53 (or another TD disclosed herein) fused at its N- and/or C-terminus to an amino acid sequence (eg, a peptide, protein domain or protein) that is not an NHR2 sequence.
(iv) Use of NHR2 as a promiscuous tetramerization domain for tetramerising peptides, protein domains, polypeptides or proteins in the manufacture of multimers that are intracellularly and/or solubly expressed from host cell.

Optionally, the amino acid is an amino acid sequence of a human peptide, protein domain or protein, eg, a TCR (eg, TCRα, TCRβ, Ca or Cβ), cytokine (eg, interleukin, eg, IL-2, IL-12, IL-12 and IFN), antibody fragments (eg, scFv, dAb or Fab), or an antibody domain (eg, V or C domain, eg, VH, VL, Vκ, Vλ, CH, CH1, CH2, CH3, hige, Cκ or Cλ domain).

Optionally, the or each polypeptide comprises a polypeptide selected from the group consisting of Quad 1-46 (ie, a polypeptide as shown in FIG. 21 but excluding any leader or tag sequence). Optionally, the invention provides a multimer (eg, a dimer, trimer, tetramer, pentamer, hexamer, septamer or octamer, preferably a tetramer or octamer) of a polypeptide selected from the group consisting of such Quad 1-46, eg, for medical or diagnostic use, eg, medical use for treating or preventing a disease or condition in a human or animal (eg, a human).

Optionally, the or each polypeptide comprises a polypeptide (excluding any leader or tag sequence) that is encoded by a nucleotide sequence selected from the group consisting of SEQ ID NOs: 13-50. Optionally, the or each polypeptide comprises a polypeptide (excluding any leader or tag sequence) that comprises an amino acid sequence selected from the group consisting of SEQ ID NOs: 83-115. Optionally, the invention provides a multimer (eg, a dimer, trimer, tetramer, pentamer, hexamer, septamer or octamer, preferably a tetramer or octamer) of such a polypeptide, eg, for medical or diagnostic use, eg, medical use for treating or preventing a disease or condition in a human or animal (eg, a human).

In an example, the TD is a TD comprised by any one of SEQ ID NOs: 1-9. In an example, the TD is a TD comprising SEQ ID NO: 10 or 126. In an example, the TD is encoded by SEQ ID NO: 124 or 125. In an example, the amino acid sequence of each TD is SEQ ID NO: 10 or 126 or is at least 80, 85, 90, 95, 96m 97, 98 or 99% identical to the SEQ ID NO: 10 or 126.

In an example, the TD is a TD comprising SEQ ID NO: 120 or 123. In an example, the TD is encoded by SEQ ID NO: 116 or 119. In an example, the amino acid sequence of each TD is SEQ ID NO: 120 or 123 or is at least 80, 85, 90, 95, 96m 97, 98 or 99% identical to the SEQ ID NO: 120 or 123.

Optionally, the domain or peptide comprised by the engineered polypeptide or monomer comprises an amino acid selected from SEQ ID NOs: 51-82.

High Purity Tetramers

As exemplified herein, the invention in one configuration is based on the surprising realization that tetramerization domains (TD), eg, p53 tetramerization domain (p53 TD), can be used to preferentially produce tetramers of effector domains over the production of lower-order structures such as dimers and monomers. This is particularly useful for secretion of tetramers is desirable yields from mammalian expression cell lines, such as CHO, HEK293 and Cos cell lines. The invention is also particularly useful for the production of tetramers no more than 200, 160, 155 or 150 kDa in size.

Thus, the invention provides the following Concepts:—

### Concepts

1. Use of a tetramerization domain (TD) (eg, p53 tetramerization domain (p53 TD) or NHR2 TD) or a homologue or orthologue thereof in a method of the manufacture of a tetramer of polypeptides, for producing a higher yield of tetramers versus monomer and/or dimer polypeptides.

The monomers and dimers comprise one or two copies of the TD, homologue or orthologue respectively

In an example, the TD, orthologue or homologue is a human domain

In an example, the yield of tetramers is higher than the yield of monomers; In an example, the yield of tetramers is higher than the yield of dimers; In an example, the yield of tetramers is higher than the yield of trimers; In an example, the yield of tetramers is higher than the yield of monomers and dimers; In an example, the yield of tetramers is higher than the yield of monomers and trimers; In an example, the yield of tetramers is higher than the yield of monomers, dimers and trimers

For example, the TD is the TD of p53 isoform 1. In an example, the TD comprises or consists of an amino acid sequence that is identical to positions 325 to 356 (or 319-360; or 321-359) of human p53 (eg, isoform 1). Optionally, the TD, orthologue or homologue comprises or consists of an amino acid sequence that is at least 80, 85, 90, 95, 96, 97, 98 or 99% identical to SEQ ID NO: 10, 126, 11 or 12. For example the sequence is identical to said selected sequence. Optionally, the TD, orthologue or homologue comprises or consists of an amino acid sequence that is at least 80, 85, 90, 95, 96, 97, 98 or 99% identical to SEQ ID NO: 120, 121, 122 or 123. For example the sequence is identical to said selected sequence.
2. The use of Concept 1, wherein first, second, third and fourth copies of an identical TD or homologue or orthologue thereof is used.
3. The use of any preceding Concept, wherein the TD is a NHR2, p53, p63 or p73 tetramerization domain.
   For example, the TD is a p53 TD. In an example, the TD is an orthologue or homologue of a p53 TD, eg, a human p53 TD.
4. The use of any preceding Concept, wherein the yield of tetramers is at least 10× the yield of monomers and/or dimers.

Optionally, the yield is at least 2× 3×, 4×, 5×, 6×, 7×, 8×, 9×, or 10× the yield of monomers and/or dimers. Optionally, the ratio of tetramers produced:monomers and/or dimers is at least 90:10, eg, at least 95:5; or 96:4; or 97:3; or 98:2; or 99:1. Optionally only tetramers are produced.

In an embodiment, each domain comprised by each monomer, dimer or tetramer is a human domain; and optionally the monomer, dimer or tetramer does not comprise non-human amino acid sequences or linkers.
5. The use of any preceding Concept, wherein the ratio of tetramers produced:monomers and/or dimers produced in the method is at least 90:10 (ie, 9× the amount of monomers; 9× the amount of dimers; or 9× the amount of the combination of monomers and dimers).
6. The use of Concept 4 or 5, wherein the yield or ratio is determinable or determined by obtaining a sample of the product of the tetramer manufacture method, using a protein separation technique on the sample to resolve tetramers, monomers and dimers and comparing the amount of tetramers with the amount of monomers and dimers.

Amounts of tetramers, monomers, dimers and trimers can be determined, for example, using Western Blot analysis of a gel described herein, eg, a native gel, ie, a gel not under denatured conditions, such as in the absence of SDS.
7. The use of Concept 4 or 5, wherein the yield or ratio is determinable or determined by
   (a) Obtaining a sample of the product of the tetramer manufacture method;
   (b) Carrying out polyacrylamide gel electrophoresis (PAGE) under non-reducing conditions to resolve the sample into a band corresponding to said tetramers and a band corresponding to said monomers and/or a band corresponding to said dimers; and
   (c) Comparing the tetramer band with the monomer and/or dimer band(s) to determine said yield or ratio, eg, by comparing the relative band intensities and/or band sizes.
8. The use of Concept 4 or 5, wherein the yield or ratio is determinable or determined by
   (d) Obtaining a sample of the product of the tetramer manufacture method;
   (e) Carrying out polyacrylamide gel electrophoresis (PAGE) under non-reducing conditions to resolve the sample into a band corresponding to said tetramers, eg, wherein the gel is under non-denatured conditions (eg, in the absence of sodium dodecylsuphate (SDS);
   (f) Determining that there is no band corresponding to said monomers and/or noband corresponding to said dimers.
9. The use of Concept 7 or 8, comprising
   (g) Obtaining a second sample of the product of the tetramer manufacture method;
   (h) Carrying out polyacrylamide gel electrophoresis (PAGE) under reducing conditions to resolve the second sample into a band corresponding to said monomers and/or a band corresponding to said dimers, eg, wherein the gel is under non-denatured conditions (eg, in the absence of sodium dodecylsuphate (SDS); and
   (i) Comparing the gel produced by step (h) with the gel of step (b) or (e) to determine the position of monomer and/or dimer band(s) in the gel of step (b) or where such gels would be expected in the gel of step (e).
10. The use of any preceding Concept, wherein each monomer has a size of no more than 40 kDa.

For example, the monomer has a size of no more than 35, 30, 25, 24, 23, 22, 21 or 20 kDa
11. The use of any preceding Concept, wherein each tetramer has a size of no more than 150 kDa.

For example, the tetramer has a size of no more than 80, 90, 100, 110, 120, 130 or 140 kDa.
12. The use of any preceding Concept, wherein the method comprises expressing the tetramers from a mammalian cell line, eg, a HEK293, CHO or Cos cell line.

For example, the cell line is a HEK293 (eg, HEK293T) cell line. In the alternative, the cell line is a yeast (eg, Saccharomyces or Pichia, eg, P pastoris) or bacterial cell line.
13. The use of any preceding Concept, wherein the method comprises secreting the tetramers from a mammalian cell line, eg, a HEK293, CHO or Cos cell line.

Thus, advantageously in an example, the use or tetramer is for expression from a mammalian cell line (eg, a HEK293, CHO or Cos cell line) or a eukaryotic cell line. This is useful for large-scale manufacture of the products, eg, tetramers, of the invention.

For example, the cell line is a HEK293 (eg, HEK293T) cell line. In the alternative, the cell line is a yeast (eg, Saccharomyces or Pichia, eg, P pastoris) or bacterial cell line.
14. The use of any preceding Concept, wherein each polypeptide or monomer comprises a said TD, homologue or orthologue and one or more protein effector domains, such as one or more antibody domains, eg, one or more antibody domains forming an antigen binding site.
15. The use of Concept 14, wherein the polypeptide comprises one or more of
   (i) an antibody single variable domain (dAb or VHH or Nanobody™) that is capable of specifically binding an antigen;
   (ii) an scFv that is capable of binding an antigen or an scTCR that is capable of binding pMHC;
   (iii) a Fab that is capable of binding an antigen; or
   (iv) a TCR variable domain or pMHC binding site.
16. The use of any preceding Concept, wherein each polypeptide or monomer comprises a said TD, homologue or orthologue and one or more incretin, insulin, GLP-1 or Exendin-4 domains.
17. The use of any preceding Concept, wherein each polypeptide or monomer comprises a said TD, homologue or orthologue; and first and second antigen binding sites.
18. The use of Concept 17, wherein each binding site is provided by
   (i) an antibody single variable domain (dAb or VHH or Nanobody™) that is capable of specifically binding an antigen;
   (ii) an scFv that is capable of binding an antigen or an scTCR that is capable of binding pMHC;
   (iii) a Fab that is capable of binding an antigen; or
   (iv) a TCR variable domain or pMHC binding site.
19. The use of Concept 18, wherein each binding site is provided by an antibody single variable domain.
20. The use of any one of Concepts 14 to 18, wherein the TD, homologue or orthologue is directly fused to said further domain(s).
21. The use of Concept 20, wherein each monomer or polypeptide comprises the TD, homologue or orthologue fused directly or via a peptide linker to the C-terminal of a said further domain.
22. A tetramer of polypeptides, wherein each polypeptide comprises
   (i) a tetramerization domain (TD) (eg, a p53 TD or a NHR2 TD) or a homologue or orthologue thereof;
   (ii) one or more protein effector domains; and
   (iii) optionally a linker linking (i) to (ii) (eg, linking the C-terminus of (ii) to the N-terminus of (i));
      wherein optionally each tetramer has a size of no more than 150 or 200 kDa.

For example, the tetramer has a size of no more than 80, 90, 100, 110, 120, 130 or 140 kDa. In an example, any multimer, dimer, trimer, tetramer or octamer herein has a size of at least 60 or 80 kDa; this may be useful for example to increase half-life in a human or animal subject administered with the multimer, dimer, trimer, tetramer or octamer (eg, to treat or prevent a disease or condition in the subject). Sizes in these ranges may be above the renal filtration size.

In an alternative, the invention provides a monomer, dimer, tetramer or octamer instead of a tetramer.
23. The tetramer of Concept 22, wherein each polypeptide comprises one or more of
   (i) an antibody single variable domain (dAb or VHH or Nanobody™) that is capable of specifically binding an antigen;
   (ii) an scFv that is capable of binding an antigen or an scTCR that is capable of binding pMHC;
   (iii) a Fab that is capable of binding an antigen; or
   (iv) a TCR variable domain or pMHC binding site.
24. The tetramer of Concept 22 or 23, wherein each polypeptide comprises a said TD, homologue or orthologue and one or more incretin, insulin, GLP-1 or Exendin-4 domains.
25. The tetramer of Concept 22 or 23, wherein each polypeptide comprises a said TD, homologue or orthologue; and first and second antigen binding sites.
26. The tetramer of Concept 25, wherein each binding site is provided by
   (i) an antibody single variable domain (dAb or VHH or Nanobody™) that is capable of specifically binding an antigen;
   (ii) an scFv that is capable of binding an antigen or an scTCR that is capable of binding pMHC;
   (iii) a Fab that is capable of binding an antigen; or
   (iv) a TCR variable domain or pMHC binding site.
27. The tetramer of Concept 26, wherein each binding site is provided by an antibody single variable domain.
28. The tetramer of any one of Concepts 22 to 27, wherein the TD, homologue or orthologue is directly fused to said effector domain(s).
29. The tetramer of any one of Concepts 22 to 27, wherein each polypeptide comprises the TD, homologue or orthologue fused directly or via a peptide linker to the C-terminal of a said effector domain.

In an embodiment, each polypeptide comprises only 2 (ie, only a first and a second, but not a third) effector domains or only 2 dAbs, VHH, scFvs, scTCRs, Fabs or antigen binding sites.
30. A pharmaceutical composition comprising a tetramer of any one of Concepts 22 to 29 and a pharmaceutically acceptable carrier, diluent or excipient.

Optionally the composition is comprised by a sterile medical container or device, eg, a syringe, vial, inhaler or injection device.
31. A cosmetic, foodstuff, beverage, cleaning product, detergent comprising a tetramer of any one of Concepts 22 to 29.
32. A mixture comprising a cell line (eg, a mammalian cell line, eg, a HEK293, CHO or Cos cell line) encoding a polypeptide as recited in any preceding Concept; and tetramers as defined in any preceding Concept.

Optionally, the mixture is comprised by a sterile container.
33. The mixture of Concept 32, wherein the cell line is in a medium comprising secretion products of the cells, wherein the secretion products comprise said tetramers.
34. The mixture of Concept 33, wherein the secretion products do not comprise monomers and/or dimers as defined in any one of Concepts 1 to 31.
35. The mixture of Concept 33, wherein the secretion products comprise said tetramers in an amount of at least 10× the amount of monomers and/or dimers.
36. The mixture of Concept 33, wherein the secretion products comprise said tetramers in a ratio of tetramers:monomers and/or dimers of at least 90:10.
37. A method for enhancing the yield of tetramers of an protein effector domain (eg, an antibody variable domain or binding site), the method comprising expressing from a cell line (eg, a mammalian cell, CHO, HEK293 or Cos cell line) tetramers of a polypeptide, wherein the polypeptide is as defined in any preceding Concept and comprises one or more effector domains; and optionally isolating said expressed tetramers.

The homologue, orthologue or equivalent has tetramerization function.

Homologue: A gene, nucleotide or protein sequence related to a second gene, nucleotide or protein sequence by descent from a common ancestral DNA or protein sequence. The term, homologue, may apply to the relationship between genes separated by the event of or to the relationship between genes separated by the event of genetic duplication.

Orthologue: Orthologues are genes, nucleotide or protein sequences in different species that evolved from a common ancestral gene, nucleotide or protein sequence by speciation. Normally, orthologues retain the same function in the course of evolution.

In an example, the TD, orthologue or homologue is a TD of any one of proteins 1 to 119 listed in Table 2. In an example, the orthologue or homologue is an orthologue or homologue of a TD of any one of proteins 1 to 119 listed in Table 2. In an embodiment, instead of the use of a p53 tetramerization domain (p53-TD) or a homologue or orthologue thereof, all aspects of the invention herein instead can be read to relate to the use or inclusion in a polypeptide, monomer, dimer, trimer or tetramer of a TD of any one of proteins 1 to 119 listed in Table 2 or a homologue or orthologue thereof. The TD may be a NHR2 (eg, a human NHR2) TD or an orthologue or homologue thereof. The TD may be a p63 (eg, a human p63) TD or an orthologue or homologue thereof. The TD may be a p73 (eg, a human p73) TD or an orthologue or homologue thereof. This may have one or more advantages as follows:—
secretion of tetramers from mammalian or other eukaryotic cells, eg, a mammalian cell disclosed herein such as CHO, HEK293 or Cos;
enhanced yield of secreted tetramers versus monomers;
enhanced yield of secreted tetramers versus dimers;
enhanced yield of secreted tetramers versus trimers;
enhanced yield of secreted tetramers versus monomers and dimers combined;
   enhanced yield of secreted tetramers versus monomers, dimers and trimers combined;
   enhanced affinity or avidity of antigen binding in tetramers comprising antigen binding sites;
   enhanced tetramer production and/or expression, wherein the tetramer is no more than 200 or no more than 160 or 150 kDa in size.
      In an embodiment, each polypeptide or monomer comprises one or more VH, VL or VH/VL binding sites of an antibody selected from REOPRO©; Abciximab; RITUXAN©; Rituximab; ZENAPAX©; Daclizumab; SIMULECT®; Basiliximab; SYNAGIS®; Palivizumab; REMICADE®; Infliximab; HERCEPTIN®; Trastuzumab; MYLOTARG®; Gemtuzumab; CAMPATH®; Alemtuzumab; ZEVALIN®; Ibritumomab; HUMIRA®; Adalimumab; XOLAIR®; Omalizumab; BEXXAR®; Tositumomab; RAPTIVA™; Efalizumab; ERBITUX®; Cetuximab; AVASTIN®; Bevacizumab; TYSABRI®; Natalizumab; ACTEMRA®; Tocilizumab;-VECTIBIX®; Panitumumab; LUCENTIS®; Ranibizumab; SOLIRIS®; Eculizumab; CIMZIA®; Certolizumab; SIMPONI®; Golimumab, ILARIS®; Canakinumab; STELARA®; Ustekinumab;-ARZERRA®; Ofatumumab; PROLIA®; Denosumab; NUMAX™; Motavizumab; ABTHRAX™; Raxibacumab; BENLYSTA®; Belimumab; YERVOY®; Ipilimumab; ADCETRIS®; Brentuximab Vedotin; PERJETA®; Pertuzumab; KADCYLA®; Ado-trastuzumab;-KEYTRUDA®, OPDIVO®, GAZYVA® and Obinutuzumab. In an alternative, (eg, for treating or preventing a cancer in a human) each polypeptide or monomer comprise one or more VH, VL or VH/VL binding sites of an antibody selected from ipilimumab (or YERVOY™), tremelimumab, nivolumab (or OPDIVO™), pembrolizumab (or KEYTRUDA™), pidilizumab, BMS-936559, durvalumab and atezolizumab.

In an example, the tetramer comprises 4 copies of the antigen binding site of a first antibody selected from the group consisting of ipilimumab (or YERVOY™), tremelimumab, nivolumab (or OPDIVO™), pembrolizumab (or KEYTRUDA™), pidilizumab, BMS-936559, durvalumab and atezolizumab and optionally 4 copies of the antigen binding site of a second antibody selected from said group, wherein the first and second antibodies are different. For example, the first antibody is ipilimumab (or YERVOY™) and optionally the second antibody is nivolumab (or OPDIVO™) or pembrolizumab (or KEYTRUDA™). This is useful for treating or preventing a cancer in a human.

In an example, the tetramer comprises 4 copies of the antigen binding site of AVASTIN®. In an example, the tetramer comprises 4 copies of the antigen binding site of HUMIRA®. In an example, the tetramer comprises 4 copies of the antigen binding site of ERBITUX®. In an example, the tetramer comprises 4 copies of the antigen binding site of ACTEMRA®. In an example, the tetramer comprises 4 copies of the antigen binding site of sarilumab. In an example, the tetramer comprises 4 copies of the antigen binding site of dupilumab. In an example, the tetramer comprises 4 copies of the antigen binding site of alirocumab or evolocumab. In an example, the tetramer comprises 4 copies of the antigen binding site of dupilumab. In an example, the tetramer comprises 4 copies of the antigen binding site of REMICADE®. In an example, the tetramer comprises 4 copies of the antigen binding site of LUCENTIS®. In an example, the tetramer comprises 4 copies of the antigen binding site of EYLEA®. Such tetramers are useful for administering to a human to treat or prevent a cancer. Such tetramers are useful for administering to a human to treat or prevent an ocular condition (eg, wet AMD or diabetic retinopathy, eg, when the binding site is an AVASTIN®, LUCENTIS® or EYLEA® site). Such tetramers are useful for administering to a human to treat or prevent angiogenesis.

In an example, the tetramer comprises 4 copies of insulin. In an example, the tetramer comprises 4 copies of GLP-1. In an example, the tetramer comprises 4 copies of GIP. In an example, the tetramer comprises 4 copies of Exendin-4. In an example, the tetramer comprises 4 copies of insulin and 4 copies of GLP-1. In an example, the tetramer comprises 4 copies of insulin and 4 copies of GIP. In an example, the tetramer comprises 4 copies of insulin and 4 copies of Exendin-4. In an example, the tetramer comprises 4 copies of GLP-1 and 4 copies of Exendin-4. Such tetramers are useful for administering to a human to treat or prevent diabetes (eg, Type II diabetes) or obesity.

### Diseases and Conditions

The monomer or multimer (eg, dimer, trimer, tetramer or octamer) of the invention can be used in a method for administration to a human or animal subject to treat or prevent a disease or condition in the subject.

Optionally, the disease or condition is selected from
(a) A neurodegenerative disease or condition;
(b) A brain disease or condition;
(c) A CNS disease or condition;
(d) Memory loss or impairment;
(e) A heart or cardiovascular disease or condition, eg, heart attack, stroke or atrial fibrillation;
(f) A liver disease or condition;
(g) A kidney disease or condition, eg, chronic kidney disease (CKD);
(h) A pancreas disease or condition;
(i) A lung disease or condition, eg, cystic fibrosis or COPD;
j) A gastrointestinal disease or condition;
(k) A throat or oral cavity disease or condition;
(l) An ocular disease or condition;
(m) A genital disease or condition, eg, a vaginal, labial, penile or scrotal disease or condition;
(n) A sexually-transmissible disease or condition, eg, gonorrhea, HIV infection, syphilis or Chlamydia infection;
(o) An ear disease or condition;
(p) A skin disease or condition;
(q) A heart disease or condition;
(r) A nasal disease or condition
(s) A haematological disease or condition, eg, anaemia, eg, anaemia of chronic disease or cancer;
(t) A viral infection;
(u) A pathogenic bacterial infection;
(v) A cancer;
(w) An autoimmune disease or condition, eg, SLE;
(x) An inflammatory disease or condition, eg, rheumatoid arthritis, psoriasis, eczema, asthma, ulcerative colitis, colitis, Crohn's disease or IBD;
(y) Autism;
(z) ADHD;
(aa) Bipolar disorder;
(bb) ALS [Amyotrophic Lateral Sclerosis];
(cc) Osteoarthritis;
(dd) A congenital or development defect or condition;
(ee) Miscarriage;
(ff) A blood clotting condition;
(gg) Bronchitis;
(hh) Dry or wet AMD;
(ii) Neovascularisation (eg, of a tumour or in the eye);
(jj) Common cold;
(kk) Epilepsy;
(ll) Fibrosis, eg, liver or lung fibrosis;
(mm) A fungal disease or condition, eg, thrush;
(nn) A metabolic disease or condition, eg, obesity, anorexia, diabetes, Type I or Type II diabetes.
(oo) Ulcer(s), eg, gastric ulceration or skin ulceration;
(pp) Dry skin;
(qq) Sjogren's syndrome;
(rr) Cytokine storm;
(ss) Deafness, hearing loss or impairment;
(tt) Slow or fast metabolism (ie, slower or faster than average for the weight, sex and age of the subject);
(uu) Conception disorder, eg, infertility or low fertility;
(vv) Jaundice;
(ww) Skin rash;
(xx) Kawasaki Disease;
(yy) Lyme Disease;
(zz) An allergy, eg, a nut, grass, pollen, dust mite, cat or dog fur or dander allergy;
(aaa) Malaria, typhoid fever, tuberculosis or cholera;
(bbb) Depression;
(ccc) Mental retardation;
(ddd) Microcephaly;
(eee) Malnutrition;
(fff) Conjunctivitis;
(ggg) Pneumonia;
(hhh) Pulmonary embolism;
(iii) Pulmonary hypertension;
(jjj) A bone disorder;
(kkk) Sepsis or septic shock;
(lll) Sinusitus;
(mmm) Stress (eg, occupational stress);
(nnn) Thalassaemia, anaemia, von Willebrand Disease, or haemophilia;
(ooo) Shingles or cold sore;
(ppp) Menstruation;
(qqq) Low sperm count.

### Neurodegenerative or CNS Diseases or Conditions for Treatment or Prevention

In an example, the neurodegenerative or CNS disease or condition is selected from the group consisting of Alzheimer disease, geriopsychosis, Down syndrome, Parkinson's disease, Creutzfeldt-jakob disease, diabetic neuropathy, Parkinson syndrome, Huntington's disease, Machado-Joseph disease, amyotrophic lateral sclerosis, diabetic neuropathy, and Creutzfeldt Creutzfeldt-Jakob disease. For example, the disease is Alzheimer disease. For example, the disease is Parkinson syndrome.

In an example, wherein the method of the invention is practised on a human or animal subject for treating a CNS or neurodegenerative disease or condition, the method causes downregulation of Treg cells in the subject, thereby promoting entry of systemic monocyte-derived macrophages and/or Treg cells across the choroid plexus into the brain of the subject, whereby the disease or condition (eg, Alzheimer's disease) is treated, prevented or progression thereof is reduced. In an embodiment the method causes an increase of IFN-gamma in the CNS system (eg, in the brain and/or CSF) of the subject. In an example, the method restores nerve fibre and//or reduces the progression of nerve fibre damage. In an example, the method restores nerve myelin and//or reduces the progression of nerve myelin damage. In an example, the method of the invention treats or prevents a disease or condition disclosed in WO2015136541 and/or the method can be used with any method disclosed in WO2015136541 (the disclosure of this document is incorporated by reference herein in its entirety, eg, for providing disclosure of such methods, diseases, conditions and potential therapeutic agents that can be administered to the subject for effecting treatment and/or prevention of CNS and neurodegenerative diseases and conditions, eg, agents such as immune checkpoint inhibitors, eg, anti-PD-1, anti-PD-L1, anti-TIM3 or other antibodies disclosed therein).

### Cancers for Treatment or Prevention

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and paediatric tumours/cancers are also included.

Haematologic cancers are cancers of the blood or bone marrow. Examples of haematological (or haematogenous) cancers include leukaemias, including acute leukaemias (such as acute lymphocytic leukaemia, acute myelocytic leukaemia, acute myelogenous leukaemia and myeloblasts, promyeiocytic, myelomonocytic, monocytic and erythroleukaemia), chronic leukaemias (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel!carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, medu!loblastoma, Schwannoma craniopharyogioma, ependymoma, pineaioma, hemangioblastoma, acoustic neuroma, oligodendroglioma, menangioma, neuroblastoma, retinoblastoma and brain metastases).

Autoimmune Diseases for Treatment or Prevention
Acute Disseminated Encephalomyelitis (ADEM)
Acute necrotizing hemorrhagic leukoencephalitis
Addison's disease
Agammaglobulinemia
Alopecia areata
Amyloidosis
Ankylosing spondylitis
Anti-GBM/Anti-TBM nephritis
Antiphospholipid syndrome (APS)
Autoimmune angioedema
Autoimmune aplastic anemia
Autoimmune dysautonomia
Autoimmune hepatitis
Autoimmune hyperlipidemia
Autoimmune immunodeficiency
Autoimmune inner ear disease (AIED)
Autoimmune myocarditis
Autoimmune oophoritis
Autoimmune pancreatitis
Autoimmune retinopathy
Autoimmune thrombocytopenic purpura (ATP)
Autoimmune thyroid disease
Autoimmune urticaria
Axonal & neuronal neuropathies
Balo disease
Behcet's disease
Bullous pemphigoid
Cardiomyopathy
Castleman disease
Celiac disease
Chagas disease
Chronic fatigue syndrome
Chronic inflammatory demyelinating polyneuropathy (CIDP)
Chronic recurrent multifocal ostomyelitis (CRMO)
Churg-Strauss syndrome
Cicatricial pemphigoid/benign mucosal pemphigoid
Crohn's disease
Cogans syndrome
Cold agglutinin disease
Congenital heart block
Coxsackie myocarditis
CREST disease
Essential mixed cryoglobulinemia
Demyelinating neuropathies
Dermatitis herpetiformis
Dermatomyositis
Devic's disease (neuromyelitis optica)
Discoid lupus
Dressler's syndrome
Endometriosis
Eosinophilic esophagitis
Eosinophilic fasciitis
Erythema nodosum
Experimental allergic encephalomyelitis
Evans syndrome
Fibromyalgia
Fibrosing alveolitis
Giant cell arteritis (temporal arteritis)
Giant cell myocarditis
Glomerulonephritis
Goodpasture's syndrome
Granulomatosis with Polyangiitis (GPA) (formerly called Wegener's Granulomatosis)
Graves' disease
Guillain-Barre syndrome
Hashimoto's encephalitis
Hashimoto's thyroiditis
Hemolytic anemia
Henoch-Schonlein purpura
Herpes gestationis
Hypogammaglobulinemia
Idiopathic thrombocytopenic purpura (ITP)
IgA nephropathy
IgG4-related sclerosing disease
Immunoregulatory lipoproteins
Inclusion body myositis
Interstitial cystitis
Juvenile arthritis
Juvenile diabetes (Type 1 diabetes)
Juvenile myositis
Kawasaki syndrome
Lambert-Eaton syndrome
Leukocytoclastic vasculitis
Lichen planus
Lichen sclerosus
Ligneous conjunctivitis
Linear IgA disease (LAD)
Lupus (SLE)
Lyme disease, chronic
Meniere's disease
Microscopic polyangiitis
Mixed connective tissue disease (MCTD)
Mooren's ulcer
Mucha-Habermann disease
Multiple sclerosis
Myasthenia gravis
Myositis
Narcolepsy
Neuromyelitis optica (Devic's)
Neutropenia
Ocular cicatricial pemphigoid
Optic neuritis
Palindromic rheumatism
PANDAS (Pediatric Autoimmune Neuropsychiatric Disorders Associated with Streptococcus)
Paraneoplastic cerebellar degeneration
Paroxysmal nocturnal hemoglobinuria (PNH)
Parry Romberg syndrome
Parsonnage-Turner syndrome
Pars planitis (peripheral uveitis)
Pemphigus
Peripheral neuropathy
Perivenous encephalomyelitis
Pernicious anemia
POEMS syndrome
Polyarteritis nodosa
Type I, II, & III autoimmune polyglandular syndromes
Polymyalgia rheumatica
Polymyositis
Postmyocardial infarction syndrome
Postpericardiotomy syndrome
Progesterone dermatitis
Primary biliary cirrhosis
Primary sclerosing cholangitis
Psoriasis
Psoriatic arthritis
Idiopathic pulmonary fibrosis
Pyoderma gangrenosum
Pure red cell aplasia
Raynauds phenomenon
Reactive Arthritis
Reflex sympathetic dystrophy
Reiter's syndrome
Relapsing polychondritis
Restless legs syndrome
Retroperitoneal fibrosis
Rheumatic fever
Rheumatoid arthritis
Sarcoidosis
Schmidt syndrome
Scleritis
Scleroderma
Sjogren's syndrome
Sperm & testicular autoimmunity
Stiff person syndrome
Subacute bacterial endocarditis (SBE)
Susac's syndrome
Sympathetic ophthalmia
Takayasu's arteritis
Temporal arteritis/Giant cell arteritis
Thrombocytopenic purpura (TTP)
Tolosa-Hunt syndrome
Transverse myelitis
Type 1 diabetes
Ulcerative colitis
Undifferentiated connective tissue disease (UCTD)
Uveitis
Vasculitis
Vesiculobullous dermatosis
Vitiligo
Wegener's granulomatosis (now termed Granulomatosis with Polyangiitis (GPA).

Inflammatory Diseases for Treatment or Prevention
Alzheimer's
ankylosing spondylitis
arthritis (osteoarthritis, rheumatoid arthritis (RA), psoriatic arthritis)
asthma
atherosclerosis
Crohn's disease
colitis
dermatitis
diverticulitis
fibromyalgia
hepatitis
irritable bowel syndrome (IBS)
systemic lupus erythematous (SLE)
nephritis
Parkinson's disease
ulcerative colitis.

### Multivalent Soluble TCR

The present configuration relates to a multivalent soluble TCR protein. In one aspect, the invention relates to tetravalent and octavalent soluble TCR analogues. The TCR proteins of the invention are capable of self-assembly from monomers and are entirely of human origin. The proteins are multimers which comprise an ETO NHR2 multimerisation domain. The invention also relates to methods of constructing multimeric soluble TCRs, and methods of using such proteins.

Attempts to exploit alternative soluble TCR formats as therapeutic molecules have lagged far behind compared to the plethora of antibody formats. This is largely due to TCR, a heterodimeric transmembrane protein having the intrinsic problem of solubility once the extracellular TCR α/β chains are dissociated from their transmembrane and cytoplasmic domain. Secondly the intrinsic low affinity and avidity of these molecules for their cognate ligand at the target site has to a large degree hampered their development as a therapeutic molecule.

In order to overcome these drawbacks, the present configuration of the invention provides a TCR protein which is both multivalent and soluble. Multivalency increases the avidity of the TCR for cognate pMHC, and solubility allows the TCR to be used outside of a transmembrane environment. Accordingly, in a first aspect there is provided a multivalent heterodimeric soluble T cell receptor capable of binding pMHC complex comprising:
(i) TCR extracellular domains;
(ii) (ii) immunoglobulin constant domains; and
(iii) (iii) an NHR2 multimerisation domain of ETO.

The use of Ig constant domains provides the TCR extracellular domains with stability and solubility; multimerisation via the NHR2 domains provides multivalency and increased avidity. Advantageously, all of the domains are of human origin or conform to human protein sequences.

Using the Ig constant domain to stabilise and render soluble the TCR avoids the use of non-native disulphide bonds. Advantageously, therefore, the TCR of the invention does not comprise a non-native disulphide bond.

In one embodiment, said complex comprises a heavy chain and a light chain, and each light chain comprises a TCR Vα domain and an immunoglobulin C_{α} domain, and each heavy chain comprises a TCR Vβ domain and an immunoglobulin C_{H}1 domain.

In one embodiment, each light chain additionally comprises a TCR Cα domain, and each heavy chain additionally comprises a TCR Cβ domain.

In embodiments, the TCR and immunoglobulin domains can be separated by a flexible linker.

The NHR2 multimerisation domain is advantageously attached to the C-terminus of an immunoglobulin domain. Thus, each dimer of heavy and light chains will be attached to one multimerisation domain, so that the heavy chain-light chain dimers associate into multivalent oligomers.

In embodiments, the multimerisation domain and the immunoglobulin domain are separated by a flexible linker. In certain embodiments, this allows the multimerisation domain to multimerise without hindrance from the immunoglobulin domain(s).

In embodiments, the TCR protein may further comprise an immunoglobulin hinge domain. Hinge domains allow dimerization of heavy chain-light chain dimers; this allows further multimerisation of the TCR proteins. For example, a multimerisation domain which forms tetramers can, using an immunoglobulin hinge domain, form multimers up to octamers. Likewise, adimerising multimerisation domain can form tetramers in the presence of a hinge domain.

In embodiments, the TCR protein of the invention is tetravalent.

In embodiments, the TCR protein of the invention is octavalent

The present invention provides a soluble TCR where it is stably assembled in a tetravalent heterodimeric format using the nervy homology region 2 (NHR2) domain found in the ETO family protein in humans (Liu et al. 2006). The NHR2 domain is found naturally to form homotetramer, which is formed from pairing of two NHR2 homodimers. NHR2 linked operably to the extracellular TCRα or TCRβ chain will preferentially form tetravalent heterodimeric soluble TCR protein molecules sequentially self-assembled from a monomer followed by a homodimer (FIG. 1).

TCR proteins assembling into octamers can be created using the NHR2 domain, by employing immunoglobulin hinge domains.

In a further aspect, the TCR proteins of the invention can be coupled to biologically active polypeptides/effector molecules. Examples of such polypeptides can include immunologically active moieties such as cytokines, binding proteins such as antibodies or targeted polypeptides, and the like.

The invention further relates to methods for making tetravalent and octavalent heterodimeric soluble TCR, the DNA vectors encoding the proteins used for transfecting host cells of interests and the use of these novel highly sensitive multivalent soluble TCR protein molecules. Applications for use could include but not limited to, therapeutics, diagnostics and drug discovery.

In a further aspect, the invention provides a method for constructing multivalent immunoglobulin molecules in an efficient manner, without employing non-human construct components.

Accordingly, there is provided a multimeric immunoglobulin comprising
(i) immunoglobulin variable domains; and
(ii) an NHR2 multimerisation domain of ETO.

The immunoglobulin variable domains are preferably antibody variable domains. Such domains are fused to the ETO NHR2 multimerisation domain, which provides means for forming tetramers of the immunoglobulin variable domains.

The ETO NHR2 domain is more efficient than p53 and similar multimerisation domains in the production of immunoglobulin multimers, and permits the production of multimeric immunoglobulin molecules without the use of non-human components in the construct.

Also provided is a method for producing a multimeric immunoglobulin, comprising expressing immunoglobulin variable domains in fusion with an NHR2 domain of ETO, and allowing the variable domains to assemble into multimers.

Preferably, the immunoglobulin variable domains are attached to one or more immunoglobulin constant domains.

Advantageously, the immunoglobulin domains are antibody domains. For example, the variable domains can be V_{H} and V_{L} antibody domains. For example, the constant domains are antibody CH1 domains.

In one embodiment, the multimeric immunoglobulin molecules according to the invention, both TCR and non-TCR immunoglobulins, are produced for screening by phage display or another display technology. For example, therefore, the multivalent immunoglobulins are produced as fusions with a phage coat protein. For each immunoglobulin produced fused to a coat protein, other immunoglobulin molecules are produced without a coat protein, such that they can assemble on the phage surface as a result of NHR2 multimerisation.

The present configuration of the invention as detailed above relates to the nucleic acid sequences and methods for producing novel multivalent, for example tetravalent and octavalent, soluble proteins. In one aspect in particular the soluble protein is a TCR assembled into a tetravalent heterodimeric format that can bind four pMHC with high sensitivity, affinity and specificity. The soluble tetravalent heterodimeric TCR is a unique protein molecule composed from either the entire or in part the extracellular TCR α/β chains. The extracellular TCR α/β chains are linked to immunoglobulin C_{H} 1 and C_{L} (either Cκ or Cλ) domains. This linkage allows stable formation of heterodimeric TCR α/β. In the context of soluble tetravalent TCR the unique feature is the NHR2 homotetramer domain of the ETO family of proteins, which is operably linked to the C-terminus of C_{H}1 or the C-terminus of C_{L}. Linkage of the NHR2 domain to the heterodimeric α/βTCR in this manner allows it to self-assemble into a tetravalent format inside cells and be subsequently secreted into the supernatant as a soluble protein.

### TCR Extracellular Domains

TCR extracellular domains are composed of variable and constant regions. These domains are present in T-cell receptors in the same way as they are present in antibodies and other immunoglobulin domains. The TCR repertoire has extensive diversity created by the same gene rearrangement mechanisms used in antibody heavy and light chain genes (Tonegawa, S. (1988) Biosci. Rep. 8:3-26). Most of the diversity is generated at the junctions of variable (V) and joining (J) (or diversity, D) regions that encode the complementarity determining region 3 (CDR3) of the α and β chains (Davis and Bjorkman (1988) Nature 334:395-402). Databases of TCR genes are available, such as the IMGT LIGM database, and methods for cloning TCRs are known in the art—for example, see Bentley and Mariuzza (1996) Ann. Rev. Immunol. 14:563-590; Moysey et al., Anal Biochem. 2004 Mar. 15; 326(2):284-6; Walchli, et al. (2011) A Practical Approach to T-Cell Receptor Cloning and Expression. PLoS ONE 6(11): e27930.

### Immunoglobulin Variable Domains

Antibody variable domains are known in the art and available from a wide variety of sources. Databases of sequences of antibody variable domains exist, such as IMGT and Kabat, and variable domains can be produced by cloning and expression of natural sequences, or synthesis of artificial nucleic acids according to established techniques.

Methods for the construction of bacteriophage antibody display libraries and lambda phage expression libraries are well known in the art (McCafferty et al. (1990) Nature, 348: 552; Kang et al. (1991) Proc. Natl. Acad. Sci. USA., 88: 4363; Clackson et al. (1991) Nature, 352: 624; Lowman et al. (1991) Biochemistry, 30: 10832; Burton et al. (1991) Proc. Natl. Acad Sci USA., 88: 10134; Hoogenboom et al. (1991) Nucleic Acids Res., 19: 4133; Chang et al. (1991) J Immunol., 147: 3610; Breitling et al. (1991) Gene, 104: 147; Marks et al. (1991) supra; Barbas et al. (1992) supra; Hawkins and Winter (1992) J Immunol., 22: 867; Marks et al., 1992, J Bioi. Chem., 267: 16007; Lerner et al. (1992) Science, 258: 1313, incorporated herein by reference).

One particularly advantageous approach has been the use of scFv phage-libraries (Huston et al., 1988, Proc. Natl. Acad. Sci U.S.A., 85: 5879-5883; Chaudhary et al. (1990) Proc. Natl. Acad. Sci U.S.A., 87: 1066-1070; McCafferty et al. (1990) supra; Clackson et al. (1991) Nature, 352: 624; Marks et al. (1991) J Mol. Bioi., 222: 581; Chiswell et al. (1992) Trends Biotech., 10: 80; Marks et al. (1992) J Bioi. Chem., 267). Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference.

Such techniques can be adapted for the production of multimeric immunoglobulins by the fusion of NHR2 multimerisation domains to the antibody variable domains

### Immunoglobulin Constant Domains

An immunoglobulin constant domain, as referred to herein, is preferably an antibody constant domain. Constant domains do vary in sequence between antibody subtypes; preferably, the constant domains are IgG constant domains. Preferably, the constant domains are CH1 constant domains. Antibody constant domains are well known in the art and available from a number of sources and databases, including the IMGT and Kabat databases.

The fusion of antibody constant domains to immunoglobulin variable domains is also known in the art, for example in the construction of engineered Fab antibody fragments.

### Linkers

Flexible linkers can be used to connect TCR variable domain-Ig constant domain to the NHR2 multimerisation domain. This allows the TCR domains and the multimerisation domain to function without steric hindrance from each other or other molecules in the multimeric complex. Suitable linkers comprise, for example, glycine repeats, glycine-alanine repeats, Gly(4)Ser linkers, or flexible polypeptide linkers as set forth in Reddy Chichili et al., 2012 Protein Science 22:153-167.

### Immunoglobulin Hinge Domain

The Ig Hinge domain, herein preferably an antibody hinge domain, is the domain which links antibody constant regions in a natural antibody. This domain therefore provides for natural dimerization of molecules which include an antibody constant domain. It is present, for example, in a F(ab)2 antibody fragment, as well as whole antibodies such as IgG. This region comprises two natural interchain disulphide bonds, which connect the two CH1 constant domains together.

The multimerisation domain, in one embodiment, may be attached to the Ig constant domain or to the hinge domain. If a hinge domain is present, the multimerisation domain will form a TRC octamer, comprising four dimers of TCR variable-Ig Constant domains joined at a hinge region. Without the hinge region, the multimerisation domain will lead to the formation of a tetramer. Preferably, the multimerisation domain is attached to the C-terminal end of the constant domain or the hinge region.

### Biologically Active Molecule

One or more biologically active molecules or effector molecules (EM) can be attached to the multimer, eg, multimeric TCR proteins, of the present invention. Such molecules may be, for example, antibodies, especially antibodies which may assist in immune recognition and functioning of the TCR, such as anti-CD3 antibodies or antibody fragments.

In some aspects, the biologically active molecule can be a cytotoxic drug, toxin or a biologically active molecule such as a cytokine, as described in more detail below. Examples of biologically active molecules include chemokines such as MIP-Ib, cytokines such as IL-2, growth factors such as GM-CSF or G-CSF, toxins such as ricin, cytotoxic agents, such as doxorubicin or taxanes, labels including radioactive and fluorescent labels, and the like. For examples of biologically active molecules conjugatable to TCRs, see US20110071919.

In other aspects, the biologically active molecule is, for example, selected from the group consisting of: a group capable of binding to a molecule which extends the half-life of the polypeptide ligand in vivo, and a molecule which extends the half-life of the polypeptide ligand in vivo. Such a molecule can be, for instance, HSA or a cell matrix protein, and the group capable of binding to a molecule which extends the half-life of the TCR molecule in vivo is an antibody or antibody fragment specific for HSA or a cell matrix protein.

In one embodiment, the biologically active molecule is a binding molecule, for example an antibody fragment. 2, 3, 4, 5 or more antibody fragments may be joined together using suitable linkers. The specificities of any two or more of these antibody fragments may be the same or different; if they are the same, a multivalent binding structure will be formed, which has increased avidity for the target compared to univalent antibody fragments.

The biologically active molecule can moreover be an effector group, for example an antibody Fc region.

Attachments to the N or C terminus may be made prior to assembly of the TCR molecule or engineered polypeptide into multimers, or afterwards. Thus, the TCR fusion with an Ig Constant domain may be produced (synthetically, or by expression of nucleic acid) with an N or C terminal biologically active molecule already in place. In certain aspects, however, the addition to the N or C terminus takes place after the TCR fusion has been produced. For example, Fluorenylmethyloxycarbonyl chloride can be used to introduce the Fmoc protective group at the N-terminus of the TCR fusion. Fmoc binds to serum albumins including HSA with high affinity, and Fmoc-Trp or FMOC-Lys bind with an increased affinity. The peptide can be synthesised with the Fmoc protecting group left on, and then coupled with the scaffold through the cysteines. An alternative is the palmitoyl moiety which also binds HSA and has, for example been used in Liraglutide to extend the half-life of this GLP-1 analogue.

Alternatively, the TCR fusion can be modified at the N-terminus, for example with the amine- and sulfhydryl-reactive linker N-e-maleimidocaproyloxy)succinimide ester (EMCS). Via this linker the TCR can be linked to other polypeptides, for example an antibody Fc fragment.

### The NHR2 Domain

AML1/ETO is the fusion protein resulting from the t(8; 21) found in acute myeloid leukemia (AML) of the M2 subtype. AML1/ETO contains the N-terminal 177 amino acids of RUNX1 fused in frame with most (575 aa) of ETO. The nervy homology domain 2 of ETO is responsible for many of the biological activities associated with AML1/ETO, including oligomerisation and protein-protein interactions. This domain is characterised in detail in Liu et al (2006). See Genbank accession number NG_023272.2.

In one aspect of the present invention, the protein assembled into a soluble multivalent format is a TCR composed of either in part or all of the extracellular domains of the TCR α and β chains. The TCR α and β chains are stabilized by immunoglobulin C_{H}1 and C_{L} domains and could be arranged in the following configurations:

1. Vα-C_{L} and VβC_{H}1

2. Vα-C_{H}1 and Vβ-C_{L}

3. VαCα-C_{L} and VβCβ-C_{H}1

4. VαCαC_{H}1 and VβCβC_{L}

In one aspect of this invention, the extracellular TCR domains are linked to immunoglobulin C_{H}1 and C_{L} domains via an optional peptide linker (L) to promote protein flexibility and facilitate optimal protein folding.

1. Vα-(L)-C_{L} and Vβ-(L)-C_{H}1

2. Vα-(L)-C_{H}1 and Vβ-(L)-C_{L}

3. VαCα-(L)-C_{L} and VβCβ-(L)-C_{H}1

4. VαCα-(L)-C_{H}1 and VβCβ-(L)-C_{L}

In another aspect of this invention, a tetramerization domain (TD) such as NHR2 homotetramer domain is linked to the C-terminus of either the immunoglobulin C_{H}I or C_{L} domain, which is linked to the extracellular TCR α and β chain. The NHR2 domain could be optionally linked to C_{H}1 or C_{L} domain via a peptide linker. The resulting tetravalent heterodimeric TCR protein could be arranged in the following configurations where (L) is an optional peptide linker:

1. Vα-(L)-CL and Vβ-(L)-C_{H}1-(L)-TD

2. Vα-(L)-C_{H}1-(L)-TD and Vβ-(L)-C_{L}

3. VαCα-(L)-CL and VβCβ-(L)-C_{H}1-(L)-TD

4. VαCα-(L)-CH11-(L)-TD and VβCβ-(L)-C_{L}

5. Vα-(L)-CL-(L)-TD and Vβp-(L)-CH1

6. Vα-(L)-C_{H}1 and Vβ-(L)-C_{L}-(L)-TD

7. VαCα-(L)-CL-(L)-TD and VβCβ-(L)-CH1

8. VαCα-(L)-C_{H}1 and VβCβ-(L)-C_{L}-(L)-TD

The sensitivity of the soluble TCR for its cognate pMHC can be enhanced by increasing the avidity effect. This is achieved by increasing the number of antigen binding sites, facilitated by the tetramerization domain. This in turn also increases the molecular weight of the protein molecule compared to a monovalent soluble TCR and thus extends serum retention in circulation. Increasing the serum half-life also enhances the likelihood of these molecules interacting with their cognate target antigens.

The tetravalent heterodimeric soluble TCR protein molecule is capable of binding simultaneously to one, two, three or four pMHC displayed on a single cell or bind simultaneously to one, two, three or four different cells displaying its cognate pMHC.

TCR α and β chain sequences used in this invention could be from a known TCR specific for a particular pMHC or identified de novo by screening using techniques known in the art, such as phage display. Furthermore, TCR sequences are not limited to α and β chain in this invention but can also incorporate TCRδ and γ or ε chain and sequence variations thereof either directly cloned from human T cells or identified by directed evolution using recombinant DNA technology.

In another aspect to this invention, the tetravalent heterodimeric soluble TCR protein molecules are preferentially produced in mammalian cells for optimal production of soluble, stable and correctly folded protein molecules.

Multimer (eg, tetramer or octamer), or multivalent TCR according to the present invention may be expressed in cells, such as mammalian cells, using any suitable vector system. The pTT5 expression vector is one example of an expression system is used to express multivalent soluble TCR. The pTT5 expression system allows for high-level transient production of recombinant proteins in suspension-adapted HEK293 EBNA cells (Zhang et al. 2009). It contains origin of replication (oriP) that is recognized by the viral protein Epstein-Barr Nuclear Antigen 1 (EBNA-1), which together with the host cell replication factor mediates episomal replication of the DNA plasmid allowing enhanced expression of recombinant protein. Other suitable vector system for mammalian cell expression known in the art and commercially available can be used with this invention.

The tetravalent heterodimeric soluble TCR protein molecules or other multimers can be produced by transiently expressing genes from an expression vector.

In another embodiment, tetravalent heterodimeric soluble TCR protein molecules or other multimers can be produced from an engineered stable cell line. Cell lines can be engineered to produce the protein molecule using genome-engineering techniques known in the art where the gene(s) encoding for the protein molecule is integrated into the genome of the host cells either as a single copy or multiple copies. The site of DNA integration can be a defined location within the host genome or randomly integrated to yield maximum expression of the desired protein molecule. Genome engineering techniques could include but not limited to, homologous recombination, transposon mediated gene transfer such as PiggyBac transposon system, site specific recombinases including recombinase-mediated cassette exchange, endonuclease mediated gene targeting such as CRISPR/Cas9, TALENs, Zinc-finger nuclease, meganuclease and virus mediated gene transfer such as Lentivirus.

Also in another aspect to the invention, the tetravalent heterodimeric soluble TCR protein molecule or other multimer is produced by overexpression in the cytoplasm of E. coli as inclusion bodies and refolded in vitro after purification by affinity chromatography to produce functional protein molecules capable of correctly binding to its cognate pMHC or antigen.

In another aspect to the invention, expression of the tetravalent heterodimeric soluble TCR protein molecule or other multimer is not limited to mammalian or bacterial cells but can also be expressed and produced in insect cells, plant cells and lower eukaryotic cells such as yeast cells.

In another aspect to this invention, the heterodimeric soluble TCR molecule or other multimer is produced as an octavalent protein complex, eg, having up to eight binding sites for its cognate pMHC (FIG. 2). The multiple antigen binding sites allow this molecule to bind up to eight pMHC displayed on one cell or bind pMHC displayed on up to eight different cells thus creating a highly sensitive soluble TCR.

The heterodimeric soluble TCR portion of the molecule is made into a bivalent molecule by fusing the immunoglobulin hinge domain to the C-terminus of either the C_{H}I or C_{L} domain, which is linked itself either to TCR α or β chain. The hinge domain allows for the connection of two heavy chains giving a structure similar to IgG. To the C-terminus of the hinge domain, a tetramerization domain such as NHR2 is linked via an optional peptide linker. By joining immunoglobulin hinge to C- and N-terminus of Ig CH1 or CL domain and NHR2 domain respectively, it allows for the assembly of two NHR2 monomers referred to as monomer². In this conformation we predict the two NHR2 domains will most likely not form a homodimer by an antiparallel association due to structural constraints unless a long flexible linker is provided between the hinge and NHR2 domain. Linkage of the tetramerization and the hinge domain to the to the heterodimeric soluble TCR via immunoglobulin C_{H}1 or C_{L} domain allows for the stepwise self-assembly of an octavalent soluble TCR formed through a NHR2 homotetramer². The self-assembly of the octavalent soluble TCR is via NHR2 monomer² and homodimer² intermediate protein complexes (FIG. 2). The resulting octavalent heterodimeric soluble TCR protein molecule will have superior sensitivity for its cognate pMHC thus giving it a distinctive advantage of identifying unknown antigen or pMHC without having to affinity mature the TCR for its pMHC ligand much beyond affinities seen naturally. In particular it would be useful for identifying pMHC recognized by uncharacterized tumour-specific T cells and T cells involved in other diseases such as autoimmune diseases. A number of different configurations of the octavalent heterodimeric soluble TCR protein molecules can be produced. Some examples are shown below.

1. Vα-(L)-CL and Vβ-(L)-CH1-Hinge-(L)-TD

2. Vα-(L)-C_{H}1-Hinge-(L)-TD and Vβ-(L)-C_{L}

3. Vα-Cα-(L)-CL and Vβ-Cβ-(L)-C_{H}1-Hinge-(L)-TD

4. VαCα(L)-CH11-Hinge-(L)-TD and VβCβ-(L)-C_{L}

5. Vα-(L)-C_{L}-(L)-TD and Vβ-(L)-C_{H}1-Hinge

6. Vα-(L)-C_{H}1-Hinge and Vβ-(L)-C_{L}-(L)-TD

7. Vα-Cα-(L)-C_{L}-(L)-TD and Vβ-Cβ(L)-C_{H}1-Hinge

8. Vα-Cα-(L)-C_{H}1-Hinge and Vβ-Cβ-(L)-C_{L}-(L)-TD

In another aspect to this invention, the self-assembled multivalent protein preferentially tetravalent and octavalent heterodimeric soluble TCR are fused or conjugated to biologically active agent/effector molecule thus allowing these molecules to be guided to the desired cell population such as cancers cells and exert their therapeutic effect specifically. The tumour targeting ability of monoclonal antibodies to guide an effector molecule such as a cytotoxic drug, toxins or a biologically active molecule such as cytokines is well established (Perez et al. 2014; Young et al. 2014). In a similar manner the multivalent soluble TCR molecules outlined in this invention can also be fused with effector proteins and polypeptide or conjugated to cytotoxic agents. Examples of effector protein molecules suitable for use as a fusion protein with the multivalent protein complexes outlined in this invention include but are not limited to, IFNα, IFNβ, IFNγ, IL-2, IL-11, IL-13, granulocyte colony-stimulating factor [G-CSF], granulocyte-macrophage colony-stimulating factor [GM-CSF], and tumor necrosis factor [TNF]α, IL-7, IL-10, IL-12, IL-15, IL-21, CD40L, and TRAIL, the costimulatory ligand is B7.1 or B7.2, the chemokines DC-CK1, SDF-1, fractalkine, lyphotactin, IP-10, Mig, MCAF, MlP-1α, MIP-1/3, IL-8, NAP-2, PF-4, and RANTES or an active fragment thereof. Examples of toxic agent suitable for use as a fusion protein or conjugated to the multivalent protein complexes described in this invention include but not limited to, toxins such as diphtheria toxin, ricin, Pseudomonas exotoxin, cytotoxic drugs such as auristatin, maytansines, calicheamicin, anthracyclines, duocarmycins, pyrrolobenzodiazepines. The cytotoxic drug can be conjugated by a select linker, which is either non-cleavable or cleavable by protease or is acid-labile.

To eliminate heterogeneity and improve conjugate stability the cytotoxic drug can be conjugated in a site-specific manner. By engineering specific cysteine residues or using enzymatic conjugation through glycotransferases and transglutaminases can achieve this (Panowski et al. 2014).

In another aspect of the invention, the multivalent protein complex is covalently linked to molecules allowing detection, such as fluorescent, radioactive or electron transfer agents.

In another aspect of the invention, an effector molecule (EM) is fused to the multivalent protein complex via the C-terminus of the tetramerization domain such as NHR2 via an optional peptide linker. Fusion via the NHR2 domain can be arranged to produce multivalent protein complexes in a number of different configurations. Examples of some of the protein configurations that can be produced using the tetravalent heterodimeric soluble TCR is shown below:

1. Vα-(L)-CL and Vβ-(L)-C_{H}1-(L)-TD-(L)-EM

2. Vα-(L)-C_{H}1-(L)-TD-(L)-EM and Vβ-(L)-C_{L}

3. Vα-Cα-(L)-CL and Vβ-Cβ-(L)-C_{H}1-(L)-TD-(L)-EM

4. Vα-Cα-(L)-CH11-(L)-TD-(L)-EM and Vβ-Cβ-(L)-C_{L}

5. Vα-(L)-C_{L}-(L)-TD-(L)-EM and Vβ-(L)-C_{H}1

6. Vα-(L)-C_{H}1 and Vβ-(L)-C_{L}-(L)-TD-(L)-EM

7. Vα-Cα-(L)-C_{L}-(L)-TD-(L)-EM and Vβ-Cβ-(L)-C_{H}1

8. Vα-Cα-(L)-C_{H}1 and VP-Cβ-(L)-C_{L}-(L)-TD-(L)-EM

In another aspect of the invention, the effector molecule (EM) is fused to the multivalent protein complex at the C-terminus of either the immunoglobulin CH1 or CL1 domain via an optional peptide linker. Fusion of the EM via the immunoglobulin domain can be arranged to produce multivalent protein complexes in a number of different configurations. Examples of some of the protein configurations that can be produced using the tetravalent heterodimeric soluble TCR is shown below:

9. Vα-(L)-C_{L}-(L)-EM and Vβ-(L)-C_{H}1-(L)-TD

10. Vα-(L)-C_{H}1-(L)-TD and Vβ-(L)-C_{L}-(L)-EM

11. Vα-Cα-(L)-C_{L}-(L)-EM and Vαβ-Cβ-(L)-C_{H}1-(L)-TD

12. Vα-Cα-(L)-CH1-(L)-TD and Vβ-Cβ-(L)-C_{L}-(L)-EM

13. Vα-(L)-C_{L}-(L)-TD and Vβ-(L)-C_{H}1-(L)-EM

14. Vα-(L)-C_{H}1-(L)-EM and Vβ-(L)-C_{L}-(L)-TD

15. Vα-Cα-(L)-C_{L}-(L)-TD and VP-Cβ-(L)-C_{H}1-(L)-EM

16. Vα-Cα-(L)-CH1-(L)-EM and VP-Cβ-(L)-C_{L}-(L)-TD

In another aspect of the invention, effector molecules (EM) are fused to the multivalent protein complex at the C-terminus of either the immunoglobulin CH1 or CL1 domain and also the C-terminus of the tetramerization domain (e.g. NHR2) via an optional peptide linkers. This approach allows for the fusion of two effector molecules to be fused per TCR heterodimer complex. Fusion of the EM via the immunoglobulin domain and the tetramerization domain can be arranged to produce multivalent protein complexes in a number of different configurations. Examples of some of the protein configurations that can be produced using the tetravalent heterodimeric soluble TCR is shown below:

17. Vα-(L)-C_{L}-(L)-EM and Vβ-(L)-C_{H}1-(L)-TD-(L)-EM

18. Vα-(L)-CH1-(L)-TD-(L)-EM and Vβ-(L)-C_{L}-(L)-EM

19. Vα-Cα-(L)-C_{L}-(L)-EM and VβCβ-(L)-C_{H}1-(L)-TD-(L)-EM

20. Vα-Cα-(L)-CH1-(L)-TD-(L)-EM and Vβ-Cβ-(L)-C_{L}-(L)-EM

21. Vα-(L)-C_{L}-(L)-TD-(L)-EM and Vβ-(L)-C_{H}1-(L)-EM

22. Vα-(L)-C_{H}1-(L)-EM and Vβ-(L)-C_{L}-(L-)TD-(L)-EM

23. Vα-Cα-(L)-C_{L}-(L)-TD-(L)-EM and VP-Cβ-(L)-C_{H}1-(L)-EM

24. Vα-(L)-CH1-(L)-EM and Vβ-Cβ-(L)-CL-(L)-TD-(L)-EM

In another aspect of the invention, the multivalent protein complex is fused to a protein tag to facilitate purification. Purification tags are known in the art and they include, without being limited to, the following tags: His, GST, TEV, MBP, Strep, FLAG.

### Non-TCR Multimers

The present invention provides a unique method for assembling proteins in a soluble multivalent format with potential to bind multiple interacting domains or antigens. The protein can be a monomer, homodimer, heterodimer or oligomer preferentially involved either directly or indirectly in the immune system, or having the potential to regulate immune responses. Examples include, but not limited to, TCR, peptide MHC class I and class II, antibodies or antigen-binding portions thereof and binding proteins having alternative non-antibody protein scaffolds.

In another aspect of the invention, the interacting domains or antigens could be any cell surface expressed or secreted proteins, peptide-associated with MHC Class I or II or any proteins associated with pathogens including viral and bacterial proteins.

Non-TCR multimers may be multimers of antibodies or antibody fragments, such as dAbs of Fabs. Examples of dAbs and Fabs in accordance with the invention include the following:

Examples of multivalent Dabs

25. VH-(L)-NHR2

26. VL(λ or κ)-(L)-NHR2

27. VH-(L)-NHR2-(L)-EM

28. VL(λ or κ)-(L)-NHR2-(L)-EM

29. VH-CH1-(L)-NHR2

30. VL(λ or κ)-CL-(L)-NHR2

31. VH-CH1-(L)-NHR2-(L)-EM

32. VL(λ or κ)-CL-(L)-NHR2-(L)-EM

Examples of multivalent Fabs

33. VH-CH1-(L)-NHR2 and VL(, or K)-CL

34. VL(λ or κ)-CL-(L)-NHR2 and VH-CH1

35. VH-CH1-Hinge-(L)-NHR2 and VL(λ or κ)-CL

36. VL(λ or κ)-CL-Hinge-(L)-NHR2 and VH-CH1

37. VH-CH1-(L)-NHR2-(L)-EM and VL(λ or κ)-CL

38. VL(λ or κ)-CL-(L)-NHR2-(L)-EM and VH-CH1

39. VH-CH1-Hinge-(L)-NHR2-(L)-EM and VL(λ or κ)-CL

40. VL(λ or κ)-CL-Hinge-(L)-NHR2-(L)-EM and VH-CH1

41. VH-CH1-(L)-NHR2 and VL(λ or κ)-CL-(L)-EM

42. VL(λ or κ)-CL-(L)-NHR2 and VH-CH1-(L)-EM

43. VH-CH1-Hinge-(L)-NHR2 and VL(λ or κ)-CL-(L)-EM

44. VL(λ or κ)-CL-Hinge-(L)-NHR2 and VH-CH1-(L)-EM

In the examples above, (L) denotes an optional peptide linker, whilst EM denotes a biologically active agent or effector molecule such as toxins, drugs or cytokines, and including binding molecules such as antibodies, Fabs and ScFv.

The variable light chain can be either Vλ or Vκ.

In one aspect of the invention, the assembled tetramerized protein molecule in one example could be a human pMHC for the application in drug discovery using animal drug discovery platforms (e.g. mice, rats, rabbits, chicken). In such a context, the tetramerization domain is preferentially expressed and produced from genes originating from the animal species it is intended for. One example of such drug discovery applications would be the use of the tetramerized human pMHC as an antigen for immunization in rats for example. Once rats are immunized with pMHC the immune response is directed specifically towards the human pMHC and not the tetramerization domain of the protein complex.

Multivalent antibodies can be produced, for example using single domain antibody sequences, fused to the NHR2 multimerisation domain.

In a related aspect to the invention, the tetravalent protein can be a peptide used as a probe for molecular imaging of tumour antigens. The multivalent binding of such a probe will have distinctive advantage over monovalent molecular probes as it will have enhanced affinity, avidity and retention time in vivo and this in turn will enhance in vivo tumour targeting.

The multimerisation domain is the NHR2 domain set forth above. Preferably, polypeptides are stabilized and/or rendered soluble by the use of Ig constant domains fused to the polypeptides, such that the fusions provide tetramers of polypeptides. Ig hinge domains can be used to provide octamers.

### Uses of Multimers

Multimeric TCR proteins according to the invention are useful in any application in which soluble TCR proteins are indicated. Particular advantages of the TCR proteins of the invention include increased avidity for the selected target, and/or the ability to bind a plurality of targets.

Thus, in one aspect, the multivalent heterodimeric soluble TCR protein molecules of the invention can be used for selectively inhibiting immune responses, for example suppression of an autoimmune response. The multivalent, for example tetravalent, nature of these soluble protein molecules gives it exquisite sensitivity and binding affinity to compete antigen-specific interactions between T cells and antigen presenting cells. This kind of neutralization effect can be therapeutically beneficial in autoimmune diseases such as rheumatoid arthritis, systemic lupus erythematosus, psoriasis, inflammatory bowel diseases, graves disease, vasculitis and type 1 diabetes.

Similarly, the tetravalent heterodimeric soluble TCR protein molecules can be used to prevent tissue transplant rejection by selectively suppressing T cell recognition of specific transplantation antigen and self antigens binding to target molecule and thus inhibiting cell-to-cell interaction.

In another aspect of the invention, the tetravalent heterodimeric soluble TCR protein molecules can be used in clinical studies such as toxicity, infectious disease studies, neurological studies, behavior and cognition studies, reproduction, genetics and xenotransplantation studies.

The tetravalent heterodimeric soluble TCR protein molecules with enhanced sensitivity for cognate pMHC can be used for the purpose of diagnostics using biological samples obtained directly from human patients. The enhanced sensitivity of the tetravalent heterodimeric soluble TCRs allows detection of potential disease-associated peptides displayed on MHC, which are naturally found to be expressed at low density. These molecules can also be used for patient stratification for enrolling patient onto relevant clinical trials.

In another aspect of the invention, octavalent heterodimeric soluble TCR protein molecules can be used in pharmaceutical preparations for the treatment of various diseases.

In another related aspect to this invention, octavalent heterodimeric soluble TCR protein molecules can be used as a probe for tumour molecular imaging or prepared as a therapeutic protein.

### EXAMPLES

### Example 1: Generation of Tetravalent and Octavalent Soluble Heterodimeric NY-ESO-1 TCR Molecules

This example demonstrates a method for generating tetravalent and octavalent soluble heterodimeric TCR molecules referred to as ts-NY-ESO-1 TCR and os-NY-ESO-1 TCR respectively. These formats overcome the problems associated with solubility and avidity for cognate ligand at the target site.

To exemplify ts-NY-ESO-1 TCR and os-NY-ESO-1 TCR as stable and soluble molecules, TCR αβ variable sequences with high affinity for NY-ESO-1 together with immunoglobulin constant domains and the NHR2 tetramerization domain are used in this example.

The high-affinity NY-ESO TCR αβ chains (composing of TCR Vα-Cα and Vβ-Cβ respectively) specific for SLLMWITQC (SEQ ID NO: 141)-HLA-A*0201 used in this example is as reported in WO 2005/113595 A2 with the inclusion of a signal peptide sequence (MGWSCIILFLVATATGVHS; SEQ ID NO: 142). To aid protein purification, a histidine tag was incorporated to the C-terminus of NHR2 domain.

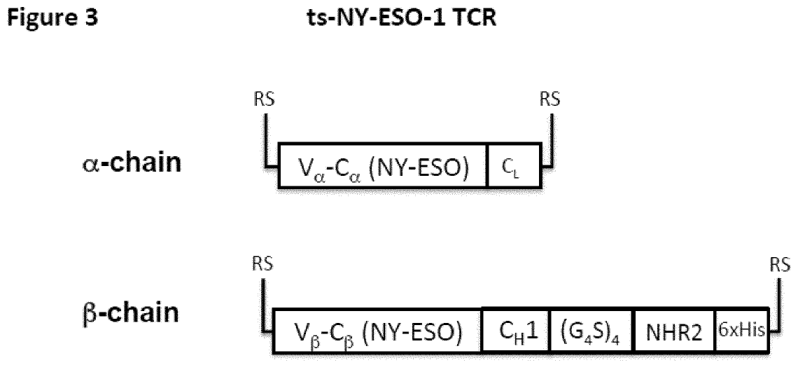

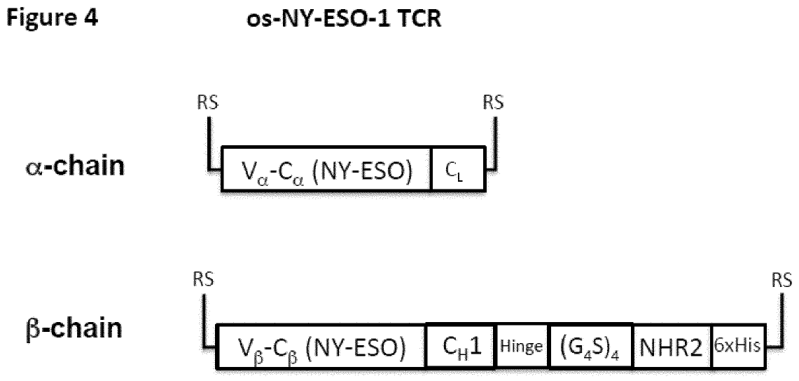

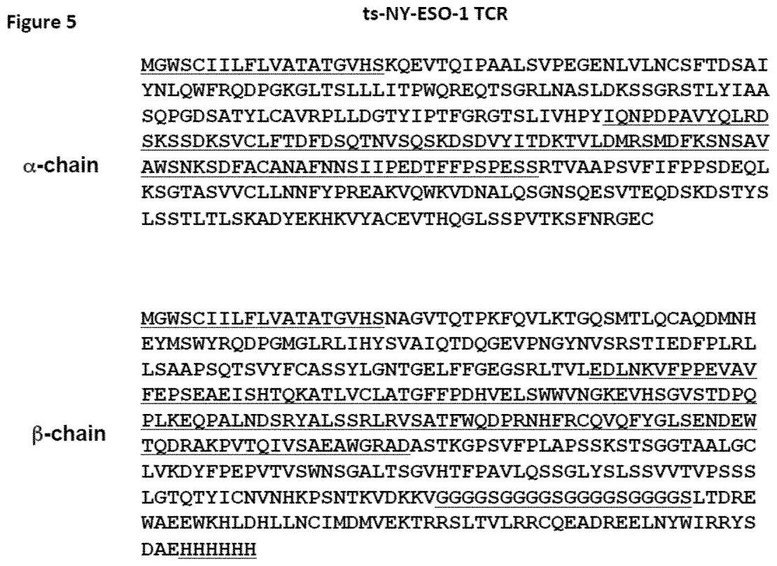

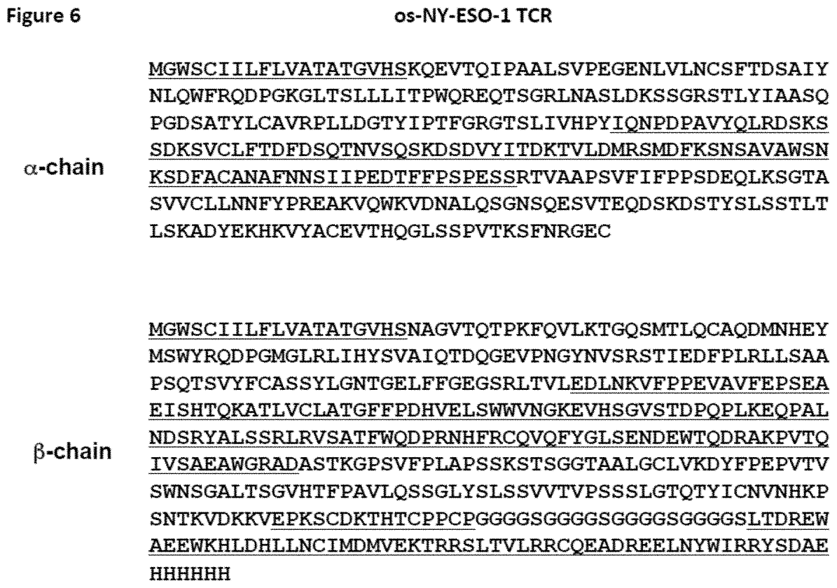

DNA constructs encoding components of ts-NY-ESO-1 TCR and os-NY-ESO-1 TCR are synthetically constructed as a two-vector system to allow for their soluble expression and functional assembly in mammalian cells. A schematic representation of the two assembled TCR chains (a and (3 chains) whose DNA sequences are synthesized for cloning into the expression vector are shown in FIGS. 3 and 4 and their amino acid sequences are shown in FIGS. 5 and 6.

The pTT5 vector system allows for high-level transient production of recombinant proteins in suspension-adapted HEK293 EBNA cells (Zhang et al., 2009). It contains origin of replication (oriP) that is recognized by the viral protein Epstein-Barr Nuclear Antigen 1 (EBNA-1), which together with the host cell replication factor mediates episomal replication of the DNA plasmid allowing enhanced expression of recombinant protein. Therefore the pTT5 expression vector is selected for cloning the components for the ts-NY-ESO-1 TCR and os-NY-ESO-1 TCR molecules.

Synthesized DNA fragments containing the TCR αβ chains are digested with restriction enzymes at the restriction sites (RS) (FastDigest, Fermantas) and the DNA separated out on a 1% agarose gel. The correct size DNA fragments is excised and the DNA purified using Qiagen gel extraction kit. The pTT5 vector was also digested with the same restriction enzymes and the linearized plasmid DNA is purified from excised agarose gel. The digested TCR αβ chains is cloned into the digested pTT5 vector to give four expression vectors (pTT5-ts-NY-ESO-1-TCRα, pTT5-ts-ESO-1-TCRβ, pTT5-os-NY-ESO-1-TCRα and pTT5-os-ESO-1-TCRβ).

Expression of Tetravalent and Octavalent Soluble NY-ESO TCR

Functional expression of ts-NY-ESO-1 TCR and os-NY-ESO-1 TCR is carried out in suspension-adapted HEK293 EBNA cells. HEK293-EBNA cells are cultured in serum-free Dulbecco's Modified Eagle Medium (DMEM, high glucose (4.5 g/L) with 2 mM L-glutamine) at 37° C., 5% CO₂ and 95% humidity.

For each transfection, HEK293-EBNA cells (3×10⁷ cells) are freshly seeded into 250 mL Erlenmeyer shaker Flask (Corning) from −60% confluent cells. Transfections are carried out using FreeStyle MAX cationic lipid base reagent (Life Technologies) according to the supplier's guidelines. For expression of ts-NY-ESO-1 TCR, 37.5 μg of total plasmid DNA (18.75 μg plasmid DNA each of pTT5-ts-NY-ESO-1-TCRα and pTT5-ts-ESO-1-TCRβ vectors are used or varying amounts of the two expression plasmids) are used per transfection. Similarly for expression of os-NY-ESO-1 TCR, 18.75 g plasmid DNA each of pTT5-os-NY-ESO-1-TCRα and pTT5-os-ESO-1-TCRβ is are used for transfection. Following transfection, cells were recovered in fresh medium and cultivated at 37° C. with 5% CO₂ in an orbital shaker at 110 rpm for between 4-8 days. Smaller scale transfections are done similarly in 6 well or 24 well plates.

Analysis of Expressed Soluble eTCR²-BiTE

The ts-NY-ESO-1 TCR and os-NY-ESO-1 TCR protein molecules secreted into the supernatant are analyzed either directly by sodium dodecyl sulfate (SDS) polyacrylamide gel electrophoresis (PAGE) or after protein purification. Protein samples and standards are prepared under both reducing and non-reducing conditions. SDS-PAGE was performed using cast mini gels for protein electrophoresis in a Mini-PROTEAN Tetra cell electrophoresis system (Bio-Rad). Coomassie blue dye was used to stain proteins in SDS-PAGE gel.

Purification of ts-NY-ESO-1 TCR and os-NY-ESO-1 TCR Protein Molecules

Soluble ts-NY-ESO-1 TCR and os-NY-ESO-1 TCR from cell supernatant are purified in two steps. In the first step immobilized metal affinity chromatography (IMAC) are used with nitrilotriacetic acid (NTA) agarose resin loaded with nickel (HisPur Ni-NTA Superflow agarose—Thermo fisher). The binding and washing buffer consists of Tris-buffer saline (TBS) at pH7.2 containing low concentration of imidazole (10-25 mM). Elution and recovery of the His-tagged ts-NY-ESO-1 TCR and os-NY-ESO-1 TCR from the IMAC column are achieved by washing with high concentration of imidazole (>200 mM). The eluted protein fractions are analysed by SDS-PAGE and the fractions containing the protein of interest are pooled. The pooled protein fraction is used directly in binding assays or further purified in a second step involving size-exclusion chromatography (SEC). Superdex 200 increase prepacked column (Gelifesciences) are used to separate out monomer, oligomer and any aggregated forms of the target protein.

### Surface Plasmon Resonance

The specific binding and affinity analysis of ts-NY-ESO-1 TCR and os-NY-ESO-1 TCR to its pMHC is performed using BIAcore. Briefly, the purified Histidine-tagged ts-NY-ESO-1 TCR and os-NY-ESO-1 TCR proteins are captured onto sensor surface via Ni²⁺ chelation of nitrilotriacetic acid (NTA). Varying concentration of the analyte solution containing NY-ESO pep_{(SLLMWITQV)}-MHC (Prolmmune) is injected and the binding signals were monitored

### Example 2: Generation of Tetravalent and Octavalent Soluble NY-ESO TCR-IL2 Fusion Molecule

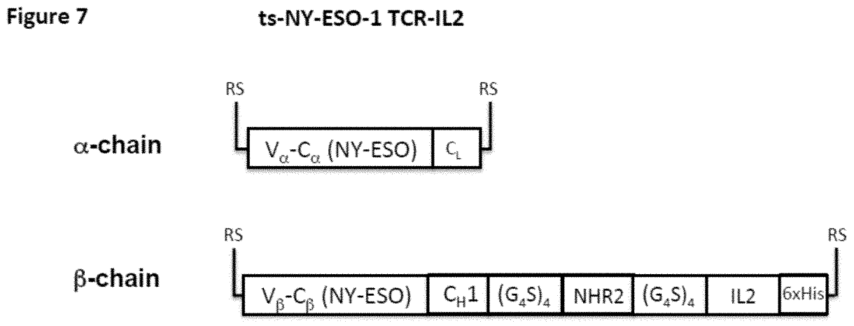

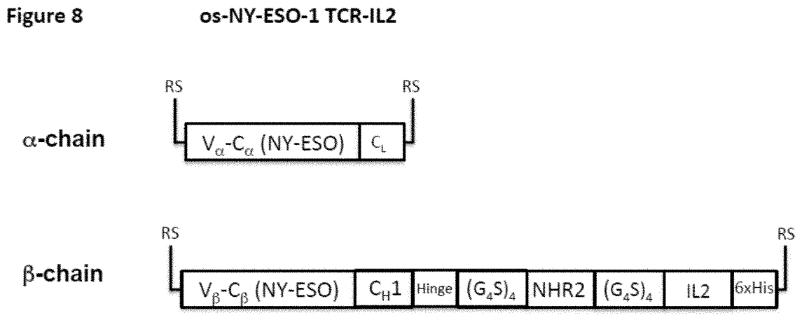

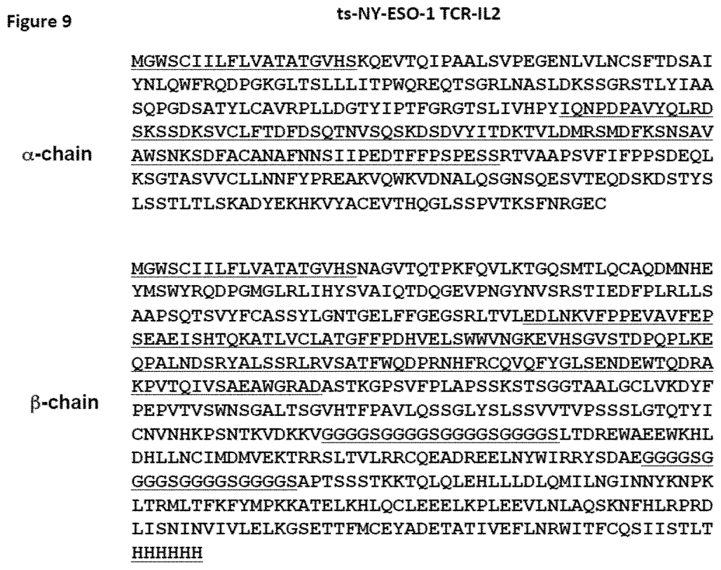

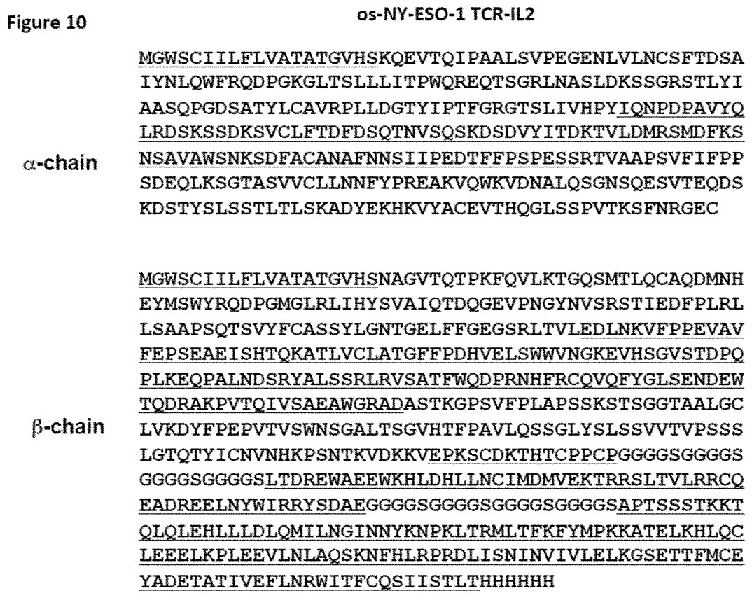

The DNA encoding the domains required for expressing ts-NY-ESO-1 TCR-IL2 and os-NY-ESO-1 TCR-IL2 protein complexes are synthesized and cloned into the expression vector pTT5 as described above. A schematic representation of the domains within the TCR αβ chains for ts-NY-ESO-1 TCR-IL2 and os-NY-ESO-1 TCR-IL2 are shown in FIGS. 7 and 8 and the amino acids sequences are shown in FIGS. 9 and 10.

Expression, purification and characterization of ts-NY-ESO-1 TCR-IL2 and os-NY-ESO-1 TCR-IL2 are carried out as described above.

### Example 3: High Yield Tetramer Secretion

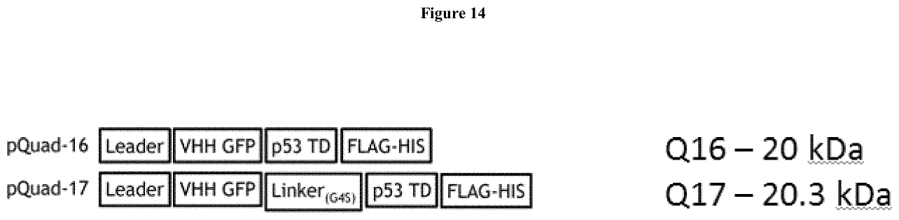

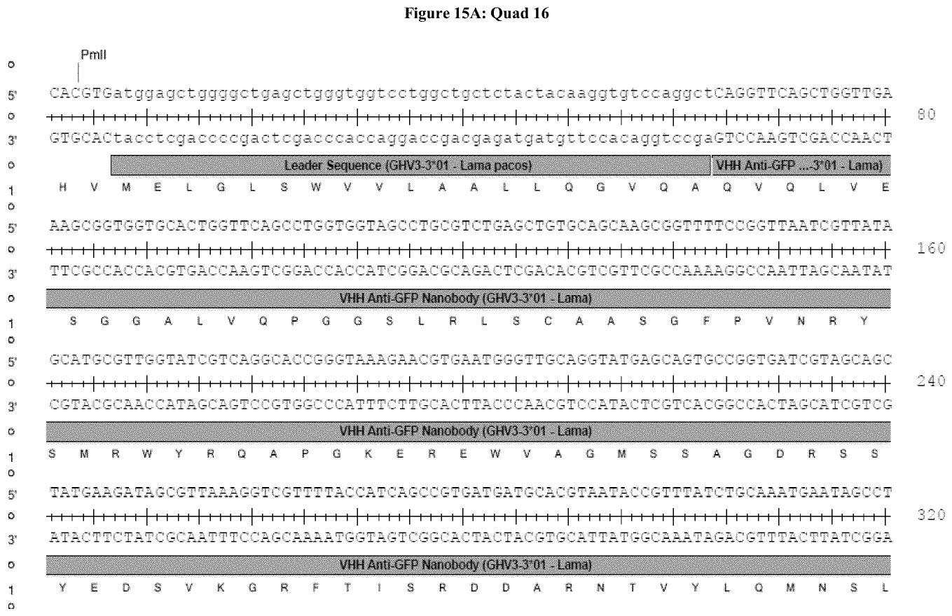

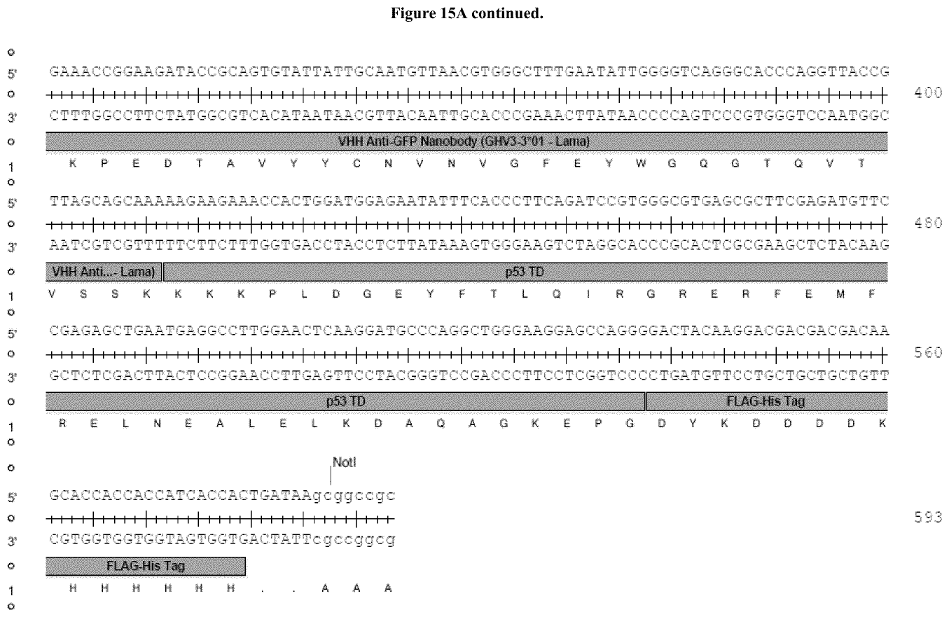

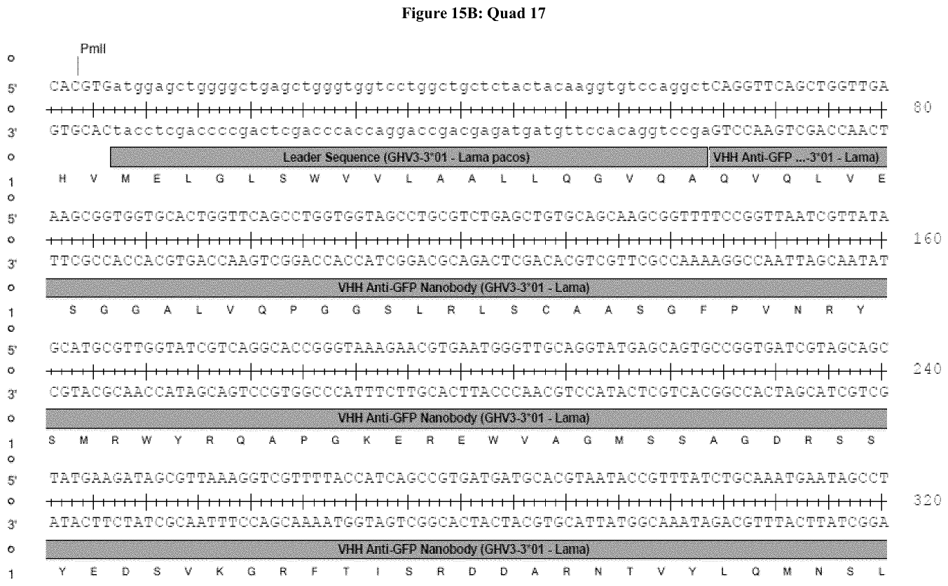

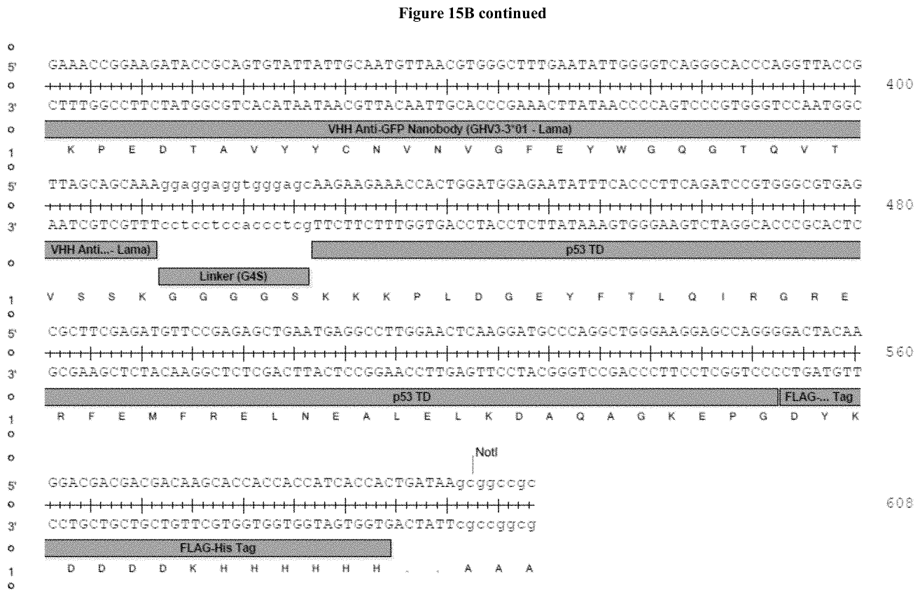

Briefly, using conventional genetic engineering techniques, a HEK293-T cell line was made that encodes Quad 16 (FIGS. 14 and 15A) and another HEK293-T cell line was made that encodes Quad 17 (FIGS. 14 and 15B).

Protein expression took place and protein was secreted from the cell lines. Samples of the medium in which the cells were incubated were subjected to PAGE under denaturing conditions (SDS-PAGE) or under native conditions (no SDS). The former was further under reduced conditions (using mercaptoethanol), whereas the latter was not.

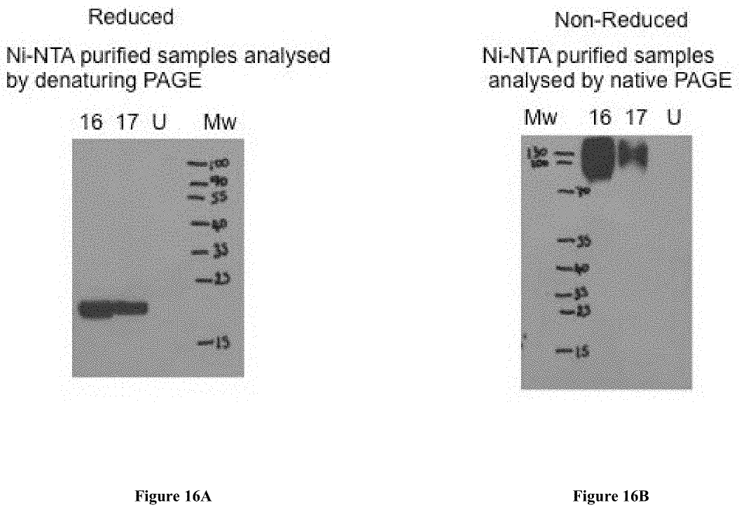

The reduced gel showed a distinct banding (FIG. 16A) at the expected monomer size. Surprisingly, the unreduced, native gel showed no detectable banding at the monomer, dimer or trimer size, but instead heavy banding was seen at the tetramer size indicating that a very high yield of tetramer had been obtained, and this was confirmed by SEC to be of high purity.

For Quad 16, the tetramer peak from SEC was run on SDS-PAGE and the obtained band was cut out for mass spectrometry. The data were obtained with trypsin digests and p53 was detected in 100% of the protein. This was conclusive evidence that the secreted Quad 16 was multimerised

### Example 4: Intracellular Protein Expression of Extracellular Portion of TCR Fused to NHR2 TD

### Expression Vector

All DNA fragments were synthesized and cloned into the expression vector, pEF/myc/cyto (Invitrogen) by Twist Bioscience (California). Schematics and sequences of the synthesized DNA fragments and Quad polypeptides are shown in FIG. 21, and the sequence tables herein.

### DNA Preparation

Lyophilised plasmid DNA synthesized by Twist Bioscience, were resuspended with MQ water to a concentration of 50 ng/gl. 50 ng of DNA was transformed into 50 μl of competent DH5a cells using a conventional heat shock method. The cells were plated on LB agar plates containing 100 gg/mL ampicillin and grown overnight at 37° C. Individual colonies were picked and grown overnight at 37° C., 220 rpm. The DNA was purified from the cells using the QIAprep Spin Miniprep Kit, according to the manufacturers instructions (Qiagen).

### Transfections in HEK293T Cell

Briefly, HEK293T cells were maintained in high glucose DMEM supplemented with 10% FBS and Pen/Strep. Cells were seeded at 6×10⁵ cells per well of a 6-well plate in 2 ml media and were allowed to adhere overnight at 37° C., 5% CO₂. 7.5 μl of Lipofectamine 2000 was diluted in 150 μl of OptiMem and incubated at room temperature for 5 mins. Plasmid DNA (2.5 μg) was diluted in 150 μl of OptiMem. Diluted DNA was combined with the diluted Lipofectamine 2000, mixed gently and incubated at R.T. for 20 mins. The 300 μl of complexes were added to one well of the 6-well plates. When analysis required the media to be serum free, the media was aspirated and replaced with CD293 media 6 hours post-transfection. The cells were incubated for 48 hours at 37° C., 5% CO₂ prior to analysis.

Accordingly, different formats of TCR-linked NHR2 tetramerization domain (TD) constructs (Quads) were transfected into HEK293T cells. Quads 3 & 4 resembling a TCR tetravalent format (structure schematically represented in FIGS. 1 & 3) and Quads 12 & 13 resembling a TCR octavalent format (Structure schematically represented in FIGS. 2 & 4) were transfected for protein expression analysis. Protein samples were prepared from transfected HEK293T cells as follows to check for intracellularly expressed protein. Briefly cells were washed once with 2m PBS, which was subsequently aspirated. 150 μl of Trypsin-EDTA (0.05%) was added to each well and the cells were incubated at R.T. for ˜1 min. The plate was tapped to lift any strongly adhering cells. 850 μl of media was added to each well to inactivate the trypsin. The cells were transferred to a 1.5 ml eppendorf and spun at 1,000 rpm for 5 mins. The supernatant was aspirated and the pellets stored on ice. The cells were resuspended in 400 μl cell lysis buffer (10 mM Tris pH 7.5, 1% SDS) containing Protease Inhibitor Cocktail Set III (Calbiochem), diluted 1/200. The samples were vortexed vigorously and incubated on ice for 20 mins. The cells were sonicated using a Branson Ultrasonics Sonifier™ (Thermo Fisher Scientific). The amplitude was set to 30% and the cells were sonicated for a total of 24 seconds (6 secs on, 3 secs off x4). The total protein concentration was quantified using the Pierce BCA Protein Assay Kit™, according to the manufacturer's instructions. 100 μg was diluted with MQ water to give a volume of 80 μl. 20 μl of 5×SDS loading buffer was then added giving samples of 1 mg/ml. Samples were incubated at 95° C. for 5 min prior to SDS-PAGE and Western blot analysis.

Protein samples were separated on SDS-PAGE under denaturing condition. Typically, 25 μg of whole cell lysate (25 μl) were loaded on to the gel for Western blot analysis. 5 μl of PageRule Prestained 10-180 kDa Protein Ladder was loaded into the gel alongside the protein samples. The gels were run in Tris-Glycine buffer containing 0.1% SDS. A constant voltage of 150 volts was used and the gels were run for ˜70 mins until the dye front has migrated fμlly.

SDS-PAGE (15% Bis-Tris) gels were prepared using the following resolving and stacking gels.

Resolving Gel:
5 ml 30% Bis-Acrylamide
2.6 ml 1.5M Tris (pH8.8)
50 μl 20% SDS
100 μl 10% APS
10 μl TEMED
2.2 ml MQ Water

Stacking Gel:
0.75 ml 30% Bis-Acrylamide
1.25 ml 1.5 M Tris (pH 8.8)
25 μl 20% SDS
50 μl 10% APS
5 μl TEMED
2.9 ml MQ Water

Western blotting was performed for the specific and sensitive detection of protein expression of TCR-NHR2 TD fusion proteins from Quads 3, 4, 12 and 13. Proteins separated out on SDS-PAGE were transferred onto Amersham Hybond™ 0.45 μM PVDF membrane as follows. Briefly, Amersham Hybond 0.45 μM PVDF membrane was activated with MeOH for ˜1 min and rinsed with transfer buffer (25 mM Tris, 190 mM Glycine, 20% MeOH) before use. The sponge, filter paper, gel, membrane, filter paper, sponge stack was prepared and placed in the cassette for transfer. Transfer was carried out on ice at 280 mA for 75 mins. The membrane was incubated for ˜2 hours in blocking buffer (TBST, 5% milk powder). The membrane was washed briefly with TBST before being incubated at 4° C. overnight with anti-human IgG HRP (Thermo, 31410) diluted 1/2500 in TBST, 1% milk powder. The membrane was washed thoroughly (three washes of TBST, 15 mins each) before being developed using the Pierce ECL Western Blotting Substrate.

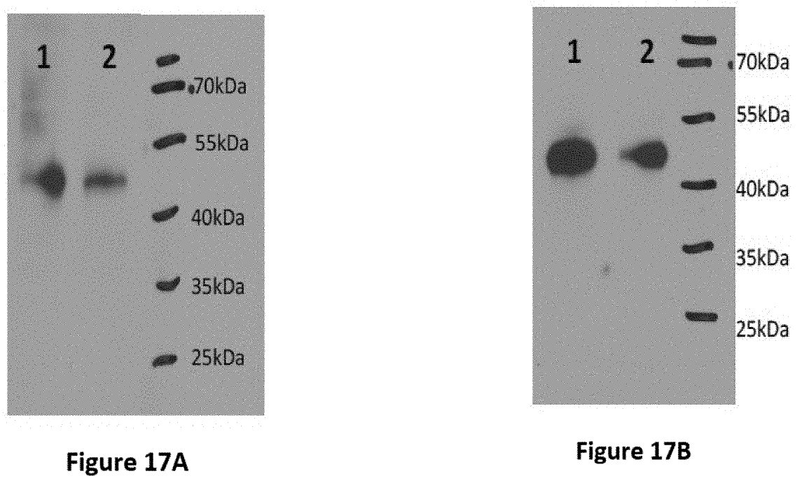

Using anti-human IgG detection antibody to probe Western blots, specific protein band at the expected molecular weight can be detected from samples prepared from Quads 3 (46.1 kDa), 4 (46.4 kDa), 12 (47.8 kDa) and 13 (48.1 kDa) (FIGS. 17A and 17B). These data confirm intracellular protein expression of TCR-NHR2 TD fusion proteins in HEK293T cells.

For all of the Quads analysed, a clear single band can be detected indicating TRVβ-TRCβ-IgG1-CH1 (+/−IgG hinge domain) fusions with the NHR2 TD are stable. These expression data also confirm the possibility of assembling tetravalent (Quads 3 & 4) and octavalent (Quads 12& 13) molecules as exemplified in this example.

The difference between Quads 3 and 4 is the presence of a small peptide linker (G4S) located between the IgG1 CH1 domain and NHR2 TD. This is also true for Quads 12 and 13 where Q13 contains a peptide linker between the IgG1 CH1 domain and NHR2 TD. From the expression data, it can be seen the peptide linker does not effect protein expression. However, it may be desirable to include a peptide linker to aid antigen binding and or stabilizing the multimerisation complex in these TCR-NHR2 TD formats.

### Example 5: Soluble Protein Expression of Extracellular Portion of TCR Fused to NHR2 TD

TCR-NHR2 TD fusion proteins were shown in Example 4 to be expressed intracellularly in HEK293T cells. Here again Quads 3, 4, 12 and 13 were used to demonstrate soluble expression of these fusion proteins. As described above, Quads 3, 4, 12 and 13 were transfected into HEK293T cells and soluble proteins from the cell supernatant were concentrated. Briefly, the media was harvested 48 hours post-transfection and centrifuged at 2,000 rpm for 5 mins to remove any cells or debris. Typically, 500 μl of media was concentrated to 100 μl using Amicon™ Ultra 0.5 Centrifugal Units with a MWCO of 10 kDa. 25 μl of 5×SDS loading buffer was added to the sample, which was then incubated at 95° C. for 5 mins prior to gel/Western blot analysis. Concentrated protein samples were separated out on SDS-PAGE gel and transferred onto Amersham Hybond 0.45 μM PVDF membrane. Western blotting and protein detection was done using anti-human IgG HRP using the methods described above.

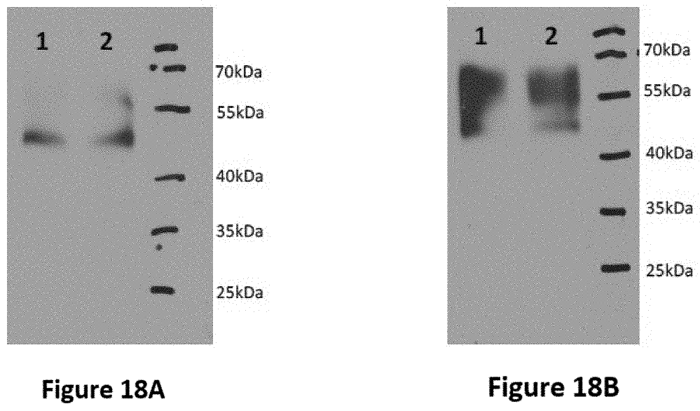

Protein samples concentrated and prepared from cell supernatants show specific protein band at the expected molecular weight on Western blots corresponding to Quads 3 (46.1 kDa), 4 (46.4 kDa), 12 (47.8 kDa) and 13 (48.1 kDa) (FIGS. 18A and 18B). The Western blot expression data unequivocally shows soluble expression of TCR-NHR2 TD fusion proteins in HEK293T. These data are the first report demonstrating soluble expression of TCR-NHR2 TD fusion proteins expressed in eukaryotic cells such as HEK293T cells.

Detection of soluble protein expression from both tetrameric (Quads 3 & 4) and octameric (Quads 12 & 13) TCR-NHR2 TD formats highlights the potential applicability of NHR2 TD in a broad setting. Use of NHR2 TD fusion molecules could be used for the preparation of therapeutic molecules and protein molecules for use in diagnostics and imaging.

### Example 6: Intracellular Protein Expression of Antibody Fragments Fused to NHR2 TD

To further exemplify the versatility of NHR2 TD, several different antibody fragment formats fused to NHR2 TD were constructed for testing their expression in HEK293T cells.

Quads 14 and 15 contain an antibody VH domain fused to NHR2 TD either with or without a peptide linker located between the VH and NHR2 TD as schematically depicted in FIGS. 11B and 21. The VH domain in Quads 14 and 15 are specific for GFP (green fluorescent protein). Several other versions of this format were also constructed and tested with VH specific for therapeutically useful drug targets. Sequences of the binding domains are listed in Table 4. Some of these include Quad 34 (specific for TNFα), Quad 38 (specific for VEGF), Quad 40 (specific for EGFR) and Quad 44 (specific for CD38).

Quads 38 and 44 were further developed to include an additional binding arm with the inclusion of a second VH domain specific for EGFR and CD138 respectively yielding Quads 42and 46. Quads 42 and 46 represent bispecific molecules with the capability to multimerise via the NHR2 TD domain to form bispecific tetramers.

In another example, an effector molecule (human IL2) was linked to the C-terminus of Quads 14 & 15 resulting in Quads 18 and 19, whereby the VH-NHR2-IL2 molecule is tetravalent and bifunctional.

In another example, antibody Fab fragment (VH-CH1) was linked to NHR2 TD (Quads 23 and 24) and as schematically depicted in FIGS. 12A-12B. Quads 23 and 24 represent tetravalent Fab molecules when co-expressed or mixed and assembled in-vitro with a second chain containing immunoglobulin light chain (e.g. Quad 25).

In yet another example, a human IgG1 hinge domain was included to Quads 23 and 24, which is referred to as Quads 26 and 27 and as schematically depicted in FIGS. 13A-13B. Quads 26 and 27 represent octavalent Fab molecules when co-expressed or mixed and assembled in-vitro with a second chain containing immunoglobulin light chain domains (e.g. Quad 25).

The following Quad vectors, Quads 14, 15, 18, 19, 23, 24, 26, 27, 34, 38, 40, 42, 44 and 46 all of which are His-tagged were transfected in HEK293T cells. Protein samples were prepared from whole-cell extracts as described above, separated out on SDS-PAGE and transferred onto Amersham Hybond 0.45 μM PVDF membrane. Specific protein expression were probed using anti-His HRP (Sigma, A7058) diluted 1/2500 in TBST, 1% milk powder.

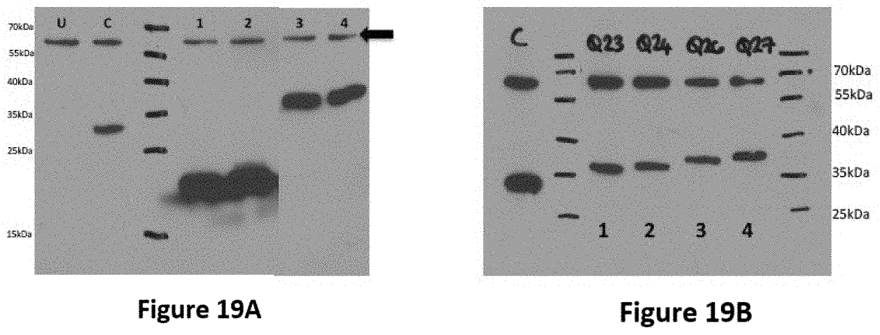

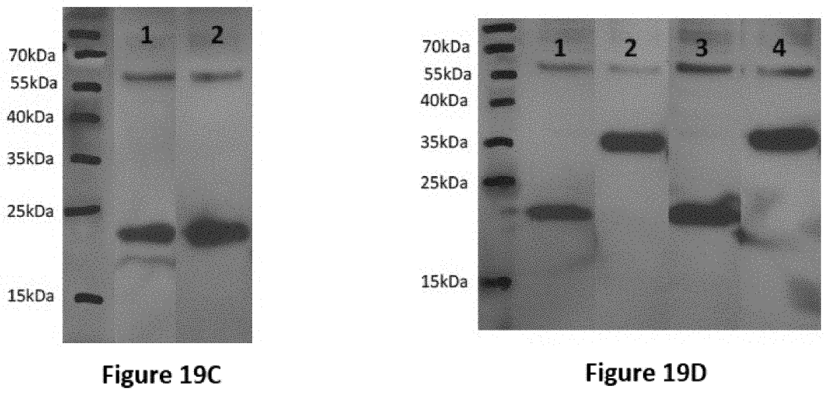

Specific protein expression in whole cell extracts could be detected for all the different antibody-NHR2 TD fusion proteins tested using Quads 14, 15, 18, 19, 23, 24, 26, 27, 34, 38, 40, 42, 44 and 46 (FIGS. 19A-19D). Interestingly, Quads 18 and 19 containing an effector domain (IL2) fused to the C-terminus of NHR2 TD domain, in addition to the VH binding domain fused to the N-terminus of NHR2 TD showed good protein expression.

Expression of Quads 23 and 24 polypeptides highlights the potential to use NHR2 TD to form tetravalent antibody Fab molecules when co-expressed or mixed in-vitro with a partner soluble Quad molecule (e.g. Quad 25). Similarly expression of Quads 26 and 27, which include human IgG1 hinge domain highlight the potential to use NHR2 TD to form octavalent antibody Fab molecules when co-expressed or mixed in-vitro with a partner soluble second partner chain (e.g. Quad 25).

Quads 42 and 46 bispecific molecules containing an additional VH domain fused to the C-terminus of NHR2 TD domain also showed good protein expression. These data highlights the versatility of the NHR2 TD domain and its ability to be fused to different binding and effector molecules for developing a vast array of protein formats. The data also suggest it is possible to fuse protein molecules to both the N-terminus and C-terminus of NHR2 TD, which allows for the development of bispecific multivalent protein molecules.

### Example 7: Multivalent Assembly of Antibody Fragments Fused to NHR2 TD

NHR2 TD is responsible for the oligomerisation of ETO into a tetrameric complex. Using the NHR2 TD domain, it is possible to fuse binding domains and effector molecules to the N-terminus or C-terminus or both N- and C-terminus without effecting expression as shown in examples 4-6. Binding domains could be TCR variable and constant domains, antibody and antibody fragments or effector molecules such as IL2. It is also possible to express proteins in a soluble format when fused to NHR2 TD (FIGS. 18A-18B) despite NHR2 TD being a part of an intracellularly expressed protein where in nature it is only expressed inside the cell.

To demonstrate whether NHR2 TD retains its potential to oligomerise once it is fused to a binding domain, Quads 14 and 15 were expressed in HEK293T cells and protein samples were prepared from whole cell extracts as described above. Protein samples were separated out on PAGE gel under denaturing and non-denaturing (native) conditions. Native gels were prepared using the protocol described above, but without SDS. Proteins from PAGE gels were transferred onto Amersham Hybond 0.45 μM PVDF membrane. Specific protein expression was probed with anti-human IgG HRP detection antibody.

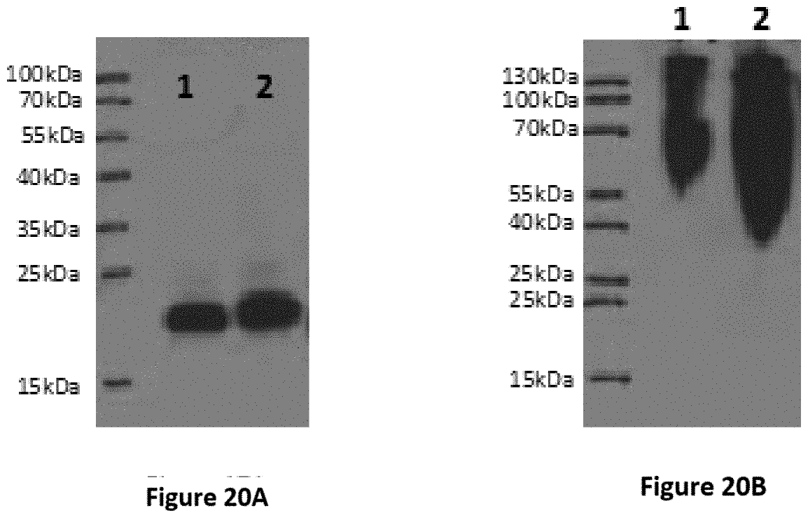

As expected under denaturing conditions, expression of VH-NHR2 TD from Quads 14 and 15 can be seen as a monomer where a specific protein band can be detected at the expected molecular weight (22 and 22.3 kDa) (FIG. 20A). Under non-denaturing and thus native conditions, interestingly no monomer or dimer of VH-NHR2 TD from Quads 14 and 15 can be detected (FIG. 20B). Only a high molecular weight protein band believed to be tetramers of VH-NHR2 TD from Quads 14 and 15 can be detected. The assembly of tetramers appears to be highly efficient and pure judging by the protein intensity and the absence of any detectable monomers and dimers of Quads 14 and 15.

Together with the data in examples 4-7, there is conclusive evidence NHR2 TD is highly versatile allowing fusion of various protein binding domains and effector molecules. NHR2 TD allows soluble expression of proteins from eukaryotic cells such as HEK293T cells and they form highly stable and pure tetrameric molecules.

### REFERENCES

Ali, S. A. et al., 1999. Transferrin Trojan Horses as a Rational Approach for the Biological Delivery of Therapeutic Peptide Domains. Journal of Biological Chemistry, 274(34), pp. 24066-24073.
Barbas et al. (1992) supra
Bentley, G. A. & Mariuzza, R. A., 1996. THE STRUCTURE OF THE T CELLANTIGEN RECEPTOR. Annual Review of Immunology, 14(1), pp. 563-590.
Binz, H. K. et al., 2003. Designing repeat proteins: well-expressed, soluble and stable proteins from combinatorial libraries of consensus ankyrin repeat proteins. Journal of molecular biology, 332(2), pp. 489-503.
Bixby, K. A. et al., 1999. Zn2+-binding and molecular determinants of tetramerizationin voltage-gated K+ channels. Nature structural biology, 6(1), pp. 38-43.
Borghouts, C., Kunz, C. & Groner, B., 2005. Peptide aptamers: recent developments for cancer therapy. Expert Opinion on Biological Therapy, 5(6), pp. 783-797.
Breitling, F. et al., 1991. A surface expression vector for antibody screening. Gene, 104(2), pp. 147-53.
Burton, D. R. et al., 1991. A large array of human monoclonal antibodies to type 1 human immunodeficiency virus from combinatorial libraries of asymptomatic seropositive individuals. Proceedings of the National Academy of Sciences of the United States of America, 88(22), pp. 10134-7.
Chang, C. N., Landolfi, N. F. & Queen, C., 1991. Expression of antibody Fab domains on bacteriophage surfaces. Potential use for antibody selection. Journal of immunology (Baltimore, Md.: 1950), 147(10), pp. 3610-4.
Chaudhary, V. K. et al., 1990. A rapid method of cloning functional variable-region antibody genes in Escherichia coli as single-chain immunotoxins. Proceedings of the National Academy of Sciences, 87(3), pp. 1066-1070.
Chiswell, D. J. & McCafferty, J., 1992. Phage antibodies: will new “coliclonal” antibodies replace monoclonal antibodies? Trends in biotechnology, 10(3), pp. 80-4.
Clackson, T. et al., 1991. Making antibody fragments using phage display libraries. Nature, 352(6336), pp. 624-8.
Davis, M. M. & Bjorkman, P. J., 1988. T-cell antigen receptor genes and T-cell recognition. Nature, 334(6181), pp. 395-402.
Ding, X.-F. et al., 2008. Characterization and Expression of a Human KCTD1 Gene Containing the BTB Domain, Which Mediates Transcriptional Repression and Homomeric Interactions. DNA and Cell Biology, 27(5), pp. 257-265.
Fujiwara, Y. & Minor, D. L., 2008. X-ray Crystal Structure of a TRPM Assembly Domain Reveals an Antiparallel Four-stranded Coiled-coil. Journal of Molecular Biology, 383(4), pp. 854-870.
Hawkins, R. E. & Winter, G., 1992. Cell selection strategies for making antibodies from variable gene libraries: trapping the memory pool. European journal of immunology, 22(3), pp. 867-70.
Herrin, B. R. & Cooper, M. D., 2010. Alternative adaptive immunity in jawless vertebrates. Journal of immunology (Baltimore, Md.: 1950), 185(3), pp. 1367-74.
Hoogenboom, H. R. et al., 1991. Multi-subunit proteins on the surface of filamentous phage: methodologies for displaying antibody (Fab) heavy and light chains. Nucleic acids research, 19(15), pp. 4133-7.
Hosse, R. J., Rothe, A. & Power, B. E., 2006. A new generation of protein display scaffolds for molecular recognition. Protein science: a publication of the Protein Society, 15(1), pp. 14-27.
Irving, R. A. et al., 2001. Ribosome display and affinity maturation: from antibodies to single V-domains and steps towards cancer therapeutics. Journal of immunological methods, 248(1-2), pp. 31-45.
Jeffrey, P. D., Gorina, S. & Pavletich, N. P., 1995. Crystal structure of the tetramerization domain of the p53 tumor suppressor at 1.7 angstroms. Science (New York, N.Y.), 267(5203), pp. 1498-502.
Kang, A. S. et al., 1991. Linkage of recognition and replication functions by assembling combinatorial antibody Fab libraries along phage surfaces. Proceedings of the National Academy of Sciences, 88(10), pp. 4363-4366.
Kohl, A. et al., 2003. Designed to be stable: Crystal structure of a consensus ankyrin repeat protein. Proceedings of the National Academy of Sciences, 100(4), pp. 1700-1705.
Lerner, R. A. et al., 1992. Antibodies without immunization. Science (New York, N.Y.), 258(5086), pp. 1313-4.
Liu, Y. et al., 2006. The tetramer structure of the Nervy homology two domain, NHR2, is critical for AML1/ETO's activity. Cancer Cell, 9(4), pp. 249-260.
Lowman, H. B. et al., 1991. Selecting high-affinity binding proteins by monovalent phage display. Biochemistry, 30(45), pp. 10832-8.
Marks et al. (1991) supra
Marks, J. D. et al., 1991. By-passing immunization. Human antibodies from V-gene libraries displayed on phage. Journal of molecular biology, 222(3), pp. 581-97.
Marks, J. D. et al., 1992. Molecular evolution of proteins on filamentous phage. Mimicking the strategy of the immune system. The Journal of biological chemistry, 267(23), pp. 16007-10.
McCafferty et al. (1990) supra
McCafferty, J. et al., 1990. Phage antibodies: filamentous phage displaying antibody variable domains. Nature, 348(6301), pp. 552-4.
Moysey, R., Vuidepot, A.-L. & Boulter, J. M., 2004. Amplification and one-step expression cloning of human T cell receptor genes. Analytical biochemistry, 326(2), pp. 284-6.
Panowski, S. et al., 2014. Site-specific antibody drug conjugates for cancer therapy. mAbs, 6(1), pp. 34-45.
Parker, M. H. et al., 2005. Antibody mimics based on human fibronectin type three domain engineered for thermostability and high-affinity binding to vascular endothelial growth factor receptor two. Protein engineering, design & selection: PEDS, 18(9), pp. 435-44.
Perez, H. L. et al., 2014. Antibody-drug conjugates: Current status and future directions. Drug Discovery Today, 19(7), pp. 869-881.
Reddy Chichili, V. P., Kumar, V. & Sivaraman, J., 2013. Linkers in the structural biology of protein-protein interactions. Protein science: a publication of the Protein Society, 22(2), pp. 153-67.
Shao, C.-Y., Secombes, C. J. & Porter, A. J., 2007. Rapid isolation of IgNAR variable single-domain antibody fragments from a shark synthetic library. Molecular immunology, 44(4), pp. 656-65.
Silverman, J. et al., 2005. Multivalent avimer proteins evolved by exon shuffling of a family of human receptor domains. Nature Biotechnology, 23(12), pp. 1556-1561.
Skerra, A., 2000. Lipocalins as a scaffold. Biochimica et Biophysica Acta (BBA)—Protein Structure and Molecular Enzymology, 1482(1-2), pp. 337-350.
Thie, H. et al., 2009. Multimerization domains for antibody phage display and antibody production. New biotechnology, 26(6), pp. 314-21.
Tonegawa, S., 1988. Somatic generation of immune diversity. Bioscience reports, 8(1), pp. 3-26.
Wälchli, S. et al., 2011. A Practical Approach to T-Cell Receptor Cloning and Expression M. M. Rodrigues, ed. PLoS ONE, 6(11), p. e27930.
Wikman, M. et al., 2004. Selection and characterization of HER2/neu-binding affibody ligands. Protein Engineering Design and Selection, 17(5), pp. 455-462.
Young, P. A., Morrison, S. L. & Timmerman, J. M., 2014. Antibody-cytokine fusion proteins for treatment of cancer: Engineering cytokines for improved efficacy and safety. Seminars in Oncology, 41(5), pp. 623-636.
Zahnd, C. et al., 2007. A designed ankyrin repeat protein evolved to picomolar affinity to Her2. Journal of molecular biology, 369(4), pp. 1015-28.
Zhang, J. et al., 2009. Transient expression and purification of chimeric heavy chain antibodies. Protein Expression and Purification, 65(1), pp. 77-82.
Expert Opinion on Investigational Drugs 16(6) 909-917 (June 2007)
Chapter 7—Non-Antibody Scaffolds from Handbook of Therapeutic Antibodies (2007, edited by Stefan Dubel)

TABLE 1
SEQUENCE LISTING

| SEQ | | | |
|---|---|---|---|
| ID | | | |
| NO: | | AMINO ACID SEQUENCE | NOTES |
| 1 | Human | MEEPQSDPSVEPPLSQETFSDLWKLLPENNVLSPLPSQAMDDLMLSPDDIEQWFTEDPGP | Also known as: p53, p53alpha |
| | p53 | DEAPRMPEAAPPVAPAPAAPTPAAPAPAPSWPLSSSVPSQKTYQGSYGFRLGFLHSGTAK | This isoform is denoted as |
| | isoform | SVTCTYSPALNKMFCQLAKTCPVQLWVDSTPPPGTRVRAMAIYKQSQHMTEVVRRCPHH | the ′canonical′ sequence. |
| | 1 | E | Tetramerization sequence in |
| | | RCSDSDGLAPPQHLIRVEGNLRVEYLDDRNTFRHSVVVPYEPPEVGSDCTTIHYNYMCNS | underline bold (amino acid |
| | | SCMGGMNRRPILTIITLEDSSGNLLGRNSFEVRVCACPGRDRRTEEENLRKKGEPHHELP | position numbers 325 to 356) |
| | | PGSTKRALPNNTSSSPQPKKKPLDGEYFTLQIRGRERFEMFRELNEALELKDAQAGKEP | |
| | | G | |
| | | GSRAHSSHLKSKKGQSTSRHKKLMFKTEGPDSD | |
| 2 | Human | MEEPQSDPSVEPPLSQETFSDLWKLLPENNVLSPLPSQAMDDLMLSPDDIEQWFTEDPGP | Also known as: I9RET, |
| | p53 | DEAPRMPEAAPPVAPAPAAPTPAAPAPAPSWPLSSSVPSQKTYQGSYGFRLGFLHSGTAK | p53beta |
| | isoform | SVTCTYSPALNKMFCQLAKTCPVQLWVDSTPPPGTRVRAMAIYKQSQHMTEVVRRCPHH | The sequence of this isoform |
| | 2 | E | differs from the canonical |
| | | RCSDSDGLAPPQHLIRVEGNLRVEYLDDRNTFRHSVVVPYEPPEVGSDCTTIHYNYMCNS | sequence (isoform 1) as |
| | | SCMGGMNRRPILTIITLEDSSGNLLGRNSFEVRVCACPGRDRRTEEENLRKKGEPHHELP | follows: |
| | | PGSTKRALPNNTSSSPQPKKKPLDGEYFTLQDQTSFQKENC | 332-341: IRGRERFEMF→ |
| | | | DQTSFQKENC |
| | | | 342-393: Missing. |
| 3 | Human | MEEPQSDPSVEPPLSQETFSDLWKLLPENNVLSPLPSQAMDDLMLSPDDIEQWFTEDPGP | Also known as: p53gamma |
| | p53 | DEAPRMPEAAPPVAPAPAAPTPAAPAPAPSWPLSSSVPSQKTYQGSYGFRLGFLHSGTAK | The sequence of this isoform |
| | isoform | SVTCTYSPALNKMFCQLAKTCPVQLWVDSTPPPGTRVRAMAIYKQSQHMTEVVRRCPHH | differs from the canonical |
| | 3 | E | sequence as follows: |
| | | RCSDSDGLAPPQHLIRVEGNLRVEYLDDRNTFRHSVVVPYEPPEVGSDCTTIHYNYMCNS | 332-346: |
| | | SCMGGMNRRPILTIITLEDSSGNLLGRNSFEVRVCACPGRDRRTEEENLRKKGEPHHELP | IRGRERFEMFRELNE→ |
| | | PGSTKRALPNNTSSSPQPKKKPLDGEYFTLQMLLDLRWCYFLINSS | MLLDLRWCYFLINSS |
| | | | 347-393: Missing |
| 4 | Human | MDDLMLSPDDIEQWFTEDPGPDEAPRMPEAAPPVAPAPAAPTPAAPAPAPSWPLSSSVPS | Also known as: Del40-p53, |
| | p53 | QKTYQGSYGFRLGFLHSGTAKSVTCTYSPALNKMFCQLAKTCPVQLWVDSTPPPGTRVRA | Del40-p53alpha, p47 |
| | isoform | MAIYKQSQHMTEVVRRCPHHERCSDSDGLAPPQHLIRVEGNLRVEYLDDRNTFRHSVVVP | The sequence of this isoform |
| | 4 | YEPPEVGSDCTTIHYNYMCNSSCMGGMNRRPILTIITLEDSSGNLLGRNSFEVRVCACPG | differs from the canonical |
| | | RDRRTEEENLRKKGEPHHELPPGSTKRALPNNTSSSPQPKKKPLDGEYFTLQIRGRERFE | sequence as follows: |
| | | MFRELNEALELKDAQAGKEPGGSRAHSSHLKSKKGQSTSRHKKLMFKTEGPDSD | 1-39: Missing. |
| 5 | Human | MDDLMLSPDDIEQWFTEDPGPDEAPRMPEAAPPVAPAPAAPTPAAPAPAPSWPLSSSVPS | Also known as: Del40- |
| | p53 | QKTYQGSYGFRLGFLHSGTAKSVTCTYSPALNKMFCQLAKTCPVQLWVDSTPPPGTRVRA | p53beta. |
| | isoform | MAIYKQSQHMTEVVRRCPHHERCSDSDGLAPPQHLIRVEGNLRVEYLDDRNTFRHSVVVP | The sequence of this isoform |
| | 5 | YEPPEVGSDCTTIHYNYMCNSSCMGGMNRRPILTIITLEDSSGNLLGRNSFEVRVCACPG | differs from the canonical |
| | | RDRRTEEENLRKKGEPHHELPPGSTKRALPNNTSSSPQPKKKPLDGEYFTLQDQTSFQKE | sequence as follows: |
| | | NC | 1-39: Missing. |
| | | | 332-341: IRGRERFEMF→ |
| | | | DQTSFQKENC |
| | | | 342-393: Missing. |
| 6 | Human | MDDLMLSPDDIEQWFTEDPGPDEAPRMPEAAPPVAPAPAAPTPAAPAPAPSWPLSSSVPS | Also known as: Del40- |
| | p53 | QKTYQGSYGFRLGFLHSGTAKSVTCTYSPALNKMFCQLAKTCPVQLWVDSTPPPGTRVRA | p53gamma. The sequence of |
| | isoform | MAIYKQSQHMTEVVRRCPHHERCSDSDGLAPPQHLIRVEGNLRVEYLDDRNTFRHSVVVP | this isoform differs from |
| | 6 | YEPPEVGSDCTTIHYNYMCNSSCMGGMNRRPILTIITLEDSSGNLLGRNSFEVRVCACPG | the canonical sequence as |
| | | RDRRTEEENLRKKGEPHHELPPGSTKRALPNNTSSSPQPKKKPLDGEYFTLQMLLDLRWC | follows: 1-39: Missing. |
| | | YFLINSS | 332-346: |
| | | | IRGRERFEMFRELNE→ |
| | | | MLLDLRWCYFLINSS |
| | | | 347-393: Missing. |
| 7 | Human | MFCQLAKTCPVQLWVDSTPPPGTRVRAMAIYKQSQHMTEVVRRCPHHERCSDSDGLAPP | Also known as: Del133-p53, |
| | p53 | Q | Del133-p53alpha. The |
| | isoform | HLIRVEGNLRVEYLDDRNTFRHSVVVPYEPPEVGSDCTTIHYNYMCNSSCMGGMNRRPIL | sequence of this isoform |
| | 7 | TIITLEDSSGNLLGRNSFEVRVCACPGRDRRTEEENLRKKGEPHHELPPGSTKRALPNNT | differs from the canonical |
| | | SSSPQPKKKPLDGEYFTLOIRGRERFEMFRELNEALELKDAOAGKEPGGSRAHSSHLKS | sequence as follows: |
| | | K | 1-132: Missing. |
| | | KGQSTSRHKKLMFKTEGPDSD | |
| 8 | Human | MFCQLAKTCPVQLWVDSTPPPGTRVRAMAIYKQSQHMTEVVRRCPHHERCSDSDGLAPP | Also known as: Del133- |
| | p53 | Q | p53beta. The sequence of |
| | isoform | HLIRVEGNLRVEYLDDRNTFRHSVVVPYEPPEVGSDCTTIHYNYMCNSSCMGGMNRRPIL | this isoform differs from |
| | 8 | TIITLEDSSGNLLGRNSFEVRVCACPGRDRRTEEENLRKKGEPHHELPPGSTKRALPNNT | the canonical sequence as |
| | | SSSPQPKKKPLDGEYFTLQDQTSFQKENC | follows: 1-132: Missing. |
| | | | 332-341: IRGRERFEMF→ |
| | | | DQTSFQKENC |
| | | | 342-393: Missing. |
| 9 | Human | MFCQLAKTCPVQLWVDSTPPPGTRVRAMAIYKQSQHMTEVVRRCPHHERCSDSDGLAPP | Also known as: Del133- |
| | p53 | Q | p53gamma. The sequence of |
| | isoform | HLIRVEGNLRVEYLDDRNTFRHSVVVPYEPPEVGSDCTTIHYNYMCNSSCMGGMNRRPIL | this isoform differs from |
| | 9 | TIITLEDSSGNLLGRNSFEVRVCACPGRDRRTEEENLRKKGEPHHELPPGSTKRALPNNT | the canonical sequence as |
| | | SSSPQPKKKPLDGEYFTLQMLLDLRWCYFLINSS | follows: 1-132: Missing. |
| | | | 332-346: |
| | | | IRGRERFEMFRELNE→ |
| | | | MLLDLRWCYFLINSS |
| | | | 347-393: Missing. |
| 10 | A human | GEYFTLQIRGRERFEMFRELNEALELKDAQAG | |
| | p53-TD | | |
| 11 | A human | RSPDDELLYLPVRGRETYEMLLKIKESLELMQYLPQHTIETYRQQQQQQH | |
| | p63-TD | | |
| 12 | A human | KKRRHGDEDTYYLQVRGRENFEILMKLKESLELMELVPQPLV | |
| | p73-TD | | |

TABLE 2
Example Human Proteins Comprising a Tetramerization Domain

| Protein | | | | |
|---|---|---|---|---|
| Number | Uniprot Entry | Entry name | Protein names | Gene names |
| 1 | P04637 | P53_HUMAN | Cellular tumor antigen p53 (Antigen NY-CO-13) | TP53 P53 |
| | | | (Phosphoprotein p53) (Tumor suppressor p53) | |
| 2 | Q719H9 | KCTD1_HUMAN | BTB/POZ domain-containing protein KCTD1 (Potassium | KCTD1 C18orf5 |
| | | | channel tetramerization domain-containing protein 1) | |
| 3 | P51787 | KCNQ1_HUMAN | Potassium voltage-gated channel subfamily KQT member 1 | KCNQ1 KCNA8 |
| | | | (IKs producing slow voltage-gated potassium channel subunit | KCNA9 KVLQT1 |
| | | | alpha KvLQT1) (KQT-like 1) (Voltage-gated potassium | |
| | | | channel subunit Kv7.1) | |
| 4 | Q06455 | MTG8_HUMAN | Protein CBFA2T1 (Cyclin-D-related protein) (Eight twenty | RUNX1T1 |
| | | | one protein) (Protein ETO) (Protein MTG8) (Zinc finger | AML1T1 CBFA2T1 |
| | | | MYND domain-containing protein 2) | CDR ETO MTG8 |
| | | | | ZMYND2 |
| 5 | Q9H3F6 | BACD3_HUMAN | BTB/POZ domain-containing adapter for CUL3-mediated | KCTD10 ULR061 |
| | | | RhoA degradation protein 3 (hBACURD3) (BTB/POZ | MSTP028 |
| | | | domain-containing protein KCTD10) (Potassium channel | |
| | | | tetramerization domain-containing protein 10) | |
| 6 | Q12809 | KCNH2_HUMAN | Potassium voltage-gated channel subfamily H member 2 (Eag | KCNH2 ERG ERG1 |
| | | | homolog) (Ether-a-go-go-related gene potassium channel 1) | HERG |
| | | | (ERG-1) (Eag-related protein 1) (Ether-a-go-go-related | |
| | | | protein 1) (H-ERG) (hERG-1) (hERGl) (Voltage-gated | |
| | | | potassium channel subunit Kv11.1) | |
| 7 | Q96SI1 | KCD15_HUMAN | BTB/POZ domain-containing protein KCTD15 (Potassium | KCTD15 |
| | | | channel tetramerization domain-containing protein 15) | |
| 8 | P02766 | TTHY_HUMAN | Transthyretin (ATTR) (Prealbumin) (TBPA) | TTR PALB |
| 9 | Q14681 | KCTD2_HUMAN | BTB/POZ domain-containing protein KCTD2 (Potassium | KCTD2 KIAA0176 |
| | | | channel tetramerization domain-containing protein 2) | |
| 10 | Q7Z5Y7 | KCD20_HUMAN | BTB/POZ domain-containing protein KCTD20 (Potassium | KCTD20 C6orf69 |
| | | | channel tetramerization domain containing 20) | |
| 11 | P50552 | VASP_HUMAN | Vasodilator-stimulated phosphoprotein (VASP) | VASP |
| 12 | Q68DU8 | KCD16_HUMAN | BTB/POZ domain-containing protein KCTD16 (Potassium | KCTD16 |
| | | | channel tetramerization domain-containing protein 16) | KIAA1317 |
| 13 | Q09470 | KCNA1_HUMAN | Potassium voltage-gated channel subfamily A member 1 | KCNA1 |
| | | | (Voltage-gated K(+) channel HuKI) (Voltage-gated | |
| | | | potassium channel HBK1) (Voltage-gated potassium channel | |
| | | | subunit Kv1.1) | |
| 14 | P13501 | CCL5_HUMAN | C-C motif chemokine 5 (EoCP) (Eosinophil chemotactic | CCL5 D17S136E |
| | | | cytokine) (SIS-delta) (Small-inducible cytokine A5) (T cell- | SCYA5 |
| | | | specific protein P228) (TCP228) (T-cell-specific protein | |
| | | | RANTES) [Cleaved into: RANTES(3-68); RANTES(4-68)] | |
| 15 | P08069 | IGF1R_HUMAN | Insulin-like growth factor 1 receptor (EC 2.7.10.1) (Insulin- | IGF1R |
| | | | like growth factor I receptor) (IGF-I receptor) (CD antigen | |
| | | | CD221) [Cleaved into: Insulin-like growth factor 1 receptor | |
| | | | alpha chain; Insulin-like growth factor 1 receptor beta chain] | |
| 16 | Q14003 | KCNC3_HUMAN | Potassium voltage-gated channel subfamily C member 3 | KCNC3 |
| | | | (KSHIIID) (Voltage-gated potassium channel subunit Kv3.3) | |
| 17 | O15350 | P73_HUMAN | Tumor protein p73 (p53-like transcription factor) (p53-related | TP73 P73 |
| | | | protein) | |
| 18 | Q12791 | KCMA1_HUMAN | Calcium-activated potassium channel subunit alpha-1 (BK | KCNMA1 KCNMA |
| | | | channel) (BKCA alpha) (Calcium-activated potassium | SLO |
| | | | channel, subfamily M subunit alpha-1) (K(VCA)alpha) | |
| | | | (KCa1.1) (Maxi K channel) (MaxiK) (Slo-alpha) (Slo1) | |
| | | | (Slowpoke homolog) (Slo homolog) (hSlo) | |
| 19 | P42261 | GRIA1_HUMAN | Glutamate receptor 1 (GluR-1) (AMPA-selective glutamate | GRIA1 GLUH1 |
| | | | receptor 1) (GluR-A) (GluR-K1) (Glutamate receptor | GLUR1 |
| | | | ionotropic, AMPA 1) (GluA1) | |
| 20 | P22303 | ACES_HUMAN | Acetylcholinesterase (AChE) (EC 3.1.1.7) | ACHE |
| 21 | P04040 | CATA_HUMAN | Catalase (EC 1.11.1.6) | CAT |
| 22 | Q9H8Y8 | GORS2_HUMAN | Golgi reassembly-stacking protein 2 (GRS2) (Golgi | GORASP2 |
| | | | phosphoprotein 6) (GOLPH6) (Golgi reassembly-stacking | GOLPH6 |
| | | | protein of 55 kDa) (GRASP55) (p59) | |
| 23 | P34897 | GLYM_HUMAN | Serine hydroxymethyltransferase, mitochondrial (SHMT) | SHMT2 |
| | | | (EC 2.1.2.1) (Glycine hydroxymethyltransferase) (Serine | |
| | | | methylase) | |
| 24 | P13760 | 2B14_HUMAN | HLA class II histocompatibility antigen, DRB1-4 beta chain | HLA-DRB1 |
| | | | (MHC class II antigen DRB1*4) (DR-4) (DR4) | |
| 25 | P04229 | 2B11_HUMAN | HLA class II histocompatibility antigen, DRB1-1 beta chain | HLA-DRB1 |
| | | | (MHC class II antigen DRB1*1) (DR-1) (DR1) | |
| 26 | P35914 | HMGCL_HUMAN | Hydroxymethylglutaryl-CoA lyase, mitochondrial (HL) | HMGCL |
| | | | (HMG-CoA lyase) (EC 4.1.3.4) (3-hydroxy-3- | |
| | | | methylglutarate-CoA lyase) | |
| 27 | Q29974 | 2B1G_HUMAN | HLA class II histocompatibility antigen, DRB1-16 beta chain | HLA-DRB1 |
| | | | (MHC class II antigen DRB1*16) (DR-16) (DR16) | |
| 28 | Q9TQE0 | 2B19_HUMAN | HLA class II histocompatibility antigen, DRB1-9 beta chain | HLA-DRB1 |
| | | | (MHC class II antigen DRB1*9) (DR-9) (DR9) | |
| 29 | Q9UDR5 | AASS_HUMAN | Alpha-aminoadipic semialdehyde synthase, mitochondrial | AASS |
| | | | (LKR/SDH) [Includes: Lysine ketoglutarate reductase (LKR) | |
| | | | (LOR) (EC 1.5.1.8); Saccharopine dehydrogenase (SDH) (EC | |
| | | | 1.5.1.9)] | |
| 30 | P13761 | 2B17_HUMAN | HLA class II histocompatibility antigen, DRB1-7 beta chain | HLA-DRB1 |
| | | | (MHC class II antigen DRB1*7) (DR-7) (DR7) | |
| 31 | P49450 | CENPA_HUMAN | Histone H3-like centromeric protein A (Centromere | CENPA |
| | | | autoantigen A) (Centromere protein A) (CENP-A) | |
| 32 | Q9Y2W7 | CSEN_HUMAN | Calsenilin (A-type potassium channel modulatory protein 3) | KCNIP3 CSEN |
| | | | (DRE-antagonist modulator) (DREAM) (Kv channel- | DREAM KCHIP3 |
| | | | interacting protein 3) (KChIP3) | |
| 33 | Q16630 | CPSF6_HUMAN | Cleavage and polyadenylation specificity factor subunit 6 | CPSF6 CFIM68 |
| | | | (Cleavage and polyadenylation specificity factor 68 kDa | |
| | | | subunit) (CFIm68) (CPSF 68 kDa subunit) (Pre-mRNA | |
| | | | cleavage factor Im 68 kDa subunit) (Protein HPBRII-4/7) | |
| 34 | O43809 | CPSF5_HUMAN | Cleavage and polyadenylation specificity factor subunit 5 | NUDT21 CFIM25 |
| | | | (Cleavage and polyadenylation specificity factor 25 kDa | CPSF25 CPSF5 |
| | | | subunit) (CFIm25) (CPSF 25 kDa subunit) (Nucleoside | |
| | | | diphosphate-linked moiety X motif 21) (Nudix motif 21) | |
| | | | (Pre-mRNA cleavage factor Im 25 kDa subunit) | |
| 35 | Q8N684 | CPSF7_HUMAN | Cleavage and polyadenylation specificity factor subunit 7 | CPSF7 |
| | | | (Cleavage and polyadenylation specificity factor 59 kDa | |
| | | | subunit) (CFIm59) (CPSF 59 kDa subunit) (Pre-mRNA | |
| | | | cleavage factor Im 59 kDa subunit) | |
| 36 | Q14999 | CUL7_HUMAN | Cullin-7 (CUL-7) | CUL7 KIAA0076 |
| 37 | Q9UI08 | EVL_HUMAN | Ena/V ASP-like protein (Ena/vasodilator-stimulated | EVL RNB6 |
| | | | phosphoprotein-like) | |
| 38 | Q05193 | DYN1_HUMAN | Dynamin-1 (EC 3.6.5.5) | DNM1 DNM |
| 39 | Q8N8S7 | ENAH_HUMAN | Protein enabled homolog | ENAH MENA |
| 40 | Q96PP8 | GBP5_HUMAN | Guanylate-binding protein 5 (EC 3.6.5.-) (GBP-TA antigen) | GBP5 |
| | | | (GTP-binding protein 5) (GBP-5) (Guanine nucleotide- | UNQ2427/PRO4987 |
| | | | binding protein 5) | |
| 41 | Q92947 | GCDH_HUMAN | Glutaryl-CoA dehydrogenase, mitochondrial (GCD) (EC | GCDH |
| | | | 1.3.8.6) | |
| 42 | Q13614 | MTMR2_HUMAN | Myotubularin-related protein 2 (Phosphatidylinositol-3,5- | MTMR2 KIAA1073 |
| | | | bisphosphate 3-phosphatase) (EC 3.1.3.95) | |
| | | | (Phosphatidylinositol-3-phosphate phosphatase) (EC | |
| | | | 3.1.3.64) | |
| 43 | Q99784 | NOE1_HUMAN | Noelin (Neuronal olfactomedin-related ER localized protein) | OLFM1 NOE1 |
| | | | (Olfactomedin-1) | NOEL1 |
| 44 | P50542 | PEX5_HUMAN | Peroxisomal targeting signal 1 receptor (PTS1 receptor) | PEX5 PXR1 |
| | | | (PTS1R) (PTS1-BP) (Peroxin-5) (Peroxisomal C-terminal | |
| | | | targeting signal import receptor) (Peroxisome receptor 1) | |
| 45 | P42262 | GRIA2_HUMAN | Glutamate receptor 2 (GluR-2) (AMPA-selective glutamate | GRIA2 GLUR2 |
| | | | receptor 2) (GluR-B) (GluR-K2) (Glutamate receptor | |
| | | | ionotropic, AMPA 2) (GluA2) | |
| 46 | P48058 | GRIA4_HUMAN | Glutamate receptor 4 (GluR-4) (GluR4) (AMPA-selective | GRIA4 GLUR4 |
| | | | glutamate receptor 4) (GluR-D) (Glutamate receptor | |
| | | | ionotropic, AMPA 4) (GluA4) | |
| 47 | P42263 | GRIA3_HUMAN | Glutamate receptor 3 (GluR-3) (AMPA-selective glutamate | GRIA3 GLUR3 |
| | | | receptor 3) (GluR-C) (GluR-K3) (Glutamate receptor | GLURC |
| | | | ionotropic, AMPA 3) (GluA3) | |
| 48 | O60741 | HCN1_HUMAN | Potassium/sodium hyperpolarization-activated cyclic | HCN1 BCNG1 |
| | | | nucleotide-gated channel 1 (Brain cyclic nucleotide-gated | |
| | | | channel 1) (BCNG-1) | |
| 49 | Q9UL51 | HCN2_HUMAN | Potassium/sodium hyperpolarization-activated cyclic | HCN2 BCNG2 |
| | | | nucleotide-gated channel 2 (Brain cyclic nucleotide-gated | |
| | | | channel 2) (BCNG-2) | |
| 50 | Q9Y3Q4 | HCN4_HUMAN | Potassium/sodium hyperpolarization-activated cyclic | HCN4 |
| | | | nucleotide-gated channel 4 | |
| 51 | P04035 | HMDH_HUMAN | 3-hydroxy-3-methylglutaryl-coenzyme A reductase (HMG- | HMGCR |
| | | | CoA reductase) (EC 1.1.1.34) | |
| 52 | Q8NCD3 | HJURP_HUMAN | Holliday junction recognition protein (14-3-3-associated | HJURP FAKTS |
| | | | AKT substrate) (Fetal liver-expressing gene 1 protein) (Up- | FLEG1 URLC9 |
| | | | regulated in lung cancer 9) | |
| 53 | Q9NZV8 | KCND2_HUMAN | Potassium voltage-gated channel subfamily D member 2 | KCND2 KIAA1044 |
| | | | (Voltage-gated potassium channel subunit Kv4.2) | |
| 54 | P48547 | KCNC1_HUMAN | Potassium voltage-gated channel subfamily C member 1 | KCNC1 |
| | | | (NGK2) (Voltage-gated potassium channel subunit Kv3.1) | |
| | | | (Voltage-gated potassium channel subunit Kv4) | |
| 55 | Q96CX2 | KCD12_HUMAN | BTB/POZ domain-containing protein KCTD12 (Pfetin) | KCTD12 C13orf2 |
| | | | (Predominantly fetal expressed T1 domain) | KIAA1778 PFET1 |
| 56 | P16389 | KCNA2_HUMAN | Potassium voltage-gated channel subfamily A member 2 | KCNA2 |
| | | | (NGK1) (Voltage-gated K(+) channel HuKIV) (Voltage- | |
| | | | gated potassium channel HBK5) (Voltage-gated potassium | |
| | | | channel subunit Kv1.2) | |
| 57 | P56696 | KCNQ4_HUMAN | Potassium voltage-gated channel subfamily KQT member 4 | KCNQ4 |
| | | | (KQT-like 4) (Potassium channel subunit alpha KvLQT4) | |
| | | | (Voltage-gated potassium channel subunit Kv7.4) | |
| 58 | Q9NXV2 | KCTD5_HUMAN | BTB/POZ domain-containing protein KCTD5 | KCTD5 |
| 59 | Q14721 | KCNB1_HUMAN | Potassium voltage-gated channel subfamily B member 1 | KCNB1 |
| | | | (Delayed rectifier potassium channel 1) (DRK1) (h-DRK1) | |
| | | | (Voltage-gated potassium channel subunit Kv2.1) | |
| 60 | Q86WG5 | MTMRD_HUMAN | Myotubularin-related protein 13 (SET-binding factor 2) | SBF2 CMT4B2 |
| | | | | KIAA1766 |
| | | | | MTMR13 |
| 61 | Q15070 | OXA1L_HUMAN | Mitochondrial inner membrane protein OXA1L (Hsa) | OXA1L |
| | | | (OXA1Hs) (Oxidase assembly 1-like protein) (OXA1-like | |
| | | | protein) | |
| 62 | P11498 | PYC_HUMAN | Pyruvate carboxylase, mitochondrial (EC 6.4.1.1) (Pyruvic | PC |
| | | | carboxylase) (PCB) | |
| 63 | P33764 | S10A3_HUMAN | Protein S100-A3 (Protein S-100E) (S100 calcium-binding | S100A3 S100E |
| | | | protein A3) | |
| 64 | P58743 | S26A5_HUMAN | Prestin (Solute carrier family 26 member 5) | SLC26A5 PRES |
| 65 | Q9UIL1 | SCOC_HUMAN | Short coiled-coil protein | SCOC SCOCO |
| | | | | HRIHFB2072 |
| 66 | P02549 | SPTA1_HUMAN | Spectrin alpha chain, erythrocytic 1 (Erythroid alpha- | SPTA1 SPTA |
| | | | spectrin) | |
| 67 | Q96QT4 | TRPM7_HUMAN | Transient receptor potential cation channel subfamily M | TRPM7 CHAK1 |
| | | | member 7 (EC 2.7.11.1) (Channel-kinase 1) (Long transient | LTRPC7 |
| | | | receptor potential channel 7) (LTrpC-7) (LTrpC7) | |
| 68 | Q9HCF6 | TRPM3_HUMAN | Transient receptor potential cation channel subfamily M | TRPM3 KIAA1616 |
| | | | member 3 (Long transient receptor potential channel 3) | LTRPC3 |
| | | | (LTrpC-3) (LTrpC3) (Melastatin-2) (MLSN2) | |
| 69 | Q7Z4N2 | TRPM1_HUMAN | Transient receptor potential cation channel subfamily M | TRPM1 LTRPC1 |
| | | | member 1 (Long transient receptor potential channel 1) | MLSN MLSN1 |
| | | | (LTrpC1) (Melastatin-1) | |
| 70 | Q9NQA5 | TRPV5_HUMAN | Transient receptor potential cation channel subfamily V | TRPV5 ECAC1 |
| | | | member 5 (TrpV5) (Calcium transport protein 2) (CaT2) | |
| | | | (Epithelial calcium channel 1) (ECaC) (ECaC1) (Osm-9-like | |
| | | | TRP channel 3) (OTRPC3) | |
| 71 | Q9BX84 | TRPM6_HUMAN | Transient receptor potential cation channel subfamily M | TRPM6 CHAK2 |
| | | | member 6 (EC 2.7.11.1) (Channel kinase 2) (Melastatin- | |
| | | | related TRP cation channel 6) | |
| 72 | P49638 | TTPA_HUMAN | Alpha-tocopherol transfer protein (Alpha-TTP) | TTPA TPP1 |
| 73 | Q8NBZ7 | UXS1_HUMAN | UDP-glucuronic acid decarboxylase 1 (EC 4.1.1.35) (UDP- | UXS1 |
| | | | glucuronate decarboxylase 1) (UGD) (UXS-1) | UNQ2538/PRO6079 |
| 74 | Q13426 | XRCC4_HUMAN | DNA repair protein XRCC4 (X-ray repair cross- | XRCC4 |
| | | | complementing protein 4) | |
| 75 | A0A0A6YY98 | A0A0A6YY98_HUMAN | Transient receptor potential cation channel subfamily V | TRPV5 |
| | | | member 5 | |
| 76 | H0YLN8 | H0YLN8_HUMAN | Transient receptor potential cation channel subfamily M | TRPM7 hCG_39859 |
| | | | member 7 (Transient receptor potential cation channel, | |
| | | | subfamily M, member 7, isoform CRA_c) | |
| 77 | A0A0C4DFW9 | A0A0C4DFW9_HUMAN | Cellular tumor antigen p53 | TP73 hCG_19088 |
| 78 | G5E9G1 | G5E9G1_HUMAN | Transient receptor potential cation channel subfamily M | TRPM3 |
| | | | member 3 (Transient receptor potential cation channel, | hCG_2042991 |
| | | | subfamily M, member 3, isoform CRA_a) | |
| 79 | H7BYP1 | H7BYP1_HUMAN | Transient receptor potential cation channel subfamily M | TRPM3 |
| | | | member 3 (Transient receptor potential cation channel, | hCG_2042991 |
| | | | subfamily M, member 3, isoform CRA_c) | |
| 80 | A0A024R4C3 | A0A024R4C3_HUMAN | Tumor protein p73, isoform CRA_a | TP73 hCG_19088 |
| 81 | A0A0S2Z4N5 | A0A0S2Z4N5_HUMAN | Tumor protein p63 isoform 1 (Tumor protein p73-like, | TP63 TP73L |
| | | | isoform CRA_a) (Fragment) | hCG_16028 |
| 82 | A0A024R5V1 | A0A024R5Vl_HUMAN | Transient receptor potential cation channel, subfamily M, | TRPM7 hCG_39859 |
| | | | member 7, isoform CRA_a | |
| 83 | X5D8S6 | X5D8S6_HUMAN | Adenylosuccinate lyase (ASL) (EC 4.3.2.2) | ADSL hCG_40060 |
| | | | (Adenylosuccinase) (Fragment) | |
| 84 | C9D7D0 | C9D7D0_HUMAN | Cellular tumor antigen p53 | TP63 |
| 85 | K7PPA8 | K7PPA8_HUMAN | Cellular tumor antigen p53 | TP53 |
| 86 | A2A3F4 | A2A3F4_HUMAN | Transient receptor potential cation channel subfamily M | TRPM3 |
| | | | member 3 | |
| 87 | Q2XSC7 | Q2XSC7_HUMAN | Cellular tumor antigen p53 | TP53 |
| 88 | A0A024R209 | A0A024R209_HUMAN | Transient receptor potential cation channel, subfamily M, | TRPM1 hCG_37570 |
| | | | member 1, isoform CRA_a | |
| 89 | Q1MSX0 | Q1MSX0_HUMAN | Cellular tumor antigen p53 (Fragment) | TP53 |
| 90 | A0A0G2JN34 | A0A0G2JN34_HUMAN | Transient receptor potential cation channel subfamily M | TRPM1 |
| | | | member 1 | |
| 91 | H6U5S3 | H6U5S3_HUMAN | Cellular tumor antigen p53 (Fragment) | |
| 92 | H2EHT1 | H2EHT1_HUMAN | Cellular tumor antigen p53 | TP53 |
| 93 | H6U5S2 | H6U5S2_HUMAN | Cellular tumor antigen p53 (Fragment) | |
| 94 | B4DNI2 | B4DNI2_HUMAN | Cellular tumor antigen p53 | |
| 95 | C9D7C9 | C9D7C9_HUMAN | Cellular tumor antigen p53 | TP63 |
| 96 | B6E4X6 | B6E4X6_HUMAN | Cellular tumor antigen p53 | |
| 97 | A0A087WZU8 | A0A087WZU8_HUMAN | Cellular tumor antigen p53 | TP53 |
| 98 | K7PPU4 | K7PPU4_HUMAN | Cellular tumor antigen p53 | TP53 |
| 99 | A0A0U1RQC9 | A0A0U1RQC9_HUMAN | Cellular tumor antigen p53 | TP53 |
| 100 | Q5U0E4 | Q5U0E4_HUMAN | Cellular tumor antigen p53 | |
| 101 | Q53GA5 | Q53GA5_HUMAN | Cellular tumor antigen p53 (Fragment) | |
| 102 | A2A3F7 | A2A3F7_HUMAN | Transient receptor potential cation channel subfamily M | TRPM3 |
| | | | member 3 | |
| 103 | B4DMH2 | B4DMH2_HUMAN | Cellular tumor antigen p53 | |
| 104 | B7Z8X6 | B7Z8X6_HUMAN | Cellular tumor antigen p53 | |
| 105 | A0A141PNN3 | A0A141PNN3_HUMAN | Cellular tumor antigen p53 | TP63 |
| 106 | A0A141PNN4 | A0A141PNN4_HUMAN | Cellular tumor antigen p53 | TP63 |
| 107 | A0A087X1Q1 | A0A087X1Q1_HUMAN | Cellular tumor antigen p53 | TP53 |
| 108 | E5RMA8 | E5RMA8_HUMAN | Cellular tumor antigen p53 | TP53 |
| 109 | A0A0S2Z4N6 | A0A0S2Z4N6_HUMAN | Tumor protein p63 isoform 2 (Fragment) | TP63 |
| 110 | E9PBI7 | E9PBI7_HUMAN | Transient receptor potential cation channel subfamily M | TRPM3 |
| | | | member 3 | |
| 111 | A0A0G2JMR4 | A0A0G2JMR4_HUMAN | Transient receptor potential cation channel subfamily M | TRPM1 |
| | | | member 1 | |
| 112 | Q9H637 | Q9H637_HUMAN | cDNA: FLJ22628 fis, clone HSI06177 | |
| 113 | A0A0G2JPN6 | A0A0G2JPN6_HUMAN | Transient receptor potential cation channel subfamily M | TRPM1 |
| | | | member 1 | |
| 114 | A0A024R212 | A0A024R212_HUMAN | Transient receptor potential cation channel, subfamily M, | TRPM1 hCG_37570 |
| | | | member 1, isoform CRA_b | |
| 115 | A0A0G2JMJ5 | A0A0G2JMJ5_HUMAN | Transient receptor potential cation channel subfamily M | TRPM1 |
| | | | member 1 | |
| 116 | HOYM61 | HOYM61_HUMAN | Transient receptor potential cation channel subfamily M | TRPM1 |
| | | | member 1 (Fragment) | |
| 117 | HOYKU7 | HOYKU7_HUMAN | Transient receptor potential cation channel subfamily M | TRPM1 |
| | | | member 1 (Fragment) | |
| 118 | A0A0A0MTQ9 | A0A0A0MTQ9_HUMAN | Transient receptor potential cation channel subfamily M | TRPM1 |
| | | | member 1 (Fragment) | |
| 119 | A2A3F3 | A2A3F3_HUMAN | Transient receptor potential cation channel subfamily M | TRPM3 |
| | | | member 3 | |

The amino acid and nucleotide sequences of each of these proteins and the TD thereof is incorporated herein by reference for use in the present invention and for potential inclusion in one or more claims herein.

TABLE 3
DNA sequences encoding Quad nolvnentides

| SEQ | | |
|---|---|---|
| ID NO: | POLYPEPTIDE | NUCLEOTIDE SEQUENCE |
| 13 | Quad 1 | ATGAGCCTCGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGGCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG |
| | | TTCCCACCCGAGGTCGCTGTGTTTGAGCCATCAGAAGCAGAGATCTCCCACACCCAAAAG |
| | | GCCACACTGGTGTGCCTGGCCACAGGCTTCTACCCCGACCATGTGGAGCTGAGCTGGTGG |
| | | GTGAATGGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCAGGATAGGGCCAAACCCGTCACCCAGATCGTCAGCGCCGAGGCCTGG |
| | | GGTAGAGCAGACGCCAGCACCAAGGGCCCCTCTGTGTTCCCTCTGGCCCCTTCCAGCAAG |
| | | TCCACCTCTGGCGGAACAGCCGCTCTGGGCTGCCTCGTGAAGGACTACTTCCCCGAGCCT |
| | | GTGACCGTGTCCTGGAACTCTGGCGCTCTGACCAGCGGAGTGCACACCTTCCCTGCTGTG |
| | | CTGCAGTCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTGGACAAG |
| | | AAGGTGCTAACAGACAGAGAATGGGCAGAAGAGTGGAAACATCTTGACCATCTGTTAAAC |
| | | TGCATAATGGACATGGTAGAAAAAACAAGGCGATCTCTCACCGTACTAAGGCGGTGTCAA |
| | | GAAGCAGACCGGGAAGAATTGAATTACTGGATCCGGCGGTACAGTGACGCCGAGGACTAC |
| | | AAGGACGACGACGACAAGCACCACCACCATCACCAC |
| 14 | Quad 2 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGGCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG |
| | | TTCCCACCCGAGGTCGCTGTGTTTGAGCCATCAGAAGCAGAGATCTCCCACACCCAAAAG |
| | | GCCACACTGGTGTGCCTGGCCACAGGCTTCTACCCCGACCATGTGGAGCTGAGCTGGTGG |
| | | GTGAATGGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCAGGATAGGGCCAAACCCGTCACCCAGATCGTCAGCGCCGAGGCCTGG |
| | | GGTAGAGCAGACGCCAGCACCAAGGGCCCCTCTGTGTTCCCTCTGGCCCCTTCCAGCAAG |
| | | TCCACCTCTGGCGGAACAGCCGCTCTGGGCTGCCTCGTGAAGGACTACTTCCCCGAGCCT |
| | | GTGACCGTGTCCTGGAACTCTGGCGCTCTGACCAGCGGAGTGCACACCTTCCCTGCTGTG |
| | | CTGCAGTCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTGGACAAG |
| | | AAGGTGGGAGGAGGTGCGAGCCTAACAGACAGAGAATGGGCAGAAGAGTGGAAACATCTT |
| | | GACCATCTGTTAAACTGCATAATGGACATGGTAGRAAAAACAAGGCGATCTCTCACCGTA |
| | | CTAAGGCGGTGTCAAGAAGCAGACCGGGAAGAATTGAATTACTGGATCCGGCGGTACAGT |
| | | GACGCCGAGGACTACAAGGACGACGACGACAAGCACCACCACCATCACCAC |
| 15 | Quad 3 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGGCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG |
| | | TTCCCACCCGAGGTCGCTGTGTTTGAGCCATCAGAAGCAGAGATCTCCCACACCCAAAAG |
| | | GCCACACTGGTGTGCCTGGCCACAGGCTTCTACCCCGACCATGTGGAGCTGAGCTGGTGG |
| | | GTGAATGGGAAGGAGGTGCACAGTGGGGTCTGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCAGGATAGGGCCAAACCCGTCACCCAGATCGTCAGCGCCGAGGCCTGG |
| | | GGTAGAGCAGACGCCAGCACCAAGGGCCCCTCTGTGTTCCCTCTGGCCCCTTCCAGCAAG |
| | | TCCACCTCTGGCGGAACAGCCGGTCTGGGCTGCCTCGTGAAGGACTACTTCCCCGAGCCT |
| | | GTGACCGTGTCCTGGAACTCTGGCGCTCTGACCAGCGGAGTGGACACCTTCCCTGCTGTG |
| | | CTGCAGTCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTGGACAAG |
| | | AAGGTGCTAACAGACAGAGAATGGGCAGAAGAGTGGAAACATCTTGACCATCTGTTAAAC |
| | | TGCATAATGGACATGGTAGAAAAAACAAGGCGATCTCTCACCGTACTAAGGCGGTGTCAA |
| | | GAAGCAGACCGGAAGAATTGAATTACTGGATCCGGCGGTACAGTGACGCCGAGGACTAC |
| | | AAGGACGACGACGACAAGCACCACCACCATCACCAC |
| 16 | Quad 4 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGGCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTIGGAGAAGGCTCTAGGCTGACCGTACTGGAGGACCTGAAAAACGTG |
| | | TTCCCACCCCAGGTCGCTGTGTTTGAGCCATCAGAAGCAGAGATCTCCCACACCCAAAAG |
| | | GCCACACTGGTGTCCCTGGCCACAGGCTTCTACCCCGACCATGTGGACCTGAGCTGGTGG |
| | | GTGAATGGGAAGGAGGTGCACAGTGGGGTCTGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCAGCATAGCGCCAAACCOGTCACCCAGATCGTCAGCGCCGAGCCCTGG |
| | | GGTAGAGCAGACGCCAGCACCAAGGGCCCCTCTGTGTTCCCTCTGGCCCCTTCCAGCAAG |
| | | TCCACCTCTGGCGGAACAGCCGCTCTGGGCTGCCTCGTGAAGGACTACTTCCCCGAGCCT |
| | | GTGACCGTGTCCTGGAACTCTGGCGCTCTGACCAGCGGAGTGCACACCTTCCCTGCTGTG |
| | | CTGCAGTCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTGGACAAG |
| | | AAGGTGGGAGGAGGTGGGAGCCTAACAGACAGAGAATGGGCAGAAGAGTGGAAACATCTT |
| | | GACCATCTGTTAAACTGCATAATGGACATGGTAGAAAAAACAAGGCGATCTCTCACCGTA |
| | | CTAAGGCGGTGTCAAGAAGCAGACCGGGAAGAATTGAATTACTGGATCCGGCGGTACAGT |
| | | GACGCCGAGGACTACAAGGACGACGACGACAAGCACCACCACCATCACCAC |
| 17 | Quad 5 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGGCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG |
| | | TTCCCACCCGAGGTCGCTGTGTTTGAGCCATCAGAAGCAGAGATCTCCCACACCCAAAAG |
| | | GCCACACTGGTGTGCCTGGCCACAGGCTTCTACCCCGACCATGTGGAGCTGAGCTGGTGG |
| | | GTGAATGGGAAGGAGGTGCACAGTGGGGTCTGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCAGGATAGGGCCAAACCCGTCACCCAGATCGTCAGCGCCGAGGCCTGG |
| | | GGTAGAGCAGACCTAACAGACAGAGAATGGGCAGAAGAGTGGAAACATCTTGACCATCTG |
| | | TTAAACTGCATAATGGACATGGTAGAAAAAACAAGGCGATCTCTCACCGTACTAAGGCGG |
| | | TGTCAAGAAGCAGACCGGGAAGAATTGAATTACTGGATCCGGCGGTACAGTGACGCCGAG |
| | | GACTACAAGGACGACGACGACAAGCACCACCACCATCACCAC |
| 18 | Quad 6 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGGCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG |
| | | TTCCCACCCGAGGTCGCTGTGTTTGAGCCATCAGAAGCAGAGATCTCCCACACCCAAAAG |
| | | GCCACACTGGTGTCCCTGGCCACAGGCTTCTACCCCGACCATGTGGACCTGAGCTGGTGG |
| | | GTGAATGGGAAGGAGGTGCACAGTGGGGTCTGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCAGGATAGGGCCAAACCCGTCACCCAGATCGTCAGCGCCGAGGCCTGG |
| | | GGTAGAGCAGACGGAGGAGGTGGGAGCCTAACAGACAGAGAATGGGCAGAAGAGTGGAAA |
| | | CATCTTGACCATCTGTTAAACTGCATAATGGACATGGTAGAAAAAACAAGGCGATCTCTC |
| | | ACCGTACTAAGGCGGTGTCAAGAAGCAGACCGGGAAGAATTGAATTACTGGATCCGGCGG |
| | | TACAGTGACGCCGAGGACTACAAGGACGACGACGACAAGCACCACCACCATCACCAC |
| 19 | Quad 7 | ATGGAGACCCTCTTGGGCCTGCTTATCCTTTGGCTGCAGCTGCAATGGGTGAGC |
| | | AGCAAACAGGAGGTGACACAGATTCCTGCAGCTCTGAGTGTCCCAGAAGGAGAAAACTTG |
| | | GTTCTCAACTGCAGTTTCACTGATAGCGCTATTTACAACCTCCAGTGGTTTAGGCAGGAC |
| | | CCTGGGAAAGGTCTCACATCTCTGTTGCTTATTACACCTTGGCAGAGAGAGCAAACAAGT |
| | | GGAAGACTTAATGCCTCGCTGGATAAATCATCAGGACGTAGTACTTTATACATTGCAGCT |
| | | TCTCAGCCTGGTGACTCAGCCACCTACCTCTGTGCTGTGAGGCCCCTGCTTGACGGAACA |
| | | TACATACCTACATTTGGAAGAGGAACCAGCCTTATTGTTCATCCGTATATCCAGAACCCT |
| | | GACCCTGCCGTGTACCAGCTGAGAGACTCTAAATCCAGTGACAAGTCTGTCTGCCTATTC |
| | | ACCGATTTTGATTCTCAAACAAATGTGTCACAAAGTAAGGATTCTGATGTGTATATCACA |
| | | GACAAAACTGTGCTAGACATGAGGTCTATGGACTTCAAGAGCAACAGTGCTGTGGCCTGG |
| | | AGCAACAAATCTGACTTTGCATGTGCAAACGCCTTCAACAACAGCATTATTCCAGAAGAC |
| | | ACCTTCTTCCCCAGCCCAGAAAGTTCCACGGTGGCCGCTCCCTCCGTGTTCATCTTCCCA |
| | | CCTTCCGACGAGCAGCTGAAGTCCGGCACCGCTTCTGTCGTGTGCCTGCTGAACAACTTC |
| | | TACCCCCGCGAGGCCAAGGTGCAGTGGNAGGTGGACAACGCCCTGCAGTCCGGCAACTCC |
| | | CAGGAATCCGTGACCGAGCAGGACTCCAAGGACAGCACCTACTCCCTGTCCTCCACCCTG |
| | | ACCCTGTCCAAGGCCGACTACGAGAAGCACAAGGTGTACGCCTGCGAAGTGACCCACCAG |
| | | GGCCTGTCTAGCCCCGTGACCAAGTCTTTCAACCGGGGCGAGTGT |
| 20 | Quad 8 | ATGGAGACCCTCTTGGGCCTGCTTATCCTTTGGCTGCAGCTGCAATGGGTGAGC |
| | | AGCAAACAGGAGGTGACACAGATTCCTGCAGCTCTGAGTGTCCCAGAAGGAGAAAACTTG |
| | | GTTCTCAACTGCAGTTTCACTGATAGCGCTATTTACAACCTCCAGTGGTTTAGGCAGGAG |
| | | CCTGGGAAAGGTCTCACATCTCTGTTGCTTATTACACCTTGGCAGAGAGAGCAAACAAGT |
| | | GGAAGACTTAATGCCTCGCTGGATAAATCATCAGGACGTAGTACTTTATACATTGCAGCT |
| | | TCTCAGCCTGGTGACTCAGCCACCTACCTCTGTGCTGTGAGGCCCCTGCTTGACGGAACA |
| | | TACATACCTACATTTGGAAGAGGAACCAGCCTTATTGTTCATCCGTATATCCAGAACCCT |
| | | GACCCTGCCGTGTACCAGCTGAGAGACTCTAAATCCAGTGACAAGTCTGTCTGCCTATTC |
| | | ACCGATTTTGATTCTCAAACAAATGTGTCACAAAGTAAGGATTCTGATGTGTATATCACA |
| | | GACAAATGTGTGCTAGACATGAGGTCTATGGACTTCAAGAGCAACAGTGCTGTGGCCTGG |
| | | AGCAACAAATCTGACTTTGCATGTGCAAACGCCTTCAACAACAGCATTATTCCAGAAGAC |
| | | ACCTTCTTCCCCAGCCCAGAAAGTTCCACGGTGGCCGCTCCCTCCGTGTTCATCTTCCCA |
| | | CCTTCCCACGAGCAGCTGAAGTCCGGCACCGCTTCTGTCGTGTCCCTGCTGAACAACTTC |
| | | TACCCCCGCGAGGCCAAGGTGCAGTGGAAGGTGGACAACGCCCTGCAGTCCGGCAACTCC |
| | | CAGGAATCCGTGACCCAGCAGGACTCCAAGGACAGCACCTACTCCCTGTCCTCCACCCTG |
| | | ACCCTGTCCAAGGCCGACTACGAGAAGCACAAGGTGTACGCCTGCGAAGTGACCCACCAG |
| | | GGCCTGTCTAGCCCCGTGACCAAGTCTTTCAACCGGGGCGAGTGT |
| 21 | Quad 9 | ATGGAGACCCTCTTGGGCCTGCTTATCCTTTGGCTGCAGCTGCAATGGGTGAGC |
| | | AGCAAACAGGAGGTGACACAGATTCCTGCAGCTCTGAGTGTCCCAGAAGGAGAAAACTTG |
| | | GTTCTCAACTGCAGTTTCACTGATAGCGCTATTTACAACCTCCAGTGGTTTAGGCAGGAC |
| | | CCTGGGAAAGGTCTCACATCTCTGTTGCTTATTACACCTTGGCAGAGAGAGCAAACAAGT |
| | | GGAAGACTTAATGCCTCGCTGGATAAATCATCAGGACGTAGTACTTTATACATTGCAGCT |
| | | TCTCAGCCTGGTGACTCAGCCACCTACCTCTGTGCTGTGAGGCCCCTGCTTGACGGAACA |
| | | TACATACCTACATTTGGAAGAGGAACCAGCCTTATTGTTCATCCGTATATCCAGAACCCT |
| | | GACCCTGCCGTGTACCAGCTGAGAGACTCTAAATCCAGTGACAAGTCTGTCTGCCTATTC |
| | | ACCGATTTTGATTCTCAAACAAATGTGTCACAAAGTAAGGATTCTGATGTGTATATCACA |
| | | GACAAATGTGTGCTAGACATGAGGTCTATGGACTTCAAGAGCAACAGTGCTGTGGCCTGG |
| | | AGCAACAAATCTCACTTTGCATGTGCAAACGCCTTCAACAACAGCATTATTCCAGAAGAC |
| | | ACCTTCTTCCCCAGCCCAGAAAGTTCC |
| 22 | Quad 10 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGCCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG |
| | | TTCCCACCCGAGGTCGCTGTGTTTGAGCCATCAGAAGCAGAGATCTCCCACACCCAAAAG |
| | | GCCACACTGGTGTGCCTGGCCACAGGCTTCTACCCCGACCATGTGGAGCTGAGCTGGTGG |
| | | GTGAATGGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCAGGATAGGGCCAAACCCGTCACCCAGATCGTCAGCGCCGAGGCCTGG |
| | | GGTAGAGCAGACGCCAGCACCAAGGGCCCCTCTGTGTTCCCTCTGGCCCCTTCCAGCAAG |
| | | TCCACCTCTGGCGGAACAGCCGCTCTGGGCTGCCTCGTGAAGGACTACTTCCCCGAGCCT |
| | | GTGACCGTGTCCTGGAACTCTGGCGCTCTGACCAGCGGAGTGCACACCTTCCCTGCTGTG |
| | | CTGCAGTCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTGGACAAG |
| | | AAGGTGCTAACAGACAGAGAATGGGCAGAAGAGTGGAAACATCTTGACCATCTGTTAAAC |
| | | TGCATAATGGACATGGTAGAAAAAACAAGGCGATCTCTCACCGTACTAAGGCGGTGTCAA |
| | | GAAGCAGACCGGGAAGAATTGAATTACTGGATCCGGCGGTACAGTGACGCCGAGGCACCT |
| | | ACTTCAAGTTCTACAAAGAAAACACAGCTACAACTGGAGCATTTACTGOTGGATTTACAG |
| | | ATGATTTTGAATGGAATTAATAATTACAAGAATCCCAAACTCACCAGGATGCTCACATTT |
| | | AAGTTTTACATGCCCAAGAAGGCCACAGAACTGAAACATCTTCAGTGTCTAGAAGAAGAA |
| | | CTCAAACCTCTGGAGGAAGTGCTAAATTTAGCTCAAAGCAAAAACTTTCACTTAAGACCC |
| | | AGGGACTTAATCAGCAATATCAACGTAATAGTTCTGGAACTAAAGGGATCTGAAACAACA |
| | | TTCATGTGTGAATATGCTGATGAGACAGCAACCATTGTAGAATTTCTGAACAGATGGATT |
| | | ACCTTTTGTCAAAGCATCATCTCAACACTCACTGACTACAAGGACGACGACGACAAGCAC |
| | | CACCACCATCACCAC |
| 23 | Quad 11 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGGOTGATTCATTACTOAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG |
| | | TTCCCACCCGAGGTCGCTGTGTTTGAGOCATCAGAAGCAGAGATCTCCCACACCCAAAAG |
| | | GCCACACTGGTGTGCCTGGCCACAGGCTTCTACCCCGACCATGTGGAGCTGAGCTGGTGG |
| | | GTGAATGGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCAGGATAGGGCCAAACCCGTCACCCAGATCGTCAGCGCCGAGGCCTGG |
| | | GGTAGAGCAGACGCCAGCACCAAGGGCCCCTCTGTGTTCCCTCTGGCCCCTTCCAGCAAG |
| | | TCCACCTCTGGCGGAACAGCCGCTCTGGCTGCCTCGTGAAGGACTACTTCCCCAGAGCCT |
| | | GTGACCGTGTCCTGGAACTCTGGCGCTCTGACCAGCGGAGTGCACACCTTCCCTGCTGTG |
| | | CTGCAGTCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTGGACAAG |
| | | AAGGTGGGAGGAGGTGGGAGCCTAACAGACAGAGAATGGGCAGAAGAGTGGAAACATCTT |
| | | GACCATCTGTTAAACTGCATAATGGACATGGTAGAAAAAACAAGGCGATCTCTCACCGTA |
| | | CTAAGGCGGTGTCAAGAAGCAGACCGGGAAGAATTGAATTACTGGATCCGGCGGTACAGT |
| | | GACGCCGAGGCACCTACTTCAAGTTCTACAAAGAAAACACAGCTACAACTGCAGCATTTA |
| | | CTGCTGGATTTACAGATGATTTTGAATGGAATTAATAATTACAAGAATCCCAAACTCACC |
| | | AGGATGCTCACATTTAAGTTTTACATGCCCAAGAAGGCCACAGAACTGAAACATCTTCAG |
| | | TGTCTAGAAGAAGAACTCAAACCTCTGGAGGAAGTGCTAAATTTAGCTCAAAGCAAAAAC |
| | | TTTCACTTAAGACCCAGGGACTTAATCAGCAATATCAACGTAATAGTTCTGGAACTAAAG |
| | | GGATCTGAAACAACATTCATGTGTGAATATGCTGATGAGACAGCAACCATTGTAGAATTT |
| | | CTGAACAGATGGATTACCTTTTGTCAAAGCATCATCTCAACACTGACTGACTACAAGGAC |
| | | GACGACGACAAGCACCACCACCATCACCAC |
| 24 | Quad 12 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGGCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG |
| | | TTCCCACCCCAGGTCGCTGTGTTTGAGCCATCAGAAGCAGAGATCTCCCACACCCAAAAG |
| | | GCCACACTGGTGTGCCTGGCCACAGGCTTCTACCCCGACCATGTGGAGCTGAGCTGGTGG |
| | | GTGAATGGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCACGATAGGGCCAAACCCGTCACCCAGATCGTCAGCGCCCAGGCCTGG |
| | | GGTAGAGCAGACGCCAGCACCAAGGGCCCCTCTGTGTTCCCTCTGGCCCCTTCCAGCAAG |
| | | TCCACCTCTGGCGGAACAGCCGCTCTGGGCTGCCTCGTGAAGGACTACTTCCCCGAGCCT |
| | | GTGACCGTGTCCTGGAACTCTGGCGCTCTGACCAGCGGAGTGCACACCTTCCCTGCTGTG |
| | | CTGCAGTCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTGGACAAG |
| | | AAGGTGGAACCCAAGTCCTGCGACAAGACCCACACCTGTCCCCCTTGTCCTCTAACAGAC |
| | | AGAGAATGGGCAGAAGAGTGGAAACATCTTGACCATCTGTTAAACTGCATAATGGACATG |
| | | GTAGAAAAAACAAGGCGATCTCTCACCGTACTAAGGCGGTGTCAAGAAGCAGACCGGGAA |
| | | GAATTGAATTACTGGATCCGGCGGTACAGTGACGCCGAGGACTACAAGGACGACGACGAC |
| | | AAGCACCACCACCATCACCAC |
| 25 | Quad 13 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGGCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG |
| | | TTCCCACCCGAGGTCGCTGTGTTTGAGCCATCAGAAGCAGAGATCTCCCACACCCAAAAG |
| | | GCCACACTGGTGTGCCTGGCCACAGGCTTCTACCCCGACCATGTGGAGCTGAGCTGGTGG |
| | | GTGAATGGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCAGGATAGGGCCAAACCCGTCACCCAGATCGTCAGCGCCGAGGCCTGG |
| | | GGTAGAGCAGACGCCAGCACCAAGGGCCCCTCTGTGTTCCCTCTGGCCCCTTCCAGCAAG |
| | | TCCACCTCTGGCGGAACAGCCGCTCTGGGCTGCCTCGTGAAGGACTACTTCCCCGAGCCT |
| | | CTGCAGTCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTGGACAAG |
| | | AAGGTGGAACCCAAGTCCTGCGACAAGACCCACACCTGTCCCCCTTGTCCTGGAGGAGGT |
| | | GGGAGCCTAACAGACAGAGAATGGGCAGAAGAGTGGAAACATCTTGACCATCTGTTAAAC |
| | | TGCATAATGGACATGGTAGAAAAAACAAGGCGATCTCTCACCGTACTAAGGCGGTGTCAA |
| | | GAAGCAGACCGGGAAGAATTGAATTACTGGATCCGGCGGTACAGTGACGCCGAGGACTAC |
| | | AAGGACGACGACGACAAGCACCACCACCATCACCATC |
| 26 | Quad 14 | ATGGAGCTGGGGCTGAGCTGGGTGGTCCTGGCTGCTCTACTACAAGGTGTCCAG |
| | | GCTCAGGTTCAGCTGGTTGAAAGCGGTGGTGCACTGGTTCAGCCTGGTGGTAGCCTGCGT |
| | | CTGAGCTGTGCAGCAAGCGGTTTTCCGGTTAATCGTTATAGCATGCGTTGGTATCGTCAG |
| | | GCACCGGGTAAAGAACGTGAATGGGTTGCAGGTATGAGCAGTGCCGGTGATCGTAGCAGC |
| | | TATGAAGATAGCGTTAAAGGTCGTTTTACCATCAGCCGTGATGATGCACGTAATACCGTT |
| | | TATCTGCAAATGAATAGCCTGAAACCGGAAGATACCGCAGTGTATTATTGCAATGTTAAC |
| | | GTGGGCTTTGAATATTGGGGTCAGGGCACCCAGGTTACCGTTAGCAGCAAACTAACAGAC |
| | | AGAGAATGGGCAGAAGAGTGGAAACATCTTGACCATCTGTTAAACTGCATAATGGACATG |
| | | GTAGAAAAAACAAGGCGATCTCTCACCGTACTAAGGCGGTGTCAAGAAGCAGACCGGGAA |
| | | GAATTGAATTACTGGATCCGGCGGTACAGTGACGCCGAGGACTACAAGGACGACGACGAC |
| | | AAGCACCACCACCATCACCAC |
| 27 | Quad 15 | ATGGAGCTGGGGCTGAGCTGGGTGGTCCTGGCTGCTCTACTACAAGGTGTCCAG |
| | | GCTCAGGTTCAGCTGGTTGAAAGCGGTGGTGCACTGGTTCAGCCTGGTGGTAGCCTGCGT |
| | | CTGAGCTGTGCAGCAAGCGGTTTTCCGCTTAATCGTTATAGCATGCCITGGTATCGTCAG |
| | | GCACCGGGTAAAGAACGTGAATGGGTTGCAGGTATGAGCAGTGCCGGTGATCGTAGCAGC |
| | | TATGAAGATAGCGTTAAAGGTCGTTTTACCATCAGCCGTGATGATGCACGTAATACCGTT |
| | | TATCTGCAAATGAATAGCCTGAAACCGGAAGATACCGCAGTGTATTATTGCAATGTTAAC |
| | | GTGCGCTTTGAATATTGGGCTCAGGGCACCCAGGTTACCGTTAGCAGCAAAGGAGGAGGT |
| | | GGGAGCCTAACAGACAGAGAATGGGCAGAAGAGTGGAAACATCTTGACCATCTGTTAAAC |
| | | TGCATAATGGACATGGTAGAAAAAACAAGGCGATCTCTCACCGTACTAAGGCGGTGTCAA |
| | | GAAGCAGACCGGGAAGAATTGAATTACTGGATCCGGCGGTACAGTGACGCCGAGGACTAC |
| | | AAGGACGACGACGACAAGCACCACCACCATCACCAC |
| 28 | Quad 18 | ATGGAGCTGGGGCTGAGCTGGGTGGTCCTGGCTGCTCTACTACAAGGTGTCCAG |
| | | GCTCAGGTTCAGCTGGTTGAAAGCGGTGGTGCACTGGTTCAGCCTGGTGGTAGCCTGCGT |
| | | CTGAGCTGTGCAGCAAGCGGTTTTCCGCTTAATCGTTATAGCATGCCITGGTATCGTCAG |
| | | GCACCGGGTAAAGAACGTGAATGGGTTGCAGGTATGAGCAGTGCCGGTGATCGTAGCAGC |
| | | TATGAAGATAGCGTTAAAGGTCGTTTTACCATCAGCCGTGATGATGCACGTAATACCGTT |
| | | TATCTGCAAATGAATAGCCTGAAACCGGAAGATACCGCAGTGTATTATTGCAATGTTAAC |
| | | GTGGGCTTTGAATATTGGGGTCAGGGCACCCAGGTTACCGTTAGCAGCAAACTAACAGAC |
| | | AGAGAATGGGCAGAAGAGTGGAAACATCTTGACCATCTGTTAAACTGCATAATGGACATG |
| | | GTAGAAAAAACAAGGCGATCTCTCACOGTACTAAGGCGGTGTCAAGAAGCAGACCGGGAA |
| | | GAATTGAATTACTGGATCCGGCGGTACACTGACGCCCAGGCACCTACTTCAACTTCTACA |
| | | AAGAAAACACAGCTACAACTGGAGCATTTACTGCTGGATTTACAGATGATTTTGAATGGA |
| | | ATTAATAATTACAAGAATCCCAAACTCACCAGGATGCTCACATTTAAGTTTTACATGCCC |
| | | AAGAAGGCCACAGAACTGAAACATCTTCAGTGTCTAGAAGAAGAACTCAAACCTCTGGAG |
| | | GAAGTGCTAAATTTAGCTCAAAGCAAAAACTTTCACTTAAGACCCAGGGACTTAATCAGC |
| | | AATATCAACGTAATAGTTCTGGAACTAAAGGGATCTGAAACAACATTGATGTGTGAATAT |
| | | GCTGATGAGACAGCAACCATTGTAGAATTTCTGAACAGATGGATTACCTTTTGTCAAAGC |
| | | ATCATCTCAACACTGACTGACTACAAGGACGACGACGACAAGCACCACCACCATCACCAC |
| 29 | Quad 19 | ATGGAGCTGGGGCTGAGCTGGGTGGTCCTGGCTGCTCTACTACAAGGTGTCCAG |
| | | GCTCAGGTTCAGCTGGTTGAAAGCGGTGGTGCACTGGTTCAGCCTGGTGGTAGCCTGCGT |
| | | CTGAGCTGTGCAGCAAGCGGTTTTCCGGTTAATCGTTATAGCATGCGTTGGTATCGTCAG |
| | | GCACCGGGTAAAGAACGTGAATGGGTTGCAGGTATGAGCAGTGCCGGTGATCGTAGCAGC |
| | | TATGAAGATAGCGTTAAAGGTCGTTTTACCATCAGCCGTGATGATGCACGTAATACCGTT |
| | | TATCTGCAAATGAATAGCCTGAAACCGGAAGATACCGCAGTGTATTATTGCAATGTTAAC |
| | | GTGGGCTTTGAATATTGGGGTCAGGGCACCCAGGTTACCGTTAGCAGCAAAGGAGGAGGT |
| | | GGGAGCCTAACAGACAGAGAATGGGCAGAAGAGTGGAAACATCTTGACCATCTGTTAAAC |
| | | TGCATAATGGACATGGTAGAAAAAACAAGGCGATCTCTCACCGTACTAAGGCGGTGTCAA |
| | | GAAGCAGACCGGGAAGAATTGAATTACTGGATCCGGCGGTACAGTGACGCCGAGGCACCT |
| | | ACTTCAAGTTCTACAAAGAAAACACAGCTACAACTGGAGCATTTACTGCTGGATTTACAG |
| | | ATGATTTTGAATGGAATTAATAATTACAAGAATCCCAAACTCACCAGGATGCTCACATTT |
| | | AAGTTTTACATGCCCAAGAAGGCCACAGAACTGAAACATCTTCAGTGTCTAGAAGAAGAA |
| | | CTCAAACCTCTGGAGGAAGTGCTAAATTTAGCTCAAAGCAAAAACTTTCACTTAAGACCC |
| | | AGGGACTTAATCAGCAATATCAACGTAATAGTTCTGGAACTAAAGGGATCTGAAACAACA |
| | | TTCATGTGTGAATATGCTGATGAGACAGCAACCATTGTAGAATTTCTGAACAGATGGATT |
| | | ACCTTTTGTCAAAGCATCATCTCAACACTGACTGACTACAAGGACGACGACGACAAGCAC |
| | | CACCACCATCACCAC |
| 30 | Quad 20 | ATGGAGACCCTCTTGGGCCTGCTTATCCTTTGGCTGCAGCTGCAATGGGTGAGC |
| | | AGCAAACAGGAGGTGACACAGATTCCTGCAGCTCTGAGTGTCCCAGAAGGAGAAAACTTG |
| | | GTTCTCAACTGCAGTTTCACTGATAGCGCTATTTACAACCTCCAGTGGTTTAGGCAGGAC |
| | | CCTGGGAAAGGTCTCACATCTCTGTTGCTTATTACACCTTGGCAGAGAGAGCAAACAAGT |
| | | GGAAGACTTAATGCCTCGCTGGATAAATCATCAGGACGTAGTACTTTATACATTGCAGCT |
| | | TCTCAGCCTGGTGACTCAGCCACCTACCTCTGTGCTGTGAGGCCCCTGCTTGACGGAACA |
| | | TACATACCTACATTTGGAAGAGGAACCAGCCTTATTGTTCATCCGTATATCCAGAACCCT |
| | | GACCCTGCCGTGTACCAGCTGAGAGACTCTAAATCCAGTGACAAGTCTGTCTGCCTATTC |
| | | ACCGATTTTGATTCTCAAACAAATGTGTCACAAAGTAAGGATTCTGATGTGTATATCACA |
| | | GACAAAACTGTGCTAGACATGAGGTCTATGGACTTCAAGAGCAACAGTGCTGTGGCCTGG |
| | | AGCAACAAATCTGACTTTGCATGTGCAAACGCCTTCAACAACAGCATTATTCCAGAAGAC |
| | | ACCTTCTTCCCCAGCCCAGAAAGTTCCGCCAGCACCAAGGGCCCCTCTGTGTTCCCTCTG |
| | | GCCCCTTCCAGCAAGTCCACCTCTGGCGGAACAGCCGCTCTGGGCTGCCTCGTGAAGGAC |
| | | TACTTCCCCGAGCCTGTGACCGTGTCCTGGAACTCTGGCGCTCTGACCAGCGGAGTGCAC |
| | | ACCTTCCCTGCTGTGCTGCAGTCCTCCGGCCTGTACTCCCTGTCCTCCGTCGTGACCGTG |
| | | CCTTCCAGCTCTCTGGGCACCCAGACCTACATCTGCAACGTGAACCACAAGCCCTCCAAC |
| | | ACCAAGGTGGACAAGAAGGTGTTGCATGGCACACGTCAAGAAGAAATGATTGATCACAGA |
| | | CTAACAGACAGAGAATGGGCAGAAGAGTGGAAACATCTTGACCATCTGTTAAACTGCATA |
| | | ATGGACATGGTAGAAAAACAAGGCGATCTCTCACCGTACTAAGGCGGTGTCAAGAAGCA |
| | | GACCGGGAAGAATTGAATTACTGGATCCGGCGGTACAGTGACGCCGAGGACTTAAAAGAC |
| | | TACAAGGACGACGACGACAAGACCACCACCATCACCAC |
| 31 | Quad 21 | ATGGAGACCCTCTTGGGCCTGCTTATCCTTTGGCTGCAGCTGCAATGGGTGAGC |
| | | AGCAAACAGGAGGTGACACAGATTCCTGCAGCTCTCAGTGTCCCAGAAGGAGAAAACTTG |
| | | GTTCTCAACTGCAGTTTCACTGATAGCGCTATTTACAACCTCCAGTGGTTTAGGCAGGAC |
| | | CCTGGGAAAGGTCTCACATCTCTGTTGCTTATTACACCTTGGCAGAGAGAGCAAACAAGT |
| | | GGAAGACTTAATGCCTCGCTGGATAAATCATCAGGACGTAGTACTTTATACATTGCAGCT |
| | | TCTCAGCCTGGTGACTCAGCCACCTACCTCTGTGCTGTGAGGCCCCTGCTTGACGGAACA |
| | | TACATACCTACATTTGGAAGAGGAACCAGCCTTATTGTTCATCCGTATATCCAGAACCCT |
| | | GACCCTGCCGTGTACCAGCTGAGAGACTCTAAATCCAGTGACAAGTCTGTCTGCCTATTC |
| | | ACCGATTTTGATTCTCAAACAAATGTGTCACAAAGTAAGGATTCTGATGTGTATATCACA |
| | | GACAAAACTGTGCTAGACATGAGGTCTATGGACTTCAAGAGCAACAGTGCTGTGGCCTGG |
| | | AGCAACAAATCTGACTTTGCATGTGCAAACGCCTTCAACAACAGCATTATTCCAGAAGAC |
| | | ACCTTCTTCCCCAGCCCAGAAAGTTCCGCCAGCACCAAGGGCCCCTCTGTGTTCCCTCTG |
| | | GCCCCTTCCAGCAAGTCCACCTCTGGCGGAACAGCCGCTCTGGGCTGCCTCGTGAAGGAC |
| | | TACTTCCCCGAGCCTGTGACCGTGTCCTGGAACTCTGGCGCTCTGACCAGCGGAGTGCAC |
| | | ACCTTCCCTGCTGTGCTGCAGTCCTCCGGCCTGTACTCCCTGTCCTCCGTCGTGACCGTG |
| | | CCTTCCAGCTCTCTGGGCACCCAGACCTACATCTGCAACGTGAACCACAAGCCCTCCAAC |
| | | ACCAAGGTGGACAAGAAGGTGGGAGGAGGTGGGAGCTTGCATGGCACACGTCAAGAAGAA |
| | | ATGATTGATCACAGACTAACAGACAGAGAATGGGCAGAAGAGTGGAAACATCTTGACCAT |
| | | CTGTTAAACTGCATAATGGACATGGTAGAAAAAACAAGGCGATCTCTCACCGTACTAAGG |
| | | CGGTGTCAAGAAGCAGACCGGGAAGAATTGAATTACTGGATCCGGCGGTACAGTGACGCC |
| | | GAGGACTTAAAAGACTACAAGGACGACGACGACAAGCACCACCACCATCCACCAC |
| 32 | Quad 22 | ATGAGCCTCGGGCTCCTGTGCTGTGGGGCCTTTTCTCTCCTGTGGGCAGGTCCA |
| | | GTGAATGCCGGTGTCACTCAGACCCCAAAATTCCAGGTCCTGAAGACAGGACAGAGCATG |
| | | ACACTGCAGTGTGCCCAGGATATGAACCATGAATACATGTCCTGGTATCGACAAGACCCA |
| | | GGCATGGGGCTGAGGCTGATTCATTACTCAGTTGCCATCCAGACAACTGACCAAGGAGAA |
| | | GTCCCCAATGGCTACAATGTCTCCAGATCAACCATCGAGGATTTCCCGCTCAGGCTGCTG |
| | | TCGGCTGCTCCCTCCCAGACATCTGTGTACTTCTGTGCCAGCAGTTACCTGGGGAACACC |
| | | GGGGAGCTGTTTTTTGGAGAAGGCTCTAGGCTGACCGTACTGGAGGACCTGAAAAACGTG |
| | | TTCCCACCCGAGGTCGCTGTGTTTGAGCCATCAGAAGCAGAGATCTCCCACACCCAAAAG |
| | | GCCACACTGGTGTGCCTGGCCACAGGCTTCTACCCCGACCATGTGGAGCTGAGCTGGTGG |
| | | GTGAATGGGAAGGAGGTGCACAGTGGGGTCAGCACAGACCCGCAGCCCCTCAAGGAGCAG |
| | | CCCGCCCTCAATGACTCCAGATACGCTCTGAGCAGCCGCCTGAGGGTCTCGGCCACCTTC |
| | | TGGCAGGACCCCCGCAACCACTTCCGCTGTCAAGTCCAGTTCTACGGGCTCTCGGAGAAT |
| | | GACGAGTGGACCCAGGATAGGGCCAAACCCGTCACCCAGATCGTCAGCGCCGAGGCCTGG |
| | | GGTAGAGCAGACACGGTGGCCGCTCCCTCCGTGTTCATCTTCCCACCTTCCGACGAGCAG |
| | | CTGAAGTCCGGCACCGCTTCTGTCGTGTGCCTGCTGAACAACTTCTACCCCCGCGAGGCC |
| | | AAGGTGCAGTGGAAGGTGGACAACGCCCTGCAGTCCGGCAACTCCCAGGAATCCGTGACC |
| | | GAGCAGGACTCCAAGGACAGCACCTACTCCCTGTCCTCCACCCTGACCCTGTCCAAGGCC |
| | | GACTACGAGAAGCACAAGGTGTACGCCTGCGAAGTGACCCACCAGGGCCTGTCTAGCCCC |
| | | GTGACCAAGTCTTTCAACCGGGGCGAGTGT |
| 33 | Quad 23 | ATGAACTTCGGGCTCAGCTTGATTTTCCTTGCCCTTATTTTAAAAGGTGTCCAG |
| | | TGTGAGGTGCAACTGGTGGAGTCTGGGGGAGGCTTAGTGAAGCCTGGGGGGTCCCTGAAA |
| | | CTCTCCTGTGCAGCCTCTGGACTCACTTTCAGTAGCTATGCCATGTCTTGGGTTCGCCAG |
| | | ACTCCAGAGAAGAGGCTGGAGTGGGTCGCATCCATTAGTAGTGGTGGTTTCACCTACTAT |
| | | CCAGACAGTGTGAAGGGCCGATTCACCATCTCCAGAGATAATGCCAGGAACATCCTGTAT |
| | | CTGCAAATGAGCAGTCTGAGGTCTGAGGACACGGCCATGTATTACTGTGCAAGAGACGAG |
| | | GTACGGGGGTACCTCGATGTCTGGGGCGCAGGGACCACGGTCACCGTTTCCTCGGCCAGC |
| | | ACCAAGGGCCCCTCTGTGTTCCCTCTGGCCCCTTCCAGCAAGTCCACCTCTGGCGGAACA |
| | | GCCGCTCTGGGCTGCCTCGTGAAGGACTACTTCCCCGAGCCTGTGACCGTGTCCTGGAAC |
| | | TCTGGCGCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACCTACATC |
| | | TGCAACGTGAACCACAAGCCCTCCAACACCAAGGTGGACAAGAAGGTGCTAACAGACAGA |
| | | GAATGGGCAGAAGAGTGGAAACATCTTGACCATCTGTTAAACTGCATAATGGACATGGTA |
| | | GAAAAAACAAGGCGATCTCTCACCGTACTAAGGCGGTGTCAAGAAGCAGACCGGGAAGAA |
| | | TTGAATTACTGGATCCGGCGGTACAGTGACGCCGAGGACTACAAGGACGACGACGACAAG |
| | | CACCACCACCATCACCAC |
| 34 | Quad 24 | ATGAACTTCGGGCTCAGCTTGATTTTCCTTGCCCTTATTTTAAAAGGTGTCCAG |
| | | TGTGAGGTGCAACTGGTGGAGTCTGGGGGAGGCTTAGTGAAGCCTGGGGGGTCCCTGAAA |
| | | CTCTCCTGTGCAGCCTCTGGACTCACTTTCAGTAGCTATGCCATGTCTTGGGTTCGCCAG |
| | | ACTCCAGAGAAGAGGCTGGAGTGGGTCGCATCCATTAGTAGTGGTGGTTTCACCTACTAT |
| | | CCAGACAGTGTGAAGGGCCGATTCACCATCTCCAGAGATAATGCCAGGAACATCCTGTAT |
| | | CTGCAAATGAGCAGTCTGAGGTCTGAGGACACGGCCATGTATTACTGTGCAAGAGACGAG |
| | | GTACGGGGGTACCTCGATGTCTGGGGCGCAGGGACCACGGTCACCGTTTCCTCGGCCAGC |
| | | ACCAAGGGCCCCTCTGTGTTCCCTCTGGCCCCTTCCAGCAAGTCCACCTCTGGCGGAACA |
| | | GCCGCTCTGGGCTGCCTCGTGAAGGACTACTTCCCCGAGCCTGTGACCGTGTCCTGGAAC |
| | | TCTGGCGCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACCTACATC |
| | | TGCAACGTGAACCACAAGCCCTCCAACACCAAGGTGGACAAGAAGGTGGGAGGAGGTGGG |
| | | AGCCTAACAGACAGAGAATGGGCAGAAGAGTGGAAACATCTTGACCATCTGTTAAACTGC |
| | | ATAATGGACATGGTAGAAAAAACAAGGCGATCTCTCACCGTACTAAGGCGGTGTCAAGAA |
| | | GCAGACCGGGAAGAATTGAATTACTGGATCCGGCGGTACAGTGACGCCGAGGACTACAAG |
| | | GACGACGACGACAAGCACCACCACCATCACCAC |
| 35 | Quad 25 | ATGAAATACCTATTGCCTACGGCAGCCGCTGGATTGTTATTACTCGCGGCCCAG |
| | | CCGGCCATGGCGGCCTACAAAGATATCCAGATGACACAGACTACATCCTCCCTGTCTGCC |
| | | TCTCTGGGAGACAGAGTCACCATCAGTTGCAGTGCAAGTCAGGGCATTAGCAATTATTTA |
| | | AACTGGTATCAGCAGAAACCAGATGGAACTGTTAAACTCCTGATCTATTACACATCAAGT |
| | | TTACACTCAGGAGTCCCATCAAGGTTCAGTGGCAGTGGGTCTGGGACAGATTATTCTCTC |
| | | ACCATCAGCAACCTGGAACCTGAAGATATTGCCACTTATTATTGTCAGCAGTATAGCAAG |
| | | CTTCCGTACACGTTCGGAGGGGGGACCAAGCTGGAAATAAAACGTACGGTGGCCGCTCCC |
| | | TCCGTGTTCATCTTCCCACCTTCCGACGAGCAGCTGAAGTCCGGCACCGCTTCTGTCGTG |
| | | TGCCTGCTGAACAACTTCTACCCCCGCGAGGCCAAGGTGCAGTGGAAGGTGGACAACGCC |
| | | CTGCAGTCCGGCAACTCCCAGGAATCCGTGACCGAGCAGGACTCCAAGGACAGCACCTAC |
| | | TCCCTGTCCTCCACCCTGACCCTGTCCAAGGCCGACTACGAGAAGCACAAGGTGTACGCC |
| | | TGCGAAGTGACCCACCAGGGCCTGTCTAGCCCCGTGACCAAGTCTTTCAACCGGGGCGAG |
| | | TGT |
| 36 | Quad 26 | ATGAACTTCGGGCTCAGCTTGATTTTCCTTGCCCTTATTTTAAAAGGTGTCCAG |
| | | TGTGAGGTGCAACTGGTGGAGTCTGGGGGAGGCTTAGTGAAGCCTGGGGGGTCCCTGAAA |
| | | CTCTCCTGTGCAGCCTCTGGACTCACTTTCAGTAGCTATGCCATGTCTTGGGTTCGCCAG |
| | | ACTCCAGAGAAGAGGCTGGAGTGGGTCGCATCCATTAGTAGTGGTGGTTTCACCTACTAT |
| | | CCAGACAGTGTGAAGGGCCGATTCACCATCTCCAGAGATAATGCCAGGAACATCCTGTAT |
| | | CTGCAAATGAGCAGTCTGAGGTCTGAGGACACGGCCATGTATTACTGTGCAAGAGACGAG |
| | | GTACGGGGGTACCTCGATGTCTGGGGCGCAGGGACCACGGTCACCGTTTCCTCGGCCAGC |
| | | ACCAAGGGCCCCTCTGTGTTCCCTCTGGCCCCTTCCAGCAAGTCCACCTCTGGCGGAACA |
| | | GCCGCTCTGGGCTGCCTCGTGAAGGACTACTTCCCCGAGCCTGTGACCGTGTCCTGGAAC |
| | | TCTGGCGCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACCTACATC |
| | | TGCAACGTGAACCACAAGCCCTCCAACACCAAGGTGGACAAGAAGGTGGAACCCAAGTCC |
| | | TGCGACAAGACCCACACCTGTCCCCCTTGTCCTCTAACAGACAGAGAATGGGCAGAAGAG |
| | | TGGAAACATCTTGACCATCTGTTAAACTGCATAATGGACATGGTAGAAAAAACAAGGCGA |
| | | TCTCTCACCGTACTAAGGCGGTGTCAAGAAGCAGACCGGGAAGAATTGAATTACTGGATC |
| | | CGGCGGTACAGTGACGCCGAGGACTACAAGGACGACGACGACAAGCACCACCACCATCAC |
| | | CAC |
| 37 | Quad 27 | ATGAACTTCGGGCTCAGCTTGATTTTCCTTGCCCTTATTTTAAAAGGTGTCCAG |
| | | TGTGAGGTGCAACTGGTGGAGTCTGGGGGAGGCTTAGTGAAGCCTGGGGGGTCCCTGAAA |
| | | CTCTCCTGTGCAGCCTCTGGACTCACTTTCAGTAGCTATGCCATGTCTTGGGTTCGCCAG |
| | | ACTCCAGAGAAGAGGCTGGAGTGGGTCGCATCCATTAGTAGTGGTGGTTTCACCTACTAT |
| | | CCAGACAGTGTGAAGGGCCGATTCACCATCTCCAGAGATAATGCCAGGAACATCCTGTAT |
| | | CTGCAAATGAGCAGTCTGAGGTCTGAGGACACGGCCATGTATTACTGTGCAAGAGACGAG |
| | | GTACGGGGGTACCTCGATGTCTGGGGCGCAGGGACCACGGTCACCGTTTCCTCGGCCAGC |
| | | ACCAAGGGCCCCTCTGTGTTCCCTCTGGCCCCTTCCAGCAAGTCCACCTCTGGCGGAACA |
| | | GCCGCTCTGGGCTGCCTCGTGAAGGACTACTTCCCCGAGCCTGTGACCGTGTCCTGGAAC |
| | | TCTGGCGCTCTGACCAGCGGAGTGCACACCTTCCCTGCTGTGCTGCAGTCCTCCGGCCTG |
| | | TACTCCCTGTCCTCCGTCGTGACCGTGCCTTCCAGCTCTCTGGGCACCCAGACCTACATC |
| | | TGCAACGTGAACCACAAGCCCTCCAACACCAAGGTGGACAAGAAGGTGGAACCCAAGTCC |
| | | TGCGACAAGACCCACACCTGTCCCCCTTGTCCTGGAGGAGGTGGGAGCCTAACAGACAGA |
| | | GAATGGGCAGAAGAGTGGAAACATCTTGACCATCTGTTAAACTGCATAATGGACATGGTA |
| | | GAAAAAACAAGGCGATCTCTCACCGTACTAAGGCGGTGTCAAGAAGCAGACCGGGAAGAA |
| | | TTGAATTACTGGATCCGGCGGTACAGTGACGCCGAGGACTACAAGGACGACGACGACAAG |
| | | CACCACCACCATCACCAC |
| 38 | Quad 28 | ATGGGATGGTCTTGTATAATTCTGTTCCTGGTGGCAACAGCAACAGGAGTGCAT |
| | | AGCGAGGTGCAGCTGGTGGAGTCTGGGGGAGGCTTGGTACAGCCCGGCAGGTCCCTGAGA |
| | | CTCTCCTGTGCGGCCTCTGGATTCACCTTTGATGATTATGCCATGCACTGGGTCCGGCAA |
| | | GCTCCAGGGAAGGGCCTGGAATGGGTCTCAGCTATCACTTGGAATAGTGGTCACATAGAC |
| | | TATGCGGACTCTGTGGAGGGCCGATTCACCATCTCCAGAGACAACGCCAAGAACTCCCTG |
| | | TATCTGCAAATGAACAGTCTGAGAGCTGAGGATACGGCCGTATATTACTGTGCGAAAGTC |
| | | TCGTACCTTAGCACCGCGTCCTCCCTTGACTATTGGGGCCAAGGTACCCTGGTCACCGTC |
| | | TCGAGTGCCAGCACCAAGGGCCCCTCTGTGTTCCCTCTGGCCCCTTCCAGCAAGTCCACC |
| | | TCTGGCGGAACAGCCGCTCTGGGCTGCCTCGTGAAGGACTACTTCCCCGAGCCTGTGACC |
| | | GTGTCCTGGAACTCTGGCGCTCTGACCAGCGGAGTGCACACCTTCCCTGCTGTGCTGCAG |
| | | TCCTCCGGCCTGTACTCCCTGTCCTCCGTCGTGACCGTGCCTTCCAGCTCTCTGGGCACC |
| | | CAGACCTACATCTGCAACGTGAACCACAAGCCCTCCAACACCAAGGTGGACAAGAAGGTG |
| | | TTGCATGGCACACGTCAAGAAGAAATGATTGATCACAGACTAACAGACAGAGAATGGGCA |
| | | GAAGAGTGGAAACATCTTGACCATCTGTTAAACTGCATAATGGACATGGTAGAAAAAACA |
| | | AGGCGATCTCTCACCGTACTAAGGCGGTGTCAAGAAGCAGACCGGGAAGAATTGAATTAC |
| | | TGGATCCGGCGGTACAGTGACGCCGAGGACTTAAAAGACTACAAGGACGACCACCACAAG |
| | | CACCACCACCATCACCAC |
| 39 | Quad 29 | ATGGGATGGTCTTGTATAATTCTGTTCCTGGTGGCAACAGCAACAGGAGTGCAT |
| | | AGCGAGGTGCAGCTGGTGGAGTCTGGGGGAGGCTTGGTACAGCCCGGCAGGTCCCTGAGA |
| | | CTCTCCTGTGCGGCCTCTGGATTCACCTTTGATGATTATGCCATGCACTGGGTCCGGCAA |
| | | GCTCCAGGGAAGGGCCTGGAATGGGTCTCAGCTATCACTTGGAATAGTGGTCACATAGAC |
| | | TATGCGGACTCTGTGGAGGGCCGATTCACCATCTCCAGAGACAACGCCAAGAACTCCCTG |
| | | TATCTGCAAATGAACAGTCTGAGAGCTGAGGATACGGCCGTATATTACTGTGCGAAAGTC |
| | | TCGTACCTTAGCACCGCGTCCTCCCTTGACTATTGGGGCCAAGGTACCCTGGTCACCGTC |
| | | TCGAGTGCCAGCACCAAGGGCCCCTCTGTGTTCCCTCTGGCCCCTTCCAGCAAGTCCACC |
| | | TCTGGCGGAACAGCCGCTCTGGGCTGCCTCGTGAAGGACTACTTCCCCGAGCCTGTGACC |
| | | GTGTCCTGGAACTCTGGCGCTCTGACCAGCGGAGTGCACACCTTCCCTGCTGTGCTGCAG |
| | | TCCTCCGGCCTGTACTCCCTGTCCTCCGTCGTGACCGTGCCTTCCAGCTCTCTGGGCACC |
| | | CAGACCTACATCTGCAACGTGAACCACAAGCCCTCCAACACCAAGGTGGACAAGAAGGTG |
| | | GGAGGAGGTGGGAGCTTGCATGGCACACGTCAAGAAGAAATGATTGATCACAGACTAACA |
| | | GACAGAGAATGGGCAGAAGAGTGGAAACATCTTGACCATCTGTTAAACTGCATAATGGAC |
| | | ATGGTAGAAAAAACAAGGCGATCTCTCACCGTACTAAGGCGGTGTCAAGAAGCAGACCGG |
| | | GAAGAATTGAATTACTGGATCCGGCGGTACAGTGACGCCGAGGACTTAAAAGACTACAAG |
| | | GACGACGACGACAAGCACCACCACCATCACCAC |
| 40 | Quad 30 | ATGGACATGAGGGTCCCTGCTCAGCTCCTGGGACTCCTGCTGCTCTGGCTCCCA |
| | | GATACCAGATGTGACATCCAGATGACCCAGTCTCCATCCTCCCTGTCTGCATCTGTAGGG |
| | | GACAGAGTCACCATCACTTGTCGGGCAASTCAGGGCATCAGAAATTACTTAGCCTGGTAT |
| | | CAGCAAAAACCAGGGAAAGCCCCTAAGCTCCTGATCTATGCTGCATCCACTTTGCAATCA |
| | | GGGGTCCCATCTCGGTTCAGTGGCAGTGGATCTGGGACAGATTTCACTCTCACCATCAGC |
| | | AGCCTACAGCCTGAAGATGTTGCAACTTATTACTGTCAAAGGTATAACCGTGCACCGTAT |
| | | ACTTTTGGCCAGGGGACCAAGGTGGAAATCAAACGTACGGTGGCCGCTCCCTCCGTGTTC |
| | | ATCTTCCCACCTTCCGACGAGCAGCTGAAGTCCGGCACCGCTTCTGTCGTGTGCCTGCTG |
| | | AACAACTTCTACCCCCGCGAGGCCAAGGTGCAGTGGAAGGTGGACAACGCCCTGCAGTCC |
| | | GGCAACTCCCAGGAATCCGTGACCGAGCAGGACTCCAAGGACAGCACCTACTCCCTGTCC |
| | | TCCACCCTGACCCTGTCGAAGGCCGACTACGAGAAGCACAAGGTGTACGCCTGCGAAGTG |
| | | ACCCACCAGGGCCTGTCTAGCCCCGTGACCAAGTCTTTCAACCGGGGCGAGTGT |
| 41 | Quad 31 | ATGGGATGGTCTTGTATAATTCTGTTCCTGGTGGCAACAGCAACAGGAGTGCAT |
| | | AGCGAGGTCCAACTTGTCGAAAGTGGCGGCGGTTTGGTTCAACCTGGAGGTTCACTTCGA |
| | | CTGTCATGTGCAGCGAGCGGTTATACATTTACGAATTATGGCATGAATTGGGTTAGACAG |
| | | GCACCAGGAAAGGGACTGGAGTGGGTAGGCTGGATCAATACCTACACAGGAGAACCAACG |
| | | TATGCCGCAGACTTCAAACGACGGTTTACATTTTCCTTGGATACCTCTAAGTCTACAGCG |
| | | TATCTCCAAATGAATTCACTTCGAGCGGAAGATACCGCGGTCTACTATTGCGCCAAATAC |
| | | CCTCATTATTATGGGTCATCTCACTGGTATTTCGATGTCTGGGGTCAGGGAACACTGGTA |
| | | ACCGTGTCATCCGCCAGCACCAAGGGCCCCTCTGTGTTCCCTCTGGCCCCTTCCAGCAAG |
| | | TCCACCTCTGGCGGAACAGCCGCTCTGGGCTGCCTCGTGAAGGACTACTTCCCCGAGCCT |
| | | GTGACCGTGTCCTGGAACTCTGGCGCTCTGACCAGCGGAGTGCACACCTTCCCTGCTGTG |
| | | CTGCAGTCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTGGACAAG |
| | | AAGGTGTTGCATGGCACACGTCAAGAAGAAATGATTGATCACAGACTAACAGACAGAGAA |
| | | TGGGCAGAAGAGTGGAAACATCTTGACCATCTGTTAAACTGCATAATGGACATGGTAGAA |
| | | AAAACAAGGCGATCTCTCACCGTACTAAGGCGGTGTCAAGAAGCAGACCGGGAAGAATTG |
| | | AATTACTGGATCCGGCGGTACAGTGACGCCGAGGACTTAAAAGACTACAAGGACGACGAC |
| | | GACAAGCACCACCACCATCACCAC |
| 42 | Quad 32 | ATGGGATGGTCTTGTATAATTCTGTTCCTGGTGGCAACAGCAACAGGAGTGCAT |
| | | AGCGAGGTCCAACTTGTCGAAAGTGGCGGCGGTTTGGTTCAACCTGGAGGTTCACTTCGA |
| | | CTGTCATGTGCAGCGAGCGGTTATACATTTACGAATTATGGCATGAATTGGGTTAGACAG |
| | | GCACCAGGAAAGGGACTGGAGTGGGTAGGCTGGATCAATACCTACACAGGAGAACCAACG |
| | | TATGCCGCAGACTTCAAACGACGGTTTACATTTTCCTTGGATACCTCTAAGTCTACAGCG |
| | | TATCTCCAAATGAATTCACTTCGAGCGGAAGATACCGCGGTCTACTATTGCGCCAAATAC |
| | | CCTCATTATTATGGGTCATCTCACTGGTATTTCGATGTCTGGGGTCAGGGAACACTGGTA |
| | | ACCGTGTCATCCGCCAGCACCAAGGGCCCCTCTGTGTTCCCTCTGGCCCCTTCCAGCAAG |
| | | TCCACCTCTGGCGGAACAGCCGCTCTGGGCTGCCTCGTGAAGGACTACTTCCCCGAGCCT |
| | | GTGACCGTGTCCTGGAACTCTGGCGCTCTGACCAGCGGAGTGCACACCTTCCCTGCTGTG |
| | | CTGCAGTCCTCCGGCCTGTACTCCCTGTCCTCCGTCGTGACCGTGCCTTCCAGCTCTCTG |
| | | GGCACCCAGACCTACATCTGCAACGTGAACCACAAGCCCTCCAACACCAAGGTGGACAAG |
| | | AAGGTGGGACGAGGTGGGAGCTTGCATGGCACACCTCAAGAAGAAATGATTCATCACAGA |
| | | CTAACAGACAGAGAATGGGCAGAAGAGTGGAAACATCTTGACCATCTGTTAAACTGCATA |
| | | ATGGACATGGTAGAAAAAACAAGGCGATCTCTCACCGTACTAAGGCGGTGTCAAGAAGCA |
| | | GACCGGGAAGAATTGAATTACTGGATCCGGCGGTACAGTGACGCCGAGGACTTAAAAGAC |
| | | TACAAGGACGACGACGACAAGCACCACCACCATCACCAC |
| 43 | Quad 33 | ATGGACATGAGGGTCCCTGCTCACCTCCTGGGGCTCCTGCAGCTCTGGCTCTCA |
| | | GGTGCCAGATGTGACATCCAAATGACCCAGAGTCCTTCCAGCCTCAGTGCGTCAGTGGGA |
| | | GATCGAGTGACGATAACGTGTTCTGCCAGCCAAGACATTTCCAACTATCTTAATTGGTAC |
| | | CAGCAGAAACCGGGAAAGGCCCCGAAAGTGCTCATATACTTTACCAGCAGTCTTCACTCT |
| | | TCTCTTCAACCGGAAGATTTTGCAACCTACTATTGTCAGCAGTATTCAACGGTACCTTGG |
| | | ACCTTCGGCCAAGGCACCAAAGTCGAGATTAAGCGTACGGTGGCCGCTCCCTCCGTGTTC |
| | | ATCTTCCCACCTTCCGACGAGCAGCTGAAGTCCGGCACCGCTTCTGTCGTGTGCCTGCTG |
| | | AACAACTTCTACCCCCGCGAGGCCAAGGTGCAGTGGAAGGTGGACAACGCCCTGCAGTCC |
| | | GGCAACTCCCAGGAATCCGTGACCGAGCAGGACTCCAAGGACAGCACCTACTCCCTGTCC |
| | | TCCACCCTGACCCTGTCCAAGGCCGACTACGAGAAGCACAAGGTGTACGCCTGCGAAGTG |
| | | ACCCACCAGGGCCTGTCTAGCCCCGTGACCAAGTCTTTCAACCGGGGCGAGTGT |
| 44 | Quad 34 | ATGGATATGCGCGTCCCGGCACAGCTGCTCGGCTTGTTGTTGCTGTGGTTGAGA |
| | | GCTAGGTGCGATATACAGATGACTCAGTCCCCTTCCAGTCTTTCAGCCAGTGTCGGCGAC |
| | | CGGGTTACCATTACTTGTCGGGCAAGTCAATCTATAGATAGTTATTTGCATTGGTATCAA |
| | | CAAAAACCAGGCAAAGCGCCTAAGTTGTTGATATATTCCGCATCTGAACTGCAATCAGGC |
| | | GTTCCTTCACGCTTTTCTGGAAGCGGCAGCGGAACCGATTTCACTCTTACCATAAGTAGT |
| | | CTCCAGCCGCAGGATTTTCCTACATACTATTGTCAACAAGTAGTGTGGCGACCGTTCACC |
| | | TTCGGACAGGGGACAAAAGTAGAAATCAAGCGGGGAGGAGGTGGGAGCTTGCATGGCACA |
| | | CGTCAAGAAGAAATGATTGATCACAGACTAACAGACAGAGAATGGGCAGAAGAGTGGAAA |
| | | CATCTTGACCATCTGTTAAACTGCATAATGGACATGGTAGAAAAAACAAGGCGATCTCTC |
| | | ACCGTACTAAGGCGGTGTCAAGAAGCAGACCGGGAAGAATTGAATTACTGGATCCGGCGG |
| | | TACAGTGACGCCGAGGACTTAAAAGACTACAAGGACGACGACGACAAGCACCACCACCAT |
| | | CACCAC |
| 45 | Quad 36 | ATGGAGTTTGGCCTCAGTTGGTTGTTTTTGGTAGCGAAAATTAAAGTACAGTGT |
| | | GAAGTCCAACTCCTGGAGAGCGGGGGGGGTCTGGTACAACCAGGCGGCTCACTGCGGCTT |
| | | AGCTGCGCAGCCTCCGGGTTCACGTTCGCACATGAAACGATGGTGTGGGTGCGCCAGGCA |
| | | CCGGGGAAGGGACTCGAATGGGTCTCACATATACCTCCTGACGGTCAGCATCCTTTTTAC |
| | | GCGGACTCTGTGAAGGGACGATTCACAATAAGTAGAGACAATAGTAAGAACACCCTTTAT |
| | | TTGCAGATGAACAGTCTGCGGGCGGAAGATACAGCAGTATATCATTGTGCCCTGCTGCCC |
| | | AAACGAGGTCCGTGGTTTGACTATTGGGGACAGGGGACTCTCGTTACTGTAAGCTCCGGA |
| | | GGAGGTGGGAGCTTGCATGGCACACGTCAAGAAGAAATGATTGATCACAGACTAACAGAC |
| | | AGAGAATGGGCAGAAGAGTGGAAACATCTTGACCATCTGTTAAACTGCATAATGGACATG |
| | | GTAGAAAAAACAAGGCGATCTCTCACCGTACTAAGGCGGTGTCAAGAAGCAGACCGGGAA |
| | | GAATTGAATTACTGGATCCGGCGGTACAGTGACGCCGAGGACTTAAAAGACTACAAGGAC |
| | | GACGACGACAAGCACCACCACCATCACCAC |
| 46 | Quad 38 | ATGGACATGAGGGTCCCCGCTCAGCTCCTGGGGCTCCTGCTACTCTGGCTCCGA |
| | | GCCAGATGTGATATACAGATGACCCAATCACCAAGCAGCTTGTCCGCTTCAGTGGGCGAC |
| | | AGGGTAACTATAACATGCCGCGCAAGCCAATGGATAGGTCCAGAACTCTCATGGTACCAA |
| | | CAAAAACCAGGGAAAGCGCCGAAACTGCTTATCTATCACACAAGCATTTTGCAATCTGGG |
| | | GTACCTAGTCGATTCAGTGGCTCTGGCAGTGGGACTGACTTTACACTCACCATAAGTTCT |
| | | CTCCAACCAGAGGACTTTGCAACATACTATTGTCAGCAATATATGTTTCAACCACGCACC |
| | | TTTGGACAAGGCACAAAAGTTGAAATCCGCGGAGGAGGTGGGAGCTTGCATGGCACACGT |
| | | CAAGAAGAAATGATTGATCACAGACTAACAGACAGAGAATGGGCAGAAGAGTGGAAACAT |
| | | CTTGACCATCTGTTAAACTGCATAATGGACATGGTAGAAAAAACAAGGCGATCTCTCACC |
| | | GTACTAAGGCGGTGTCAAGAAGCAGACCGGGAAGAATTGAATTACTGGATCCGGCGGTAC |
| | | AGTGACGCCGAGGACTTAAAAGACTACAAGGACGACGACGACAAGCACCACCACCATCAC |
| | | CAC |
| 47 | Quad 40 | ATGGACATGAGGGTCCCCGCTCAGCCCTGGGGCTCCTGCTACTCTGGCTCCGA |
| | | GCCAGATGTGATATTCAAATGACACAGTCACCAACGAGTCTTTCCGCGAGCGTTGGGGAC |
| | | CGAGTGACAATAACTTGTCGAGCCTCTCAGTGGATTGGCAACTTGCTGGACTGGTATCAG |
| | | CAAAAGCCGGGAGAAGCCCCGAAGCTGCTCATATACTATGCTTCCTTCCTCCAGAGTGGA |
| | | GTACCTAGCAGATTCAGCGGGGGGGGATTCGGCACTGATTTCACTCTTACAATCAGCTCT |
| | | CTTCAACCCGAGGACTTCGCAACGTACTACTGTCAACAAGCTAACCCTGCGCCGCTTACT |
| | | TTCGGACAAGGCACTAAGGTCGAAATTAAGCGAGGAGGAGGTGGGAGCTTGCATGGCACA |
| | | CGTCAAGAAGAAATGATTGATCACAGACTAACAGACAGAGAATGGGCAGAAGAGTGGAAA |
| | | CATCTTGACCATCTGTTAAACTGCATAATGGACATGGTAGAAAAAACAAGGCGATCTCTC |
| | | ACCCTACTAAGGCGGTGTCAAGAAGCACACCGGGAAGAATTGAATTACTGGATCCGGCGC |
| | | TACAGTGACGCCGAGGACTTAAAAGACTACAAGGACGACGACGACAAGCACCACCACCAT |
| | | CACCAC |
| 48 | Quad 42 | ATGGACATGAGGGTCCCCGCTCAGCTCCTGGGGCTCCTGCTACTCTGGCTCCGA |
| | | GCCAGATGTGATATACAGATGACCCAATCACCAAGCAGCTTGTCCGCTTCAGTGGGCGAC |
| | | AGGGTAACTATAACATGCCGCGCAAGCCAATGGATAGGTCCAGAACTCTCATGGTACCAA |
| | | CAAAAACCAGGGAAAGCGCCGAAACTGCTTATCTATCACACAAGCATTTTGCAATCTGGG |
| | | GTACCTAGTCGATTCAGTGGCTCTGGCAGTGGGACTGACTTTACACTCACCATAAGTTCT |
| | | CTCCAACCAGAGGACTTTCCAACATACTATTGTCAGCAATATATCTTTCAACCACCCACC |
| | | TTTGGACAAGGCACAAAAGTTGAAATCCGCGGAGGAGGTGGGAGCTTGCATGGCACACGT |
| | | CAAGAAGAAATGATTGATCACAGACTAACAGACAGAGAATGGGCAGAACAGTGGAAACAT |
| | | CTTGACCATCTGTTAAACTGCATAATGGACATGGTACAAAAAACAAGGCGATCTCTCACC |
| | | GTACTAAGGCGGTGTCAAGAAGCAGACCGGGAAGAATTGAATTACTGGATCCGGCGGTAC |
| | | AGTGACGCCGAGGACTTAAAAGGTGGAGGAGGTAGCGATATTCAAATGACACAGTCACCA |
| | | ACGAGTCTTTCCGCGAGCGTTGGGGACCGAGTGACAATAACTTGTCGAGCCTCTCAGTGG |
| | | ATTGGCAACTTGCTGGACTGGTATCAGCAAAAGCCGGGAGAAGCCCCGAAGCTGCTCATA |
| | | TACTATGCTTCCTTCCTCCAGAGTGGAGTACCTAGCAGATTCAGCGGGGGGGGATTCGGG |
| | | ACTGATTTCACTCTTACAATCAGCTCTCTTCAACCCGAGGACTTCGCAACGTACTACTGT |
| | | CAACAAGCTAACCCTGCGCCGCTTACTTTCGGACAAGGCACTAAGGTCGAAATTAAGCGA |
| | | GACTACAAGGACGACGACGACAAGCACCACCACCATCACCAC |
| 49 | Quad 44 | ATGGACATGAGGGTCCCCGCTCAGCTCCTGGGGCTCCTGCTACTCTGGCTCCGA |
| | | GCCAGATGTGACATTCAGATGACTCAGTCACCATCCTCATTGTCTGCATCAGTTGGTGAC |
| | | CGAGTTACGATCACATGTCGAGCAAGCCAAAATATAGATTCCAGACTTTCATGGTACCAG |
| | | CAGAAGCCTGGTAAAGCGCCGAAACTCCTCATATATCGCACGAGCGTATTGCAATCTGGT |
| | | GTGCCTTCTCGATTTTCAGGATCTGGGTCTGGCACTGACTTCACCTTGACAATATCTTCT |
| | | CTTCAGCCCGAAGATTTCGCTACCTACTACTGCCAACAATGGGACATGTTTCCTCTGACC |
| | | TTCGGACAGGGTACAAAGGTCGAGATTAAACGGGGAGGAGGTGGGAGCTTGCATGGCACA |
| | | CGTCAAGAAGAAATGATTGATCACAGACTAACAGACAGAGAATGGGCAGAAGAGTGGAAA |
| | | CATCTTGACCATCTGTTAAACTGCATAATGGACATGGTAGAAAAAACAAGGCGATCTCTC |
| | | ACCGTACTAAGGCGGTGTCAAGAAGCAGACCGGGAAGAATTGAATTACTGGATCCGGCGG |
| | | TACAGTGACGCCGAGGACTTAAAAGACTACAAGGACGACGACGAGAAGCACCACCACCAT |
| | | CACCAC |
| 50 | Quad 46 | ATGGACATGAGGGTCCCCGCTCAGCTCCTGGGGCTCCTGCTACTCTGGCTCCGA |
| | | GCCAGATGTGACATTCAGATGACTCAGTCACCATCCTCATTGTCTGCATCAGTTGGTGAC |
| | | CGAGTTACGATCACATGTCGAGCAAGCCAAAATATAGATTCCAGACTTTCATGGTACCAG |
| | | CAGAAGCCTGGTAAAGCGCCGAAACTCCTCATATATCGCACGAGCGTATTGCAATCTGGT |
| | | GTGCCTTCTCGATTTTCAGGATCTGGGTCTGGCACTGACTTCACCTTGACAATATCTTCT |
| | | CTTCAGCCCGAAGATTTCGCTACCTACTACTGCCAACAATGGGACATGTTTCCTCTGACC |
| | | TTCGGACAGGGTACAAAGGTCGAGATTAAACGGGGAGGAGGTGGGAGCTTGCATGGCACA |
| | | CGTCAAGAAGAAATGATTGATCACAGACTAACAGACAGAGRATGGGCAGAAGAGTGGAAA |
| | | CATCTTGACCATCTGTTAAACTGCATAATGGACATGGTAGAAAAAACAAGGCGATCTCTC |
| | | ACCGTACTAAGGCGGTGTCAAGAAGCAGACCGGGAAGAATTGAATTACTGGATCCGGCGG |
| | | TACAGTGACGCCGAGGACTTAAAAGGTGGAGGAGGTAGCGATATACAGATGACTCAATCC |
| | | CCTTCATCCCTCTCAGCTTCCGTAGGGGACAGAGTTACTATAACGTGTCGAGCTAGTCAA |
| | | GACATAGGTGATCGCCTGAGGTGGTATCAGCAAAAACCGGGTAAAGCACCTAAACTCCTC |
| | | ATATATCATGGTTCCAGGTTGGAGTCAGGCGTGCCGTCACGATTCTCTGGGTCACGCTCT |
| | | GGCACTGACTTCACATTGACGATTAGTTCTOTCCAGOCCGAAGACTTCGCCACCTACTAC |
| | | TGTCAACAGCAATGGTTTCGCCCGTATACTTTTGGGCAGGGTACAAAGGTTGAGATTAAA |
| | | CGGGACTACAAGGACGACGACGACAAGCACCACCACCATCACCAC |

TABLE 4
Amino acid sequences of binding moieties, domains and peptides

| SEQ | | | |
|---|---|---|---|
| ID NO: | Domain/petptide | Product* | Domain/peptide Amino Acid Sequence |
| 51 | Anti-TNF alpha H | HUMIRA® | EVQLVESGGGLVQPGRSLRLSCAASGFTFDDYAMHWVRQAPGKGLEWVSAITWNSGHIDY |
| | | | ADSVEGRFTISRDNAKNSLYLQMNSLRAEDTAVYYCAKVSYLSTASSLDYWGQGTLVTVS |
| | | | S |
| 52 | Anti TNF alpha VL | HUMIRA® | DIQMTQSPSSLSASVGDRVTITCRASQGIRNYLAWYQQKPGKAPKLLIYAASTLQSGVPS |
| | | | RFSGSGSGTDFTLTISSLQPEDVATYYCQRYNRAPYTFGQGTKVEIKR |
| 53 | Anti-CD20 VH | Rituximab | QVQLQQPGAELVKPGASVKMSCKASGYTFTSYNMHWVKQTPGRGLEWIGAIYPGNGDTSY |
| | | | NQKFKGKATLTADKSSSTAYMQLSSLTSEDSAVYYCARSTYYGGDWYFNVWGAGTTVTVS |
| | | | A |
| 54 | Anti-CD20 VL | Rituximab | QIVLSQSPAILSASPGEKVTMTCRASSSVSYIHWFQQKPGSSPKPWIYATSNLASGVPVR |
| | | | FSGSGSGTSYSLTISRVEAEDAATYYCQQWTSNPPTFGGGTKLEIKR |
| 55 | Anti-VEGF VH | AVASTIN® | EVQLVESGGGLVQPGGSLRLSCAASGYTFTNYGMNWVRQAPGKGLEWVGWINTYTGEPTY |
| | | | AADFKRRFTFSLDTSKSTAYLQMNSLRAEDTAVYYCAKYPHYYGSSHWYFDVWGQGTLVT |
| | | | VSS |
| 56 | Anti-VEGF VL | AVASTIN® | DIQMTQSPSSLSASVGDRVTITCSASQDISNYLNWYQQKPGKAPKVLIYFTSSLHSGVPS |
| | | | RFSGSGSGTDFTLTISSLQPEDFATYYCQQYSTVPWTFGQGTKVEIKR |
| 57 | Anti-HER2 VH | HERCEPTIN® | EVQLVESGGGLVQPGGSLRLSCAASGFTFTDYTMDWVRQAPGKGLEWVADVNPNSGGSIY |
| | | | NQRFKGRFTLSVDRSKNTLYLQMNSLRAEDTAVYYCARNLGPSFYFDYWGQGTLVTVSS |
| 58 | Anti-HER2 VL | HERCEPTIN® | DIQMTQSPSSLSASVGDRVTITCKASQDVSIGVAWYQQKPGKAPKLLIYSASYRYTGVPS |
| | | | RFSGSGSGTDFTLTISSLQPEDFATYYCQQYYIYPYTFGQGTKVEIKR |
| 59 | Anti-IL6R VH | ACTEMRA® | EVQLQESGPGLVRPSQTLSLTCTVSGYSITSDHAWSWVRQPPGRGLEWIGYISYSGITTY |
| | | | NPSLKSRVTMLRDTSKNQFSLRLSSVTAADTAVYYCARSLARTTAMDYWGQGSLVTVSS |
| 60 | Anti-IL6R VL | ACTEMRA® | DIQMTQSPSSLSASVGDRVTITCRASQDISSYLNWYQQKPGKAPKLLIYYTSRLHSGVPS |
| | | | RFSGSGSGTDFTFTISSLQPEDIATYYCQQGNTLPYTFGQGTKVEIKR |
| 61 | Anti-PD-1 VH | Nivolumab | QVQLVESGGGVVQPGRSLRLDCKASGITFSNSGMHWVRQAPGKGLEWVAVIWYDGSKRYY |
| | | | ADSVKGRFTISRDNSKNTLFLQMNSLRAEDTAVYYCATNDDYWGQGTLVTVSS |
| 62 | Anti-PD-1 VL | Nivolumab | EIVLTQSPATLSLSPGERATLSCRASQSVSSYLAWYQQKPGQAPRLLIYDASNRATGIPA |
| | | | RFSGSGSGTDFTLTISSLEPEDFAVYYCQQSSNWPRTFGQGTKVEIKR |
| 63 | Anti-CTLA4 VH | Ipilimumab | QVQLVESGGGVVQPGRSLRLSCAASGFTFSSYTMHWVRQAPGKGLEWVTFISYDGNNKYY |
| | | | ADSVKGRFTISRDNSKNTLYLQMNSLRAEDTAIYYCARTGWLGPFDYWGQGTLVTVSS |
| 64 | Anti-CTLA4 VL | Ipilimumab | EIVLTQSPGTLSLSPGERATLSCRASQSVGSSYLAWYQQKPGQAPRLLIYGAFSRATGIP |
| | | | DRFSGSGSGTDFTLTISRLEPEDFAVYYCQQYGSSPWTFGQGTKVEIKR |
| 65 | Anti-TNFR1 dAb | DOM1h-131- | EVQLLESGGGLVQPGGSLRLSCAASGFTFAHETMVWVRQAPGKGLEWVSHIPPDGQDPFY |
| | | 206 VH | ADSVKGRFTISRDNSKNTLYLQMNSLRAEDTAVYHCALLPKRGPWFDYWGQGTLVTVSS |
| 66 | Anti-TNFα dAb | TAR 1-5- | DIQMTQSPSSLSASVGDRVTITCRASQSIDSYLHWYQQKPGKAPKLLIYSASELQSGVPS |
| | | 19 Vk | RFSGSGSGTDFTLTISSLQPEDFATYYCQQVVWRPFTFGQGTKVEIKR |
| 67 | Anti-VEGF dAb | TAR15-10 Vk | DIQMTQSPSSLSASVGDRVTITCRASQWIGPELSWYQQKPGKAPKLLIYHTSILQSGVPS |
| | | | RFSGSGSGTDFTLTISSLQPEDFATYYCQQYMFQPRTFGQGTKVEIR |
| 68 | Anti-EGFR dAb | DOM16-39- | DIQMTQSPTSLSASVGDRVTITCRASQWIGNLLDWYQQKPGEAPKLLIYYASFLQSGVPS |
| | | 109 Vk | RFSGGGFGTDFTLTISSLQPEDFATYYCQQANPAPLTFGQGTKVEIKR |
| 69 | Anti-CD38 dAb | DOM 11-3 Vk | DIQMTQSPSSLSASVGDRVTITCRASQNIDSRLSWYQQKPGKAPKLLIYRTSVLQSGVPS |
| | | | RFSGSGSGTDFTLTISSLQPEDFATYYCQQWDMFPLTFGQGTKVEIKR |
| 70 | Anti-CD138 dAb | DOM 12-45 Vk | DIQMTQSPSSLSASVGDRVTITCRASQDIGDRLRWYQQKPGKAPKLLIYHGSRLESGVPS |
| | | | RFSGSRSGTDFTLTISSLQPEDFATYYCQQQWFRPYTFGQGTKVEIKR |
| 71 | Anti-NY•ESO-1 Vα | | KQEVTQIPAALSVPEGENLVLNCSFTDSAIYNLQWFRQDPGKGLTSLLLITPWQREQTSG |
| | | | RLNASLDKSSGRSTLYIAASQPGDSATYLCAVRPLLDGTYIPTFGRGTSLIVHPY |
| 72 | Anti-NY•ESO-1 Vβ | | NAGVTQTPKFQVLKTGQSMTLQCAQDMNHEYMSWYRQDPGMGLRLIHYSVAIQTDQGEVP |
| | | | NGYNVSRSTIEDFPLRLLSAAPSQTSVYFCASSYLGNTGELFFGEGSRLTVL |
| 73 | IL2 | Human IL2 | APTSSSTKKTQLQLEHLLLDLQMILNGINNYKNPKLTRMLTFKFYMPKKATELKHLQCLE |
| | | | EELKPLEEVLNLAQSKNFHLRPRDLISNINVIVLELKGSETTFMCEYADETATIVEFLNR |
| | | | WITFCQSIISTLT |
| 74 | Anti-GFP VH | NANOBODY™ | QVQLVESGGALVQPGGSLRLSCAASGFPVNRYSMRWYRQAPGKEREWVAGMSSAGDRSSY |
| | | | EDSVKGRFTISRDDARNTVYLQMNSLKPEDTAVYYCNVNVGFEYWGQGTQVTVSS |
| 75 | Anti-IL1R1 | DOM4-122- | DIQMTQSPSSLSASVGDRVTITCRASQSIIKHLKWYQQKPGKAPKLLIYGASRLQSGVPS |
| | | 23 Vk | RFSGSGSGTDFTLTISSLQPEDFATYYCQQGARWPQTFGQGTKVEIKR |
| 76 | FLT1 | EYLEA™ | SDTGRPFVEMYSEIPEIIHMTEGRELVIPCRVTSPNITVTLKKFPLDTLIPDGKRIIWDS |
| | | | RKGFHSNATYKEIGLLTCEATVNGHLYKTNYLTHRQTNTIIDV |
| 77 | KDR | EYLEA™ | VLSPSHGIELSVGEKLVLNCTARTELNVGIDFNWEYPSSKHQHKKLVNRDLKTQSGSEMK |
| | | | KFLSTLTIDGVTRSDQGLYTCAASSGLMTKKNSTFVRVHEK |
| 78 | | Aflibercept | SDTGRPFVEMYSEIPEIIHMTEGRELVIPCRVTSPNITVTLKKFPLDTLIPDGKRIIWDS |
| | | | RKGFIISNATYKEIGLLTCEATVNGHLYKTNYLTHRQTNTIIDVVLSPSHGIELSVGEKL |
| | | | VLNCTARTELNVGIDFNWEYPSSKHQHKKLVNRDLKTQSGSEMKKFLSTLTIDGVTRSDQ |
| | | | GLYTCAASSGLMTKKNSTFVRVHEKDKTHTCPPCPAPELLGGPSVFLFPPKPKDTLMISR |
| | | | TPEVTCVVVDVSHEDPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKNQVSLTCLVKGFYPS |
| | | | DIAVEWESNGQPENNYKTTPPVLDSDGSFFLYSKLTVDKSRWQQGNVFSCSVMHEALHNH |
| | | | YTQKSLSLSPG |
| 79 | GLP-1(7-37)-Pro9 | | HAPGTFTSDVSSYLEGQAAKEFIAWLVKGRG |
| 80 | Peptide YY | | IKPEAPGEDASPEELNRYYASLRHYLNLVTRQRY |
| 81 | EXENDIN-4 | | HGEGTFTSDLSKQMEEEAVRLFIEWLKNGGPSSGAPPPS |
| 82 | | DURAGLUTIDE™ | HGEGTFTSDVSSYLEEQAAKEFIAWLVKGGGGGGGSGGGGSGGGGSAESKYGPPCPPCPA |
| | | | PEAAGGPSVFLFPPKPKDTLMISRTPEVTCVVVDVSQEDPEVQFNWYVDGVEVHNAKTKP |
| | | | REEQFNSTYRVVSVLTVLHQDWLNGKEYKCKVSNKGLPSSIEKTISKAKGQPREPQVYTL |
| | | | PPSQEEMTKNGVSLTCLVKGFYPSDIAVEWESNGQPENNYKTTPPVLDSDGSFFLYSRLT |
| | | | VDKSRWQEGNVFSCSVMHEALHNHYTQKSLSLSLG |

*indicates product comprising the domain.

TABLE 5
Amino acid sequences of Quad polypeptides

| SEQ | POLY- | |
|---|---|---|
| ID NO: | PEPTIDE | AMINO ACID SEQUENCE |
| 83 | Quad 1 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVSTDPQPLKEQPALNDSRYALSSRLRVSATFWQDPRNHFRCQVQFYGLSENDE |
| | | WTQDRAKPVTQIVSAEAWGRADASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV |
| | | LTDREWAEEWKHLDHLLNCIMDMVEKTRRSLTVLRRCQEADREELNYWIRRYSDAE |
| 84 | Quad 2 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVSTDPQPLKEQPALNDSRYALSSRLRVSATFWQDPRNHFRCQVQFYGLSENDE |
| | | WTQDRAKPVTQIVSAEAWGRADASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV |
| | | GGGGSLTDREWAEEWKHLDHLLNCIMDMVEKTRRSLTVLRRCQEADREELNYWIRRYSDA |
| | | E |
| 85 | Quad 3 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVCTDPQPLKEQPALNDSRYALSSRLRVSATFWQDPRNHFRCQVQFYGLSENDE |
| | | WTQDRAKPVTQIVSAEAWGRADASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV |
| | | LTDREWAEEWKHLDHLLNCIMDMVEKTRRSLTVLRRCQEADREELNYWIRRYSDAE |
| 86 | Quad 4 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVCTDPQPLKEQPALNDSRYALSSRLRVSATFWQDPRNHFRCQVQFYGLSENDE |
| | | WTQDRAKPVTQIVSAEAWGRADASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV |
| | | GGGGSLTDREWAEEWKHLDHLLNCIMDMVEKTRRSLTVLRRCQEADREELNYWIRRYSDA |
| | | E |
| 87 | Quad 5 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVCTDPQPLKEQPALNDSRYALSSRLRVSATFWQDPRNHFRCQVQFYGLSENDE |
| | | WTQDRAKPVTQIVSAEAWGRADLTDREWAEEWKHLDHLLNCIMDMVEKTRRSLTVLRRCQ |
| | | EADREELNYWIRRYSDAE |
| 88 | Quad 6 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVCTDPQPLKEQPALNDSRYALSSRLRVSATFWQDPRNHFRCQVQFYGLSENDE |
| | | WTQDRAKPVTQIVSAEAWGRADGGGGSLTDREWAEEWKHLDHLLNCIMDMVEKTRRSLTV |
| | | LRRCQEADREELNYWIRRYSDAE |
| 89 | Quad 7 | KQEVTQIPAALSVPEGENLVLNCSFTDSAIYNLQWFRQDPG |
| | | KGLTSLLLITPWQREQTSGRLNASLDKSSGRSTLYIAASQPGDSATYLCAVRPLLDGTYI |
| | | PTFGRGTSLIVHPYIQNPDPAVYQLRDSKSSDKSVCLFTDFDSQTNVSQSKDSDVYITDK |
| | | TVLDMRSMDFKSNSAVAWSNKSDFACANAFNNSIIPEDTFFPSPESSTVAAPSVFIFPPS |
| | | DEQLKSGTASVVCLLNNFYPREAKVQWKVDNALQSGNSQESVTEQDSKDSTYSLSSTLTL |
| | | SKADYEKHKVYACEVTHQGLSSPVTKSFNRGEC |
| 90 | Quad 8 | KQEVTQIPAALSVPEGENLVLNCSFTDSAIYNLQWFRQDPG |
| | | KGLTSLLLITPWQREQTSGRLNASLDKSSGRSTLYIAASQPGDSATYLCAVRPLLDGTYI |
| | | PTFGRGTSLIVHPYIQNPDPAVYQLRDSKSSDKSVCLFTDFDSQTNVSQSKDSDVYITDK |
| | | CVLDMRSMDFKSNSAVAWSNKSDFACANAFNNSIIPEDTFFPSPESSTVAAPSVFIFPPS |
| | | DEQLKSGTASVVCLLNNFYPREAKVQWKVDNALQSGNSQESVTEQDSKDSTYSLSSTLTL |
| | | SKADYEKHKVYACEVTHQGLSSPVTKSFNRGEC |
| 91 | Quad 9 | KQEVTQIPAALSVPEGENLVLNCSFTDSAIYNLQWFRQDPG |
| | | KGLTSLLLITPWQREQTSGRLNASLDKSSGRSTLYIAASQPGDSATYLCAVRPLLDGTYI |
| | | PTFGRGTSLIVHPYIQNPDPAVYQLRDSKSSDKSVCLFTDFDSQTNVSQSKDSDVYITDK |
| | | CVLDMRSMDFKSNSAVAWSNKSDFACANAFNNSIIPEDTFFPSPESS |
| 92 | Quad 10 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVSTDPQPLKEQPALNDSRYALSSRLRVSATFWQDPRNHFRCQVQFYGLSENDE |
| | | WTQDRAKPVTQIVSAEAWGRADASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV |
| | | LTDREWAEEWKHLDHLLNCIMDMVEKTRRSLTVLRRCQEADREELNYWIRRYSDAEAPTS |
| | | SSTKKTQLQLEHLLLDLQMILNGINNYKNPKLTRMLTFKFYMPKKATELKHLQCLEEELK |
| | | PLEEVLNLAQSKNFHLRPRDLISNINVIVLELKGSETTFMCEYADETATIVEFLNRWITF |
| | | CQSIISTLT |
| 93 | Quad 11 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVSTDPQPLKEQPALNDSRYALSSRLRVSATFWQDPRNHFRCQVQFYGLSENDE |
| | | WTQDRAKPVTQIVSAEAWGRADASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV |
| | | GGGGSLTDREWAEEWKHLDHLLNCIMDMVEKTRRSLTVLRRCQEADREELNYWIRRYSDA |
| | | EAPTSSSTKKTQLQLEHLLLDLQMILNGINNYKNPKLTRMLTFKFYMPKKATELKHLQCL |
| | | EEELKPLEEVLNLAQSKNFHLRPRDLISNINVIVLELKGSETTFMCEYADETATIVEFLN |
| | | RWITFCQSIISTLT |
| 94 | Quad 12 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVSTDPQPLKEQPALNDSRYALSSRLRVSATFWQDPRNHFRCQVQFYGLSENDE |
| | | WTQDRAKPVTQIVSAEAWGRADASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV |
| | | EPKSCDKTHTCPPCPLTDREWAEEWKHLDHLLNCIMDMVEKTRRSLTVLRRCQEADREEL |
| | | NYWIRRYSDAE |
| 95 | Quad 13 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVSTDPQPLKEQPALNDSRYALSSRLRVSATFWQDPRNHFRCQVQFYGLSENDE |
| | | WTQDRAKPVTQIVSAEAWGRADASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV |
| | | EPKSCDKTHTCPPCPGGGGSLTDREWAEEWKHLDHLLNCIMDMVEKTRRSLTVLRRCQEA |
| | | DREELNYWIRRYSDAE |
| 96 | Quad 14 | QVQLVESGGALVQPGGSLRLSCAASGFPVNRYSMRWYRQAP |
| | | GKEREWVAGMSSAGDRSSYEDSVKGRFTISRDDARNTVYLQMNSLKPEDTAVYYCNVNVG |
| | | FEYWGQGTQVTVSSKLTDREWAEEWKHLDHLLNCIMDMVEKTRRSLTVLRRCQEADREEL |
| | | NYWIRRYSDAE |
| 97 | Quad 15 | QVQLVESGGALVQPGGSLRLSCAASGFPVNRYSMRWYRQAP |
| | | GKEREWVAGMSSAGDRSSYEDSVKGRFTISRDDARNTVYLQMNSLKPEDTAVYYCNVNVG |
| | | FEYWGQGTQVTVSSKGGGGSLTDREWAEEWKHLDHLLNCIMDMVEKTRRSLTVLRRCQEA |
| | | DREELNYWIRRYSDAE |
| 98 | Quad 18 | QVQLVESGGALVQPGGSLRLSCAASGFPVNRYSMRWYRQAP |
| | | GKEREWVAGMSSAGDRSSYEDSVKGRFTISRDDARNTVYLQMNSLKPEDTAVYYCNVNVG |
| | | FEYWGQGTQVTVSSKLTDREWAEEWKHLDHLLNCIMDMVEKTRRSLTVLRRCQEADREEL |
| | | NYWIRRYSDAEAPTSSSTKKTQLQLEHLLLDLQMILNGINNYKNPKLTRMLTFKFYMPKK |
| | | ATELKHLQCLEEELKPLEEVLNLAQSKNFHLRPRDLISNINVIVLELKGSETTFMCEYAD |
| | | ETATIVEFLNRWITFCQSIISTLT |
| 99 | Quad 19 | QVQLVESGGALVQPGGSLRLSCAASGFPVNRYSMRWYRQAP |
| | | GKEREWVAGMSSAGDRSSYEDSVKGRFTISRDDARNTVYLQMNSLKPEDTAVYYCNVNVG |
| | | FEYWGQGTQVTVSSKGGGGSLTDREWAEEWKHLDHLLNCIMDMVEKTRRSLTVLRRCQEA |
| | | DREELNYWIRRYSDAEAPTSSSTKKTQLQLEHLLLDLQMILNGINNYKNPKLTRMLTFKF |
| | | YMPKKATELKHLQCLEEELKPLEEVLNLAQSKNFHLRPRDLISNINVIVLELKGSETTFM |
| | | CEYADETATIVEFLNRWITFCQSIISTLT |
| 100 | Quad 20 | KQEVTQIPAALSVPEGENLVLNCSFTDSAIYNLQWFRQDPG |
| | | KGLTSLLLITPWQREQTSGRLNASLDKSSGRSTLYIAASQPGDSATYLCAVRPLLDGTYI |
| | | PTFGRGTSLIVHPYIQNPDPAVYQLRDSKSSDKSVCLFTDFDSQTNVSQSKDSDVYITDK |
| | | TVLDMRSMDFKSNSAVAWSNKSDFACANAFNNSIIPEDTFFPSPESSASTKGPSVFPLAP |
| | | SSKSTSGGTAALGCLVKDYFPEPVTVSWNSGALTSGVHTFPAVLQSSGLYSLSSVVTVPS |
| | | SSLGTQTYICNVNHKPSNTKVDKKVLHGTRQEEMIDHRLTDREWAEEWKHLDHLLNCIMD |
| | | MVEKTRRSLTVLRRCQEADREELNYWIRRYSDAEDLK |
| 101 | Quad 21 | KQEVTQIPAALSVPEGENLVLNCSFTDSAIYNLQWFRQDPG |
| | | KGLTSLLLITPWQREQTSGRLNASLDKSSGRSTLYIAASQPGDSATYLCAVRPLLDGTYI |
| | | PTFGRGTSLIVHPYIQNPDPAVYQLRDSKSSDKSVCLFTDFDSQTNVSQSKDSDVYITDK |
| | | TVLDMRSMDFKSNSAVAWSNKSDFACANAFNNSIIPEDTFFPSPESSASTKGPSVFPLAP |
| | | SSKSTSGGTAALGCLVKDYFPEPVTVSWNSGALTSGVHTFPAVLQSSGLYSLSSVVTVPS |
| | | SSLGTQTYICNVNHKPSNTKVDKKVGGGGSLHGTRQEEMIDHRLTDREWAEEWKHLDHLL |
| | | NCIMDMVEKTRRSLTVLRRCQEADREELNYWIRRYSDAEDLK |
| 102 | Quad 22 | NAGVTQTPKFQVLKTGQSMTLQCAQDMNHEYMSWYRQDPGM |
| | | GLRLIHYSVAIQTTDQGEVPNGYNVSRSTIEDFPLRLLSAAPSQTSVYFCASSYLGNTGE |
| | | LFFGEGSRLTVLEDLKNVFPPEVAVFEPSEAEISHTQKATLVCLATGFYPDHVELSWWVN |
| | | GKEVHSGVSTDPQPLKEQPALNDSRYALSSRLRVSATFWQDPRNHFRCQVQFYGLSENDE |
| | | WTQDRAKPVTQIVSAEAWGRADTVAAPSVFIFPPSDEQLKSGTASVVCLLNNFYPREAKV |
| | | QWKVDNALQSGNSQESVTEQDSKDSTYSLSSTLTLSKADYEKHKVYACEVTHQGLSSPVT |
| | | KSFNRGEC |
| 103 | Quad 28 | EVQLVESGGGLVQPGRSLRLSCAASGFTFDDYAMHWVRQAP |
| | | GKGLEWVSAITWNSGHIDYADSVEGRFTISRDNAKNSLYLQMNSLRAEDTAVYYCAKVSY |
| | | LSTASSLDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKVDKKVLH |
| | | GTRQEEMIDHRLTDREWAEEWKHLDHLLNCIMDMVEKTRRSLTVLRRCQEADREELNYWI |
| | | RRYSDAEDLK |
| 104 | Quad 29 | EVQLVESGGGLVQPGRSLRLSCAASGFTFDDYAMHWVRQAP |
| | | GKGLEWVSAITWNSGHIDYADSVEGRFTISRDNAKNSLYLQMNSLRAEDTAVYYCAKVSY |
| | | LSTASSLDYWGQGTLVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTKVDKKVGG |
| | | GGSLHGTRQEEMIDHRLTDREWAEEWKHLDHLLNCIMDMVEKTRRSLTVLRRCQEADREE |
| | | LNYWIRRYSDAEDLK |
| 105 | Quad 30 | DIQMTQSPSSLSASVGDRVTITCRASQGIRNYLAWYQQ |
| | | KPGKAPKLLIYAASTLQSGVPSRFSGSGSGTDFTLTISSLQPEDVATYYCQRYNRAPYTF |
| | | GQGTKVEIKRTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSFNRGEC |
| 106 | Quad 31 | EVQLVESGGGLVQPGGSLRLSCAASGYTFTNYGMNWVRQAP |
| | | GKGLEWVGWINTYTGEPTYAADFKRRFTFSLDTSKSTAYLQMNSLRAEDTAVYYCAKYPH |
| | | YYGSSHWYFDVWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV |
| | | LHGTRQEEMIDHRLTDREWAEEWKHLDHLLNCIMDMVEKTRRSLTVLRRCQEADREELNY |
| | | WIRRYSDAEDLK |
| 107 | Quad 32 | EVQLVESGGGLVQPGGSLRLSCAASGYTFTNYGMNWVRQAP |
| | | GKGLEWVGWINTYTGEPTYAADFKRRFTFSLDTSKSTAYLQMNSLRAEDTAVYYCAKYPH |
| | | YYGSSHWYFDVWGQGTLVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNTKVDKKV |
| | | GGGGSLHGTRQEEMIDHRLTDREWAEEWKHLDHLLNCIMDMVEKTRRSLTVLRRCQEADR |
| | | EELNYWIRRYSDAEDLK |
| 108 | Quad 33 | DIQMTQSPSSLSASVGDRVTITCSASQDISNYLNWYQQ |
| | | KPGKAPKVLIYFTSSLHSGVPSRFSGSGSGTDFTLTISSLQPEDFATYYCQQYSTVPWTF |
| | | GQGTKVEIKRTVAAPSVFIFPPSDEQLKSGTASVVCLLNNFYPREAKVQWKVDNALQSGN |
| | | SQESVTEQDSKDSTYSLSSTLTLSKADYEKHKVYACEVTHQGLSSPVTKSFNRGEC |
| 109 | Quad 34 | DIQMTQSPSSLSASVGDRVTITCRASQSIDSYLHWYQQK |
| | | PGKAPKLLIYSASELQSGVPSRFSGSGSGTDFTLTISSLQPEDFATYYCQQVVWRPFTFG |
| | | QGTKVEIKRGGGGSLHGTRQEEMIDHRLTDREWAEEWKHLDHLLNCIMDMVEKTRRSLTV |
| | | LRRCQEADREELNYWIRRYSDAEDLK |
| 110 | Quad 36 | EVQLLESGGGLVQPGGSLRLSCAASGFTFAHETMVWVRQAPG |
| | | KGLEWVSHIPPDGQDPFYADSVKGRFTISRDNSKNTLYLQMNSLRAEDTAVYHCALLPKR |
| | | GPWFDYWGQGTLVTVSSGGGGSLHGTRQEEMIDHRLTDREWAEEWKHLDHLLNCIMDMVE |
| | | KTRRSLTVLRRCQEADREELNYWIRRYSDAEDLK |
| 111 | Quad 38 | DIQMTQSPSSLSASVGDRVTITCRASQWIGPELSWYQQK |
| | | PGKAPKLLIYHTSILQSGVPSRFSGSGSGTDFTLTISSLQPEDFATYYCQQYMFQPRTFG |
| | | QGTKVEIRGGGGSLHGTRQEEMIDHRLTDREWAEEWKHLDHLLNCIMDMVEKTRRSLTVL |
| | | RRCQEADREELNYWIRRYSDAEDLK |
| 112 | Quad 40 | DIQMTQSPTSLSASVGDRVTITCRASQWIGNLLDWYQQK |
| | | PGEAPKLLIYYASFLQSGVPSRFSGGGFGTDFTLTISSLQPEDFATYYCQQANPAPLTFG |
| | | QGTKVEIKRGGGGSLHGTRQEEMIDHRLTDREWAEEWKHLDHLLNCIMDMVEKTRRSLTV |
| | | LRRCQEADREELNYWIRRYSDAEDLK |
| 113 | Quad 42 | DIQMTQSPSSLSASVGDRVTITCRASQWIGPELSWYQQK |
| | | PGKAPKLLIYHTSILQSGVPSRFSGSGSGTDFTLTISSLQPEDFATYYCQQYMFQPRTFG |
| | | QGTKVEIRGGGGSLHGTRQEEMIDHRLTDREWAEEWKHLDHLLNCIMDMVEKTRRSLTVL |
| | | RRCQEADREELNYWIRRYSDAEDLKGGGGSDIQMTQSPTSLSASVGDRVTITCRASQWIG |
| | | NLLDWYQQKPGEAPKLLIYYASFLQSGVPSRFSGGGFGTDFTLTISSLQPEDFATYYCQQ |
| | | ANPAPLTFGQGTKVEIKR |
| 114 | Quad 44 | DIQMTQSPSSLSASVGDRVTITCRASQNIDSRLSWYQQK |
| | | PGKAPKLLIYRTSVLQSGVPSRFSGSGSGTDFTLTISSLQPEDFATYYCQQWDMFPLTFG |
| | | QGTKVEIKRGGGGSLHGTRQEEMIDHRLTDREWAEEWKHLDHLLNCIMDMVEKTRRSLTV |
| | | LRRCQEADREELNYWIRRYSDAEDLK |
| 115 | Quad 46 | DIQMTQSPSSLSASVGDRVTITCRASQNIDSRLSWYQQK |
| | | PGKAPKLLIYRTSVLQSGVPSRFSGSGSGTDFTLTISSLQPEDFATYYCQQWDMFPLTFG |
| | | QGTKVEIKRGGGGSLHGTRQEEMIDHRLTDREWAEEWKHLDHLLNCIMDMVEKTRRSLTV |
| | | LRRCQEADREELNYWIRRYSDAEDLKGGGGSDIQMTQSPSSLSASVGDRVTITCRASQDI |
| | | GDRLRWYQQKPGKAPKLLIYHGSRLESGVPSRFSGSRSGTDFTLTISSLQPEDFATYYCQ |
| | | QQWFRPYTFGQGTKVEIKR |

TABLE 6
DNA and amino acid sequences of NHR2 TDs

| SEQ | | |
|---|---|---|
| ID NO: | NHR2 TD | |
| | | DNA SEQUENCE |
| 116 | NHR2 TD | CTAACAGACAGAGAATGGGCAGAAGAGTGGAAACATCTTGACCATCTGTTAAACTGCATA |
| | | ATGGACATGGTAGAAAAAACAAGGCGATCTCTCACCGTACTAAGGCGGTGTCAAGAAGCA |
| | | GACCGGGAAGAATTGAATTACTGGATCCGGCGGTACAGTGACGCCGAG |
| 117 | NHR2* TD | TTGCATGGCACACGTCAAGAAGAAATGATTGATCACAGACTAACAGACAGAGAATGGGCA |
| | | GAAGAGTGGAAACATCTTGACCATCTGTTAAACTGCATAATGGACATGGTAGAAAAAACA |
| | | AGGCGATCTCTCACCGTACTAAGGCGGTGTCAAGAAGCAGACCGGGAAGAATTGAATTAC |
| | | TGGATCCGGCGGTACAGTGACGCCGAGGACTTAAAA |
| 118 | NHR2** TD | GGCACACGTCAAGAAGAAATGATTGATCACAGACTAACAGACAGAGAATGGGCAGAAGAG |
| | | TGGAAACATCTTGACCATCTGTTAAACTGCATAATGGACATGGTAGAAAAAACAAGGCGA |
| | | TCTCTCACCGTACTAAGGCGGTGTCAAGAAGCAGACCGGGAAGAATTGAATTACTGGATC |
| | | CGGCGGTACAGTGACGCCGAG |
| 119 | NHR2*** TD | CAAGAAGAAATGATTGATCACAGACTAACAGACAGAGAATGGGCAGAAGAGTGGAAACAT |
| | | CTTGACCATCTGTTAAACTGCATAATGGACATGGTAGAAAAAACAAGGCGATCTCTCACC |
| | | GTACTAAGGCGGTGTCAAGAA |
| | | AMINO ACID SEQUENCE |
| 120 | NHR2 TD | LTDREWAEEWKHLDHLLNCIMDMVEKTRRSLTVLRRCQEADREELNYWIRRYSDAE |
| 121 | NHR2* TD | LHGTRQEEMIDHRLTDREWAEEWKHLDHLLNCIMDMVEKTRRSLTVLRRCQEADREELNY |
| | | WIRRYSDAEDLK |
| 122 | NHR2** TD | GTRQEEMIDHRLTDREWAEEWKHLDHLLNCIMDMVEKTRRSLTVLRRCQEADREELNYWI |
| | | RRYSDAE |
| 123 | NHR2*** TD | QEEMIDHRLTDREWAEEWKHLDHLLNCIMDMVEKTRRSLTVLRRCQE |

NHR2* TD and NHR2** TD include additional amino acid residues at the N- and/or C-terminus.
NHR2*** only includes amino acid residues of the annotated NHR2 domain according to Pubmed (Reference: UniProtKB-Q06455 (MTG8_HUMAN)

TABLE 7
Human p53 TD sequences

| SEQ | | |
|---|---|---|
| ID NO: | p53 TD | DNA SEQUENCE |
| 124 | p53 TD | AAGAAGAAACCACTGGATGGAGAATATTTCACCC |
| | | TTCAGATCCGTGGGCGTGAGCGCTTCGAGATGTT |
| | | CCGAGAGCTGAATGAGGCCTTGGAACTCAAGGAT |
| | | GCCCAGGCTGGGAAGGAGCCAGGG |
| 125 | p53* TD | GGAGAATATTTCACCCTTCAGATCCGTGGGCGTG |
| | | AGCGCTTCGAGATGTTCCGAGAGCTGAATGAGGC |
| | | CTTGGAACTCAAGGATGCCCAGGCTGGG |
| | | AMINO ACID SEQUENCE |
| 126 | p53 TD | KKKPLDGEYFTLQIRGRERFEMFRELNEALELKD |
| | | AQAGKEPG |
| 10 | p53* TD | GEYFTLQIRGRERFEMFRELNEALELKDAQAG |

p53* TD is a truncated version of p53 TD

1-50. (canceled)

51: A composition comprising a protein multimer of at least first, second, third and fourth copies of an effector domain or a peptide,
wherein the multimer is multimerized by first, second, third and fourth self-associating tetramerization domains (TDs) which are associated together,
wherein each tetramerization domain is comprised by a respective engineered polypeptide comprising one or more copies of said effector domain or peptide,
wherein the multimer is soluble in an aqueous medium and is obtainable by extracellular secretion from a eukaryotic cell; and
wherein the multimer comprises eukaryotic cell glycosylation, and the composition comprises the multimer with a purity greater than 90%.

52: The composition of claim 51, wherein:
(i) the multimer comprises a tetramer or an octamer of said domain or peptide;
(ii) the multimer comprises a tetramer or octamer of an immunoglobulin superfamily binding site;
(iii) the effector domain is an immunoglobulin superfamily domain; or
(iv) the effector domain or peptide is an antibody variable or constant domain, a TCR variable or constant domain, an incretin, an insulin peptide, or a hormone peptide.

53: The composition of claim 51, wherein each TD is a p53 TD or a homologue or orthologue thereof.

54: The composition of claim 51, wherein each engineered polypeptide comprises first and second copies of said effector domain or peptide, wherein each engineered polypeptide comprises in N- to C-terminal direction (i) a first of said copies-TD-the second of said copies; (ii) TD-the first and second copies; or (iii) said first and second copies-TD.

55: The composition of claim 51, wherein the engineered polypeptide comprises one or more copies of a second type of effector domain or peptide, wherein the second type of effector domain or peptide is different from the first effector domain or peptide.

56: The composition of claim 51, wherein the multimer:
(i) comprises 4 TDs and 4, 8, 12 or 16 copies of the effector domain or peptide;
(ii) comprises first, second, third and fourth identical copies of an engineered polypeptide, the polypeptide comprising a TD and one, two or more copies of the effector domain or peptide;
(iii) consists of copies of the engineered polypeptide; or
(iv) is bispecific for antigen or pMHC binding.

57: The composition of claim 51, wherein each TD comprises (i) an amino acid sequence identical to SEQ ID NO: 10 or 126 or at least 80% identical thereto.

58: The composition of claim 51, wherein the multimer comprises a tetramer or octamer of an antigen binding site of an antibody selected from the group consisting of REOPRO®; Abciximab; RITUXAN®; Rituximab; ZENAPAX®; Daclizumab; SIMULECT®; Basiliximab; SYNAGIS®; Palivizumab; REMICADE®; Infliximab; HERCEPTIN®; MYLOTARG®; Gemtuzumab; CAMPATH®; Alemtuzumab; ZEVALIN®; Ibritumomab; HUMIRA®; Adalimumab; XOLAIR®; Omalizumab; BEXXAR®; Tositumomab; RAPTIVA™; Efalizumab; ERBITUX®; Cetuximab; AVASTIN®; Bevacizumab; TYSABRI®; Natalizumab; ACTEMRA®; Tocilizumab; VECTIBIX®; Panitumumab; LUCENTIS®; Ranibizumab; SOLIRIS®; Eculizumab; CIMZIA®; Certolizumab; SIMPONI®; Golimumab, ILARIS®; Canakinumab; STELARA®; Ustekinumab; ARZERRA®; Ofatumumab; PROLIA®; Denosumab; NUMAX™; Motavizumab; ABTHRAX™; Raxibacumab; BENLYSTA®; Belimumab; YERVOY®; Ipilimumab; ADCETRIS®; Brentuximab Vedotin; PERJETA®; Pertuzumab; KADCYLA®; Ado-trastuzumab; KEYTRUDA®, OPDIVO®, GAZYVA® and Obinutuzumab.

59: A composition comprising a tetramer or octamer of
(a) TCR V domains or TCR binding sites, wherein the tetramer or octamer is soluble in aqueous solution;
(b) antibody single variable domains, wherein the tetramer or octamer is soluble in aqueous solution;
(c) TCR V domains or TCR binding sites, wherein the tetramer or octamer is capable of being extracellularly expressed by HEK293 cells; or
(d) antibody variable domains, wherein the tetramer or octamer is capable of being extracellularly expressed by HEK293 cells,
wherein the tetramer or octamer comprises eukaryotic cell glycosylation, and the composition comprises the tetramer or octamer with a purity greater than 90%.

60: The composition of claim 51, wherein the composition is a pharmaceutical composition comprising a pharmaceutically acceptable carrier, diluent or excipient.

61: The composition of claim 51, wherein each engineered polypeptide, comprises in N- to C-terminal direction:
(a) TCR V1-TCR C1-antibody CH1-optional linker-TD, wherein
(i) V1 is a Vα and C1 is a Cα;
(ii) V1 is a V and C1 is a Cβ;
(iii) V1 is a Vγ and C1 is a Cγ; or
(iv) V1 is a Vδ and C1 is a Cδ;
or
(b) TCR V1-antibody CH1-optional linker-TD, wherein
(i) V1 is a Vα;
(ii) V1 is a Vβ;
(iii) V1 is a Vγ; or
(iv) V1 is a Vδ;
or
(c) antibody V1-antibody CH1-optional linker-TD, wherein
(i) V1 is a VH; or
(ii) V1 is a VL; or
(d) antibody V1-optional antibody CH1-antibody Fc-optional linker-TD, wherein
(i) V1 is a VH; or
(ii) V1 is a VL; or
(e) antibody V1-antibody CL-optional linker-TD, wherein
(i) V1 is a V_{H}; or
(ii) V1 is a VL; or
(f) TCR V1-TCR C1-optional linker-TD, wherein
(i) V1 is a Vα and C1 is a Cα;
(ii) V1 is a Vβ and C1 is a Cβ;
(iii) V1 is a Vγ and C1 is a Cγ; or
(iv) V1 is a Vδ and C1 is a Cδ.

62: A method of making a composition comprising glycosylated protein multimers in greater than 90% purity, wherein the composition is the composition of claim 51, the method comprising secreting multimers from eukaryotic cells to produce the composition comprising the multimers having eukaryotic glycosylation in greater than 90% purity.

63: The multimer of claim 51, wherein the eukaryotic cell is a mammalian cell.

64: The multimer of claim 51, wherein the eukaryotic cell is a Chinese Hamster Ovary (CHO) cell, HEK293 cell, or Cos cell.

65: A composition comprising a protein multimer of at least first, second, third and fourth copies of an effector domain or a peptide,
wherein the multimer is multimerized by first, second, third and fourth self-associating tetramerization domains (TDs) which are associated together,
wherein each tetramerization domain is comprised by a respective engineered polypeptide comprising one or more copies of said effector domain or peptide,
wherein the multimer is soluble in an aqueous medium and is obtainable by extracellular secretion from a eukaryotic cell, wherein the composition comprises the multimer with a purity greater than 90%;
wherein each engineered polypeptide comprises first and second copies of said effector domain or peptide; and
wherein each engineered polypeptide comprises in N- to C-terminal direction (i) a first of said copies-TD-the second of said copies; (ii) TD-the first and second copies; or (iii) said first and second copies-TD.

66: The composition of claim 65, wherein each TD comprises (i) an amino acid sequence identical to SEQ ID NO: 10 or 126 or at least 95% identical thereto.

67: The composition of claim 65, wherein the engineered polypeptide comprises one or more copies of a second type of effector domain or peptide, wherein the second type of effector domain or peptide is different from the first effector domain or peptide.

68: The composition of claim 59, wherein the composition is a pharmaceutical composition comprising a pharmaceutically acceptable carrier, diluent or excipient.

69: The composition of claim 65, wherein the composition is a pharmaceutical composition comprising a pharmaceutically acceptable carrier, diluent or excipient.

70: The composition of claim 51, wherein each TD is a transthyretin (TTR) TD or a homologue or orthologue thereof.

71: A method of treating or preventing a disease in a human or animal subject, comprising administering to the subject the composition of claim 51.


SEQUENCE LISTING (144 sequences)

SEQ ID NO: 1 - protein, 393 aa, Homo sapiens [sequence omitted]

SEQ ID NO: 2 - protein, 341 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 3 - protein, 346 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 4 - protein, 354 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 5 - protein, 302 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 6 - protein, 307 aa, Homo sapiens
MDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 7 - protein, 261 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 8 - protein, 209 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQDQTSFQKENC

SEQ ID NO: 9 - protein, 214 aa, Homo sapiens
MFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQMLLDLRWCYFLINSS

SEQ ID NO: 10 - protein, 32 aa, Homo sapiens
GEYFTLQIRGRERFEMFRELNEALELKDAQAG

SEQ ID NO: 11 - protein, 50 aa, Homo sapiens
RSPDDELLYLPVRGRETYEMLLKIKESLELMQYLPQHTIETYRQQQQQQH

SEQ ID NO: 12 - protein, 42 aa, Homo sapiens
KKRRHGDEDTYYLQVRGRENFEILMKLKESLELMELVPQPLV

SEQ ID NO: 13 - DNA, 1290 bp, synthetic construct
  misc_feature 1..1290: note = Synthetic Construct
  source 1..1290: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 14 - DNA, 1305 bp, synthetic construct
  misc_feature 1..1305: note = Synthetic Construct
  source 1..1305: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 15 - DNA, 1290 bp, synthetic construct
  misc_feature 1..1290: note = Synthetic Construct
  source 1..1290: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 16 - DNA, 1305 bp, synthetic construct
  misc_feature 1..1305: note = Synthetic Construct
  source 1..1305: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 17 - DNA, 996 bp, synthetic construct
  misc_feature 1..996: note = Synthetic Construct
  source 1..996: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 18 - DNA, 1011 bp, synthetic construct
  misc_feature 1..1011: note = Synthetic Construct
  source 1..1011: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 19 - DNA, 999 bp, synthetic construct
  misc_feature 1..999: note = Synthetic Construct
  source 1..999: mol_type = other DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattacaccttggcagagagagcaaacaagtggaagacttaatgcctcgctggataaatcatcaggacgtagtactttatacattgcagcttctcagcctggtgactcagccacctacctctgtgctgtgaggcccctgcttgacggaacatacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 20 - DNA, 999 bp, synthetic construct
  misc_feature 1..999: note = Synthetic Construct
  source 1..999: mol_type = other DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattacaccttggcagagagagcaaacaagtggaagacttaatgcctcgctggataaatcatcaggacgtagtactttatacattgcagcttctcagcctggtgactcagccacctacctctgtgctgtgaggcccctgcttgacggaacatacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttccacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 21 - DNA, 681 bp, synthetic construct
  misc_feature 1..681: note = Synthetic Construct
  source 1..681: mol_type = other DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattacaccttggcagagagagcaaacaagtggaagacttaatgcctcgctggataaatcatcaggacgtagtactttatacattgcagcttctcagcctggtgactcagccacctacctctgtgctgtgaggcccctgcttgacggaacatacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcc

SEQ ID NO: 22 - DNA, 1689 bp, synthetic construct
  misc_feature 1..1689: note = Synthetic Construct
  source 1..1689: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactgactacaaggacgacgacgacaagcaccaccaccatcaccac

SEQ ID NO: 23 - DNA, 1704 bp, synthetic construct
  misc_feature 1..1704: note = Synthetic Construct
  source 1..1704: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactgactacaaggacgacgacgacaagcaccaccaccatcaccac

SEQ ID NO: 24 - DNA, 1335 bp, synthetic construct
  misc_feature 1..1335: note = Synthetic Construct
  source 1..1335: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 25 - DNA, 1350 bp, synthetic construct
  misc_feature 1..1350: note = Synthetic Construct
  source 1..1350: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 26 - DNA, 615 bp, synthetic construct
  misc_feature 1..615: note = Synthetic Construct
  source 1..615: mol_type = other DNA
atggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 27 - DNA, 630 bp, synthetic construct
  misc_feature 1..630: note = Synthetic Construct
  source 1..630: mol_type = other DNA
atggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaaggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 28 - DNA, 594 bp, synthetic construct
  misc_feature 1..594: note = Synthetic Construct
  source 1..594: mol_type = other DNA
atggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggcacctacttcaagttctaca

SEQ ID NO: 29 - DNA, 1029 bp, synthetic construct
  misc_feature 1..1029: note = Synthetic Construct
  source 1..1029: mol_type = other DNA
atggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaaggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactgactacaaggacgacgacgacaagcaccaccaccatcaccac

SEQ ID NO: 30 - DNA, 1233 bp, synthetic construct
  misc_feature 1..1233: note = Synthetic Construct
  source 1..1233: mol_type = other DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattacaccttggcagagagagcaaacaagtggaagacttaatgcctcgctggataaatcatcaggacgtagtactttatacattgcagcttctcagcctggtgactcagccacctacctctgtgctgtgaggcccctgcttgacggaacatacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 31 - DNA, 1248 bp, synthetic construct
  misc_feature 1..1248: note = Synthetic Construct
  source 1..1248: mol_type = other DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattacaccttggcagagagagcaaacaagtggaagacttaatgcctcgctggataaatcatcaggacgtagtactttatacattgcagcttctcagcctggtgactcagccacctacctctgtgctgtgaggcccctgcttgacggaacatacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttccgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 32 - DNA, 1104 bp, synthetic construct
  misc_feature 1..1104: note = Synthetic Construct
  source 1..1104: mol_type = other DNA
atgagcctcgggctcctgtgctgtggggccttttctctcctgtgggcaggtccagtgaatgccggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttgccatccagacaactgaccaaggagaagtccccaatggctacaatgtctccagatcaaccatcgaggatttcccgctcaggctgctgtcggctgctccctcccagacatctgtgtacttctgtgccagcagttacctggggaacaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccatgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagacacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 33 - DNA, 912 bp, synthetic construct
  misc_feature 1..912: note = Synthetic Construct
  source 1..912: mol_type = other DNA
atgaacttcgggctcagcttgattttccttgcccttattttaaaaggtgtccagtgtgaggtgcaactggtggagtctgggggaggcttagtgaagcctggggggtccctgaaactctcctgtgcagcctctggactcactttcagtagctatgccatgtcttgggttcgccagactccagagaagaggctggagtgggtcgcatccattagtagtggtggtttcacctactatccagacagtgtgaagggccgattcaccatctccagagataatgccaggaacatcctgtatctgcaaatgagcagtctgaggtctgaggacacggccatgtattactgtgcaagagacgaggtacgggggtacctcgatgtctggggcgcagggaccacggtcaccgtttcctcggccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 34 - DNA, 927 bp, synthetic construct
  misc_feature 1..927: note = Synthetic Construct
  source 1..927: mol_type = other DNA
atgaacttcgggctcagcttgattttccttgcccttattttaaaaggtgtccagtgtgaggtgcaactggtggagtctgggggaggcttagtgaagcctggggggtccctgaaactctcctgtgcagcctctggactcactttcagtagctatgccatgtcttgggttcgccagactccagagaagaggctggagtgggtcgcatccattagtagtggtggtttcacctactatccagacagtgtgaagggccgattcaccatctccagagataatgccaggaacatcctgtatctgcaaatgagcagtctgaggtctgaggacacggccatgtattactgtgcaagagacgaggtacgggggtacctcgatgtctggggcgcagggaccacggtcaccgtttcctcggccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 35 - DNA, 717 bp, synthetic construct
  misc_feature 1..717: note = Synthetic Construct
  source 1..717: mol_type = other DNA
atgaaatacctattgcctacggcagccgctggattgttattactcgcggcccagccggccatggcggcctacaaagatatccagatgacacagactacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttattattgtcagcagtatagcaagcttccgtacacgttcggaggggggaccaagctggaaataaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 36 - DNA, 957 bp, synthetic construct
  misc_feature 1..957: note = Synthetic Construct
  source 1..957: mol_type = other DNA
atgaacttcgggctcagcttgattttccttgcccttattttaaaaggtgtccagtgtgaggtgcaactggtggagtctgggggaggcttagtgaagcctggggggtccctgaaactctcctgtgcagcctctggactcactttcagtagctatgccatgtcttgggttcgccagactccagagaagaggctggagtgggtcgcatccattagtagtggtggtttcacctactatccagacagtgtgaagggccgattcaccatctccagagataatgccaggaacatcctgtatctgcaaatgagcagtctgaggtctgaggacacggccatgtattactgtgcaagagacgaggtacgggggtacctcgatgtctggggcgcagggaccacggtcaccgtttcctcggccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 37 - DNA, 972 bp, synthetic construct
  misc_feature 1..972: note = Synthetic Construct
  source 1..972: mol_type = other DNA
atgaacttcgggctcagcttgattttccttgcccttattttaaaaggtgtccagtgtgaggtgcaactggtggagtctgggggaggcttagtgaagcctggggggtccctgaaactctcctgtgcagcctctggactcactttcagtagctatgccatgtcttgggttcgccagactccagagaagaggctggagtgggtcgcatccattagtagtggtggtttcacctactatccagacagtgtgaagggccgattcaccatctccagagataatgccaggaacatcctgtatctgcaaatgagcagtctgaggtctgaggacacggccatgtattactgtgcaagagacgaggtacgggggtacctcgatgtctggggcgcagggaccacggtcaccgtttcctcggccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtggaacccaagtcctgcgacaagacccacacctgtcccccttgtcctggaggaggtgggagcctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggactacaaggacgacgacgacaagcaccaccaccatcaccac

SEQ ID NO: 38 - DNA, 972 bp, synthetic construct
  misc_feature 1..972: note = Synthetic Construct
  source 1..972: mol_type = other DNA
atgggatggtcttgtataattctgttcctggtggcaacagcaacaggagtgcatagcgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 39 - DNA, 987 bp, synthetic construct
  misc_feature 1..987: note = Synthetic Construct
  source 1..987: mol_type = other DNA
atgggatggtcttgtataattctgttcctggtggcaacagcaacaggagtgcatagcgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 40 - DNA, 708 bp, synthetic construct
  misc_feature 1..708: note = Synthetic Construct
  source 1..708: mol_type = other DNA
atggacatgagggtccctgctcagctcctgggactcctgctgctctggctcccagataccagatgtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaacgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 41 - DNA, 978 bp, synthetic construct
  misc_feature 1..978: note = Synthetic Construct
  source 1..978: mol_type = other DNA
atgggatggtcttgtataattctgttcctggtggcaacagcaacaggagtgcatagcgaggtccaacttgtcgaaagtggcggcggtttggttcaacctggaggttcacttcgactgtcatgtgcagcgagcggttatacatttacgaattatggcatgaattgggttagacaggcaccaggaaagggactggagtgggtaggctggatcaatacctacacaggagaaccaacgtatgccgcagacttcaaacgacggtttacattttccttggatacctctaagtctacagcgtatctccaaatgaattcacttcgagcggaagataccgcggtctactattgcgccaaataccctcattattatgggtcatctcactggtatttcgatgtctggggtcagggaacactggtaaccgtgtcatccgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 42 - DNA, 993 bp, synthetic construct
  misc_feature 1..993: note = Synthetic Construct
  source 1..993: mol_type = other DNA
atgggatggtcttgtataattctgttcctggtggcaacagcaacaggagtgcatagcgaggtccaacttgtcgaaagtggcggcggtttggttcaacctggaggttcacttcgactgtcatgtgcagcgagcggttatacatttacgaattatggcatgaattgggttagacaggcaccaggaaagggactggagtgggtaggctggatcaatacctacacaggagaaccaacgtatgccgcagacttcaaacgacggtttacattttccttggatacctctaagtctacagcgtatctccaaatgaattcacttcgagcggaagataccgcggtctactattgcgccaaataccctcattattatgggtcatctcactggtatttcgatgtctggggtcagggaacactggtaaccgtgtcatccgccagcaccaagggcccctctgtgttccctctggccccttccagcaagtccacctctggcggaacagccgctctgggctgcctcgtgaaggactacttccccgagcctgtgaccgtgtcctggaactctggcgctctgaccagcggagtgcacaccttccctgctgtgctgcagtcctccggcctgtactccctgtcctccgtcgtgaccgtgccttccagctctctgggcacccagacctacatctgcaacgtgaaccacaagccctccaacaccaaggtggacaagaaggtgggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 43 - DNA, 708 bp, synthetic construct
  misc_feature 1..708: note = Synthetic Construct
  source 1..708: mol_type = other DNA
atggacatgagggtccctgctcagctcctggggctcctgcagctctggctctcaggtgccagatgtgacatccaaatgacccagagtccttccagcctcagtgcgtcagtgggagatcgagtgacgataacgtgttctgccagccaagacatttccaactatcttaattggtaccagcagaaaccgggaaaggccccgaaagtgctcatatactttaccagcagtcttcactctggagttcctagccggtttagcggctcaggtagtggcaccgatttcactctgaccattagttctcttcaaccggaagattttgcaacctactattgtcagcagtattcaacggtaccttggaccttcggccaaggcaccaaagtcgagattaagcgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgt

SEQ ID NO: 44 - DNA, 660 bp, synthetic construct
  misc_feature 1..660: note = Synthetic Construct
  source 1..660: mol_type = other DNA
atggatatgcgcgtcccggcacagctgctcggcttgttgttgctgtggttgagagctaggtgcgatatacagatgactcagtccccttccagtctttcagccagtgtcggcgaccgggttaccattacttgtcgggcaagtcaatctatagatagttatttgcattggtatcaacaaaaaccaggcaaagcgcctaagttgttgatatattccgcatctgaactgcaatcaggcgttccttcacgcttttctggaagcggcagcggaaccgatttcactcttaccataagtagtctccagccggaggattttgctacatactattgtcaacaagtagtgtggcgaccgttcaccttcggacaggggacaaaagtagaaatcaagcggggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 45 - DNA, 684 bp, synthetic construct
  misc_feature 1..684: note = Synthetic Construct
  source 1..684: mol_type = other DNA
atggagtttggcctcagttggttgtttttggtagcgaaaattaaagtacagtgtgaagtccaactcctggagagcggggggggtctggtacaaccaggcggctcactgcggcttagctgcgcagcctccgggttcacgttcgcacatgaaacgatggtgtgggtgcgccaggcaccggggaagggactcgaatgggtctcacatatacctcctgacggtcaggatcctttttacgcggactctgtgaagggacgattcacaataagtagagacaatagtaagaacaccctttatttgcagatgaacagtctgcgggcggaagatacagcagtatatcattgtgccctgctgcccaaacgaggtccgtggtttgactattggggacaggggactctcgttactgtaagctccggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 46 - DNA, 657 bp, synthetic construct
  misc_feature 1..657: note = Synthetic Construct
  source 1..657: mol_type = other DNA
atggacatgagggtccccgctcagctcctggggctcctgctactctggctccgagccagatgtgatatacagatgacccaatcaccaagcagcttgtccgcttcagtgggcgacagggtaactataacatgccgcgcaagccaatggataggtccagaactctcatggtaccaacaaaaaccagggaaagcgccgaaactgcttatctatcacacaagcattttgcaatctggggtacctagtcgattcagtggctctggcagtgggactgactttacactcaccataagttctctccaaccagaggactttgcaacatactattgtcagcaatatatgtttcaaccacgcacctttggacaaggcacaaaagttgaaatccgcggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 47 - DNA, 660 bp, synthetic construct
  misc_feature 1..660: note = Synthetic Construct
  source 1..660: mol_type = other DNA
atggacatgagggtccccgctcagctcctggggctcctgctactctggctccgagccagatgtgatattcaaatgacacagtcaccaacgagtctttccgcgagcgttggggaccgagtgacaataacttgtcgagcctctcagtggattggcaacttgctggactggtatcagcaaaagccgggagaagccccgaagctgctcatatactatgcttccttcctccagagtggagtacctagcagattcagcggggggggattcgggactgatttcactcttacaatcagctctcttcaacccgaggacttcgcaacgtactactgtcaacaagctaaccctgcgccgcttactttcggacaaggcactaaggtcgaaattaagcgaggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 48 - DNA, 996 bp, synthetic construct
  misc_feature 1..996: note = Synthetic Construct
  source 1..996: mol_type = other DNA
atggacatgagggtccccgctcagctcctggggctcctgctactctggctccgagccagatgtgatatacagatgacccaatcaccaagcagcttgtccgcttcagtgggcgacagggtaactataacatgccgcgcaagccaatggataggtccagaactctcatggtaccaacaaaaaccagggaaagcgccgaaactgcttatctatcacacaagcattttgcaatctggggtacctagtcgattcagtggctctggcagtgggactgactttacactcaccataagttctctccaaccagaggactttgcaacatactattgtcagcaatatatgtttcaaccacgcacctttggacaaggcacaaaagttgaaatccgcggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaaggtggaggaggtagcgatattcaaatgacacagtcaccaacgagtctttccgcgagcgttggggaccgagtgacaataacttgtcgagcctctcagtggattggcaacttgctggactggtatcagcaaaagccgggagaagccccgaagctgctcatatactatgcttccttcctccagagtggagtacctagcagattcagcggggggggattcgggactgatttcactcttacaatcagctctcttcaacccgaggacttcgcaacgtactactgtcaacaagctaaccctgcgccgcttactttcggacaaggcactaaggtcgaaattaagcgagactacaaggacgacgacgacaagcaccaccaccatcaccac

SEQ ID NO: 49 - DNA, 660 bp, synthetic construct
  misc_feature 1..660: note = Synthetic Construct
  source 1..660: mol_type = other DNA
atggacatgagggtccccgctcagctcctggggctcctgctactctggctccgagccagatgtgacattcagatgactcagtcaccatcctcattgtctgcatcagttggtgaccgagttacgatcacatgtcgagcaagccaaaatatagattccagactttcatggtaccagcagaagcctggtaaagcgccgaaactcctcatatatcgcacgagcgtattgcaatctggtgtgccttctcgattttcaggatctgggtctggcactgacttcaccttgacaatatcttctcttcagcccgaagatttcgctacctactactgccaacaatgggacatgtttcctctgaccttcggacagggtacaaaggtcgagattaaacggggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaagactacaaggacgacgacgacaagcaccaccaccatcaccac

SEQ ID NO: 50 - DNA, 999 bp, synthetic construct
  misc_feature 1..999: note = Synthetic Construct
  source 1..999: mol_type = other DNA
atggacatgagggtccccgctcagctcctggggctcctgctactctggctccgagccagatgtgacattcagatgactcagtcaccatcctcattgtctgcatcagttggtgaccgagttacgatcacatgtcgagcaagccaaaatatagattccagactttcatggtaccagcagaagcctggtaaagcgccgaaactcctcatatatcgcacgagcgtattgcaatctggtgtgccttctcgattttcaggatctgggtctggcactgacttcaccttgacaatatcttctcttcagcccgaagatttcgctacctactactgccaacaatgggacatgtttcctctgaccttcggacagggtacaaaggtcgagattaaacggggaggaggtgggagcttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaaggtggaggaggtagcgatatacagatgactcaatccccttcatccctctcagcttccgtaggggacagagttactataacgtgtcgagctagtcaagacataggtgatcgcctgaggtggtatcagcaaaaaccgggtaaagcacctaaactcctcatatatcatggttccaggttggagtcaggcgtgccgtcacgattctctgggtcacgctctggcactgacttcacattgacgattagttctctccagcccgaagacttcgccacctactactgtcaacagcaatggtttcgcccgtatacttttgggcagggtacaaaggttgagattaaacgggactacaaggacgacgacgacaagcaccaccaccatcaccac

SEQ ID NO: 51 - protein, 121 aa, synthetic construct [sequence omitted]

SEQ ID NO: 52 - protein, 108 aa, synthetic construct
  REGION 1..108: note = Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKR

SEQ ID NO: 53 - protein, 121 aa, synthetic construct [sequence omitted]

SEQ ID NO: 54 - protein, 107 aa, synthetic construct
  REGION 1..107: note = Synthetic Construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 55 - protein, 123 aa, synthetic construct [sequence omitted]

SEQ ID NO: 56 - protein, 108 aa, synthetic construct
  REGION 1..108: note = Synthetic Construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 57 - protein, 119 aa, synthetic construct [sequence omitted]

SEQ ID NO: 58 - protein, 108 aa, synthetic construct
  REGION 1..108: note = Synthetic Construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKR

SEQ ID NO: 59 - protein, 119 aa, synthetic construct
  REGION 1..119: note = Synthetic Construct
EVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSS

SEQ ID NO: 60 - protein, 108 aa, synthetic construct
  REGION 1..108: note = Synthetic Construct
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKR

SEQ ID NO: 61 - protein, 113 aa, synthetic construct [sequence omitted]

SEQ ID NO: 62 - protein, 108 aa, synthetic construct
  REGION 1..108: note = Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKR

SEQ ID NO: 63 - protein, 118 aa, synthetic construct
  REGION 1..118: note = Synthetic Construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 64 - protein, 109 aa, synthetic construct
  REGION 1..109: note = Synthetic Construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKR

SEQ ID NO: 65 - protein, 119 aa, synthetic construct
  REGION 1..119: note = Synthetic Construct
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSS

SEQ ID NO: 66 - protein, 108 aa, synthetic construct
  REGION 1..108: note = Synthetic Construct
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKR

SEQ ID NO: 67 - protein, 107 aa, synthetic construct
  REGION 1..107: note = Synthetic Construct
DIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIR

SEQ ID NO: 68 - protein, 108 aa, synthetic construct
  REGION 1..108: note = Synthetic Construct
DIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKR

SEQ ID NO: 69 - protein, 108 aa, synthetic construct
  REGION 1..108: note = Synthetic Construct
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGQGTKVEIKR

SEQ ID NO: 70 - protein, 108 aa, synthetic construct
  REGION 1..108: note = Synthetic Construct
DIQMTQSPSSLSASVGDRVTITCRASQDIGDRLRWYQQKPGKAPKLLIYHGSRLESGVPSRFSGSRSGTDFTLTISSLQPEDFATYYCQQQWFRPYTFGQGTKVEIKR

SEQ ID NO: 71 - protein, 115 aa, synthetic construct
  REGION 1..115: note = Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPY

SEQ ID NO: 72 - protein, 112 aa, synthetic construct
  REGION 1..112: note = Synthetic Construct
NAGVTQTPKFQVLKTGQSMTLQCAQDMNHEYMSWYRQDPGMGLRLIHYSVAIQTDQGEVPNGYNVSRSTIEDFPLRLLSAAPSQTSVYFCASSYLGNTGELFFGEGSRLTVL

SEQ ID NO: 73 - protein, 133 aa, Homo sapiens [sequence omitted]

SEQ ID NO: 74 - protein, 115 aa, synthetic construct [sequence omitted]

SEQ ID NO: 75 - protein, 108 aa, synthetic construct
  REGION 1..108: note = Synthetic Construct
DIQMTQSPSSLSASVGDRVTITCRASQSIIKHLKWYQQKPGKAPKLLIYGASRLQSGVPSRFSGSGSGTDFTLTISSLQPEDFATYYCQQGARWPQTFGQGTKVEIKR

SEQ ID NO: 76 - protein, 104 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDV

SEQ ID NO: 77 - protein, 101 aa, Homo sapiens
VLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 78 - protein, 431 aa, synthetic construct
  REGION 1..431: note = Synthetic Construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 31 aa, synthetic construct
  REGION 1..31: note = Synthetic Construct
HAPGTFTSDVSSYLEGQAAKEFIAWLVKGRG

SEQ ID NO: 80 - protein, 34 aa, synthetic construct
  REGION 1..34: note = Synthetic Construct
IKPEAPGEDASPEELNRYYASLRHYLNLVTRQRY

SEQ ID NO: 81 - protein, 39 aa, synthetic construct
  REGION 1..39: note = Synthetic Construct
HGEGTFTSDLSKQMEEEAVRLFIEWLKNGGPSSGAPPPS

SEQ ID NO: 82 - protein, 275 aa, synthetic construct
  REGION 1..275: note = Synthetic Construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 83 - protein, 397 aa, synthetic construct
  REGION 1..397: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLTDREWAEEWKHLDHLLNCIMDMVEKTRRSLTVLRRCQEADREELNYWIRRYSDAE

SEQ ID NO: 84 - protein, 402 aa, synthetic construct
  REGION 1..402: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLTDREWAEEWKHLDHLLNCIMDMVEKTRRSLTVLRRCQEADREELNYWIRRYSDAE

SEQ ID NO: 85 - protein, 397 aa, synthetic construct
  REGION 1..397: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLTDREWAEEWKHLDHLLNCIMDMVEKTRRSLTVLRRCQEADREELNYWIRRYSDAE

SEQ ID NO: 86 - protein, 402 aa, synthetic construct
  REGION 1..402: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLTDREWAEEWKHLDHLLNCIMDMVEKTRRSLTVLRRCQEADREELNYWIRRYSDAE

SEQ ID NO: 87 - protein, 299 aa, synthetic construct
  REGION 1..299: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADLTDREWAEEWKHLDHLLNCIMDMVEKTRRSLTVLRRCQEADREELNYWIRRYSDAE

SEQ ID NO: 88 - protein, 304 aa, synthetic construct
  REGION 1..304: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRADGGGGSLTDREWAEEWKHLDHLLNCIMDMVEKTRRSLTVLRRCQEADREELNYWIRRYSDAE

SEQ ID NO: 89 - protein, 314 aa, synthetic construct
  REGION 1..314: note = Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 314 aa, synthetic construct
  REGION 1..314: note = Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 208 aa, synthetic construct
  REGION 1..208: note = Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 92 - protein, 530 aa, synthetic construct
  REGION 1..530: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLTDREWAEEWKHLDHLLNCIMDMVEKTRRSLTVLRRCQEADREELNYWIRRYSDAEAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 93 - protein, 535 aa, synthetic construct
  REGION 1..535: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLTDREWAEEWKHLDHLLNCIMDMVEKTRRSLTVLRRCQEADREELNYWIRRYSDAEAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 94 - protein, 412 aa, synthetic construct
  REGION 1..412: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPLTDREWAEEWKHLDHLLNCIMDMVEKTRRSLTVLRRCQEADREELNYWIRRYSDAE

SEQ ID NO: 95 - protein, 417 aa, synthetic construct
  REGION 1..417: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGGGGSLTDREWAEEWKHLDHLLNCIMDMVEKTRRSLTVLRRCQEADREELNYWIRRYSDAE

SEQ ID NO: 96 - protein, 172 aa, synthetic construct
  REGION 1..172: note = Synthetic Construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKLTDREWAEEWKHLDHLLNCIMDMVEKTRRSLTVLRRCQEADREELNYWIRRYSDAE

SEQ ID NO: 97 - protein, 177 aa, synthetic construct
  REGION 1..177: note = Synthetic Construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKGGGGSLTDREWAEEWKHLDHLLNCIMDMVEKTRRSLTVLRRCQEADREELNYWIRRYSDAE

SEQ ID NO: 98 - protein, 305 aa, synthetic construct
  REGION 1..305: note = Synthetic Construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKLTDREWAEEWKHLDHLLNCIMDMVEKTRRSLTVLRRCQEADREELNYWIRRYSDAEAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 99 - protein, 310 aa, synthetic construct
  REGION 1..310: note = Synthetic Construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKGGGGSLTDREWAEEWKHLDHLLNCIMDMVEKTRRSLTVLRRCQEADREELNYWIRRYSDAEAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 100 - protein, 378 aa, synthetic construct
  REGION 1..378: note = Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLHGTRQEEMIDHRLTDREWAEEWKHLDHLLNCIMDMVEKTRRSLTVLRRCQEADREELNYWIRRYSDAEDLK

SEQ ID NO: 101 - protein, 383 aa, synthetic construct
  REGION 1..383: note = Synthetic Construct
KQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLHGTRQEEMIDHRLTDREWAEEWKHLDHLLNCIMDMVEKTRRSLTVLRRCQEADREELNYWIRRYSDAEDLK

SEQ ID NO: 102 - protein, 349 aa, synthetic construct
  REGION 1..349: note = Synthetic Construct
NAGVTQTPKFQVLKTGQSMTLQCAQDMNHEYMSWYRQDPGMGLRLIHYSVAIQTTDQGEVPNGYNVSRSTIEDFPLRLLSAAPSQTSVYFCASSYLGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 291 aa, synthetic construct
  REGION 1..291: note = Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLHGTRQEEMIDHRLTDREWAEEWKHLDHLLNCIMDMVEKTRRSLTVLRRCQEADREELNYWIRRYSDAEDLK

SEQ ID NO: 104 - protein, 296 aa, synthetic construct
  REGION 1..296: note = Synthetic Construct
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLHGTRQEEMIDHRLTDREWAEEWKHLDHLLNCIMDMVEKTRRSLTVLRRCQEADREELNYWIRRYSDAEDLK

SEQ ID NO: 105 - protein, 214 aa, synthetic construct
  REGION 1..214: note = Synthetic Construct
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 293 aa, synthetic construct
  REGION 1..293: note = Synthetic Construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVLHGTRQEEMIDHRLTDREWAEEWKHLDHLLNCIMDMVEKTRRSLTVLRRCQEADREELNYWIRRYSDAEDLK

SEQ ID NO: 107 - protein, 298 aa, synthetic construct
  REGION 1..298: note = Synthetic Construct
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSLHGTRQEEMIDHRLTDREWAEEWKHLDHLLNCIMDMVEKTRRSLTVLRRCQEADREELNYWIRRYSDAEDLK

SEQ ID NO: 108 - protein, 214 aa, synthetic construct
  REGION 1..214: note = Synthetic Construct
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 185 aa, synthetic construct
  REGION 1..185: note = Synthetic Construct
DIQMTQSPSSLSASVGDRVTITCRASQSIDSYLHWYQQKPGKAPKLLIYSASELQSGVPSRFSGSGSGTDFTLTISSLQPEDFATYYCQQVVWRPFTFGQGTKVEIKRGGGGSLHGTRQEEMIDHRLTDREWAEEWKHLDHLLNCIMDMVEKTRRSLTVLRRCQEADREELNYWIRRYSDAEDLK

SEQ ID NO: 110 - protein, 196 aa, synthetic construct
  REGION 1..196: note = Synthetic Construct
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSSGGGGSLHGTRQEEMIDHRLTDREWAEEWKHLDHLLNCIMDMVEKTRRSLTVLRRCQEADREELNYWIRRYSDAEDLK

SEQ ID NO: 111 - protein, 184 aa, synthetic construct
  REGION 1..184: note = Synthetic Construct
DIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIRGGGGSLHGTRQEEMIDHRLTDREWAEEWKHLDHLLNCIMDMVEKTRRSLTVLRRCQEADREELNYWIRRYSDAEDLK

SEQ ID NO: 112 - protein, 185 aa, synthetic construct
  REGION 1..185: note = Synthetic Construct
DIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKRGGGGSLHGTRQEEMIDHRLTDREWAEEWKHLDHLLNCIMDMVEKTRRSLTVLRRCQEADREELNYWIRRYSDAEDLK

SEQ ID NO: 113 - protein, 297 aa, synthetic construct
  REGION 1..297: note = Synthetic Construct
DIQMTQSPSSLSASVGDRVTITCRASQWIGPELSWYQQKPGKAPKLLIYHTSILQSGVPSRFSGSGSGTDFTLTISSLQPEDFATYYCQQYMFQPRTFGQGTKVEIRGGGGSLHGTRQEEMIDHRLTDREWAEEWKHLDHLLNCIMDMVEKTRRSLTVLRRCQEADREELNYWIRRYSDAEDLKGGGGSDIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKR

SEQ ID NO: 114 - protein, 185 aa, synthetic construct
  REGION 1..185: note = Synthetic Construct
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGQGTKVEIKRGGGGSLHGTRQEEMIDHRLTDREWAEEWKHLDHLLNCIMDMVEKTRRSLTVLRRCQEADREELNYWIRRYSDAEDLK

SEQ ID NO: 115 - protein, 298 aa, synthetic construct
  REGION 1..298: note = Synthetic Construct
DIQMTQSPSSLSASVGDRVTITCRASQNIDSRLSWYQQKPGKAPKLLIYRTSVLQSGVPSRFSGSGSGTDFTLTISSLQPEDFATYYCQQWDMFPLTFGQGTKVEIKRGGGGSLHGTRQEEMIDHRLTDREWAEEWKHLDHLLNCIMDMVEKTRRSLTVLRRCQEADREELNYWIRRYSDAEDLKGGGGSDIQMTQSPSSLSASVGDRVTITCRASQDIGDRLRWYQQKPGKAPKLLIYHGSRLESGVPSRFSGSRSGTDFTLTISSLQPEDFATYYCQQQWFRPYTFGQGTKVEIKR

SEQ ID NO: 116 - DNA, 168 bp, Homo sapiens
  source 1..168: mol_type = unassigned DNA
ctaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 117 - DNA, 216 bp, synthetic construct
  misc_feature 1..216: note = Synthetic Construct
  source 1..216: mol_type = other DNA
ttgcatggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgaggacttaaaa

SEQ ID NO: 118 - DNA, 201 bp, synthetic construct
  misc_feature 1..201: note = Synthetic Construct
  source 1..201: mol_type = other DNA
ggcacacgtcaagaagaaatgattgatcacagactaacagacagagaatgggcagaagagtggaaacatcttgaccatctgttaaactgcataatggacatggtagaaaaaacaaggcgatctctcaccgtactaaggcggtgtcaagaagcagaccgggaagaattgaattactggatccggcggtacagtgacgccgag

SEQ ID NO: 119 - DNA, 141 bp, synthetic construct
  misc_feature 1..141: note = Synthetic Construct
  source 1..141: mol_type = other DNA
caagaagaaatgattgatcacagactaacagacagagaatgggcagaagagtggaaacatcttgaccatctgttaaactgcataatggacatggtagaaaaaacaaggcgatctctcaccgtactaaggcggtgtcaagaa

SEQ ID NO: 120 - protein, 56 aa, Homo sapiens
LTDREWAEEWKHLDHLLNCIMDMVEKTRRSLTVLRRCQEADREELNYWIRRYSDAE

SEQ ID NO: 121 - protein, 72 aa, synthetic construct
  REGION 1..72: note = Synthetic Construct
LHGTRQEEMIDHRLTDREWAEEWKHLDHLLNCIMDMVEKTRRSLTVLRRCQEADREELNYWIRRYSDAEDLK

SEQ ID NO: 122 - protein, 67 aa, synthetic construct
  REGION 1..67: note = Synthetic Construct
GTRQEEMIDHRLTDREWAEEWKHLDHLLNCIMDMVEKTRRSLTVLRRCQEADREELNYWIRRYSDAE

SEQ ID NO: 123 - protein, 47 aa, synthetic construct
  REGION 1..47: note = Synthetic Construct
QEEMIDHRLTDREWAEEWKHLDHLLNCIMDMVEKTRRSLTVLRRCQE

SEQ ID NO: 124 - DNA, 126 bp, Homo sapiens
  source 1..126: mol_type = unassigned DNA
aagaagaaaccactggatggagaatatttcacccttcagatccgtgggcgtgagcgcttcgagatgttccgagagctgaatgaggccttggaactcaaggatgcccaggctgggaaggagccaggg

SEQ ID NO: 125 - DNA, 96 bp, synthetic construct
  misc_feature 1..96: note = Synthetic Construct
  source 1..96: mol_type = other DNA
ggagaatatttcacccttcagatccgtgggcgtgagcgcttcgagatgttccgagagctgaatgaggccttggaactcaaggatgcccaggctggg

SEQ ID NO: 126 - protein, 42 aa, Homo sapiens
KKKPLDGEYFTLQIRGRERFEMFRELNEALELKDAQAGKEPG

SEQ ID NO: 127 - protein, 593 aa, synthetic construct
  REGION 1..593: note = Synthetic Construct
CACGTGATGGAGCTGGGGCTGAGCTGGGTGGTCCTGGCTGCTCTACTACAAGGTGTCCAGGCTCAGGTTCAGCTGGTTGAAAGCGGTGGTGCACTGGTTCAGCCTGGTGGTAGCCTGCGTCTGAGCTGTGCAGCAAGCGGTTTTCCGGTTAATCGTTATAGCATGCGTTGGTATCGTCAGGCACCGGGTAAAGAACGTGAATGGGTTGCAGGTATGAGCAGTGCCGGTGATCGTAGCAGCTATGAAGATAGCGTTAAAGGTCGTTTTACCATCAGCCGTGATGATGCACGTAATACCGTTTATCTGCAAATGAATAGCCTGAAACCGGAAGATACCGCAGTGTATTATTGCAATGTTAACGTGGGCTTTGAATATTGGGGTCAGGGCACCCAGGTTACCGTTAGCAGCAAAAAGAAGAAACCACTGGATGGAGAATATTTCACCCTTCAGATCCGTGGGCGTGAGCGCTTCGAGATGTTCCGAGAGCTGAATGAGGCCTTGGAACTCAAGGATGCCCAGGCTGGGAAGGAGCCAGGGGACTACAAGGACGACGACGACAAGCACCACCACCATCACCACTGATAAGCGGCCGC

SEQ ID NO: 128 - protein, 193 aa, synthetic construct
  REGION 1..193: note = Synthetic Construct
HVMELGLSWVVLAALLQGVQAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKKKKPLDGEYFTLQIRGRERFEMFRELNEALELKDAQAGKEPGDYKDDDDKHHHHHH

SEQ ID NO: 129 - protein, 608 aa, synthetic construct
  REGION 1..608: note = Synthetic Construct
CACGTGATGGAGCTGGGGCTGAGCTGGGTGGTCCTGGCTGCTCTACTACAAGGTGTCCAGGCTCAGGTTCAGCTGGTTGAAAGCGGTGGTGCACTGGTTCAGCCTGGTGGTAGCCTGCGTCTGAGCTGTGCAGCAAGCGGTTTTCCGGTTAATCGTTATAGCATGCGTTGGTATCGTCAGGCACCGGGTAAAGAACGTGAATGGGTTGCAGGTATGAGCAGTGCCGGTGATCGTAGCAGCTATGAAGATAGCGTTAAAGGTCGTTTTACCATCAGCCGTGATGATGCACGTAATACCGTTTATCTGCAAATGAATAGCCTGAAACCGGAAGATACCGCAGTGTATTATTGCAATGTTAACGTGGGCTTTGAATATTGGGGTCAGGGCACCCAGGTTACCGTTAGCAGCAAAGGAGGAGGTGGGAGCAAGAAGAAACCACTGGATGGAGAATATTTCACCCTTCAGATCCGTGGGCGTGAGCGCTTCGAGATGTTCCGAGAGCTGAATGAGGCCTTGGAACTCAAGGATGCCCAGGCTGGGAAGGAGCCAGGGGACTACAAGGACGACGACGACAAGCACCACCACCATCACCACTGATAAGCGGCCGC

SEQ ID NO: 130 - protein, 198 aa, synthetic construct
  REGION 1..198: note = Synthetic Construct
HVMELGLSWVVLAALLQGVQAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSKGGGGSKKKPLDGEYFTLQIRGRERFEMFRELNEALELKDAQAGKEPGDYKDDDDKHHHHHH

SEQ ID NO: 131 - protein, 334 aa, synthetic construct
  REGION 1..334: note = Synthetic Construct
MGWSCIILFLVATATGVHSKQEVTQIPAALSVPEGENLVLNCSFTDSAIYNLQWFRQDPGKGLTSLLLITPWQREQTSGRLNASLDKSSGRSTLYIAASQPGDSATYLCAVRPLLDGTYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 441 aa, synthetic construct
  REGION 1..441: note = Synthetic Construct
MGWSCIILFLVATATGVHSNAGVTQTPKFQVLKTGQSMTLQCAQDMNHEYMSWYRQDPGMGLRLIHYSVAIQTDQGEVPNGYNVSRSTIEDFPLRLLSAAPSQTSVYFCASSYLGNTGELFFGEGSRLTVLEDLNKVFPPEVAVFEPSEAEISHTQKATLVCLATGFFPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSGGGGSGGGGSLTDREWAEEWKHLDHLLNCIMDMVEKTRRSLTVLRRCQEADREELNYWIRRYSDAEHHHHHH

SEQ ID NO: 133 - protein, 334 aa, synthetic construct; identical to SEQ ID NO: 131
  REGION 1..334: note = Synthetic Construct

SEQ ID NO: 134 - protein, 456 aa, synthetic construct
  REGION 1..456: note = Synthetic Construct
MGWSCIILFLVATATGVHSNAGVTQTPKFQVLKTGQSMTLQCAQDMNHEYMSWYRQDPGMGLRLIHYSVAIQTDQGEVPNGYNVSRSTIEDFPLRLLSAAPSQTSVYFCASSYLGNTGELFFGEGSRLTVLEDLNKVFPPEVAVFEPSEAEISHTQKATLVCLATGFFPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGGGGSGGGGSGGGGSGGGGSLTDREWAEEWKHLDHLLNCIMDMVEKTRRSLTVLRRCQEADREELNYWIRRYSDAEHHHHHH

SEQ ID NO: 135 - protein, 334 aa, synthetic construct; identical to SEQ ID NO: 131
  REGION 1..334: note = Synthetic Construct

SEQ ID NO: 136 - protein, 594 aa, synthetic construct
  REGION 1..594: note = Synthetic Construct
MGWSCIILFLVATATGVHSNAGVTQTPKFQVLKTGQSMTLQCAQDMNHEYMSWYRQDPGMGLRLIHYSVAIQTDQGEVPNGYNVSRSTIEDFPLRLLSAAPSQTSVYFCASSYLGNTGELFFGEGSRLTVLEDLNKVFPPEVAVFEPSEAEISHTQKATLVCLATGFFPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSGGGGSGGGGSLTDREWAEEWKHLDHLLNCIMDMVEKTRRSLTVLRRCQEADREELNYWIRRYSDAEGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 137 - protein, 334 aa, synthetic construct; identical to SEQ ID NO: 131
  REGION 1..334: note = Synthetic Construct

SEQ ID NO: 138 - protein, 609 aa, synthetic construct
  REGION 1..609: note = Synthetic Construct
MGWSCIILFLVATATGVHSNAGVTQTPKFQVLKTGQSMTLQCAQDMNHEYMSWYRQDPGMGLRLIHYSVAIQTDQGEVPNGYNVSRSTIEDFPLRLLSAAPSQTSVYFCASSYLGNTGELFFGEGSRLTVLEDLNKVFPPEVAVFEPSEAEISHTQKATLVCLATGFFPDHVELSWWVNGKEVHSGVSTDPQPLKEQPALNDSRYALSSRLRVSATFWQDPRNHFRCQVQFYGLSENDEWTQDRAKPVTQIVSAEAWGRADASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGGGGSGGGGSGGGGSGGGGSLTDREWAEEWKHLDHLLNCIMDMVEKTRRSLTVLRRCQEADREELNYWIRRYSDAEGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTHHHHHH

SEQ ID NO: 139 - DNA, 593 bp, synthetic construct
  misc_feature 1..593: note = Synthetic Construct
  source 1..593: mol_type = other DNA
gcggccgcttatcagtggtgatggtggtggtgcttgtcgtcgtcgtccttgtagtcccctggctccttcccagcctgggcatccttgagttccaaggcctcattcagctctcggaacatctcgaagcgctcacgcccacggatctgaagggtgaaatattctccatccagtggtttcttctttttgctgctaacggtaacctgggtgccctgaccccaatattcaaagcccacgttaacattgcaataatacactgcggtatcttccggtttcaggctattcatttgcagataaacggtattacgtgcatcatcacggctgatggtaaaacgacctttaacgctatcttcatagctgctacgatcaccggcactgctcatacctgcaacccattcacgttctttacccggtgcctgacgataccaacgcatgctataacgattaaccggaaaaccgcttgctgcacagctcagacgcaggctaccaccaggctgaaccagtgcaccaccgctttcaaccagctgaacctgagcctggacaccttgtagtagagcagccaggaccacccagctcagccccagctccatcacgtg

SEQ ID NO: 140 - DNA, 608 bp, synthetic construct
  misc_feature 1..608: note = Synthetic Construct
  source 1..608: mol_type = other DNA
gcggccgcttatcagtggtgatggtggtggtgcttgtcgtcgtcgtccttgtagtcccctggctccttcccagcctgggcatccttgagttccaaggcctcattcagctctcggaacatctcgaagcgctcacgcccacggatctgaagggtgaaatattctccatccagtggtttcttcttgctcccacctcctcctttgctgctaacggtaacctgggtgccctgaccccaatattcaaagcccacgttaacattgcaataatacactgcggtatcttccggtttcaggctattcatttgcagataaacggtattacgtgcatcatcacggctgatggtaaaacgacctttaacgctatcttcatagctgctacgatcaccggcactgctcatacctgcaacccattcacgttctttacccggtgcctgacgataccaacgcatgctataacgattaaccggaaaaccgcttgctgcacagctcagacgcaggctaccaccaggctgaaccagtgcaccaccgctttcaaccagctgaacctgagcctggacaccttgtagtagagcagccaggaccacccagctcagccccagctccatcacgtg

SEQ ID NO: 141 - protein, 9 aa, synthetic construct
  REGION 1..9: note = Synthetic Construct
SLLMWITQC

SEQ ID NO: 142 - protein, 19 aa, synthetic construct
  REGION 1..19: note = Synthetic Construct
MGWSCIILFLVATATGVHS

SEQ ID NO: 143 - DNA, 593 bp, synthetic construct
  misc_feature 1..593: note = Synthetic Construct
  source 1..593: mol_type = other DNA
cacgtgatggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaaaagaagaaaccactggatggagaatatttcacccttcagatccgtgggcgtgagcgcttcgagatgttccgagagctgaatgaggccttggaactcaaggatgcccaggctgggaaggagccaggggactacaaggacgacgacgacaagcaccaccaccatcaccactgataagcggccgc

SEQ ID NO: 144 - DNA, 608 bp, synthetic construct
  misc_feature 1..608: note = Synthetic Construct
  source 1..608: mol_type = other DNA
cacgtgatggagctggggctgagctgggtggtcctggctgctctactacaaggtgtccaggctcaggttcagctggttgaaagcggtggtgcactggttcagcctggtggtagcctgcgtctgagctgtgcagcaagcggttttccggttaatcgttatagcatgcgttggtatcgtcaggcaccgggtaaagaacgtgaatgggttgcaggtatgagcagtgccggtgatcgtagcagctatgaagatagcgttaaaggtcgttttaccatcagccgtgatgatgcacgtaataccgtttatctgcaaatgaatagcctgaaaccggaagataccgcagtgtattattgcaatgttaacgtgggctttgaatattggggtcagggcacccaggttaccgttagcagcaaaggaggaggtgggagcaagaagaaaccactggatggagaatatttcacccttcagatccgtgggcgtgagcgcttcgagatgttccgagagctgaatgaggccttggaactcaaggatgcccaggctgggaaggagccaggggactacaaggacgacgacgacaagcaccaccaccatcaccactgataagcggccgc